SEQUENCE LISTING (161 sequences)

SEQ ID NO: 1 - DNA, 1326 bp, Homo sapiens
atgtcccccatctcaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaaggtgagctcctctgaggtggcggtgactgggatggcctcaagtgccatcgcagctcaaagtgggcaggcctgggtctgggctcataggcacattggggaggaacgggatgtgggttgttggtggtggctgctggcctcagaggttgacgcccacctgctccctgtccccggccttccacagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctga

SEQ ID NO: 2 - DNA, 2365 bp, Homo sapiens
tcccagccttcccatccccccaccgaaagcaaatcattcaacgacccccgaccctccgacggcaggagccccccgacctcccaggcggaccgcccttccctccccgcgcgggttccgggcccggcgagagggcgcgacgacagccgaggccatggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccctactacggaaactcggtcagggccacggtgcagaggtaccctccgacccaccacgggagccaggtgtgccgcccgcctctgcttcatggatccctaccctggctggacggcggcaaagccctgggcagccaccacaccgcctccccctggaatctcagccccttctccaagacgtccatccaccacggctccccggggcccctctccgtctaccccccggcctcgtcctcctccttgtcggggggccacgccagcccgcacctcttcaccttcccgcccaccccgccgaaggacgtctccccggacccatcgctgtccaccccaggctcggccggctcggcccggcaggacgagaaagagtgcctcaagtaccaggtgcccctgcccgacagcatgaagctggagtcgtcccactcccgtggcagcatgaccgccctgggtggagcctcctcgtcgacccaccaccccatcaccacctacccgccctacgtgcccgagtacagctccggactcttcccccccagcagcctgctgggcggctcccccaccggcttcggatgcaagtccaggcccaaggcccggtccagcacaggcagggagtgtgtgaactgtggggcaacctcgaccccactgtggcggcgagatggcacgggacactacctgtgcaacgcctgcgggctctatcacaaaatgaacggacagaaccggcccctcattaagcccaagcgaaggctgtctgcagccaggagagcagggacgtcctgtgcgaactgtcagaccaccacaaccacactctggaggaggaatgccaatggggaccctgtctgcaatgcctgtgggctctactacaagcttcacaatattaacagacccctgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagcctgtcctttggaccacaccacccctccagcatggtcaccgccatgggttagagccctgctcgatgctcacagggcccccagcgagagtccctgcagtccctttcgacttgcatttttgcaggagcagtatcatgaagcctaaacgcgatggatatatgtttttgaaggcagaaagcaaaattatgtttgccactttgcaaaggagctcactgtggtgtctgtgttccaaccactgaatctggaccccatctgtgaataagccattctgactcatatcccctatttaacagggtctctagtgctgtgaaaaaaaaaaatcctgaacattgcatataacttatattgtaagaaatactgtacaatgactttattgcatctgggtagctgtaaggcatgaaggatgccaagaagtttaaggaatatgggagaaatagtgtggaaattaagaagaaactaggtctgatattcaaatggacaaactgccagttttgtttcctttcactggccacagttgtttgatgcattaaaagaaaataaaaaaaagaaaaaagagaaaagaaaaaaaaagaaaaaagttgtaggcgaatcatttgttcaaagctgttggccctctgcaaaggaaataccagttctgggcaatcagtgttaccgttcaccagttgccattgagggtttcagagagcctttttctaggcctacatgctttgtgaacaagtccctgtaattgttgtttgtatgtataattcaaagcaccaaaataagaaaagatgtagatttatttcatcatattatacagaccgaactgttgtataaatttatttactgctagtcttaagaactgctttctttcgtttgtttgtttcaatattttccttctctctcaattttcggttgaataaactagattacattcagttggcaaaaaaaaaaaaa

SEQ ID NO: 3 - DNA, 2589 bp, Homo sapiens
cggcccgctggagaggaagcccgagagctgccgcgcgcctgccggacgagggcgtagaagccaggcgtcagagcccgggctccggtggggtcccccacccggccctcgggtcccccgccccctgctccctgcccatcccagcccacgcgaccctctcgcgcgcggaggggcgggtcctcgacggctacgggaaggtgccagcccgccccggatgggcatcgtggagccgggttgcggagacatgctgacgggcaccgagccgatgccggggagcgacgagggccgggcgcctggcgccgacccgcagcaccgctacttctacccggagccgggcgcgcaggacgcggacgagcgtcgcgggggcggcagcctggggtctccctacccggggggcgccttggtgcccgccccgccgagccgcttccttggagcctacgcctacccgccgcgaccccaggcggccggcttccccggcgcgggcgagtccttcccgccgcccgcggacgccgagggctaccagccgggcgagggctacgccgccccggacccgcgcgccgggctctacccggggccgcgtgaggactacgcgctacccgcgggactggaggtgtcggggaaactgagggtcgcgctcaacaaccacctgttgtggtccaagtttaatcagcaccagacagagatgatcatcaccaagcagggacggcggatgttcccattcctgtcatttactgtggccgggctggagcccaccagccactacaggatgtttgtggacgtggtcttggtggaccagcaccactggcggtaccagagcggcaagtgggtgcagtgtggaaaggccgagggcagcatgccaggaaaccgcctgtacgtccacccggactcccccaacacaggagcgcactggatgcgccaggaagtttcatttgggaaactaaagctcacaaacaacaagggggcgtccaacaatgtgacccagatgattgtgctccagtccctccataagtaccagccccggctgcatatcgttgaggtgaacgacggagagccagaggcagcctgcaacgcttccaacacgcatatctttactttccaagaaacccagttcattgccgtgactgcctaccagaatgccgagattactcagctgaaaattgataataacccctttgccaaaggattccgggagaactttgagtccatgtacacatctgttgacaccagcatcccctccccgcctggacccaactgtcaattccttgggggagatcactactctcctctcctacccaaccagtatcctgttcccagccgcttctaccccgaccttcctggccaggcgaaggatgtggttccccaggcttactggctgggggccccccgggaccacagctatgaggctgagtttcgagcagtcagcatgaagcctgcattcttgccctctgcccctgggcccaccatgtcctactaccgaggccaggaggtcctggcacctggagctggctggcctgtggcaccccagtaccctcccaagatgggcccggccagctggttccgccctatgcggactctgcccatggaacccggccctggaggctcagagggacggggaccagaggaccagggtccccccttggtgtggactgagattgcccccatccggccggaatccagtgattcaggactgggcgaaggagactctaagaggaggcgcgtgtccccctatccttccagtggtgacagctcctcccctgctggggccccttctccttttgataaggaagctgaaggacagttttataactattttcccaactgagcagatgacatgatgaaaggaacagaaacagtgttattaggttggaggacaccgactaatttgggaaacggatgaaggactgagaaggcccccgctccctctggcccttctctgtttagtagttggttggggaagtggggctcaagaaggattttggggttcaccagatgcttcctggcccacgatgaaacctgagaggggtgtccccttgccccatcctctgccctaactacagtcgtttacctggtgctgcgtcttgcttttggtttccagctggagaaaagaagacaagaaagtcttgggcatgaaggagctttttgcatctagtgggtgggaggggtcaggtgtgggacatgggagcaggagactccactttcttcctttgtacagtaactttcaaccttttcgttggcatgtgtgttaatccctgatccaaaaagaacaaatacacgtatgttataaccatcagcccgccagggtcagggaaaggactcacctgactttggacagctggcctgggctccccctgctcaaacacagtggggatcagagaaaaggggctggaaaggggggaatggcccacatctcaagaagcaagatattgtttgtggtggttgtgtgtgggtgtgtgttttttctttttctttctttttattttttttgaatgggggaggctatttattgtactgagagtggtgtctggatatattccttttgtcttcatcactttctgaaaataaacataaaactgttaaaaaaaaaaaaaaaaa

SEQ ID NO: 4 - DNA, 7033 bp, Homo sapiens
cacagagcgacagagacatttattgttatttgttttttggtggcaaaaagggaaaatggcgaacgactcccctgcaaaaagtctggtggacatcgacctctcctccctgcgggatcctgctgggatttttgagctggtggaagtggttggaaatggcacctatggacaagtctataagggtcgacatgttaaaacgggtcagttggcagccatcaaagttatggatgtcactgaggatgaagaggaagaaatcaaactggagataaatatgctaaagaaatactctcatcacagaaacattgcaacatattatggtgctttcatcaaaaagagccctccaggacatgatgaccaactctggcttgttatggagttctgtggggctgggtccattacagaccttgtgaagaacaccaaagggaacacactcaaagaagactggatcgcttacatctccagagaaatcctgaggggactggcacatcttcacattcatcatgtgattcaccgggatatcaagggccagaatgtgttgctgactgagaatgcagaggtgaaacttgttgactttggtgtgagtgctcagctggacaggactgtggggcggagaaatacgttcataggcactccctactggatggctcctgaggtcatcgcctgtgatgagaacccagatgccacctatgattacagaagtgatctttggtcttgtggcattacagccattgagatggcagaaggtgctccccctctctgtgacatgcatccaatgagagcactgtttctcattcccagaaaccctcctccccggctgaagtcaaaaaaatggtcgaagaagttttttagttttatagaagggtgcctggtgaagaattacatgcagcggccctctacagagcagcttttgaaacatccttttataagggatcagccaaatgaaaggcaagttagaatccagcttaaggatcatatagatcgtaccaggaagaagagaggcgagaaagatgaaactgagtatgagtacagtgggagtgaggaagaagaggaggaagtgcctgaacaggaaggagagccaagttccattgtgaacgtgcctggtgagtctactcttcgccgagatttcctgagactgcagcaggagaacaaggaacgttccgaggctcttcggagacaacagttactacaggagcaacagctccgggagcaggaagaatataaaaggcaactgctggcagagagacagaagcggattgagcagcagaaagaacagaggcgacggctagaagagcaacaaaggagagagcgggaagctagaaggcagcaggaacgtgaacagcgaaggagagaacaagaagaaaagaggcgtctagaggagttggagagaaggcgcaaagaagaagaggagaggagacgggcagaagaagaaaagaggagagttgaaagagaacaggagtatatcaggcgacagctagaagaggagcagcggcacttggaagtccttcagcagcagctgctccaggagcaggccatgttactgcatgaccataggaggccgcacccgcagcactcgcagcagccgccaccaccgcagcaggaaaggagcaagccaagcttccatgctcccgagcccaaagcccactacgagcctgctgaccgagcgcgagaggttcctgtgagaacaacatctcgctcccctgttctgtcccgtcgagattccccactgcagggcagtgggcagcagaatagccaggcaggacagagaaactccaccagcagtattgagcccaggcttctgtgggagagagtggagaagctggtgcccagacctggcagtggcagctcctcagggtccagcaactcaggatcccagcccgggtctcaccctgggtctcagagtggctccggggaacgcttcagagtgagatcatcatccaagtctgaaggctctccatctcagcgcctggaaaatgcagtgaaaaaacctgaagataaaaaggaagttttcagacccctcaagcctgctggcgaagtggatctgaccgcactggccaaagagcttcgagcagtggaagatgtacggccacctcacaaagtaacggactactcctcatccagtgaggagtcggggacgacggatgaggaggacgacgatgtggagcaggaaggggctgacgagtccacctcaggaccagaggacaccagagcagcgtcatctctgaatttgagcaatggtgaaacggaatctgtgaaaaccatgattgtccatgatgatgtagaaagtgagccggccatgaccccatccaaggagggcactctaatcgtccgccagactcagtccgctagtagcacactccagaaacacaaatcttcctcctcctttacaccttttatagaccccagattactacagatttctccatctagcggaacaacagtgacatctgtggtgggattttcctgtgatgggatgagaccagaagccataaggcaagatcctacccggaaaggctcagtggtcaatgtgaatcctaccaacactaggccacagagtgacaccccggagattcgtaaatacaagaagaggtttaactctgagattctgtgtgctgccttatggggagtgaatttgctagtgggtacagagagtggcctgatgctgctggacagaagtggccaagggaaggtctatcctcttatcaaccgaagacgatttcaacaaatggacgtacttgagggcttgaatgtcttggtgacaatatctggcaaaaaggataagttacgtgtctactatttgtcctggttaagaaataaaatacttcacaatgatccagaagttgagaagaagcagggatggacaaccgtaggggatttggaaggatgtgtacattataaagttgtaaaatatgaaagaatcaaatttctggtgattgctttgaagagttctgtggaagtctatgcgtgggcaccaaagccatatcacaaatttatggcctttaagtcatttggagaattggtacataagccattactggtggatctcactgttgaggaaggccagaggttgaaagtgatctatggatcctgtgctggattccatgctgttgatgtggattcaggatcagtctatgacatttatctaccaacacatgtaagaaagaacccacactctatgatccagtgtagcatcaaaccccatgcaatcatcatcctccccaatacagatggaatggagcttctggtgtgctatgaagatgagggggtttatgtaaacacatatggaaggatcaccaaggatgtagttctacagtggggagagatgcctacatcagtagcatatattcgatccaatcagacaatgggctggggagagaaggccatagagatccgatctgtggaaactggtcacttggatggtgtgttcatgcacaaaagggctcaaagactaaaattcttgtgtgaacgcaatgacaaggtgttctttgcctctgttcggtctggtggcagcagtcaggtttatttcatgaccttaggcaggacttctcttctgagctggtagaagcagtgtgatccagggattactggcctccagagtcttcaagatcctgagaacttggaattccttgtaactggagctcggagctgcaccgagggcaaccaggacagctgtgtgtgcagacctcatgtgttgggttctctcccctccttcctgttcctcttatataccagtttatccccattctttttttttttcttactccaaaataaatcaaggctgcaatgcagctggtgctgttcagattctaccatcaggtgctataagtgtttgggattgagcatcatactggaaagcaaacacctttcctccagctccagaattccttgtctctgaatgactctgtcttgtgggtgtctgacagtggcgacgatgaacatgccgttggttttattggcagtgggcacaaggaggtgagaagtggtggtaaaaggagcggagtgctgaagcagagagcagatttaatatagtaacattaacagtgtatttaattgacatttcttttttgtaatgtgacgatatgtggacaaagaagaagatgcaggtttaagaagttaatatttataaaatgtgaaagacacagttactaggataacttttttgtgggtggggcttgggagatggggtggggtgggttaaggggtcccattttgtttctttggatttggggtgggggtcctggccaagaactcagtcatttttctgtgtaccaggttgcctaaatcatgtgcagatggttctaaaaaaaaaaaaaaaaaaaaaaaaaaaaggaaaaaaaaaaagaaaaagaaaacgtgtgcattttgtataatggccagaactttgtcgtgtgacagtattagcactgcctcagttaaaggtttaatttttgtttaaacctagacgtgcaacaaaagttttaccacagtctgcacttgcagaagaaagaaaaaaattcaaaccacatgtttattttttttttgcctacctcattgttcttaatgcattgagaggtgatttagtttatatgtttttggaagaaaccattaatgtttaatttaatcttaataccaaaacgaccagattgaagtttgacttttattgtcacaaatcagcaggcacaagaactgtccatgaagatgggaaatagccttaaggctgatgcagtttacttacaagtttagaaaccagaatgctttgtttttaccagattcaccattagaggttgatggggcaactgcagcccatgacacaagatctcattgttctcgatgtagaggggttggtagcagacaggtggttacattagaatagtcacacaaactgttcagtgttgcaggaaccttttcttgggggtgggggagtttcccttttctaaaaatgcaatgcactaaaactattttaagaatgtagttaattctgcttattcataaagtgggcatcttctgtgttttaggtgtaatatcgaagtcctggcttttctcgttttctcacttgctctcttgttctctgtttttttaaaccaattttactttatgaatatattcatgacatttgtaataaatgtcttgagaaagaatttgtttcatggcttcatggtcatcactcaagctcccgtaaggatattaccgtctcaggaaaggatcaggactccatgtcacagtcctgccatcttactttcctcttgtcgagttctgagtggaaataactgcattatggctgctttaacctcagtcatcaaaagaaacttgctgttttttaggcttgatctttttcctttgtggttaattttcctgtatattgtgaaaatgggggattttccctctgctcccacccacctaaacacagcagccatttgtacctgtttgcttcccatcccacttggcacccactctgacctcttgtcagtttcctgttcctggttccatctttttgaaaaaggccctcctttgagctacaaacatctggtaagacaagtacatccactcatgaatgcagacacagcagctggtggttttgtgtatacctgtaaagacaagctgagaagcttactttttggggaagtaaaagaagatggaaatggatgtttcatttgtatgagtttggagcagtgctgaaggccaaagccgcctactggtttgtagttaacctagagaaggttgaaaaattaatcctacctttaaagggatttgaggtaggctggattccatcgccacaggactttagttagaattaaattcctgcttgtaatttatatccatgtttaggcttttcataagatgaaacatgccacagtgaacacactcgtgtacatatcaagagaagaaggaaaggcacaggtggagaacagtaaaaggtgggcagatgtctttgaagaaatgctcaatgtctgatgctaagtgggagaaggcagagaacaaaggatgtggcataatggtcttaacattatccaaagacttgaagctccatgtctgtaagtcaaatgttacacaaaaaaaaatgcaaatggtgtttcattggaattaccaagtgcttagaacttgctggctttcccataggtggtaaaggggtctgagctcacaccgagttgtgcttggcttgcttgtgcagctccaggcacccggtgggcactctggtggtgtttgtggtgaactgaattgaatccattgttgggcttaagttactgaaattggaacaccctttgtccttctcggcgggggcttcctggtctgtgctttacttggcttttttccttcccgtcttagcctcacccccttgtcaaccagattgagttgctatagcttgatgcagggacccagtgaagtttctccgttaaagattgggagtcgtcgaaatgtttagattcttttaggaaaggaattattttccccccttttacagggtagtaacttctccacagaagtgccaatatggcaaaattacacaagaaaacagtattgcaatgacaccattacataaggaacattgaactgttagaggagtgctcttccaaacaaaacaaaaatgtctctaggtttagtcagagctttcacaagtaataacctttctgtattaaaatcagagtaaccctttctgtattgagtgcagtgttttttactcttttctcatgcacatgttacgttggagaaaatgtttacaaaaatggttttgttacactaatgcgcaccacatatttatggtttattttaagtgactttttatgggttatttaggttttcgtcttagttgtagcacacttaccctaattttgccaattattaatttgctaaatagtaatacaaatgacaaactgcattaaatttactaattataaaagctgcaaagcagactggtggcaagtacacagcccttttttttgcagtgctaacttgtctactgtgtattatgaaaattactgttgtccccccacccttttttccttaaataaagtaaaaatgacacctaaaaaaaaaaaaaaaaaaaaaaaaaaaaaaaaaaaaa

SEQ ID NO: 5 - DNA, 3603 bp, Homo sapiens
acaaagggaggaggaagaagggagcggggtcggagccgtcggggccaaaggagacggggccaggaacaggcagtctcggcccaactgcggacgctccctccaccccctgcgcaaaaagacccaaccggagttgaggcgctgcccctgaaggccccaccttacacttggcgggggccggagccaggctcccaggactgctccagaaccgagggaagctcgggtccctccaagctagccatggtgaggcgccggaggccccggggccccacccccccggcctgaccacactgccctgggtgccctcctccagaagcccgagatgcggggggccgggagacaacactcctggctccccagagaggcgtgggtctggggctgagggccagggcccggatgcccaggttccgggactagggccttggcagccagcgggggtggggaccacgggcacccagagaaggtcctccacacatcccagcgccggctcccggccatggagcccttgaagagcctcttcctcaagagccctctagggtcatggaatggcagtggcagcgggggtggtgggggcggtggaggaggccggcctgaggggtctccaaaggcagcgggttatgccaacccggtgtggacagccctgttcgactacgagcccagtgggcaggatgagctggccctgaggaagggtgaccgtgtggaggtgctgtcccgggacgcagccatctcaggagacgagggctggtgggcgggccaggtgggtggccaggtgggcatcttcccgtccaactatgtgtctcggggtggtggcccgcccccctgcgaggtggccagcttccaggagctgcggctggaggaggtgatcggcattggaggctttggcaaggtgtacaggggcagctggcgaggtgagctggtggctgtgaaggcagctcgccaggaccccgatgaggacatcagtgtgacagccgagagcgttcgccaggaggcccggctcttcgccatgctggcacaccccaacatcattgccctcaaggctgtgtgcctggaggagcccaacctgtgcctggtgatggagtatgcagccggtgggcccctcagccgagctctggccgggcggcgcgtgcctccccatgtgctggtcaactgggctgtgcagattgcccgtgggatgcactacctgcactgcgaggccctggtgcccgtcatccaccgtgatctcaagtccaacaacattttgctgctgcagcccattgagagtgacgacatggagcacaagaccctgaagatcaccgactttggcctggcccgagagtggcacaaaaccacacaaatgagtgccgcgggcacctacgcctggatggctcctgaggttatcaaggcctccaccttctctaagggcagtgacgtctggagttttggggtgctgctgtgggaactgctgaccggggaggtgccataccgtggcattgactgccttgctgtggcctatggcgtagctgttaacaagctcacactgcccatcccatccacctgccccgagcccttcgcacagcttatggccgactgctgggcgcaggacccccaccgcaggcccgacttcgcctccatcctgcagcagttggaggcgctggaggcacaggtcctacgggaaatgccgcgggactccttccattccatgcaggaaggctggaagcgcgagatccagggtctcttcgacgagctgcgagccaaggaaaaggaactactgagccgcgaggaggagctgacgcgagcggcgcgcgagcagcggtcacaggcggagcagctgcggcggcgcgagcacctgctggcccagtgggagctagaggtgttcgagcgcgagctgacgctgctgctgcagcaggtggaccgcgagcgaccgcacgtgcgccgccgccgcgggacattcaagcgcagcaagctccgggcgcgcgacggcggcgagcgtatcagcatgccactcgacttcaagcaccgcatcaccgtgcaggcctcacccggccttgaccggaggagaaacgtcttcgaggtcgggcctggggattcgcccacctttccccggttccgagccatccagttggagcctgcagagccaggccaggcatggggccgccagtccccccgacgtctggaggactcaagcaatggagagcggcgagcatgctgggcttggggtcccagttcccccaagcctggggaagcccagaatgggaggagaaggtcccgcatggacgaagccacatggtacctggattcagatgactcatcccccttaggatctccttccacacccccagcactcaatggtaaccccccgcggcctagcctggagcccgaggagcccaagaggcctgtccccgcagagcgcggtagcagctctgggacgcccaagctgatccagcgggcgctgctgcgcggcaccgccctgctcgcctcgctgggccttggccgcgacctgcagccgccgggaggcccaggacgcgagcgcggggagtccccgacaacaccccccacgccaacgcccgcgccctgcccgaccgagccgcccccttccccgctcatctgcttctcgctcaagacgcccgactccccgcccactcctgcacccctgttgctggacctgggtatccctgtgggccagcggtcagccaagagcccccgacgtgaggaggagccccgcggaggcactgtctcacccccaccggggacatcacgctctgctcctggcaccccaggcaccccacgttcaccacccctgggcctcatcagccgacctcggccctcgccccttcgcagccgcattgatccctggagctttgtgtcagctgggccacggccttctcccctgccatcaccacagcctgcaccccgccgagcaccctggaccttgttcccggactcagaccccttctgggactccccacctgccaaccccttccaggggggcccccaggactgcagggcacagaccaaagacatgggtgcccaggccccgtgggtgccggaagcggggccttgagtgggccaggccactcccccgagctccagctgccttaggaggagtcacagcatacactggaacaggagctgggtcagcctctgcagctgcctcagtttccccagggaccccacccccctttgggggtcaggaacactacactgcacaggaagccttcacactggaagggggacctgcgcccccacatctgaaacctgtaggtccccccagctcacctgccctactggggcccaacactgtacccagctggttgggaggaccagagcctgtctcagggaattgcctgctggggtgatgcagggaggaggggaggtgcagggaagaggggccggcctcagctgtcaccagcacttttgaccaagtcctgctactgcggcccctgccctagggcttagagcatggacctcctgccctgggggtcatctggggccagggctctctggatgccttcctgctgccccagccagggttggagtcttagcctcgggatccagtgaagccagaagccaaataaactcaaaagctgtctccccaaaaaaaaaaaaaaaaaaaaaaaaaaaaaaaaa

SEQ ID NO: 6 - DNA, 2732 bp, Homo sapiens
gtctttatttcagtcccggatccgcgggcgcaggcccagctcaggcccccagggatggacgtcgtggaccctgacattttcaatagagacccccgggaccactatgacctgctacagcggctgggtggcggcacgtatggggaagtctttaaggctcgagacaaggtgtcaggggacctggtggcactgaagatggtgaagatggagcctgatgatgatgtctccacccttcagaaggaaatcctcatattgaaaacttgccggcacgccaacatcgtggcctaccatgggagttatctctggttgcagaaactctggatctgcatggaattctgtggggctggttctctccaggacatctaccaagtgacaggctccctgtcagagctccagattagctatgtctgccgggaagtgctccagggactggcctatttgcactcacagaagaagatacacagggacatcaagggagctaacatcctcatcaatgatgctggggaggtcagattggctgactttggcatctcggcccagattggggctacactggccagacgcctctctttcattgggacaccctactggatggctccggaagtggcagctgtggccctgaagggaggatacaatgagctgtgtgacatctggtccctgggcatcacggccatcgaactggccgagctacagccaccgctctttgatgtgcaccctctcagagttctcttcctcatgaccaagagtggctaccagcctccccgactgaaggaaaaaggcaaatggtcggctgccttccacaacttcatcaaagtcactctgactaagagtcccaagaaacgacccagcgccaccaagatgctcagtcatcaactggtatcccagcctgggctgaatcgaggcctgatcctggatcttcttgacaaactgaagaatcccgggaaaggaccctccattggggacattgaggatgaggagcccgagctaccccctgctatccctcggcggatcagatccacccaccgctccagctctctggggatcccagatgcagactgctgtcggcggcacatggagttcaggaagctccgaggaatggagaccagacccccagccaacaccgctcgcctacagcctcctcgagacctcaggagcagcagccccaggaagcaactgtcagagtcgtctgacgatgactatgacgacgtggacatccccacccctgcagaggacacacctcctccacttccccccaagcccaagttccgttctccatcagacgagggtcctgggagcatgggggatgatgggcagctgagcccgggggtgctggtccggtgtgccagtgggcccccaccaaacagcccccgtcctgggcctcccccatccaccagcagcccccacctcaccgcccattcagaaccctcactctggaacccaccctcccgggagcttgacaagcccccacttctgccccccaagaaggaaaagatgaagagaaagggatgtgcccttctcgtaaagttgttcaatggctgccccctccggatccacagcacggccgcctggacacatccctccaccaaggaccagcacctgctcctgggggcagaggaaggcatcttcatcctgaaccggaatgaccaggaggccacgctggaaatgctctttcctagccggactacgtgggtgtactccatcaacaacgttctcatgtctctctcaggaaagaccccccacctgtattctcatagcatccttggcctgctggaacggaaagagaccagagcaggaaaccccatcgctcacattagcccccaccgcctactggcaaggaagaacatggtttccaccaagatccaggacaccaaaggctgccgggcgtgctgtgtggcggagggtgcgagctctgggggcccgttcctgtgcggtgcattggagacgtccgttgtcctgcttcagtggtaccagcccatgaacaaattcctgcttgtccggcaggtgctgttcccactgccgacgcctctgtccgtgttcgcgctgctgaccgggccaggctctgagctgcccgctgtgtgcatcggcgtgagccccgggcggccggggaagtcggtgctcttccacacggtgcgctttggcgcgctctcttgctggctgggcgagatgagcaccgagcacaggggacccgtgcaggtgacccaggtagaggaagatatggtgatggtgttgatggatggctctgtgaagctggtgaccccggaggggtccccagtccggggacttcgcacacctgagatccccatgaccgaagcggtggaggccgtggctatggttggaggtcagcttcaggccttctggaagcatggagtgcaggtgtgggctctaggctcggatcagctgctacaggagctgagagaccctaccctcactttccgtctgcttggctcccccaggctggagtgcagtggcacgatctcgcctcactgcaacctcctcctcccaggttcaagcaattctcctgcctcagcctcccgagtagctgggattacaggcctgtagtggtggagacacgcccagtggatgatcctactgctcccagcaacctctacatccaggaatgagtccctaggggggtgtcaggaactagtccttgcaccccctcccccatagacacactagtggtcatggcatgtcctcatctcccaataaacatgactttagcctctgcaaaaaaa

SEQ ID NO: 7 - DNA, 3466 bp, Homo sapiens
ggactgtgtgtgtctggctgtagcagacgcgaggcggcgacgaggcgccggggacccgcgcgaggggcggccgggaggcggcggcggcggccgccagaagtagcagcaggaccggcggcggcgacggcagccctgaaatgcattttcctctccagcggccatgttaaccaggaaaccttcggccgccgctcccgccgcctacccgaccgattggcggcagtaagcacacaatgaatgatcacctgcatgtcggcagccacgctcacggacagatccaggttcaacagttgtttgaggataacagtaacaagcggacagtgctcacgacacaaccaaatgggcttacaacagtgggcaaaacgggcttgccagtggtgccagagcggcagctggacagcattcatagacggcaggggagctccacctctctaaagtccatggaaggcatggggaaggtgaaagccacccccatgacacctgaacaagcaatgaagcaatacatgcaaaaactcacagccttcgaacaccatgagattttcagctaccctgaaatatatttcttgggtctaaatgctaagaagcgccagggcatgacaggtgggcccaacaatggtggctatgatgatgaccagggatcatatgtgcaggtgccccacgatcacgtggcttacaggtatgaggtcctcaaggtcattgggaaggggagctttgggcaggtggtcaaggcctacgatcacaaagtccaccagcacgtggccctaaagatggtgcggaatgagaagcgcttccaccggcaagcagcggaggagatccgaatcctggaacacctgcggaagcaggacaaggataacacaatgaatgtcatccatatgctggagaatttcaccttccgcaaccacatctgcatgacgtttgagctgctgagcatgaacctctatgagctcatcaagaagaataaattccagggcttcagtctgcctttggttcgcaagtttgcccactcgattctgcagtgcttggatgctttgcacaaaaacagaataattcactgtgaccttaagcccgagaacattttgttaaagcagcagggtagaagcggtattaaagtaattgattttggctccagttgttacgagcatcagcgtgtctacacgtacatccagtcgcgtttttaccgggctccagaagtgatccttggggccaggtatggcatgcccattgatatgtggagcctgggctgcattttagcagagctcctgacgggttaccccctcttgcctggggaagatgaaggggaccagctggcctgtatgattgaactgttgggcatgccctcacagaaactgctggatgcatccaaacgagccaaaaattttgtgagctccaagggttatccccgttactgcactgtcacgactctctcagatggctctgtggtcctaaacggaggccgttcccggagggggaaactgaggggcccaccggagagcagagagtgggggaacgcgctgaaggggtgtgatgatccccttttccttgacttcttaaaacagtgtttagagtgggatcctgcagtgcgcatgaccccaggccaggctttgcggcacccctggctgaggaggcggttgccaaagcctcccaccggggagaaaacgtcagtgaaaaggataactgagagcaccggtgctatcacatctatatccaagttacctccaccttctagctcagcttccaaactgaggactaatttggcgcagatgacagatgccaatgggaatattcagcagaggacagtgttgccaaaacttgttagctgagctcacgtcccctgatgctggtaacctgaaagatacgacattgctgagccttactgggttgaaaaggagtagctcagacctgtttttatttgctcaataactctactcatttgtatcttttcagcacttaattttaatgtaagaaagttgttcattttgtttttataaaatacatgaggacaatgctttaagtttttatactttcagaaactttttgtgttctaaaagtacaatgagccttactgtatttagtgtggcagaataataacatcagtggcaggccactgattacttcatgactgccacgcatttacagattggtgtcaaagacattcactatgtttttatggttcatgttatatcctccccagggtgacagccccttaaggccctccttttccctccatgctccaggtccatgcacaggtgtagcatgtcctgcttccgtttttcataaattaatctgggtgttgggggtagtgggaggagaacggtcagaatcaaagtgacattctaagaaaaactgtaccttagagattttcctctagtgctcaaacaaatacaaaataagatccccaaggtttaaactgcccagttagcattctgacattctaaaagccggcaaagcagcttttagtggataaatgggaatggaaacgtgtgtgttcctccaaattttctagtatgatcggtgagctgttttgtaaagaagcctcatattacagagttgcttttgcacctaaatttagaattgtattccatgaactgttcctcccttttctctgcttttctcctctctgttcctcttttaataccacacgtctgttgcttgcatttagtttgtcttcttccttcagctgtgtatcccagactgttaatacagaaaagagacatttcagctgtgattatgaccattgtttcatattccaattaaaaaaagaacagcagcctagctacttaaggtggggatttcatagttccaaagaagatttagcagattagagtgagttcacacttttcaggtgccactgtaaggttctctcagcctgggaaactatcaactctttctttaaaaagaaagagggttgaaaatcctctggacgaacagaagtcactttggctgttcagtaaggccaatgttaacaacacgtttagaggaggaaaagttcaacctcaagttaaatggtttgacttattcttcgtatcattagaagaaccccagagatagcattcctctattttattttactttcttttggattgcactgattgtttttgtgggaatgacactttatctggcaaagtaactgagagtttggtaaaagaatattttcttctctgaataataattattttcacagtgaaaatttcagtattttatcactaatgtatgagcaatgatctatatcaatttcaaggcacgtgaaaaaaattttttagtatgtgcaatttaatatagaaagatttctgcctgtttggacaataggttttgggtagtacagattaggataagtaagcttatatatgcacagagattattgtattacctgtaaattgatttacaagtacttaaaagcgtggtccccagtgaggccaagaaagtttccggttaagttctttaataataatcctacagtttatcttaagaa

SEQ ID NO: 8 - DNA, 3615 bp, Homo sapiens
ggactgtgtgtgtctggctgtagcagacgcgaggcggcgacgaggcgccggggacccgcgcgaggggcggccgggaggcggcggcggcggccgccagaagtagcagcaggaccggcggcggcgacggcagccctgaaatgcattttcctctccagcggccatgttaaccaggaaaccttcggccgccgctcccgccgcctacccgaccggccgaggtggggacagcgccgttcgtcagcttcaggcttccccggggctcggtgcagggcccacccggagcggagtggggactggcccgccctcccccatcgccctgccgcctctccgggccagcaacgctgccgccgcagcccacacgattggcggcagtaagcacacaatgaatgatcacctgcatgtcggcagccacgctcacggacagatccaggttcaacagttgtttgaggataacagtaacaagcggacagtgctcacgacacaaccaaatgggcttacaacagtgggcaaaacgggcttgccagtggtgccagagcggcagctggacagcattcatagacggcaggggagctccacctctctaaagtccatggaaggcatggggaaggtgaaagccacccccatgacacctgaacaagcaatgaagcaatacatgcaaaaactcacagccttcgaacaccatgagattttcagctaccctgaaatatatttcttgggtctaaatgctaagaagcgccagggcatgacaggtgggcccaacaatggtggctatgatgatgaccagggatcatatgtgcaggtgccccacgatcacgtggcttacaggtatgaggtcctcaaggtcattgggaaggggagctttgggcaggtggtcaaggcctacgatcacaaagtccaccagcacgtggccctaaagatggtgcggaatgagaagcgcttccaccggcaagcagcggaggagatccgaatcctggaacacctgcggaagcaggacaaggataacacaatgaatgtcatccatatgctggagaatttcaccttccgcaaccacatctgcatgacgtttgagctgctgagcatgaacctctatgagctcatcaagaagaataaattccagggcttcagtctgcctttggttcgcaagtttgcccactcgattctgcagtgcttggatgctttgcacaaaaacagaataattcactgtgaccttaagcccgagaacattttgttaaagcagcagggtagaagcggtattaaagtaattgattttggctccagttgttacgagcatcagcgtgtctacacgtacatccagtcgcgtttttaccgggctccagaagtgatccttggggccaggtatggcatgcccattgatatgtggagcctgggctgcattttagcagagctcctgacgggttaccccctcttgcctggggaagatgaaggggaccagctggcctgtatgattgaactgttgggcatgccctcacagaaactgctggatgcatccaaacgagccaaaaattttgtgagctccaagggttatccccgttactgcactgtcacgactctctcagatggctctgtggtcctaaacggaggccgttcccggagggggaaactgaggggcccaccggagagcagagagtgggggaacgcgctgaaggggtgtgatgatccccttttccttgacttcttaaaacagtgtttagagtgggatcctgcagtgcgcatgaccccaggccaggctttgcggcacccctggctgaggaggcggttgccaaagcctcccaccggggagaaaacgtcagtgaaaaggataactgagagcaccggtgctatcacatctatatccaagttacctccaccttctagctcagcttccaaactgaggactaatttggcgcagatgacagatgccaatgggaatattcagcagaggacagtgttgccaaaacttgttagctgagctcacgtcccctgatgctggtaacctgaaagatacgacattgctgagccttactgggttgaaaaggagtagctcagacctgtttttatttgctcaataactctactcatttgtatcttttcagcacttaattttaatgtaagaaagttgttcattttgtttttataaaatacatgaggacaatgctttaagtttttatactttcagaaactttttgtgttctaaaagtacaatgagccttactgtatttagtgtggcagaataataacatcagtggcaggccactgattacttcatgactgccacgcatttacagattggtgtcaaagacattcactatgtttttatggttcatgttatatcctccccagggtgacagccccttaaggccctccttttccctccatgctccaggtccatgcacaggtgtagcatgtcctgcttccgtttttcataaattaatctgggtgttgggggtagtgggaggagaacggtcagaatcaaagtgacattctaagaaaaactgtaccttagagattttcctctagtgctcaaacaaatacaaaataagatccccaaggtttaaactgcccagttagcattctgacattctaaaagccggcaaagcagcttttagtggataaatgggaatggaaacgtgtgtgttcctccaaattttctagtatgatcggtgagctgttttgtaaagaagcctcatattacagagttgcttttgcacctaaatttagaattgtattccatgaactgttcctcccttttctctgcttttctcctctctgttcctcttttaataccacacgtctgttgcttgcatttagtttgtcttcttccttcagctgtgtatcccagactgttaatacagaaaagagacatttcagctgtgattatgaccattgtttcatattccaattaaaaaaagaacagcagcctagctacttaaggtggggatttcatagttccaaagaagatttagcagattagagtgagttcacacttttcaggtgccactgtaaggttctctcagcctgggaaactatcaactctttctttaaaaagaaagagggttgaaaatcctctggacgaacagaagtcactttggctgttcagtaaggccaatgttaacaacacgtttagaggaggaaaagttcaacctcaagttaaatggtttgacttattcttcgtatcattagaagaaccccagagatagcattcctctattttattttactttcttttggattgcactgattgtttttgtgggaatgacactttatctggcaaagtaactgagagtttggtaaaagaatattttcttctctgaataataattattttcacagtgaaaatttcagtattttatcactaatgtatgagcaatgatctatatcaatttcaaggcacgtgaaaaaaattttttagtatgtgcaatttaatatagaaagatttctgcctgtttggacaataggttttgggtagtacagattaggataagtaagcttatatatgcacagagattattgtattacctgtaaattgatttacaagtacttaaaagcgtggtccccagtgaggccaagaaagtttccggttaagttctttaataataatcctacagtttatcttaagaa

SEQ ID NO: 9 - DNA, 2104 bp, Homo sapiens
agcaaaatcaagcagttggttagcgaagggattcggtgcagatggatggaccgtctgggctgcggagtccacgggcctgaaacgcggtcgctcctgacccgtcctccgcaaccctcgtcccaccaggccgcggtccccggagcaggccggcgggtggcaggaagacaccaggctgcagggggctggagaagcaacacccctctccctccccgccagagaactggagcgcggggacctcggttgcggatgctggcggcgagccgggttgctaggcgacatcgagagaggaagaggagaaagtcttcatggggtgctagagcatcttaaatgtccctctcatgcagggacatttccctgctgaacgaagagaaggcagacctcggcgagggacccgtggtcagcagcagcttctggtgagcccccgcttcctccgagcctggcacccccctcccgtctcagccaggatgccaacgaggcgctgggccccgggcacccagtgtatcaccaaatgcgagcacacccgccccaagccaggggagctggccttccgcaagggcgacgtggtcaccatcctggaggcctgcgagaacaagagctggtaccgcgtcaagcaccacaccagtggacaggaggggctgctggcagctggggcgctgcgggagcgggaggccctctccgcagaccccaagctcagcctcatgccgtggttccacgggaagatctcgggccaggaggctgtccagcagctgcagcctcccgaggatgggctgttcctggtgcgggagtccgcgcgccaccccggcgactacgtcctgtgcgtgagctttggccgcgacgtcatccactaccgcgtgctgcaccgcgacggccacctcacaatcgatgaggccgtgttcttctgcaacctcatggacatggtggagcattacagcaaggacaagggcgctatctgcaccaagctggtgagaccaaagcggaaacacgggaccaagtcggccgaggaggagctggccagggcgggctggttactgaacctgcagcatttgacattgggagcacagatcggagagggagagtttggagctgtcctgcagggtgagtacctggggcaaaaggtggccgtgaagaatatcaagtgtgatgtgacagcccaggccttcctggacgagacggccgtcatgacgaagatgcaacacgagaacctggtgcgtctcctgggcgtgatcctgcaccaggggctgtacattgtcatggagcacgtgagcaagggcaacctggtgaactttctgcggacccggggtcgagccctcgtgaacaccgctcagctcctgcagttttctctgcacgtggccgagggcatggagtacctggagagcaagaagcttgtgcaccgcgacctggccgcccgcaacatcctggtctcagaggacctggtggccaaggtcagcgactttggcctggccaaagccgagcggaaggggctagactcaagccggctgcccgtcaagtggacggcgcccgaggctctcaaacacgggaagttcaccagcaagtcggatgtctggagttttggggtgctgctctgggaggtcttctcatatggacgggctccgtaccctaaaatgtcactgaaagaggtgtcggaggccgtggagaaggggtaccgcatggaaccccccgagggctgtccaggccccgtgcacgtcctcatgagcagctgctgggaggcagagcccgcccgccggccacccttccgcaaactggccgagaagctggcccgggagctacgcagtgcaggtgccccagcctccgtctcagggcaggacgccgacggctccacctcgccccgaagccaggagccctgaccccacccggtggggcccttggccccagaggaccgagagagtggagagtgcggcgtgggggcactgaccaggcccaaggagggtccaggcgggcaagtcatcctcctggtgcccacagcaggggctggcccacgtagggggctctgggcggcccgtggacaccccagacctgcgaaggatgatcgcccgataaagacggattctaaggactctaaaaaaaaaaaaaaaaaaaaaaaaaa

SEQ ID NO: 10 - DNA, 1933 bp, Homo sapiens
ggccggcggcggcggcggcgcgcggaggagaccgcagtgcggccggcgctaggacccgcgggggcctcccaggccgcggcgcctcgcgctttcccccactccccgacccttcttcgcccccaaaatgaggaaacggagcaactcgctccaagttgtgcagccgggaccgcctcggggtgtgcagccggctcgcggaggccctcctgggggcgggcgcggggcgcggctcgggggcgccccctgagcaggtgagcccccgcttcctccgagcctggcacccccctcccgtctcagccaggatgccaacgaggcgctgggccccgggcacccagtgtatcaccaaatgcgagcacacccgccccaagccaggggagctggccttccgcaagggcgacgtggtcaccatcctggaggcctgcgagaacaagagctggtaccgcgtcaagcaccacaccagtggacaggaggggctgctggcagctggggcgctgcgggagcgggaggccctctccgcagaccccaagctcagcctcatgccgtggttccacgggaagatctcgggccaggaggctgtccagcagctgcagcctcccgaggatgggctgttcctggtgcgggagtccgcgcgccaccccggcgactacgtcctgtgcgtgagctttggccgcgacgtcatccactaccgcgtgctgcaccgcgacggccacctcacaatcgatgaggccgtgttcttctgcaacctcatggacatggtggagcattacagcaaggacaagggcgctatctgcaccaagctggtgagaccaaagcggaaacacgggaccaagtcggccgaggaggagctggccagggcgggctggttactgaacctgcagcatttgacattgggagcacagatcggagagggagagtttggagctgtcctgcagggtgagtacctggggcaaaaggtggccgtgaagaatatcaagtgtgatgtgacagcccaggccttcctggacgagacggccgtcatgacgaagatgcaacacgagaacctggtgcgtctcctgggcgtgatcctgcaccaggggctgtacattgtcatggagcacgtgagcaagggcaacctggtgaactttctgcggacccggggtcgagccctcgtgaacaccgctcagctcctgcagttttctctgcacgtggccgagggcatggagtacctggagagcaagaagcttgtgcaccgcgacctggccgcccgcaacatcctggtctcagaggacctggtggccaaggtcagcgactttggcctggccaaagccgagcggaaggggctagactcaagccggctgcccgtcaagtggacggcgcccgaggctctcaaacacgggaagttcaccagcaagtcggatgtctggagttttggggtgctgctctgggaggtcttctcatatggacgggctccgtaccctaaaatgtcactgaaagaggtgtcggaggccgtggagaaggggtaccgcatggaaccccccgagggctgtccaggccccgtgcacgtcctcatgagcagctgctgggaggcagagcccgcccgccggccacccttccgcaaactggccgagaagctggcccgggagctacgcagtgcaggtgccccagcctccgtctcagggcaggacgccgacggctccacctcgccccgaagccaggagccctgaccccacccggtggggcccttggccccagaggaccgagagagtggagagtgcggcgtgggggcactgaccaggcccaaggagggtccaggcgggcaagtcatcctcctggtgcccacagcaggggctggcccacgtagggggctctgggcggcccgtggacaccccagacctgcgaaggatgatcgcccgataaagacggattctaaggactctaaaaaaaaaaaaaaaaaaaa

SEQ ID NO: 11 - DNA, 2183 bp, Homo sapiens
ggccggcggcggcggcggcgcgcggaggagaccgcagtgcggccggcgctaggacccgcgggggcctcccaggccgcggcgcctcgcgctttcccccactccccgacccttcttcgcccccaaaatgaggaaacggagcaactcgctccaagttgtgcagccgggaccgcctcggggtgtgcagccggctcgcggaggccctcctgggggcgggcgcggggcgcggctcgggggcgccccctgagcagaaaacaggaagaaccaggctcggtccagtggcacccagctccctacctcctgtgccagccgcctggcctgtggcaggccattcccagcgtccccgactgtgaccacttgctcagtgtgcctctcacctgcctcagtttccctctgggggcgatggcggggcgaggctctctggtttcctggcgggcatttcacggctgtgattctgctgaggaacttccccgggtgagcccccgcttcctccgagcctggcacccccctcccgtctcagccaggatgccaacgaggcgctgggccccgggcacccagtgtatcaccaaatgcgagcacacccgccccaagccaggggagctggccttccgcaagggcgacgtggtcaccatcctggaggcctgcgagaacaagagctggtaccgcgtcaagcaccacaccagtggacaggaggggctgctggcagctggggcgctgcgggagcgggaggccctctccgcagaccccaagctcagcctcatgccgtggttccacgggaagatctcgggccaggaggctgtccagcagctgcagcctcccgaggatgggctgttcctggtgcgggagtccgcgcgccaccccggcgactacgtcctgtgcgtgagctttggccgcgacgtcatccactaccgcgtgctgcaccgcgacggccacctcacaatcgatgaggccgtgttcttctgcaacctcatggacatggtggagcattacagcaaggacaagggcgctatctgcaccaagctggtgagaccaaagcggaaacacgggaccaagtcggccgaggaggagctggccagggcgggctggttactgaacctgcagcatttgacattgggagcacagatcggagagggagagtttggagctgtcctgcagggtgagtacctggggcaaaaggtggccgtgaagaatatcaagtgtgatgtgacagcccaggccttcctggacgagacggccgtcatgacgaagatgcaacacgagaacctggtgcgtctcctgggcgtgatcctgcaccaggggctgtacattgtcatggagcacgtgagcaagggcaacctggtgaactttctgcggacccggggtcgagccctcgtgaacaccgctcagctcctgcagttttctctgcacgtggccgagggcatggagtacctggagagcaagaagcttgtgcaccgcgacctggccgcccgcaacatcctggtctcagaggacctggtggccaaggtcagcgactttggcctggccaaagccgagcggaaggggctagactcaagccggctgcccgtcaagtggacggcgcccgaggctctcaaacacgggaagttcaccagcaagtcggatgtctggagttttggggtgctgctctgggaggtcttctcatatggacgggctccgtaccctaaaatgtcactgaaagaggtgtcggaggccgtggagaaggggtaccgcatggaaccccccgagggctgtccaggccccgtgcacgtcctcatgagcagctgctgggaggcagagcccgcccgccggccacccttccgcaaactggccgagaagctggcccgggagctacgcagtgcaggtgccccagcctccgtctcagggcaggacgccgacggctccacctcgccccgaagccaggagccctgaccccacccggtggggcccttggccccagaggaccgagagagtggagagtgcggcgtgggggcactgaccaggcccaaggagggtccaggcgggcaagtcatcctcctggtgcccacagcaggggctggcccacgtagggggctctgggcggcccgtggacaccccagacctgcgaaggatgatcgcccgataaagacggattctaaggactctaaaaaaaaaaaaaaaaaaaaaaaaaaaaaaaaaaaaaaaaaaaaaaa

SEQ ID NO: 12 - DNA, 3273 bp, Homo sapiens
agtccccgcgcagtccccgcgcagtcccagcgccaccgggcagcagcggcgccgtgctcgctccagggcgcaaccatgtcgccatttcttcggattggcttgtccaactttgactgcgggtcctgccagtcttgtcagggcgaggctgttaacccttactgtgctgtgctcgtcaaagagtatgtcgaatcagagaacgggcagatgtatatccagaaaaagcctaccatgtacccaccctgggacagcacttttgatgcccatatcaacaagggaagagtcatgcagatcattgtgaaaggcaaaaacgtggacctcatctctgaaaccaccgtggagctctactcgctggctgagaggtgcaggaagaacaacgggaagacagaaatatggttagagctgaaacctcaaggccgaatgctaatgaatgcaagatactttctggaaatgagtgacacaaaggacatgaatgaatttgagacggaaggcttctttgctttgcatcagcgccggggtgccatcaagcaggcaaaggtccaccacgtcaagtgccacgagttcactgccaccttcttcccacagcccacattttgctctgtctgccacgagtttgtctggggcctgaacaaacagggctaccagtgccgacaatgcaatgcagcaattcacaagaagtgtattgataaagttatagcaaagtgcacaggatcagctatcaatagccgagaaaccatgttccacaaggagagattcaaaattgacatgccacacagatttaaagtctacaattacaagagcccgaccttctgtgaacactgtgggaccctgctgtggggactggcacggcaaggactcaagtgtgatgcatgtggcatgaatgtgcatcatagatgccagacaaaggtggccaacctttgtggcataaaccagaagctaatggctgaagcgctggccatgattgagagcactcaacaggctcgctgcttaagagatactgaacagatcttcagagaaggtccggttgaaattggtctcccatgctccatcaaaaatgaagcaaggccgccatgtttaccgacaccgggaaaaagagagcctcagggcatttcctgggagtctccgttggatgaggtggataaaatgtgccatcttccagaacctgaactgaacaaagaaagaccatctctgcagattaaactaaaaattgaggattttatcttgcacaaaatgttggggaaaggaagttttggcaaggtcttcctggcagaattcaagaaaaccaatcaatttttcgcaataaaggccttaaagaaagatgtggtcttgatggacgatgatgttgagtgcacgatggtagagaagagagttctttccttggcctgggagcatccgtttctgacgcacatgttttgtacattccagaccaaggaaaacctcttttttgtgatggagtacctcaacggaggggacttaatgtaccacatccaaagctgccacaagttcgacctttccagagcgacgttttatgctgctgaaatcattcttggtctgcagttccttcattccaaaggaatagtctacagggacctgaagctagataacatcctgttagacaaagatggacatatcaagatcgcggattttggaatgtgcaaggagaacatgttaggagatgccaagacgaataccttctgtgggacacctgactacatcgccccagagatcttgctgggtcagaaatacaaccactctgtggactggtggtccttcggggttctcctttatgaaatgctgattggtcagtcgcctttccacgggcaggatgaggaggagctcttccactccatccgcatggacaatcccttttacccacggtggctggagaaggaagcaaaggaccttctggtgaagctcttcgtgcgagaacctgagaagaggctgggcgtgaggggagacatccgccagcaccctttgtttcgggagatcaactgggaggaacttgaacggaaggagattgacccaccgttccggccgaaagtgaaatcaccatttgactgcagcaatttcgacaaagaattcttaaacgagaagccccggctgtcatttgccgacagagcactgatcaacagcatggaccagaatatgttcaggaacttttccttcatgaaccccgggatggagcggctgatatcctgaatcttgcccctccagagacaggaaagaatttgccttctccctgggaactggttcaagagacactgcttgggttcctttttcaacttggaaaaagaaagaaacactcaacaataaagactgagacccgttcgcccccatgtgacttttatctgtagcagaaaccaagtctacttcactaatgacgatgccgtgtgtctcgtctcctgacatgtctcacagacgctcctgaagttaggtcattactaaccatagttatttacttgaaagatgggtctccgcacttggaaaggtttcaagacttgatactgcaataaattatggctcttcacctgggcgccaactgctgatcaatgaaatgcttgttgaatcaggggcaaacggagtacagacgtctcaagactgaaacggccccattgcctggtctagtagcggatctcactcagccgcagacaagtaatcactaacccgttttattctattcctatctgtggatgtgtaaatggctggggggccagccctggataggtttttatgggaattctttacaataaacatagcttgtaacttgagatctacaaatccattcatcctgattgggcatgaaatccatggtcaagaggacaagtggaaagtgagagggaaggtttgctagacaccttcgcttgttatcttgtcaagatagaaaagatagtatcatttcacccttgccagtaaaaacctttccatccacccattctcagcagactccagtattggcacagtcactcactgccattctcacactataacaagaaaagaaatgaagtgcataagtctcctgggaaaagaaccttaaccccttctcgtgccatgactggtgatttcatgactcataagcccctccgtaggcatcattcaagatcaatggcccatgcatgctgtttgcagcagtcaattgagttgaattagaattccaaccatacattttaaaggtatttgtgctgtgtgtatattttgataaaatgttgtgacttcatggcaaacaggtggatgtgtaaaaatggaataaaaaaaaaaaaagagtcaaaaaaaaaaaaa

SEQ ID NO: 13 - DNA, 2387 bp, Homo sapiens
ggggcggcgggcgacggggcgggcgcaggatgagggcggccattgctggggctccgcttcggggaggaggacgctgaggaggcgccgagccgcgcagcgctgcgggggaggcgcccgcgccgacgcggggcccatggccaggaccaccagccagctgtatgacgccgtgcccatccagtccagcgtggtgttatgttcctgcccatccccatcaatggtgaggacccagactgagtccagcacgccccctggcattcctggtggcagcaggcagggccccgccatggacggcactgcagccgagcctcggcccggcgccggctccctgcagcatgcccagcctccgccgcagcctcggaagaagcggcctgaggacttcaagtttgggaaaatccttggggaaggctctttttccacggttgtcctggctcgagaactggcaacctccagagaatatgcgattaaaattctggagaagcgacatatcataaaagagaacaaggtcccctatgtaaccagagagcgggatgtcatgtcgcgcctggatcaccccttctttgttaagctttacttcacatttcaggacgacgagaagctgtatttcggccttagttatgccaaaaatggagaactacttaaatatattcgcaaaatcggttcattcgatgagacctgtacccgattttacacggctgagattgtgtctgctttagagtacttgcacggcaagggcatcattcacagggaccttaaaccggaaaacattttgttaaatgaagatatgcacatccagatcacagattttggaacagcaaaagtcttatccccagagagcaaacaagccagggccaactcattcgtgggaacagcgcagtacgtttctccagagctgctcacggagaagtccgcctgtaagagttcagacctttgggctcttggatgcataatataccagcttgtggcaggactcccaccattccgagctggaaacgagtatcttatatttcagaagatcattaagttggaatatgactttccagaaaaattcttccctaaggcaagagacctcgtggagaaacttttggttttagatgccacaaagcggttaggctgtgaggaaatggaaggatacggacctcttaaagcacacccgttcttcgagtccgtcacgtgggagaacctgcaccagcagacgcctccgaagctcaccgcttacctgccggctatgtcggaagacgacgaggactgctatggcaattatgacaatctcctgagccagtttggctgcatgcaggtgtcttcgtcctcctcctcacactccctgtcagcctccgacacgggcctgccccagaggtcaggcagcaacatagagcagtacattcacgatctggactcgaactcctttgaactggacttacagttttccgaagatgagaagaggttgttgttggagaagcaggctggcggaaacccttggcaccagtttgtagaaaataatttaatactaaagatgggcccagtggataagcggaagggtttatttgcaagacgacgacagctgttgctcacagaaggaccacatttatattatgtggatcctgtcaacaaagttctgaaaggtgaaattccttggtcacaagaacttcgaccagaggccaagaattttaaaactttctttgtccacacgcctaacaggacgtattatctgatggaccccagcgggaacgcacacaagtggtgcaggaagatccaggaggtttggaggcagcgataccagagccacccggacgccgctgtgcagtgacgtggcctgcggccgggctgcccttcgctgccaggacacctgccccagcgcggcttggccgccatccgggacgcttccagaccacctgccagccatcacaaggggaacgcagaggcggaaaccttgcagcatttttatttaaaagaaaagaagaaaaaaaacacccaaccacacaaagaacaaaaccagtaacaaacacaaaggaattcagggtcgctttgcttgctctctgtgctccgtggaggcctccgtgtgccctcgttgccgtggggacccagctccatgcacgtcaacccagtcccgcccagactagtggacagacctggtgtcaccagtttttcctagcatcagtccgaaccatgcgcccgccctgccccaactgtgtgctggtcctgctgtggccgaggggaccgggtgtgtttggctctttatgcccctcccgctgtggtcctggaactcttcaccagggagggagccctgcgggggccgcagctttgtggagggagccgccgtgcttctgtcacctgctccctttcttgcgtctccctgtgatgggcccttaggcctggctgggcccattaca

SEQ ID NO: 14 - DNA, 4373 bp, Homo sapiens
ggcgccgacccatgctggctgggaacgtgtctcccggtgacgcagccccgggtggggaacgtggtgcggcggcggcggcggcggcgactgtacgcgcctccgccgcccccgagaggacgcgccgtgcagcggctgagtggcggcggcggcgacggcaaacccggagctgccggccggcgcgcgggaggaggacgcgggtgcggtctaggaaacggagctgcgggcggaggctccatgttgggaagcggcgccgttcgtgcttgttagcgggaatccgggagccgcggggtgagctggcgggggccgggccctaagtgaagatggaggccccgctgcggcctgccgcggacatcctgaggcggaacccgcagcaggactacgaactcgtccagagggtcggcagcggcacctacggggacgtctataaggccagaaatgtacacacaggagagctggctgcagtaaaaatcattaaattggagcctggagatgatttttctttgattcaacaagaaatatttatggttaaagaatgtaaacattgtaacatcgttgcctactttgggagttatcttagtcgggaaaaactatggatttgtatggaatactgtggtggcggatcacttcaagatatttaccatgttactggaccattatcagaattgcaaatagcctatgtatgcagagaaaccttacagggtcttgcctatttgcatactaaaggcaaaatgcatagagatatcaaaggtgctaatattttattgacagaccatggcgatgtaaaattagctgactttggtgtggctgcaaaaataacagctaccattgcaaaacgaaaatctttcattggcaccccttactggatggccccagaagttgcagcagtagagaagaatggtggctacaaccaactctgtgatatctgggcagtaggaataacagcaattgaacttggagaacttcagccacctatgtttgatctccacccaatgagggctctcttcttaatgtcaaaaagtaattttcagcctccaaaactaaaggacaaaacaaaatggtcatcaacattccataattttgtcaaaatagcactaaccaaaaacccaaaaaaaagaccaactgctgaaagacttctgactcacacttttgttgcacagccaggtctctctagagccctagcagttgaactgttagacaaagtgaacaatccagataaccacgcacattacactgaagcagatgacgatgactttgagccccatgcaatcattcgtcataccattagatctacaaacaggaatgccagagctgaacggacagcttcagaaataaattttgacaaattacaatttgaacctcctctgagaaaagaaacagaagcacgagatgaaatgggattgtcatcagacccaaatttcatgttacagtggaatccttttgttgatggtgcaaatactggcaaatcaacctcaaaacgtgcaataccacctcccctacctcctaagccaaggataagcagttaccctgaagacaactttccggatgaagaaaaagcatcaaccataaaacattgtcctgattcagaaagcagagctccccaaattctcagaagacagagtagcccaagttgtgggcctgtggcagagacttcttctattggaaatggtgatggtatttcaaaactgatgagtgaaaatacagaaggatcagcacaagcaccacagttaccacgaaaaaaggacaaacgagacttccctaaaccagccatcaatggccttccacccaccccaaaagttctgatgggagcatgcttttcaaaagtttttgatggctgtcctttgaaaattaattgtgcaacatcctggatacatcctgatacaaaagatcagtacattatttttggaactgaagatggtatttacacactgaatctcaatgagctacatgaggcaacgatggaacagttatttccacggaagtgtacttggctgtatgttatcaataatactttaatgtcattatcagaaggaaaaacctttcagctctactctcacaatcttatagctttgtttgaacatgccaaaaaaccaggattagctgcccatattcaaactcacaggtttccagaccgaatactaccaagaaaattcgctttaacaacaaagattcctgatacaaaaggctgccacaaatgttgcatagtcagaaacccttacacgggacataaatacctctgtggagctttacagtctggaattgttttacttcagtggtatgagccaatgcagaaattcatgttgataaagcactttgattttcctttgccaagtcctttgaatgtttttgaaatgctggtgatacctgaacaggaataccctatggtctgtgtagctattagcaaaggcactgaatcgaatcaggtagttcagtttgagacaatcaatttgaactctgcatcttcatggtttacagaaattggtgcaggcagccagcagttagattccattcatgtaacacagttggagagagataccgttttagtgtgtttagacaaatttgtgaaaattgtaaatctacaaggaaaattaaaatcaagtaagaaactggcctctgagttaagttttgattttcgcattgaatctgtagtatgccttcaagacagtgtgttggctttctggaaacatgggatgcagggtaaaagcttcaagtcagatgaggttacccaggagatttcagatgaaacaagagttttccgcttattaggatcagacagggttgtcgttttggaaagtaggccaacagaaaatcctactgcacacagcaatctctacatcttggctggacatgaaaatagttactaagcaacagaaactgatctcaaatgacaggaaaatgaatatactccattgaaaggaaaaataaggaaattcaatacaaactgcactatgatttgctttaactattatgggttatattgcaaatgatctgtactttagggtagaattcaatattttctgcagctggaaacagctagtctatctcttgccactgtgtggtggttatatcaagtttgcttaataaaagctatgagacaaatagtcctctagttccaggaaacacagtctttttttaaaaaaaacaatgtttgtaacaagggtgccatggtatttttagataactcgtgattatcttaagagaggtaaatttagtgatcattttatatcatgtcttattccttcttaatgaacataatttgttaaattctcaagcaaggttttcacttttatattggccattctgtatgtttttgtaaaacagaatatttaatccttatttattaatctcttgctggagtggtgtaatgtatctaacttttagcaaaggagggttgcagagcagcttaaattttttttataatgtataagaattttgtttatcttttaagagtagtaaagtactttgagtgtttgggggttcaacacacacatgcaattttgcttaacaaaagtattttataatacagtttcatacagaattaccttaaaagggagtcttatgttttcaactacagatagttgtaagggatcatacagaagatattgatgatagttgaaatattcttagaaggggtgtgtatgtctagctgtgtctaccatgtgtatgtattcttgacaagcagtataaaatacctgtgatttttctttacattagggataatgcataaggaattaatcttcatatatattatcatccctaatgtagcagggggaagtatttaattgcccatgatatgtattttacttatactatgccagagaggaaactataaagtaattacacatgtaatcttgggtttttcacatatgtaggtattcattttgagtaggttgaagaagaaaaaaaatatttaaatgaattgaattcctgatgggatagtatcaataagtatttaaaagccagtattctaaaaataataaagggtagggtcatttttgagtttgtttttcttttgctattgttaatattcaaaattaaagtgttacattggtacctgttgtcttaatgcatttattgagaacagcattgagatgatgaacaaggggttagcaatagcaaactctataattattttgactaattacttaagaggaaaacagtataagtatctcattcagtatttagcaattctgtaaaataagtattatctctatttttcagatgaggaagtaagggtttagcaaggttaagagatctatccaatttacacagcaagttagtagttgagcctgaccatgagtcttctgactctgttcttttcactatgcaatacgcaaacaataaaatgttatacaaataaaaaaaaaaaaaaaaaa

SEQ ID NO: 15 - DNA, 1588 bp, Homo sapiens
gcggtgtttgtctgccggactgacgggcggccgggcggtgcgcggcggcggtggcggcggggaaaatggcggcgtcctccctggaacagaagctgtcccgcctggaagcaaagctgaagcaggagaaccgggaggcccggcggaggatcgacctcaacctggatatcagcccccagcggcccaggcccaccctgcagctcccgctggccaacgatgggggcagccgctcgccatcctcagagagctccccgcagcaccccacgccccccgcccggccccgccacatgctggggctcccgtcaaccctgttcacaccccgcagcatggagagcattgagattgaccagaagctgcaggagatcatgaagcagacgggctacctgaccatcgggggccaggtaccaccttcactgtggcggggagagggaggaggcccagccaggctggacccatcctgggagcgccagtggggggcagggggcggtggcagagccccagggaccctccaaccctccctctcctcccagcgctaccaggcagaaatcaacgacctggagaacttgggcgagatgggcagcggcacctgcggccaggtgtggaagatgcgcttccggaagaccggccacgtcattgccgttaagcaaatgcggcgctccgggaacaaggaggagaacaagcgcatcctcatggacctggatgtggtgctgaagagccacgactgcccctacatcgtgcagtgctttgggacgttcatcaccaacacggacgtcttcatcgccatggagctcatgggcacctgcgctgagaagctcaagaagcggatgcagggccccatccccgagcgcattctgggcaagatgacagtggcgattgtgaaggcgctgtactacctgaaggagaagcacggtgtcatccaccgcgacgtcaagccctccaacatcctgctggacgagcggggccagatcaagttctgcgacttcggcatcagcggccgcctggtggactccaaagccaagacgcggagcgccggctgtgccgcctacatggcacccgagcgcattgaccccccagaccccaccaagccggactatgacatccgggccgacgtatggagcctgggcatctcgttggtggagctggcaacaggacagtttccctacaagaactgcaagacggactttgaggtcctcaccaaagtcctacaggaagagcccccgcttctgcccggacacatgggcttctcgggggacttccagtccttcgtcaaagactgccttactaaagatcacaggaagagaccaaagtataataagctacttgaacacagcttcatcaagcgctacgagacgctggaggtggacgtggcgtcctggttcaaggatgtcatggcgaagactgagtcaccgcggactagcggcgtcctgagccagccccacctgcccttcttcaggtagctgcttggcggcggccagccccacagggggccaggggcatggccacaggcccccctccccacttggccacccagctgcctgccaggggagacctgggacctggacggccacctaggactgaggacagagagtgg

SEQ ID NO: 16 - DNA, 4836 bp, Homo sapiens
agaagccccgggctcgtcagtcaaaccggttctctgtttgcactcggcagcacgggcaggcaagtggtccctaggttcgggagcagagcagcagcgcctcagtcctggtcccccagtcccaagcctcacctgcctgcccagcgccaggatggccaccatcacctgcacccgcttcacggaagagtaccagctcttcgaggaattgggcaagggagccttctcggtggtgcgaaggtgtgtgaaggtgctggctggccaggagtatgctgccaagatcatcaacacaaagaagctgtcagccagagaccatcagaagctggagcgtgaagcccgcatctgccgcctgctgaagcaccccaacatcgtccgactacatgacagcatctcagaggagggacaccactacctgatcttcgacctggtcactggtggggaactgtttgaagatatcgtggcccgggagtattacagtgaggcggatgccagtcactgtatccagcagatcctggaggctgtgctgcactgccaccagatgggggtggtgcaccgggacctgaagcctgagaatctgttgctggcctccaagctcaagggtgccgcagtgaagctggcagactttggcctggccatagaggtggagggggagcagcaggcatggtttgggtttgcagggactcctggatatctctccccagaagtgctgcggaaggacccgtacgggaagcctgtggacctgtgggcttgtggggtcatcctgtacatcctgctggttgggtaccccccgttctgggatgaggaccagcaccgcctgtaccagcagatcaaagccggcgcctatgatttcccatcgccggaatgggacactgtcaccccggaagccaaggatctgatcaataagatgctgaccattaacccatccaaacgcatcacagctgccgaagcccttaagcacccctggatctcgcaccgctccaccgtggcatcctgcatgcacagacaggagaccgtggactgcctgaagaagttcaatgccaggaggaaactgaagggagccattctcaccacgatgctggccaccaggaacttctccggagggaagagtgggggaaacaagaagagcgatggtgtgaagaaaagaaagtccagttccagcgttcagttaatggaatcctcagagagcaccaacaccaccatcgaggatgaagacaccaaagtgcggaaacaggaaattataaaagtgacagagcagctgattgaagccataagcaatggagattttgagtcctacacgaagatgtgcgaccctggcatgacagccttcgaacctgaggccctggggaacctggttgagggcctggacttccatcgattctattttgaaaacctgtggtcccggaacagcaagcccgtgcacaccaccatcctgaatccccacatccacctgatgggcgacgagtcagcctgcatcgcctacatccgcatcacgcagtacctggacgctggcggcatcccacgcaccgcccagtcggaggagacccgtgtctggcaccgccgggatggcaaatggcagatcgtccacttccacagatctggggcgccctccgtcctgccccactgagggaccaggctggggtcgctgcgttgctgtgccgcagagatccactctgtccgtggagtggagctgctggttctcccaggtggattttgctggaattctcccatgtcatcaccccaccaccgtcacttctgtacctgcatcaagaaaacctgcttgttcacaaaagtcatcgcaacttcagagcgaacggccacatctccccacctctcacccccaccctctcccctgccaggctggggcttcctcaggcatgggtgtccacagcactggccccctctccccagcctcagctgctgtccgcctgatctgtcttgggctgtaggctagaatgcccgggctggtgcccaccaggggctggggagaaggaggggtggcatgatgaggaaggcagcatccgtccgtccctctcccagacctctcctcttccagtgtccccggggaagggcagatgacactcccttccccctaagccaaccgcactgaaggagtggggagaagagcatacgccaggagcctcctgcctcaaagtgctcccctaagtcttcttcctcctgtgctgacctcagggtggtctgacccttccctcggtgtgggggatgtggccctctcaggtgcccctacttgctttctgcttccttctggtgaagtccacctccaacattaacctgcccaccccacccccgtcatccctggagaattccagctttgtcgtatctcagagagggaatctaattgtttttggggggcaaaagaaagcaacgtttaggtatcacttctacttggaccgcatgcctttttatagccaaatttctgtgtatttcgtaaatggatttcgcgttaatggatatttatgtaataactagacttctcagattattgtgagaagggtcaggttggaaggggtgtaggaagaggggtgaggggtagtttttttctgttctagtttttttttttttttttgtcatctctgaggtggaccttgtcacctgtggttattggggccaaggtggactcagctccggggagaagggcctctctgccatttcggtcccaaggtgagctgacacaggcgttccttttgggactgtggaagcatcagatgccagcactgactcaggaacagcaagtcagggcagagaggaggagggaggctgtcaggatggaaatacctggacttttctttgcttccctcgcaaactggggtcttctctaccgaacttcccaggatttcatctcaccatatctgtgtgccgcccccagcaccccccacccacctctggggggcccgtgagcgtgtgtcttcattgcctctctccccttggcgtctgatgaccacagcaaagcactgggaatttctactcttcatgcctcatcctgcagcctcgggttcgcattctctctttcttttcctctttccctctttccctgggattgactctgagtggaataccttggcacatccactaggatctactgtctgcactgttttctttgcatgactttatacgcagtaagtatgttgaaaacaaacaaaaagaagaaaacactcaacaaaaccaatctacatgttttggactaaaaaaaaaaatagaggttgtattctcagtgtccgactcggaattatgttgctgcctctctgtgcttttggcctctgtgtggccgtgttttgccagcatgagatactgtcccctctggaggattttaggggaggaagagccacgtccccagggattggaggaggctccggtaccctcgaccctcctgggtgttggttggagcagaactggtgaggatgtttgatccgagattttctgagctctccccaatcaccagctgtctgctgggttcttttctcaagtcctgctgcccaggcccaggtgagacaggcaacgccaggtctgcaggccaggagagatgctgcccaggcctcctggtttccaagctggtccatcactggcctctgtccttggcagagaccttgctgcccaggcccaggggcaggctcttggcctgccccaggcccagagggcttcccagtaaggcccagtgatcccattatcccaggggcaaaaccacctgtccccttttgagctgccagttccctacagccatccccagtcaagggtgagggtgtggccttcaccaggggctgctgtaattaccgagcaaggtctgagctcttcttcagcctcagttccctcattggttaaaagggttctttgttcccatccagccgatgaaggagcaaacgtctggctatgtgaagcctaatttacctgcaggaactggcagggatagtcactggctggactcctgtttacttctagacctggtcaggctccatcccctcccccacctgcccctgattcccctcgtcggtgcctgtcaactgcttttcagcagtggactgcaggggaaagagcagtgatttggggtgagtaggcttcaattcccagctctgaccagacttgctgtgtgaccttgggcaagttcctttccctctttggagcttggtttccctgccagaggaaactgagctggaggagcctgaggtcctgcctttcattggctgacacacctcctgtccactgtgtcactctccaagtgccagagaagtggaggcagatcgctaccccaggctgagatggcccccactgtgaaggccacgcctgtgggtgggcagccacctggtgccaccacagggcaccagggatgatcctgatgtggcaggcaggggagactcacagaaaaatctgcccagagcctaccctcaccagacaaactctgtgctcctccaaaacatcctttagatgcaaaataataataataataataataaataaataaataaaaatccaaacccaagtcaaaaccttggctccagcatgaaaacacgtttacaggaaagtgttctcctgggtttgtgcccaccatggtgcgaatcctgacccaaggcctcctgtctcccttcaaagggagacccttttgggggatgagtttgccagactccccgtgctggtttctttgttactatttgtttggggttttgttttagttctttttttttttcttttcttttttaaaaatatgtggctgtgaacttgaatgaacactgctcaaactttctgctattggggggggcgggtgggatgggaagaaggggcgtttgttttattcttggtgttttcagtgcaataaatagctacaaacttctgtgc

SEQ ID NO: 17 - DNA, 13109 bp, Homo sapiens
ggggaagcagtggccgtgtgagcgtgaggagctgccgccaccgcctgctcctcgtcctcctcgtcctccggggccccagcgtcgtgggccgcgcacggccctggaagagacgtcgcctcgccttcatccgcctctctcaccgcgccgctccctcgtcctgccctgcgggctcaggcggaacccggaacggccgtcctcttcccccgccctccgccgccgcctcctcctcctccttctcggcttcctcctcagccccgggccggagcggggtgtcggcggcggccggttcgggcggcggcgcttggccatgtcgtgtcggggaaggtaatgagccgcagagccccggggtctcggctgagcagcggcggcaccaactattcgcggagctggaatgactggcaacccagaactgatagtgcatcagctgacccaggtaatttaaaatattcttcatccagagatagaggtggttcttcctcttacggactgcaaccttcaaattcagctgtggtgtctcggcaaaggcacgatgataccagagtccacgctgacatacagaatgacgaaaagggtggctacagtgtcaatggaggatctggggaaaatacttatggtcggaagtcgttggggcaagagctgagggttaacaatgtgaccagccctgagttcaccagtgttcagcatggcagtcgtgctttagccaccaaagacatgaggaaatcacaggagagatcgatgtcttattctgatgagtctcgactgtcgaatcttcttcggaggatcacccgggaagacgacagagaccgaagattggctactgtaaagcagttgaaagaatttattcagcaaccagaaaataagctggtactagttaaacaattggataatatcttggctgctgtacatgacgtgcttaatgaaagtagcaaattgcttcaggagttgagacaggagggagcttgctgtcttggccttctttgtgcttctctgagctatgaggctgagaagatcttcaagtggatttttagcaaatttagctcatctgcaaaagatgaagttaaactcctctacttatgtgccacctacaaagcactagagactgtaggagaaaagaaagccttttcatctgtaatgcagcttgtaatgaccagcctgcagtctattcttgaaaatgtggatacaccagaattgctttgtaaatgtgttaagtgcattcttttggtggctcgatgttaccctcatattttcagcactaattttagggatacagttgatatattagttggatggcatatagatcatactcagaaaccttcgctcacgcagcaggtatctgggtggttgcagagtttggagccattttgggtagctgatcttgcattttctactactcttcttggtcagtttctggaagacatggaagcatatgctgaggacctcagccatgtggcctctggggaatcagtggatgaagatgtccctcctccatcagtgtcattaccaaagctggctgcacttctccgggtatttagtactgtggtgaggagcattggggaacgcttcagcccaattcggggtcctccaattactgaggcatatgtaacagatgttctgtacagagtaatgagatgtgtgacggctgcaaaccaggtgtttttttctgaggctgtgttgacagctgctaatgagtgtgttggtgttttgctcggcagcttggatcctagcatgactatacattgtgacatggtcattacatatggattagaccaactggagaattgccagacttgtggtaccgattatatcatctcagtcttgaatttactcacgctgattgttgaacagataaatacgaaactgccatcatcatttgtagaaaaactgtttataccatcatctaaactactattcttgcgttatcataaagaaaaagaggttgttgctgtagcccatgctgtttatcaagcagtgctcagcttgaagaatattcctgttttggagactgcctataagttaatattgggagaaatgacttgtgccctaaacaacctcctacacagtctacaacttcctgaggcctgttctgaaataaaacatgaggcttttaagaatcatgtgttcaatgtagacaatgcaaaatttgtagttaaatttgacctcagtgccctgactacaattggaaatgccaaaaactcactaatagggatgtgggcgctatctccaactgtctttgcacttctgagtaagaatctgatgattgtgcacagtgacctggctgttcacttccctgccattcagtatgctgtgctctacacattgtattctcattgtaccaggcatgatcactttatctctagtagcctcagttcttcctctccttctttgtttgatggagctgtgattagcactgtaactacggctacaaagaaacatttctcaattatattaaatcttctgggaatattacttaagaaagataaccttaaccaggacacgaggaaactgttaatgacttgggctttggaagcagctgttttaatgaagaagtctgaaacatacgcacctttattctctcttccgtctttccataaattttgcaaaggccttttagccaacactctcgttgaagatgtgaatatctgtctgcaggcatgcagcagtctacatgctctgtcctcttccttgccagatgatcttttacagagatgtgtcgatgtttgccgtgttcaactagtgcacagtggaactcgtattcgacaagcatttggaaaactgttgaaatcaattcctttagatgttgtcctaagcaataacaatcacacagaaattcaagaaatttctttagcattaagaagtcacatgagtaaagcaccaagtaatacattccacccccaagatttctctgatgttattagttttattttgtatgggaactctcatagaacagggaaggacaattggttggaaagactgttctatagctgccagagactggataagcgtgaccagtcaacaattccacgcaatctcctgaagacagatgctgtcctttggcagtgggccatatgggaagctgcacaattcactgttctttctaagctgagaaccccactgggcagagctcaagacaccttccagacaattgaaggtatcattcgaagtctcgcagctcacacattaaaccctgatcaggatgttagtcagtggacaactgcagacaatgatgaaggccatggtaacaaccaacttagacttgttcttcttctgcagtatctggaaaatctggagaaattaatgtataatgcatacgagggatgtgctaatgcattaacttcacctcccaaggtcattagaacttttttctataccaatcgccaaacttgtcaggactggctaacgcggattcgactctccatcatgagggtaggattgttggcaggccagcctgcagtgacagtgagacatggctttgacttgcttacagagatgaaaacaaccagcctatctcaggggaatgaattggaagtaaccattatgatggtggtagaagcattatgtgaacttcattgtcctgaagctatacagggaattgctgtctggtcatcatctattgttggaaaaaatcttctgtggattaactcagtggctcaacaggctgaagggaggtttgaaaaggcctctgtggagtaccaggaacacctgtgtgccatgacaggtgttgattgctgcatctccagctttgacaaatcggtgctcaccttagccaatgctgggcgtaacagtgccagcccgaaacattctctgaatggtgaatccagaaaaactgtgctgtccaaaccgactgactcttcccctgaggttataaattatttaggaaataaagcatgtgagtgctacatctcaattgccgattgggctgctgtgcaggaatggcagaacgctatccatgacttgaaaaagagtaccagtagcacttccctcaacctgaaagctgacttcaactatataaaatcattaagcagctttgagtctggaaaatttgttgaatgtaccgagcagttagaattgttaccaggagaaaatatcaatctacttgctggaggatcaaaagaaaaaatagacatgaaaaaactgcttcctaacatgttaagtccggatccgagggaacttcagaaatccattgaagttcaattgttaagaagttctgtttgtttggcaactgctttaaacccgatagaacaagatcagaagtggcagtctataactgaaaatgtggtaaagtacttgaagcaaacatcccgcatcgctattggacctctgagactttctactttaacagtttcacagtctttgccagttctaagtaccttgcagctgtattgctcatctgctttggagaacacagtttctaacagactttcaacagaggactgtcttattccactcttcagtgaagctttacgttcatgtaaacagcatgacgtgaggccatggatgcaggcattaaggtatactatgtaccagaatcagttgttggagaaaattaaagaacaaacagtcccaattagaagccatctcatggaattaggtctaacagcagcaaaatttgctagaaaacgagggaatgtgtcccttgcaacaagactgctggcacagtgcagtgaagttcagctgggaaagaccaccactgcacaggatttagtccaacattttaaaaaactatcaacccaaggtcaagtggatgaaaaatgggggcccgaacttgatattgaaaaaaccaaattgctttatacagcaggccagtcaacacatgcaatggaaatgttgagttcttgtgccatatctttctgcaagtctgtgaaagctgaatatgcagttgctaaatcaattctgacactggctaaatggatccaggcagaatggaaagagatttcaggacagctgaaacaggtttacagagctcagcaccaacagaacttcacaggtctttctactttgtctaaaaacatactcactctaatagaactgccatctgttaatacgatggaagaagagtatcctcggatcgagagtgaatctacagtgcatattggagttggagaacctgacttcattttgggacagttgtatcacctgtcttcagtacaggcacctgaagtagccaaatcttgggcagcgttggccagctgggcttataggtggggcagaaaggtggttgacaatgccagtcagggagaaggtgttcgtctgctgcctagagaaaaatctgaagttcagaatctacttccagacactataactgaggaagagaaagagagaatatatggtattcttggacaggctgtgtgtcggccggcggggattcaggatgaagatataacacttcagataactgagagtgaagacaacgaagaagatgacatggttgatgttatctggcgtcagttgatatcaagctgcccatggctttcagaacttgatgaaagtgcaactgaaggagttattaaagtgtggaggaaagttgtagatagaatattcagcctgtacaaactctcttgcagtgcatactttactttccttaaactcaacgctggtcaaattcctttagatgaggatgaccctaggctgcatttaagtcacagagtggaacagagcactgatgacatgattgtgatggccacattgcgcctgctgcggttgctcgtgaagcacgctggtgagcttcggcagtatctggagcacggcttggagacaacacccactgcaccatggagaggaattattccgcaacttttctcacgcttaaaccaccctgaagtgtatgtgcgccaaagtatttgtaaccttctctgccgtgtggctcaagattccccacatctcatattgtatcctgcaatagtgggtaccatatcgcttagtagtgaatcccaggcttcaggaaataaattttccactgcaattccaactttacttggcaatattcaaggagaagaattgctggtttctgaatgtgagggaggaagtcctcctgcatctcaggatagcaataaggatgaacctaaaagtggattaaatgaagaccaagccatgatgcaggattgttacagcaaaattgtagataagctgtcctctgcaaaccccaccatggtattacaggttcagatgctcgtggctgaactgcgcagggtcactgtgctctgggatgagctctggctgggagttttgctgcaacaacacatgtatgtcctgagacgaattcagcagcttgaagatgaggtgaagagagtccagaacaacaacaccttacgcaaagaagagaaaattgcaatcatgagggagaagcacacagctttgatgaagcccatcgtatttgctttggagcatgtgaggagtatcacagcggctcctgcagaaacacctcatgaaaaatggtttcaggataactatggtgatgccattgaaaatgccctagaaaaactgaagactccattgaaccctgcaaagcctgggagcagctggattccatttaaagagataatgctaagtttgcaacagagagcacagaaacgtgcaagttacatcttgcgtcttgaagaaatcagtccatggttggctgccatgactaacactgaaattgctcttcctggggaagtctcagccagagacactgtcacaatccatagtgtgggcggaaccatcacaatcttaccgactaaaaccaagccaaagaaacttctctttcttggatcagatgggaagagctatccttatcttttcaaaggactggaggatttacatctggatgagagaataatgcagttcctatctattgtgaataccatgtttgctacaattaatcgccaagaaacaccccggttccatgctcgacactattctgtaacaccactaggaacaagatcaggactaatccagtgggtagatggagccacacccttatttggtctttacaaacgatggcaacaacgggaagctgccttacaagcacaaaaggcccaagattcctaccaaactcctcagaatcctggaattgtaccccgtcctagtgaactttattacagtaaaattggccctgctttgaaaacagttgggcttagcctggatgtgtcccgtcgggattggcctcttcatgtaatgaaggcagtattggaagagttaatggaggccacacccccgaatctccttgccaaagagctctggtcatcttgcacaacacctgatgaatggtggagagttacgcagtcttatgcaagatctactgcagtcatgtctatggttggatacataattggccttggagacagacatctggataatgttcttatagatatgacgactggagaagttgttcacatagattacaatgtttgctttgaaaaaggtaaaagccttagagttcctgagaaagtaccttttcgaatgacacaaaacattgaaacagcactgggtgtaactggagtagaaggtgtatttaggctttcatgtgagcaggttttacacattatgcggcgtggcagagagaccctgctgacgctgctggaggcctttgtgtacgaccctctggtggactggacagcaggaggcgaggctgggtttgctggtgctgtctatggtggaggtggccagcaggccgagagcaagcagagcaagagagagatggagcgagagatcacccgcagcctgttttcttctagagtagctgagattaaggtgaactggtttaagaatagagatgagatgctggttgtgcttcccaagttggacggtagcttagatgaatacctaagcttgcaagagcaactgacagatgtggaaaaactgcagggcaaactactggaggaaatagagtttctagaaggagctgaaggggtggatcatccttctcatactctgcaacacaggtattctgagcacacccaactacagactcagcaaagagctgttcaggaagcaatccaggtgaagctgaatgaatttgaacaatggataacacattatcaggctgcattcaataatttagaagcaacacagcttgcaagcttgcttcaagagataagcacacaaatggaccttggtcctccaagttacgtgccagcaacagcctttctgcagaatgctggtcaggcccacttgattagccagtgcgagcagctggagggggaggttggtgctctcctgcagcagaggcgctccgtgctccgtggctgtctggagcaactgcatcactatgcaaccgtggccctgcagtatccgaaggccatatttcagaaacatcgaattgaacagtggaagacctggatggaagagctcatctgtaacaccacagtagagcgttgtcaagagctctataggaaatatgaaatgcaatatgctccccagccacccccaacagtgtgtcagttcatcactgccactgaaatgaccctgcagcgatacgcagcagacatcaacagcagacttattagacaagtggaacgcttgaaacaggaagctgtcactgtgccagtttgtgaagatcagttgaaagaaattgaacgttgcattaaagttttccttcatgagaatggagaagaaggatctttgagtctagcaagtgttattatttctgccctttgtacccttacaaggcgtaacctgatgatggaaggtgcagcgtcaagtgctggagaacagctggttgatctgacttctcgggatggagcctggttcttggaggaactctgcagtatgagcggaaacgtcacctgcttggttcagttactgaagcagtgccacctggtgccacaggacttagatatcccgaaccccatggaagcgtctgagacagttcacttagccaatggagtgtatacctcacttcaggaattgaattcgaatttccggcaaatcatatttccagaagcacttcgatgtttaatgaaaggggaatacacgttagaaagtatgctgcatgaactggacggtcttattgagcagaccaccgatggcgttcccctgcagactctagtggaatctcttcaggcctacttaagaaacgcagctatgggactggaagaagaaacacatgctcattacatcgatgttgccagactactacatgctcagtacggtgaattaatccaaccgagaaatggttcagttgatgaaacacccaaaatgtcagctggccagatgcttttggtagcattcgatggcatgtttgctcaagttgaaactgctttcagcttattagttgaaaagttgaacaagatggaaattcccatagcttggcgaaagattgacatcataagggaagccaggagtactcaagttaatttttttgatgatgataatcaccggcaggtgctagaagagattttctttctaaaaagactacagactattaaggagttcttcaggctctgtggtaccttttctaaaacattgtcaggatcaagttcacttgaagatcagaatactgtgaatgggcctgtacagattgtcaatgtgaaaaccctttttagaaactcttgtttcagtgaagaccaaatggccaaacctatcaaggcattcacagctgactttgtgaggcagctcttgatagggctacccaaccaagccctcggactcacactgtgcagttttatcagtgctctgggtgtagacatcattgctcaagtagaggcaaaggactttggtgccgaaagcaaagtttctgttgatgatctctgtaagaaagcggtggaacataacatccagatagggaagttctctcagctggttatgaacagggcaactgtgttagcaagttcttacgacactgcctggaagaagcatgacttggtgcgaaggctagaaaccagtatttcttcttgtaagacaagcctgcagcgggttcagctgcatattgccatgtttcagtggcaacatgaagatctacttatcaatagaccacaagccatgtcagtcacacctcccccacggtctgctatcctaaccagcatgaaaaagaagctgcataccctgagccagattgaaacttctattgcgacagttcaggagaagctagctgcacttgaatcaagtattgaacagcgactcaagtgggcaggtggtgccaaccctgcattggcccctgtactacaagattttgaagcaacgatagctgaaagaagaaatcttgtccttaaagagagccaaagagcaagtcaggtcacatttctctgcagcaatatcattcattttgaaagtttacgaacaagaactgcagaagccttaaacctggatgcggcgttatttgaactaatcaagcgatgtcagcagatgtgttcgtttgcatcacagtttaacagttcagtgtctgagttagagcttcgtttattacagagagtggacactggtcttgaacatcctattggcagctctgaatggcttttgtcagcacacaaacagttgacccaggatatgtctactcagagggcaattcagacagagaaagagcagcagatagaaacggtctgtgaaacaattcagaatctggttgataatataaagactgtgctcactggtcataaccgacagcttggagatgtcaaacatctcttgaaagctatggctaaggatgaagaagctgctctggcagatggtgaagatgttccctatgagaacagtgttaggcagtttttgggtgaatataaatcatggcaagacaacattcaaacagttctatttacattagtccaggctatgggtcaggttcgaagtcaagaacacgttgaaatgctccaggaaatcactcccaccttgaaagaactgaaaacacaaagtcagagtatctataataatttagtgagttttgcatcacccttagtcaccgatgcaacaaatgaatgttcgagtccaacgtcatctgctacttatcagccatccttcgctgcagcagtccggagtaacactggccagaagactcagcctgatgtcatgtcacagaatgctagaaagctgatccagaaaaatcttgctacatcagctgatactccaccaagcaccgttccaggaactggcaagagtgttgcttgtagtcctaaaaaggcagtcagagaccctaaaactgggaaagcggtgcaagagagaaactcctatgcagtgagtgtgtggaagagagtgaaagccaagttagagggccgagatgttgatccgaataggaggatgtcagttgctgaacaggttgactatgtcattaaggaagcaactaatctagataacttggctcagctgtatgaaggttggacagcctgggtgtgaatggcaagacagtagatgagtctggttaagcgaggtcagacatccaccagaatcaactcagcctcaggcatccaaagccacaccacagtcggtggtgatgcaactgggggcttactctgaggaaacctaggaaatctcggtgcactaggaagtgaatcccgcaggacagctgcactcagggatacgcccaacaccatggcctgcaaccccagggtcaagggtgaaggaaagcaagctcaccgcctgaacacggagattgtctttctgccacagaacagcagcagacgtgtcgggaggttagctgcggaaagaaatcgggatgccgcggagcacagagtgatttggaactccattccacctgaccctgtgtgtacaatccaggaaaaaaacaaaccccactcagaaacagagaaaactggggtcgcgaagaaatcacagccaaggaagatttgatgcattcagattctcgtgtaacacttgttgcttggcaacagtactggttgggttgaccagtaagtagaaaaaggctaaaggctatgcgatatgaatttcagaaatggactgaaaatggagagctatgtaacagatacactacagtagaagaacttacttctgaaatgaagggaaaaaaaccaccccatcgttccctactcctccccaccacttacccgttccccctttacctaatctagtagattagccatctttcaaattcacttttatttcagtccttatatttcatatacttccgtctcgatgctgttaacaacttctgataacatggaaaattcaaggattgtttaaaggtctgatgatcacacacaaaatgtaattccggttatttaagtcatttctgtgattctatcatgtacagtttccagaattgtcactgtgcattcaaaagtaatgaatctaacagacatttgatttaatgtacactcccttttgcttatagtgtgcattttttttggaggtcattcaaattttccctcttctgtgatagctgtagtttctttcatagaaagtagctaatccagtgtaatcttttacctttttaaaaaccaagatagagtatctattagagttttacattgttgatgatagattaacaataaagtgatgttctggtggaggtagactgaaatttttttaattcatgtttttcatttgatacttttaatttacacttagtaaattaaaagttgtttaatttacttggcattttaggacatgtacatgaaacagtgaaaatgagatccaccaacatcttttattaagttcagttattagtctgtgaagtgctttactttttgcacaattttaatagcttgctattcagtaatacattatagtgaattcatgatcaaggtttccttaaatttagcattgcatttcagtactgactgtgtaagctaaattgctgatccaaaataaaaacccagactagaatagggttcttaaaatcaagtatcaatacaaaatagaacacaattaaaatcttaattgttggctgggcacagtggctcacgcctgtaatcccagcactttgggaggccgaggcgggcggatcatgaggttaggagagcgagaccatcctggctaacacggtgaaaccccgtctttactaaaatacaaaaaaaattagccgggcgtggtggcgggcgcctgtagtcccagctactcgggaggctgaggcaggagaatggcgtgaacccaggaggcggagcttgcagtgagccgagattgtgccactgcactccagcctgggcaacagagctagactctgtgtcaaaaataaatgactagat

SEQ ID NO: 18 - DNA, 1952 bp, Homo sapiens
tggggagctgctccggcttcggcgcggaggggcggcggccggggaggcggcggcggcggcaggattcccaggagccatgttgtcagaagtcctactggtgtctgctccggggaaagtcatccttcatggagaacatgccgtggtacatggcaaggtagcactggctgtatccttgaacttgagaacattcctccggcttcaaccccacagcaatgggaaagtggacctcagcttacccaacattggtatcaagcgggcctgggatgtggccaggcttcagtcactggacacaagctttctggagcaaggtgatgtcacaacacccacctcagagcaagtggagaagctaaaggaggttgcaggcttgcctgacgactgtgctgtcaccgagcgcctggctgtgctggcctttctttacttatacctgtccatctgccggaagcagagggccctgccgagcctggatatcgtagtgtggtcggagctgccccccggggcgggcttgggctccagcgccgcctactcggtgtgtctggcagcagccctcctgactgtgtgcgaggagatcccaaacccgctgaaggacggggattgcgtcaacaggtggaccaaggaggatttggagctaattaacaagtgggccttccaaggggagagaatgattcacgggaacccctccggagtggacaatgctgtcagcacctggggaggagccctccgataccatcaagggaagatttcatccttaaagaggtcgccagctctccagatcctgctgaccaacaccaaagtccctcgcaataccagggcccttgtggctggcgtcagaaacaggctgctcaagttcccagagatcgtggcccccctcctgacctcaatagatgccatctccctggagtgtgagcgcgtgctgggagagatgggggaagccccagccccggagcagtacctcgtgctggaagagctcattgacatgaaccagcaccatctgaatgccctcggcgtgggccacgcctctctggaccagctctgccaggtgaccagggcccgcggacttcacagcaagctgactggcgcaggcggtggtggctgtggcatcacactcctcaagccagggctggagcagccagaagtggaggccacgaagcaggccctgaccagctgtggctttgactgcttggaaaccagcatcggtgcccccggcgtctccatccactcagccacctccctggacagccgagtccagcaagccctggatggcctctgagaggagcccacgacactgcagccccacccagatgcccctttctggattattctgggggctgcagttcgactctgtgctggccagcgagcgcccagctcctgacactgctggagaggccccagccgcttggcgatgccagccaagctctgcagtcccagcggtgggacctagggaggcatggtctgccctctgcatcctctggagccagccgagcaggaggcctaggagggtcctctgagactccagacctgaggcgagaagggctgcttccctgaagctcccacagtcccatctgcttcaggcccccgccttggcctgtgttcttcctggccgcctgggtccaatgctcaggtgctggggcctggttcccggagaagtgtgccttctctctcccttttcagggacggccccctgtctctcagggccaggcctctccctcctccaggaagccttcccctaccccttgtcgcccctccctcccagagcacctgctgtctgggtggctcactcagcacttggcccttctacctagcgggatggggctcccccaggggctgtcccggaggcggtgggcctggttaaataaggcagtgtggccttggtttatatgcactttcttccgatctgtacctgagaggtttgtggaaaagatggcaaatggggaataaaaagattttgtgtcaac

SEQ ID NO: 19 - DNA, 5876 bp, Homo sapiens
ggaattcatctgtaaaaatcactacatgtaacgtaggagacaagaaaaatattaatgacagaagatctgcgaacatgatgcacgtgaataattttccctttagaaggcattcctggatatgttttgatgtggacaatggcacatctgcgggacggagtcccttggatcccatgaccagcccaggatccgggctaattctccaagcaaattttgtccacagtcaacgacgggagtccttcctgtatcgatccgacagcgattatgacctctctccaaagtctatgtcccggaactcctccattgccagtgatatacacggagatgacttgattgtgactccatttgctcaggtcttggccagtctgcgaactgtacgaaacaactttgctgcattaactaatttgcaagatcgagcacctagcaaaagatcacccatgtgcaaccaaccatccatcaacaaagccaccataacagaggaggcctaccagaaactggccagcgagaccctggaggagctggactggtgtctggaccagctagagaccctacagaccaggcactccgtcagtgagatggcctccaacaagtttaaaaggatgcttaatcgggagctcacccatctctctgaaatgagtcggtctggaaatcaagtgtcagagtttatatcaaacacattcttagataagcaacatgaagtggaaattccttctccaactcagaaggaaaaggagaaaaagaaaagaccaatgtctcagatcagtggagtcaagaaattgatgcacagctctagtctgactaattcaagtatcccaaggtttggagttaaaactgaacaagaagatgtccttgccaaggaactagaagatgtgaacaaatggggtcttcatgttttcagaatagcagagttgtctggtaaccggcccttgactgttatcatgcacaccatttttcaggaacgggatttattaaaaacatttaaaattccagtagatactttaattacatatcttatgactctcgaagaccattaccatgctgatgtggcctatcacaacaatatccatgctgcagatgttgtccagtctactcatgtgctattatctacacctgctttggaggctgtgtttacagatttggagattcttgcagcaatttttgccagtgcaatacatgatgtagatcatcctggtgtgtccaatcaatttctgatcaatacaaactctgaacttgccttgatgtacaatgattcctcagtcttagagaaccatcatttggctgtgggctttaaattgcttcaggaagaaaactgtgacattttccagaatttgaccaaaaaacaaagacaatctttaaggaaaatggtcattgacatcgtacttgcaacagatatgtcaaaacacatgaatctactggctgatttgaagactatggttgaaactaagaaagtgacaagctctggagttcttcttcttgataattattccgataggattcaggttcttcagaatatggtgcactgtgcagatctgagcaacccaacaaagcctctccagctgtaccgccagtggacggaccggataatggaggagttcttccgccaaggagaccgagagagggaacgtggcatggagataagccccatgtgtgacaagcacaatgcttccgtggaaaaatcacaggtgggcttcatagactatattgttcatcccctctgggagacatgggcagacctcgtccaccctgacgcccaggatattttggacactttggaggacaatcgtgaatggtaccagagcacaatccctcagagcccctctcctgcacctgatgacccagaggagggccggcagggtcaaactgagaaattccagtttgaactaactttagaggaagatggtgagtcagacacggaaaaggacagtggcagtcaagtggaagaagacactagctgcagtgactccaagactctttgtactcaagactcagagtctactgaaattccccttgatgaacaggttgaagaggaggcagtaggggaagaagaggaaagccagcctgaagcctgtgtcatagatgatcgttctcctgacacgtaacagtgcaaaaactttcatgccttttttttttttaagtagaaaaattgtttccaaagtgcatgtcacatgccacaaccacggtcacacctcactgtcatctgccaggacgtttgttgaacaaaactgaccttgactactcagtccagcgctcaggaatatcgtaaccagttttttcacctccatgtcatccgagcaaggtggacatcttcacgaacagcgtttttaacaagatttcagcttggtagagctgacaaagcagataaaatctactccaaattattttcaagagagtgtgactcatcaggcagcccaaaagtttattggacttggggtttctattcctttttatttgtttgcaatattttcagaagaaaggcattgcacagagtgaacttaatggacgaagcaacaaatatgtcaagaacaggacatagcacgaatctgttaccagtaggaggaggatgagccacagaaattgcataattttctaatttcaagtcttcctgatacatgactgaatagtgtggttcagtgagctgcactgacctctacattttgtatgatatgtaaaacagattttttgtagagcttacttttattattaaatgtattgaggtattatatttaaaaaaaactatgttcagaacttcatctgccactggttatttttttctaaggagtaacttgcaagttttcagtacaaatctgtgctacactggataaaaatctaatttatgaattttacttgcaccttatagttcatagcaattaactgatttgtagtgattcattgtttgttttatataccaatgacttccatattttaaaagagaaaaacaactttatgttgcaggaaaccctttttgtaagtctttattatttactttgcattttgtttcactctttccagataagcagagttgctcttcaccagtgtttttcttcatgtgcaaagtgactatttgttctataatacttttatgtgtgttatatcaaatgtgtcttaagcttcatgcaaactcagtcatcagttcgtgttgtctgaagcaagtgggaaatatataaatacccagtagctaaaatggtcagtcttttttagatgttttcctacttagtatctcctaataacgttttgctgtgtcactagatgttcatttcacaagtgcatgtctttctaataatccacacatttcatgctctaataatccacacatttcatgctcatttttattgtttttacagccagttatagcaagaaaaaggtttttccccttgtgctgctttataatttagcgtgtgtctgaaccttatccatgtttgctagatgaggtcttgtcaaatatatcactaccattgtcaccggtgaaaagaaacaggtagttaagttagggttaacattcatttcaaccacgaggttgtatatcatgactagcttttactcttggtttacagagaaaagttaaacaaccaactaggcagtttttaagaatattaacaatatattaacaaacaccaatacaactaatcctatttggttttaatgatttcaccatgggattaagaactatatcaggaacatccctgagaaacggctttaagtgtagcaactactcttccttaatggacagccacataacgtgtaggaagtcctttatcacttatcctcgatccataagcatatcttgcagaggggaactacttctttaaacacatggagggaaagaagatgatgccactggcaccagagggttagtactgtgatgcatcctaaaatatttattatattggtaaaaattctggttaaataaaaaattagagatcactcttggctgatttcagcaccaggaactgtattacagttttagagattaattcctagtgtttacctgattatagcagttggcatcatggggcatttaattctgactttatccccacgtcagccttaataaagtcttctttaccttctctatgaagactttaaagcccaaataatcatttttcacattgatattcaagaattgagatagatagaagccaaagtgggtatctgacaagtggaaaatcaaacgtttaagaagaattacaactctgaaaagcatttatatgtggaacttctcaaggagcctcctggggactggaaagtaagtcatcagccaggcaaatgactcatgctgaagagagtccccatttcagtcccctgagatctagctgatgcttagatcctttgaaataaaaattatgtctttataactctgatcttttacataaagcagaagaggaatcaactagttaattgcaaggtttctactctgtttcctctgtaaagatcagatggtaatctttcaaataagaaaaaaataaagacgtatgtttgaccaagtagtttcacaagaatatttgggaacttgtttcttttaattttatttgtccctgagtgaagtctagaaagaaaggtaaagagtctagagtttattcctctttccaaaacattctcattcctctcctccctacacttagtatttcccccacagagtgcctagaatcttaataatgaataaaataaaaagcagcaatatgtcattaacaaatccagacctgaaagggtaaagggtttataactgcactaataaagagaggctctttttttttcttccagtttgttggtttttaatggtaccgtgttgtaaagatacccactaatggacaatcaaattgcagaaaaggctcaatatccaagagacagggactaatgcactgtacaatctgcttatccttgcccttctctcttgccaaagtgtgcttcagaaatatatactgctttaaaaaagaataaaagaatatccttttacaagtggctttacatttcctaaaatgccataagaaaatgcaatatctgggtactgtatggggaaaaaaatgtccaagtttgtgtaaaaccagtgcatttcagcttgcaagttactgaacacaataatgctgttttaattttgttttatatcagttaaaattcacaataatgtagatagaacaaattacagacaaggaaagaaaaaacttgaatgaaatggattttacagaaagctttatgataatttttgaatgcattatttattttttgtgccatgcattttttttctcaccaaatgaccttacctgtaatacagtcttgtttgtctgtttacaaccatgtatttattgcaatgtacatactgtaatgttaattgtaaattatctgttcttattaaaacatcatcccatgatggggtggtgttgatatatttggaaactcttggtgagagaatgaatggtgtgtatacatactctgtacatttttcttttctcctgtaatatagtcttgtcaccttagagcttgtttatggaagattcaagaaaactataaaatacttaaagatatataaatttaaaaaaacatagctgcaggtctttggtcccagggctgtgccttaactttaaccaatattttcttctgttttgctgcatttgaaaggtaacagtggagctagggctgggcattttacatccaggcttttaattgattagaattctgccaataggtggattttacaaaaccacagacaacctctgaaagattctgagacccttttgagacagaagctcttaagtacttcttgccagggagcagcactgcatgtgtgatggttgtttgccatctgttgatcaggaactacttcagctacttgcatttgattatttcctttttttttttttttaactcggaaacacaactgggggaat

SEQ ID NO: 20 - DNA, 4242 bp, Homo sapiens
gaattcggcgctgagtgacccgagtcgggacgcgggctgcgcgcgcgggaccccggagcccaaacccggggcaggcgggcagctgtgcccgggcggcacggccagcttcctgatttctcccgattccttccttctccctggagcggccgacaatgtccaccacggtcaatgtagattcccttgcggaatatgagaagagccagatcaagagagccctggagctggggacggtgatgactgtgttcagcttccgcaagtccacccccgagcggagaaccgtccaggtgatcatggagacgcggcaggtggcctggagcaagaccgccgacaagatcgagggcttcttggatatcatggaaataaaagaaatccgcccagggaagaactccaaagatttcgagcgagcaaaagcagttcgccagaaagaagactgctgcttcaccatcctatatggcactcagttcgtcctcagcacgctcagcttggcagctgactctaaagaggatgcagttaactggctctctggcttgaaaatcttacaccaggaagcgatgaatgcgtccacgcccaccattatcgagagttggctgagaaagcagatatattctgtggatcaaaccagaagaaacagcatcagtctccgagagttgaagaccatcttgcccctgatcaactttaaagtgagcagtgccaagttccttaaagataagtttgtggaaataggagcacacaaagatgagctcagctttgaacagttccatctcttctataaaaaacttatgtttgaacagcaaaaatcgattctcgatgaattcaaaaaggattcgtccgtgttcatcctggggaacactgacaggccggatgcctctgctgtttacctgcatgacttccagaggtttctcatacatgaacagcaggagcattgggctcaggatctgaacaaagtccgtgagcggatgacaaagttcattgatgacaccatgcgtgaaactgctgagcctttcttgtttgtggatgagttcctcacgtacctgttttcacgagaaaacagcatctgggatgagaagtatgacgcggtggacatgcaggacatgaacaaccccctgtctcattactggatctcctcgtcacataacacgtaccttacaggtgaccagctgcggagcgagtcgtccccagaagcttacatccgctgcctgcgcatgggctgtcgctgcattgaactggactgctgggacgggcccgatgggaagccggtcatctaccatggctggacgcggactaccaagatcaagtttgatgacgtcgtgcaggccatcaaagaccacgcctttgttacctcgagcttcccagtgatcctgtccatcgaggagcactgcagcgtggagcaacagcgtcacatggccaaggccttcaaggaagtatttggcgacctgctgttgacgaagcccacggaggccagtgctgaccagctgccctcgcccagccagctgcgggagaagatcatcatcaagcataagaagctgggcccccgaggcgatgtggatgtcaacatggaggacaagaaggacgaacacaagcaacagggggagctgtacatgtgggattccattgaccagaaatggactcggcactactgcgccattgctgatgccaagctgtccttcagtgatgacattgaacagactatggaggaggaagtgccccaggatataccccctacagaactacattttggggagaaatggttccacaagaaggtggagaagaggacgagtgccgagaagttgctgcaggaatactgcatggagacggggggcaaggatggcaccttcctggttcgggagagcgagaccttccccaatgactacaccctgtccttctggcggtcaggccgggtccagcactgccggatccgctccaccatggagggcgggaccctgaaatactacttgactgacaacctgaggttcaggaggatgtatgccctcatccagcactaccgcgagacgcacctgccgtgcgccgagttcgagctgcggctcacggaccctgtgcccaaccccaacccccacgagtccaagccgtggtactatgacagcctgagccgcggagaggcagaggacatgctgatgaggattccccgggacggggccttcctgatccggaagcgagaggggagcgactcctatgccatcaccttcagggctaggggcaaggtaaagcattgtcgcatcaaccgggacggccggcactttgtgctggggacctccgcctattttgagagtctggtggagctcgtcagttactacgagaagcattcactctaccgaaagatgagactgcgctaccccgtgacccccgagctcctggagcgctacaatacggaaagagatataaactccctctacgacgtcagcagaatgtatgtggatcccagtgaaatcaatccgtccatgcctcagagaaccgtgaaagctctgtatgactacaaagccaagcgaagcgatgagctgagcttctgccgtggtgccctcatccacaatgtctccaaggagcccgggggctggtggaaaggagactatggaaccaggatccagcagtacttcccatccaactacgtcgaggacatctcaactgcagacttcgaggagctagaaaagcagattattgaagacaatcccttagggtctctttgcagaggaatattggacctcaatacctataacgtcgtgaaagcccctcagggaaaaaaccagaagtcctttgtcttcatcctggagcccaaggagcagggcgatcctccggtggagtttgccacagacagggtggaggagctctttgagtggtttcagagcatccgagagatcacgtggaagattgacagcaaggagaacaacatgaagtactgggagaagaaccagtccatcgccatcgagctctctgacctggttgtctactgcaaaccaaccagcaaaaccaaggacaacttagaaaatcctgacttccgagaaatccgctcctttgtggagacgaaggctgacagcatcatcagacagaagcccgtcgacctcctgaagtacaatcaaaagggcctgacccgcgtctacccaaagggacaaagagttgactcttcaaactacgaccccttccgcctctggctgtgcggttctcagatggtggcactcaatttccagacggcagataagtacatgcagatgaatcacgcattgttttctctcaacgggcgcacgggctacgttctgcagcctgagagcatgaggacagagaaatatgacccgatgccacccgagtcccagaggaagatcctgatgacgctgacagtcaaggttctcggtgctcgccatctccccaaacttggacgaagtattgcctgtccctttgtagaagtggagatctgtggagccgagtatggcaacaacaagttcaagacgacggttgtgaatgataatggcctcagccctatctgggctccaacacaggagaaggtgacatttgaaatttatgacccaaacctggcatttctgcgctttgtggtttatgaagaagatatgttcagcgatcccaactttcttgctcatgccacttaccccattaaagcagtcaaatcaggattcaggtccgttcctctgaagaatgggtacagcgaggacatagagctggcttccctcctggttttctgtgagatgcggccagtcctggagagcgaagaggaactttactcctcctgtcgccagctgaggaggcggcaagaagaactgaacaaccagctctttctgtatgacacacaccagaacttgcgcaatgccaaccgggatgccctggttaaagagttcagtgttaatgagaaccactccagctgtaccaggagaaatgcaacaagaggttaagagagaagagagtcagcaacagcaagttttactcatagaagctggggtatgtgtgtaagggtattgtgtgtgtgcgcatgtgtgtttgcatgtaggagaacgtgccctattcacactctgggaagacgctaatctgtgacatcttttcttcaagcctgccatcaaggacatttcttaagacccaactggcatgagttggggtaatttcctattattttcatcttggacaacttctaacttatatctttatagaggattccccaaaatgtgctcctcatttttggcctctcatgttccaaacctcattgaataaaaagcaatgaaaaccttg

SEQ ID NO: 21 - DNA, 5000 bp, Homo sapiens
ggcacgagcagaagcaacaataattgtgaaaaatacttcagcagttatggactcatctgtcattcaaaggaaaaaagtagctgtcattggtggtggcttggttggctcattacaagcatgctttcttgcaaagaggaatttccagattgatgtatatgaagctagggaagatactcgagtggctaccttcacacgtggaagaagcattaacttagccctttctcatagaggacgacaagccttgaaagctgttggcctggaagatcagattgtatcccaaggtattcccatgagagcaagaatgatccactctctttcaggaaaaaagtctgcaattccctatgggacaaagtctcagtatattctttctgtaagcagagaaaatctaaacaaggatctattgactgctgctgagaaataccccaatgtgaaaatgcactttaaccacaggctgttgaaatgtaatccagaggaaggaatgatcacagtgcttggatctgacaaagttcccaaagatgtcacttgtgacctcattgtaggatgtgatggagcctattcaactgtcagatctcacctgatgaagaaacctcgctttgattacagtcagcagtacattcctcatgggtacatggagttgactattccacctaagaacggagattatgccatggaacctaattatctgcatatttggcctagaaatacctttatgatgattgcacttcctaacatgaacaaatcattcacatgtactttgttcatgccctttgaagagtttgaaaaacttctaaccagtaatgatgtggtagatttcttccagaaatactttccggatgccatccctctaattggagagaaactcctagtgcaagatttcttcctgttgcctgcccagcccatgatatctgtaaagtgctcttcatttcactttaaatctcactgtgtactgctgggagatgcagctcatgctatagtgccgttttttgggcaaggaatgaatgcgggctttgaagactgcttggtatttgatgagttaatggataaattcagtaacgaccttagtttgtgtcttcctgtgttctcaagattgagaatcccagatgatcacgcgatttcagacctatccatgtacaattacatagagatgcgagcacatgtcaactcaagctggttcatttttcagaagaacatggagagatttcttcatgcgattatgccatcgacctttatccctctctatacaatggtcactttttccagaataagataccatgaggctgtgcagcgttggcattggcaaaaaaaggtgataaacaaaggactctttttcttgggatcactgatagccatcagcagtacctacctacttatacactacatgtcaccacgatctttcctctgcttgagaagaccatggaactggatagctcacttccggaatacaacatgtttccccgcaaaggccgtggactccctagaacaaatttccaatctcattagcaggtgatagaaaggttttgtggtagcaaatgcatgatttctctgtgaccaaaattaagcatgaaaaaaatgtttccattgccatatttgattcactagtggaagatagtgttctgcttataattaaactgaatgtagagtatctctgtatgttaattgcaattactggttggggggtgcattttaaaagatgaaacatgcagcttccctacattacacacactcaggttgagtcattctaactataaaagtgcaatgactaagatccttcacttctctgaaagtaaggccctagatgcctcagggaagacagtaatcatgccttttctttaaaagacacaataggactcgcaacagcattgactcaacacctaggactaaaaatcacaacttaactagcatgttaactgcacttttcattacgtgaatggaacttacctaaccacagggctcagacttactagataaaaccagaaatggaaataaggaattcaggggagttccagagacttacaaaatgaactcattttattttcccaccttcaaatataagtattatcatctatctgtttatcgtctatctatctatcatctatctatctatctatcatctatctatctatctatctatctatctatctatctatctatctatctatctctatttatttatgtatttagagatcaggtctcactctgttgaccaggctggagtgcagtggtgagatctgggttcactgcaacctctgcctcctgggctcaagcaatcctcccacttcagcctcccaaatagctggggctaccatggtatttttcagtagagaccgggtcttgccatgctgcccaggccagtctcaaactcctggcctcatgtgatctgcccacctcagcctcccaaagtacagggattagagttgtgagccaccgctgccagcccagagttaccctctaaagataagaaaaaggctattaatatcatactaagtgaaggacaggaaagggttttattcataaattaaatgtctacatgtgccagaatggaaaggaaacaaggggagacaacttttatagaaatacaaagccattactttattcaatttcagaccctcagaagcaatttactaatttattcttcgactacatactgcagcagaaccagcaatacacttgatttttaaaagcacatttagtgaaatgttttctttggttcatccttctttaacaggctgctgagtcactcagaaatccttcaaacatgattaattatgaagatgaaacactagagtcatataagaaataaaaattgggcaataaaataaaatgattcagtgtttcttttctatattgtcaatgaaaaccttgagttctaataatccatgttcagtttgtagggaaagaaaaaataatttttccttctacccactttaggttccttggctggggcccctataacaaaagacagattgacaagagaaaaacaaacataaatttattagcgggtatatgtaatatatatgtgggaaatacaggggaatgagcaaatctcaaagagctggcgtcttagaactccctggcttatatagcatcgacaaagaacagtaaatttttagagaaacaacaaaacaaagaaaaagagctttgagtctgtaggggcagcaatttgggggaagcaaatatatgggagtttgccttgtagattcctctggtggtggtctccaggctgacaaggattcaaagttgtctctgaaactcctctttgtcatactgcacatataaaacgtcttttgtttccaacaagaggatttctttttcattctagaattatctccttgataacttgatcagatataggacatgacactgaatagagtccaacagtacaaaaaaaattcagtatgttctagctacttcacacatgtgtacgcgacagttatttttacagtaaggtattttcgagaaaaatgcattacgtgttttggaaaatagagtaatttaaaaaatatatttgaaatgaaaatctccaacacattagaagatgatgatgttagatgcccatcgtgtgccacaagtggttttttcattatgtaaagcacccgttgaattaaaagaatttgtttttgttcaacctcttcctgaggcccaagagcatatgggcaattcggatttcctgctggaccacaaggttctgttgatattacatagaaacgggtattccagacacttcttatgatgaaagtccaaaagtggcatccaatttaaggccccatctttcgttgccattcttcattcctacaaaggacgaacttggattacatcaactttggacccattggttttgtcgctgtcgtcaactgacagtgattcaccactggtgatgataaaaatgatggaagaagagttgaaagtcacttttttctttggcctgtccccatctttctgtgacatcacaatgggtctgatctgcatttcacttccagctgctggtaggtctttagcaggcctctggcacctcagcagtcggaggcacagaagctgcaaaagggatcttcgaaactgggcagagaaaaaataaagtggaatattaagtaaaagttgggcactaatctggattaacattcgaggaaatcagttgagctgatttaagttgttttttgtttgttagcaggtgtggatgtggggttatgtggtcatgctcagatctacctaaatcaccccagagctttatgtcttttattcattctaaatcttattaaccggaatatgtaggaccatttcaataccttgtaatcctccaagcttcaatctgcacacactttctatgagggcaggtacaactattaagagattttgaacattaagttagtccacaaatattcagtgggcatctactaggtgacagccactgtgctataattagagactttttactataagcatcaaaaacagataaggctcttcctggcagagtttacagcctggtgtacttgctaatgtctctttaattaggtgaagaattttttttttctatcgaaattactaatcagttggggaaaaaaatactatagcagacagcactaatgtcatcaacaaacattgttcttctccgtgtcctgggtacaacatcgaataatatttcttggcctcctttccgcttctcctctctgctgttcctctctacaagaacctgggaggccaacgcctaaagatcataatatcacaatggaaggaacctagattcctaaatgactgcataggacagatcccatctcctccacccaatacattattagactgaactgtgacctgaaatgagcaataaactctgtattaattcactgaaatgttggggttgcttgttatagtagtcggtccatcatgaccagtaaaacataaatcaaaagttaatgtaattgttatcccattatttagagcgaaataaatgttgaatatatggactttctcagattaggaaataccaattaaaaatataataaatagct

SEQ ID NO: 22 - DNA, 5605 bp, Homo sapiens
  misc_feature 2235: n = A,T,C or G
ggagtgggccaggccgccagccccgccagccccgccagccccgccagccccgcgatggcttgggccgcgctcctcggcctcctggccgcactgttgctgctgctgctactgagccgccgccgcacgcggcgacctggtgagcctcccctggacctgggcagcatcccctggttggggtatgccttggactttggaaaagatgctgccagcttcctcacgaggatgaaggagaagcacggtgacatctttactatactggttgggggcaggtatgtcaccgttctcctggacccacactcctacgacgcggtggtgtgggagcctcgcaccaggctcgacttccatgcctatgccatcttcctcatggagaggatttttgatgtgcagcttccacattacagccccagtgatgaaaaggccaggatgaaactgactcttctccacagagagctccaggcactcacagaagccatgtataccaacctccatgcagtgctgttgggcgatgctacagaagcaggcagtggctggcacgagatgggtctcctcgacttctcctacagcttcctgctcagagccggctacctgactctttacggaattgaggcgctgccacgcacccatgaaagccaggcccaggaccgcgtccactcagctgatgtcttccacacctttcgccagctcgaccggctgctccccaaactggcccgtggctccctgtcagtgggggacaaggaccacatgtgcagtgtcaaaagtcgcctgtggaagctgctatccccagccaggctggccaggcgggcccaccggagcaaatggctggagagttacctgctgcacctggaggagatgggtgtgtcagaggagatgcaggcacgggccctggtgctgcagctgtgggccacacaggggaatatgggtcccgctgccttctggctcctgctcttccttctcaagaatcctgaagccctggctgctgtccgcggagagctcgagagtatcctttggcaagcggagcagcctgtctcgcagacgaccactctcccacagaaggttctagacagcacacctgtgcttgatagcgtgctgagtgagagcctcaggcttacagctgcccccttcatcacccgcgaggttgtggtggacctggccatgcccatggcagacgggagagaattcaacctgcgacgtggtgaccgcctcctcctcttccccttcctgagcccccagagagacccagaaatctacacagacccagaggtatttaaatacaaccgattcctgaaccctgacggatcagagaagaaagacttttacaaggatgggaaacggctgaagaattacaacatgccctggggggcggggcacaatcactgcctggggaggagttatgcggtcaacagcatcaaacaatttgtgttccttgtgctggtgcacttggacttggagctgatcaacgcagatgtggagatccctgagtttgacctcagcaggtacggcttcggtctgatgcagccggaacacgacgtgcccgtccgctaccgcatccgcccatgacacagggagcagatggatccacgtgctcgcctctgcccagcctgccccagcctgccccagcctcccagctttctgtgtgcacagttggcccgggtgcaggtgctagcattaccacttccctgcttttctcccagaaggctgggtccaggggagggaaaagctaagagggtgaacaaagaaaagacattgaaagctctatggattatccactgcaaagttttctttccaaaatcaggctttgtctgctcccaattcacctcgttactctcacctcgtgatatccacaaatgctattcagataaggcagaactaggagtcttcactgctctgcccccaactcccggaggtgtcaccttcctagttcttatgagctagcatggcccgggccttatccagtcaaagcggatgctggccacagaaaggccactcaggatgtcctttgtgtccattgatgtcattcagcagtcagtcccccaataatccttaaactagctaaaaccaaaggagtcccttagaagatctgcttccctggggccccatttgccagattgccccattgctcacactacttgagaaaatgcaggagagcttcccccaaggctgatgcattcccggtgcagaacaggggcaccctccaaacactgggctctgaggagtggagttctctgttctagagtnacaggcaccagatgggatgggctttctcagtgtcagcactcaggtagggagctaaggaagacacagcccagacaagatggctggaaggagccagccaggactccttagactgatcaagccaaaaaagaaggtgccgatttcatgcattctagtgcagaagccccaactgtgatcacgatccagtctgcagacgtgttttgtttggacttcacttaaaaaaatgccttagttgttatcatctttgggagagttcattcaaaatgtccagcttctcttgaaaacttggtatatctggccacactgggctcacattcccaagggtaactcttggccagagctgagtggcagccgcctcccttatgcaggacatgtgctctcggcttcaccagggttctgaccgggtctgcttctgcattcacagcgsctcctggrcctgaaggcatctgagtgtgagaccctgttctaactcttagaagtgacattgtaagaggtggtggggaccagctaattggtccaacccagcctgagtgcaccaccctttgaacaaatgtatcagtgatgaaaatttgcctttgccccggcttgcctgtaatcccagcactttgggaggccgaggtgggcggatcacttgaggtcgggagttcaagaccagcctggccaacatggcgaaaccccgtctctactaaacataaaaaaattagtcaggtgtggcggtgccgtgcctgtaatcccagctattcaggaggctgaggcaccagaattgcttgaacccaggaggtggaggttgcagtgaactgaagactgcgccacggcactccagcctgggcgacagagcaagactctgtctcaataaataaataattaattaaaataaaaaacagcttaaagagaaaaatggcctgcaaaccttttttatgatgctatttttattaatataaagtcctgtttattgagaccctttaaatgcctgcgagagaccctacagacagtatgtctactcctcacagcatctctatgaagagaaggagggttgtgcccacttcatagatgagaaaactgagaggtgaggtgacttgcctggggccacatagctcataarctgtagaactctgcaggcagatttactgtcccaggagcaaatgctggatgagcaactcctgttctttgggctcaaggggactggtgatgggacaattcttctcgacttcaggagctgacagagccagaggcacctaaacttgggtacatcttgtaagacacataatgaggtccctctagctttagctggagggagataaaagaaccccagacctctgaatgtcccagaggctagtctcttctcagagcagcactggggtttgggggcttccctgggcctcagcctccaggcacccccaggattcccagagagacgctgtgattggcaggaggcaggatatccccagggaaacccatcttcacgcctgggtgacccccactgccgcctccttcttaactctgcaggaaatcagagctggcagcctccagtgggaggacagagccggtttccgtggcaaccctcagctgcctcatcgtggctgggaagggaaggaagcaagccggcagaaatagctatggaaggtcttgcgcaggctgcaaccctggtgtgctgggcgaaaacccttatgactccccccttccaaatcaggctgggttgtcactgagactagattctcacctgccttcaaagaagggccaattccctttaaaggtcgcacctccttggaaccacagtcattagtgaattacactcaaggaaaagatgtgctcccaccaggcagctccagctgttacctgagatactgaagtgcagctcaggagacgattatttaaacctgcccttgttttaatcgttatttttctctttaaaaaaaatagaagctataaagaaaagagggagagatgagtgggttagctacctgctatgcgctagttaggaagttacctggatgccattgtatttcttcatcccttgcttaagaatcaaaattactggacatatgttaggaatactctttctttctttctttctttttaagctcagtggcaagaatgaaatactctttttttttttttttttttgagatggagtctcgctctgctgcccaggctagagtgcagtggcgtgatctcggctcactgcaagctctgcctcccgtgttcacaccattctcctgcctcagcctcctgagtagctgggactacaggcacccgccaccacacccggctaatttttggatttttagtagagatgggatttcaccgtattagccaggatggtcttgatctcctgagctcgtgatctgcccgcctcggcctcccaaagtgctgggattacaggtgtgagccaccgcgcccagccaagaataaaatactcttaagttgatctaatgaagtgtttccttaccattgtgattattgttactattatttgctatattttaatattgttgtttaccaaatattctcctttaaacagactcgctttttaaacttttttttttttttttgagacggagtttcacgcttattgcccaggctggagtgcaatggcacgatctcagctcaccacaacctccacctcctgggtttaagcaatgcttctgcctcagcttcccaagtagctgagattacaggcgcacaccaccacggccaactggtttttgtatttttagtagagacggggttcccccatgttggtcaggctggtctcgaaactcctgaccctcgtgatttgcctgcctggggcctcccaaagtgctaggattacaggcatgagccaccatgcccggcctaaactttgtttttaaaatgaactttttttccccccaattgctgccaatagtggataacatgtatcactcactgccaaaaatagaaagtgaccatgaaaaataaattcgctggggaagggggctccatgctggtgtggccaaggctgagagctctctcttctctgttacaaaacgagataagcaagtrttagaattgccttaaggccacactggcatctccctgaccttctccagggacagaagcaggagtaagtttctcatcccatgggcgaccagggccatctcctcccaccagtggcccccactcacagggagctggcaatgccctacctgcctgttctccagatggagaaacaggctctgagatttcacaggtcttgcccaaagtcattgattttaatgattaaaaagaataaacacagtgtttcctgagtagcagtgattgttatgccttgctattttaataaagattctattttcgtataacattgtcaagtggaaacatgctgaaatctattaaaccatctttgtttgtg

SEQ ID NO: 23 - DNA, 1484 bp, Homo sapiens
actcagcccattgagttttaaaacctccctaatgacagatataacagttggagaattctgttaaggtttaatgacagtattttatatcatgcccagatggagctaagcaaaataagtagatttaactgtgtttatactaagttgcacagccagttttggaatctgaaaagcatttaataaattgctgcctttattctgtccatttcattattgtttatagttattaaatttaagaataaccaggggttactataaggagatatatttataatgtgttaaatatgatatatcttaaagaagatggttaaactaaatttaagatatttcaatatataaaaatggctcttgaagtccctatcttaaatgaaaaaatttttatatatcaaatctgtctttcatactctcagaatggcacagagaagacataaaagagtaagaacatggactcgtcacataaacatttttaataaagattacatctttgtacctgtaaatgagtcgtctcactggtatctcgcagtcatttgttttccatggttagaagaagctgtgtatgaagattttccacaaactgtatcccagcagtcccaggctcagcagtcccaaaatgacaacaaaacaatagataatgatctacgtactacttcgacactgtctttgagtgcagaggattcccaaagtaccgagtcgaatatgtcagtaccaaagaaaatgtgtaaaaggccatgtattcttatactagactccttgaaagctgcttctgtacaaaacacagttcagaatttacgagagtatttagaggtagagtgggaagttaaactaaaaactcatcgtcaattcagcaaaacaaacatggtggatctatgccctaaagttcctaaacaggacaatagcagtgattgtggagtatatttattgcagtatgtggaaagcttcttcaaggatcctattgttaactttgaacttccaattcatttggagaagtggtttcctcgtcatgtaataaagaccaaacgggaagatattcgagagctcatcttgaaacttcatttacagcaacagaagggcagcagtagctagttaatctgtacaaacatgacacagatgttctctaagattactggaaagcctcttaccagcatttgtgttagccagctcacagagaagaaaataacttgcagtagttttataataagtcattggaacattatttaaaatatgtaggacacattattagaattgttgggatctcatagatggaatgggaatgggggtgatatagataaacttactagatataaattaaaattttataaatatttcatatttttctgagtaaatatgattggattatgcaacagcatatgtaatatgggaatgttttgtagataataaaacttacatgatctgtacttccacgtgactgggttctgaggggagttaaagcctcccgggtgccagccccagtgcttgtccaaatttgc

SEQ ID NO: 24 - DNA, 2518 bp, Homo sapiens
ccagcagtggctgcaccatgcacgtgaacggcaaagtggcgctggtgaccggcgcggctcagggcataggcagagcctttgcagaggcgctgctgcttaagggcgccaaggtagcgctggtggattggaatcttgaagcaggtgtacagtgtaaagctgccctgcatgagcaatttgaacctcagaagactctgttcatccagtgcgatgtggctgaccagcaacaactgagagacacttttagaaaagttgtagaccactttggaagactggacattttggtcaataatgctggagtgaataatgagaaaaactgggaaaaaactctgcaaattaatttggtttctgttatcagtggaacctatcttggtttggattacatgagtaagcaaaatggaggtgaaggcggcatcattatcaatatgtcatctttagcaggactcatgcccgttgcacagcagccggtttattgtgcttcaaagcatggcatagttggattcacacgctcagcagcgttggctgctaatcttatgaacagtggtgtgagactgaatgccatttgtccaggctttgttaacacagccatccttgaatcaattgaaaaagaagaaaacatgggacaatatatagaatataaggatcatatcaaggatatgattaaatactatggaattttggacccaccattgattgccaatggattgataacactcattgaagatgatgctttaaatggtgctattatgaagatcacaacttctaagggaattcattttcaagactatgatacaactccatttcaagcaaaaacccaatgaacagcttatgtgttagccatagctgaaaataagcacaaatagcttatattcagatcctatcttcatttgaatatagcttttaaatgaaatgttacagtttgaagttttccttcatgcacttggtgataaacgttttctaaatttttagttaagtatatggataaaaagttatgaactattaaaaatgtgatgtggaccaaaggctaggttgtaatcttgatagtctaaaaaatgatcaaaacaaatgattttcaaggaatattcaatattctgcctttcagaaagtgtatttatatctgtgcttcataaatattaatgttcttcagaacatcattttaaaggagatacttgaattgttatttaaatcaaaccagatgtaaaacactcacatacaagttcatactttaaaagaggaaagctacttaacaatgacaaatatttcacaataataatttttacttatataccatctttcaactgaacatttcagttcttccaagagcttcttagagtagtatattttgggggcagtcaaggaataaactacagtgtaaacatatcccagatgaaaactgctgtatggaaaaatgacagaaagtaactgattgacactgttgattcacagttcagcctcctatctgggaaagacatttctttcctctgctcactttaagaacttttaccgactccaaaaatctcaggaattaaacttttaacagttacagcaataaagaatagttagtactccaaaaatattatatttaagatgctcaacaagaaaaaaatgcaaatgtaatatttttttcaaattacttctttattgacttgtccaaatttcaaaagtgcctacccttcaataaaacttttttattctgatctccataaattacttagtcttctatgtatagctatcaaggaaataaaaccaattttgccacagccacaactgtaaatgtttttgtacccatgctgaaactcataacaacacagacataaaaatagctgtgaggttttgctttttttgttgtcagctatcttaagaatcattaaatacacctgctttgggtaaaactctttgcaagcagtaattaacactagtaacagtgaaagcacaagatttccaaatcagtcgttttctcaaaaaaatatcgtataagtgactcatcctgtctgctaactccagacctcccagcttgaagccaaatctttccatgtgagattgatatggatttcctagaagtactggaatgttgtcatatcttgccctattttaattctgctatagaaaacaattgccttcacttttaaggagtaatttgaatattaataactctggtctagattttcatataatgtattaaagacaaagtagtgaacatcaatgaacatctgatagagataaactgtaatcaggcataagcttgtttgtatgttctggcagtgactaatcagtaaatgatgtcggtttgcccagtatcacttatcttctgtatttttcctctgtcgtgtaaatagtataaccttttcatttatggacaattttttggactagtagccttcaatatacattctgctttgaattaattttttcaaatcaataaattatgtagacatttaaaatcaaatatcaagtagaattgaaaaatgtgagttacataagttaaaaacttactttaaatcttaccttctataggtagctc

SEQ ID NO: 25 - DNA, 2032 bp, Homo sapiens
cgcctggaccatgtgaatggggccagagggctcccgggctgggcagggaccatgggctgtggctgcagctcacacccggaagatgactggatggaaaacatcgatgtgtgtgagaactgccattatcccatagtccgactggatgggaagggcaggctgctcatccgaaatggctctgaggtgcgggacccactggttacctacgaaggctccaatccgccggcttccccactgcaagacaacctggttatcgctctgcacagctatgagccctctcacgacggagatctgggctttgagaagggggaaccactccgcatcctggagcagagcggcgagtggtggaaggcgcagtccctgaccacgggccaggaaggcttcatccccttcaattttgtggccaaagcgaacagcctggagcccgaaccctggttcttcaagaacctgagccgcaaggacgcggagcggcagctcctggcgcccgggaacactcacggctccttcctcatccgggagagcgagagcaccgccgggtccttttcactgtcggtccgggacttcgaccaaaaccagggagaggtggtgaaacattacaagatccgtaatctggacaacggtggcttctacatctcccctcgaatcacttttcccggcctgcatgaactggtccgccattacaccaatgcttcagatgggctgtgcacacggttgagccgcccctgccagacccagaagccccagaagccgtggtgggaggacgagtgggaggttcccagggagacgctgaagctggtggagcggctgggggctgcacagttcggggaggtgtggatggggtactacaacgggcacacgaaggtggcggtgaagagcctgaagcagggcagcatgtccccggacgccttcctggccgaggccaacctcatgaagcagctgcaacaccagcggctggttcggctctacgctgtggtcacccaggagcccatctacatcatcactgaatacatggagaatgggagtctagtggattttctcaagaccccttcaggcatcaagttgaccatcaacaaactcctggacatggcagcccaaattgcagaaggcatggcattcattgaagagcggaattatattcatcgtgaccttcgggctgccaacattctggtgtctgacaccctgagctgcaagattgcagactttggcctagcacgcctcattgaggacaacgagtacacagccagggagggggccaagtttcccattaagtggacagcgccagaagccattaactacgggacattcaccatcaagtcagatgtgtggtcttttgggatcctgctgacggaaattgtcacccacggccgcatcccttacccagggatgaccaacccggaggtgattcagaacctggagcgaggctaccgcatggtgcgccctgacaactgtccagaggagctgtaccaactcatgaggctgtgctggaaggagcgcccagaggaccggcccacctttgactacctgcgcagtgtgctggaggacttcttcacggccacagagggccagtaccagcctcagccttgagaggaggccttgagaggccctggggttctccccctttctctccagcctgacttggggagatggagttcttgtgccatagtcacatggcctatgcacatatggactctgcacatgaatcccacccacatgtgacacatatgcaccttgtgtctgtacacgtgtcctgtagttgcgtggactctgcacatgtcttgtgcatgtgtagcctgtgcatgtatgtcttggacactgtacaaggtacccctttctggctctcccatttcctgagaccaccagagagaggggagaagcctgggattgacagaagcttctgcccacctacttttctttcctcagatcatccagaagttcctgaagggccaggactttatctaatacctctgtgtgctcctccttggtgcctggcctggcacacatcaggagttcaataaatgtctgttgatgactgccg

SEQ ID NO: 26 - DNA, 8456 bp, Homo sapiens
  misc_feature 3848, 5680, 5735, 5750, 5858, 8099, 8100, 8108, 8130,8448: n = A,T,C or G
ctctcgcggcgcgaccgctcagtcacttcgcccagagacccggacctggtccgctggggagcaggcggccataaaccccctctctcccggttccctgacgccgcggcaggagctgttacaaacaccctgcggttggtctccgatgcccttcagtgaggtggggacgcctggaccctggtgagcgaaccccaagccaccccccaccccaactcagtgtcttcgccggcccccggcccgtacgcctgtctggtcgccatggctgaaaacacagagggggatctgaactccaacctgctccacgccccctaccacaccggggaccctcagctggacacggccatcgggcagtggctccgctgggataagaatcccaaaacaaaagagcagattgaaaacctgttacggaatgggatgaacaaggagctgcgagatcgtctttgttgccgaatgacttttgggactgcaggacttcgttctgccatgggggcagggttttgctatattaatgaccttacagtaatacagtcaacacaggggatgtacaaataccttgagagatgtttctcagacttcaagcagagaggctttgtggttgggtatgacactcggggtcaagtaactagcagctgcagcagccagaggcttgctaaactcactgctgcagtcttgctggccaaagatgttcctgtgtaccttttttcaagatatgttcctacaccttttgtaccatatgcagttcagaagctcaaagcagttgcaggtgtgatgattactgcctctcacaaccgcaaggaagacaatggatacaaggtttactgggaaactggtgctcagatcacatctcctcatgataaagaaattctaaaatgtatagaagaatgtgtggaaccctggaatggttcctggaatgataatttagtggataccagcccgctgaagagagaccctctgcaggacatttgcaggagatacatggaagatctgaaaaagatctgtttttacagggagttaaactcgaagaccaccttgaaatttgtgcacacatcttttcatggggtcggacatgactatgtgcagttggcttttaaagtgtttggttttaagcctccaattccagtaccagaacaaaaagatcctgatccagacttttctaccgttaaatgtccaaatcctgaagaaggagaatctgtgctggaactttccttgagactggcagagaaagaaaatgcccgggtagtgctagccacagatcctgatgcagacagactggcagcagcagaacttcaggagaatggttgttggaaagttttcacagggaatgagttggcagctttgtttggatggtggatgtttgattgctggaagaaaaataaatcaagaaatgctgatgtgaagaacgtttatatgttagccaccacagtctcttctaaaattctgaaggcaattgcacttaaagaaggatttcattttgaagaaacattaccaggttttaaatggattggaagtaggataatagacctcctggaaaatgggaaagaagtcctttttgcatttgaagagtctattggttttctctgtggaacttcagttttggataaagatggggtgagtgcagctgttgtggttgctgagatggcatcttacctggaaaccatgaatataacattgaaacagcaactggttaaggtttatgaaaaatatggttatcatatttcaaaaacttcctatttcttgtgttatgaaccacctaccatcaaaagtatatttgaaaggcttcgtaattttgattctccaaaagaatatccaaaattttgtggaacatttgctatattgcatgtacgggacgttaccactggatatgacagtagccagcctaataagaaatcagtgctgcctgtgagtaaaaacagccaaatgattacatttacttttcaaaatggctgtgttgctacccttcggacaagtggaacagaaccaaagataaagtattatgcagagatgtgtgcgtcacctgaccagagtgacactgctttactggaggaagaactgaagaaactcattgatgctctgatagagaattttcttcagcctagtaagaatggactgatctggcgttctgtttaggggtacaccaatatgtcatgacactgtgtgggcatatggaacagagcaaccgagaactgcatacattgaaccttgtgttagcattctctctctatctcatctggccgagtatctttttccttttattttctttctttttggtcaactggctagactaaacaaagtataagtttgaaatgaaatggtctagagggaaatgattcaaattttttcataattcaaacaaaagttattacactaaaagtattatagtaacgtattgtccttccgttaacagaaacatcacaattcaaaagtgagttttcttattatagtgtgattaagcctagttctgtatctcgtaattgtgtatagcatggtaaaagaaaaaaaaaaagacaggtaaatgttatataattgtagggttagttaaggaaattagtccataaaattggcaagaaaatggtactttccttgattgttatgaaaaagtgtcattgtgacatcacaggtattaatttacaaattagagtatttgaaataggatatttagcatttctgtagtttagaacagatgttacagtgtgtggttttggcatgtctgtttcaggtgcccttggtgcagtataaatttactgtatgtatttaaaattctgatgtttactgaacataaaacaatagtggcaacactcctattggctttgttcttatcaaatttctcagtgccttctcagggtctggtatgaacagaaaaaacagtgttcctgcatttaccataatttaatcagtcaatttgcagatagctaaggaaaatcttccaaaatctgtaggtgcttcctctatatcaagtgagtttactgattttaaaatcttaggaatataaataaagatcttaataaaagcaagcttaatgtagttaaggatcttcatattagaattgtgtcatttatcaaaccttttattacagaaaaaaatctgaactgagaaaggagctctgaatttatttgacagtctaacttttgaattacatttttgtaagtagatttatcttttattttatgaagtaatgttaattttactaagaataaaacatccaaattttaagtattatgaaatactaaaactacatgttttaaaacagaggtccagtttattctaacatcttgctgacttctgtagaatttctgattgcatgactattatttatcttcttttagtgtagaaaataattttttacaaaaagtcacttttgggctgggcgcagtggctcacgcctgtaatcccagcactttgggaggctgaggcaggtggatcacaatgtcaggagtttgagactagcctggccaatatggtgaaaccctgtctctactaaaaatacaaaaattagccgggtgtggtggtgggcacctgtagtcccagctactcgggaggctgaggcaggagaattgcttgaacctaggaggctgagattgcagtgagccgaaatcacgccactacactccagcctgggcaacagagggagactccatctcaaaaaaaaaaaaagtcacttttggctaatcatgcagtcttctgaaaaggtttaataaaaaaatgctgtttaggctgggcttggtggctcacatctgtaataccagcgctttgggaggctgaggcaggaggattgcttgaggccaggagtttaagttacagaactatgatcacactgccgcactgaagcctggatgacagagtgagaccttatntcaaaaaaaaaaaaaaaaagagagagagagagaaaaagaaatgctgtgtttaatcttcttaaaatgtatcttgtcctacatagttctcagaatacatatggggtatagttaggagataacaataacaagacctaaacaaattgcttatgggagatagcactgtaattagagaatttatttcatataaaataataatataaagtttgaactgattagaatgttccttaaatcatggtttcgtttgttgcagctgtcagctaacatttggttgagctcatataatccaaacctaattgtccaactattgtattgccactattgttaatttgttccttccttactttcttattgttggatcttatatgactgtatgactgtagataatagacagctagctaaatgtagagcaccatttttctcaatgagtttgttttcaaaggctttgcactggagaagatatgtgtagaatttttgttttatttttaaaatgtgtaaaacatcttttgttttatatgcttttaagaatgtagattgtaatttaggaaatggcatatattctgcaaagtggtataatgttgtatatatgaaaaacaggtatatttggtttagttctgtgtttcagtgactgataaacaaaaatgaagctaaaatgaatcaacggctcattcatagttactaaagccatgtcatgactgtcattctattatgaagaaaacagttttatttgagtgctttaatataatgcaacatttaagtcatcttaaggtgaaaagttattgaagtgttcttctctcaagttaacaaaattttacaaatgttcctgaagtgtatcgattgagggaattattttattctagcctgtcatgagtctgtctgagcatgatacttaacatgaacttgatgtagtaggtgcacatttgcctaacaggataaattcagtgatagcccaagctggtcaggagagacagaagtgctggctaggaggtctcagcctcgggcactacaatgtttatctttttctttttgaacaatattgtgaaaatgattgcattctcattgagcatgaaagtcaaaactggaattttctaaagctttggagaaaaggagagaattagtagggcaagatcaaagcatacattggaaataaacagtagaaataagcaaacaatataaagagacacgggaaatgcttctcgtgaagggcaaatgtaacaccataacagtagggaataaactgtgtcccacctgtttacttgtgtttatttgttgttatgatgagtatgcacctaccattttcctttattgtgatcactaaaatgaggccaaggtttaggaggtgagaaaaagtatcccttttacggattcagttcctgctaggatgttgtatcagaatcatgtgagaactcaggaagaggtagagcagaaataaaaaggaagaaaatgtagattagaaaataagacttctgagaaaaagttcaaggaataaagattgtttgactctggagaggagaaagctgttgagatagtttgctaagtttgctaataaagctttattgagtcttttattgaagggttcaaagtaagaaatactatgccatagtgttctccatctctagtgaagacagaaccagagggaataaggatcttttttttaaagacagcatggaaagtttaagatttaggaacttcttgagaaatcttcataatatcttctattcaaaatcttttttttatttttaagggaataaaaattttaaaaattaagagatatacagatgactttttaaggtccttctttccagcacattttttttaaaaactcaggttagaanaaaaatgtcataaagggcactaaaagtaacatttgggtttcactccatatttagnagaatttagctcacnaaacaatttttgtgtgttttgagtaactgatgcataagtgcaatgaataattttcctgcttcactgttatttttaatttaacctggcaaattaagtgagacttgaacnaaattttgttcatcttgaagaatgtatagtggcttaggttatttgaggttttcgttttgaatatatatgttgttattttgatgtagatacgttagcagatggagaggttctaagagattggatactttggaatgtctttatataggaaatagacctcccatttcttaagcaagttgcttctggaattgatgcaacaggggatattagctgtagtttaatggataagttgttctatgagctgctgctatcagtacttttttccacattttccattgatatttgtgatgcttgaaaaatccaaacttcacttggatacatttaattttatgcctttttctctctacctcccacttttcctaggcacccttgcacattttgttactgtgaaggactattgccctgttacctcactgataagaaatctcaactgtattcattcagaatgctttaagcttagtcataaactacatttgcctatggacctttggttattttgttaatgttgtcattatatttaagacttattgtaggaagtaattttaaatctatttcataacctgttttgatctgtctgcatactgctatatgttaatttattacggcttttacaattcacttgtcatctgagattgttctagaaactgatgaggaaagagtcgtactaaccaaaatgaatgggcaattgacataacattctatttcaggtttccttgtacttggggagccaaagcacaagatctaaggaaatcttattcacatttgtccctcagagcatcttaccctaagccacagaggtataaatagtaagatgagaattgctcattgtctgactccttgcacagcatgaatttaattctagttggaaacaaagttagtgttgactactggttaccttaaagtattcaccatactatatatttattagaccatctttcctctttgtttattcaagactatgtacttataaaagagaacctataagaatgtgtagagtgaccaaatagggacaaagtgacatacctagagtttgctgctcaaatttttccactagcgtaaccctaaagcagaggggcatgatgaagggggaaggggttctgagagcaaaatcagaatcagcctaccactgacatgaagtgtgttctgtctttaaaatgtaagtgaattttgcattctctataataaaacttttaatataaaaatgatatgtttaatctttactgaaacttatgccaccacaaaggagcaccatgttcatcacgtggctgctcagttttttcccccagtgccctcaccgccagtggaggcaagtccttgagctacacacactggctgtagtgcaagatatataaacataggccgggctcggtggctcatgcctgtaatcctagcactttgggaggccgaagcaggtggatcatctgaggtcgggagattgagactagcctgaccgacatggagaaaccctgtgtccactaaaaatacaaaattagccgggtgtggtggcacatgcctgtaatcccagctactcgggaggctgaggcaggagaatcacttgaacccaggaggtggaggttgtggtgagccaagatcgcgccgttgcactccagcctgggcaacaagagtgaaactccgtctcaaaaaaaaaaaaaaaagatatataaacatagaacatatccagactatttcctgcccttggaatctgaccttcagaaggattaacgccaaactaaactgacctgaaacagttttgttactattggcattccttgactaaaaaggaccacctgatgctcagtcccgagagtaggaatcgtggtttattatttttcatatccataccacctagttcagtacctgttatagagtgtttaattgctgatactaggaattaagggattttttaaaagtagcaaatagtttaaggaacaatcaaaaaatgaattttctaattcagttttttcaatgaagtcccatcactgaactttataatgtaatgtttattaagcagacatccccttaaatactcagtttctttggagagagcaaagcatatctccctgtggaaattatatataatgcctggtattttacactcaaagcaaggtgttggccaggaaggtaaagaaatggtgtctatgtagacttaatatcactagtctaagtgtcattttgagcctagtagcactaccttccaagtgagtcacaacaaatttgatcctatttgtaggttttttccactgnnatgattcntcatgtatcttacaagagccantcaagcaagactctgcttctatgtatggtgaggccttgttgttctagggtagaataaactctttgtatgcctcattgaatatgccaggtaaaatatatgcagtcaagaatgaattatttttctgactaaagtgtgtagcagtagttcaaaattgtgcccttgttttaacagtttctgtcaacatcttctcatttttccctacaaaaacaccagggtgtattataagtactgcctgtgagaatttgcactttatgtatttgtgtgtggatttcttgtggttttagccaaatgaagtgttatcagtaataaacaggtctnttcatagg

SEQ ID NO: 27 - DNA, 3372 bp, Homo sapiens
tacaaccaggctcaactgttgcatggtagcagatttgcaaacatgagtgctgaggggtaccagtacagagcgctgtatgattataaaaaggaaagagaagaagatattgacttgcacttgggtgacatattgactgtgaataaagggtccttagtagctcttggattcagtgatggacaggaagccaggcctgaagaaattggctggttaaatggctataatgaaaccacaggggaaaggggggactttccgggaacttacgtagaatatattggaaggaaaaaaatctcgcctcccacaccaaagccccggccacctcggcctcttcctgttgcaccaggttcttcgaaaactgaagcagatgttgaacaacaagctttgactctcccggatcttgcagagcagtttgcccctcctgacattgccccgcctcttcttatcaagctcgtggaagccattgaaaagaaaggtctggaatgttcaactctatacagaacacagagctccagcaacctggcagaattacgacagcttcttgattgtgatacaccctccgtggacttggaaatgatcgatgtgcacgttttggctgacgctttcaaacgctatctcctggacttaccaaatcctgtcattccagcagccgtttacagtgaaatgatttctttagctccagaagtacaaagctccgaagaatatattcagctattgaagaagcttattaggtcgcctagcatacctcatcagtattggcttacgcttcagtatttgttaaaacatttcttcaagctctctcaaacctccagcaaaaatctgttgaatgcaagagtactctctgaaattttcagccctatgcttttcagattctcagcagccagctctgataatactgaaaacctcataaaagttatagaaattttaatctcaactgaatggaatgaacgacagcctgcaccagcactgcctcctaaaccaccaaaacctactactgtagccaacaacggtatgaataacaatatgtccttacaaaatgctgaatggtactggggagatatctcgagggaagaagtgaatgaaaaacttcgagatacagcagacgggacctttttggtacgagatgcgtctactaaaatgcatggtgattatactcttacactaaggaaagggggaaataacaaattaatcaaaatatttcatcgagatgggaaatatggcttctctgacccattaaccttcagttctgtggttgaattaataaaccactaccggaatgaatctctagctcagtataatcccaaattggatgtgaaattactttatccagtatccaaataccaacaggatcaagttgtcaaagaagataatattgaagctgtagggaaaaaattacatgaatataacactcagtttcaagaaaaaagtcgagaatatgatagattatatgaagaatatacccgcacatcccaggaaatccaaatgaaaaggacagctattgaagcatttaatgaaaccataaaaatatttgaagaacagtgccagacccaagagcggtacagcaaagaatacatagaaaagtttaaacgtgaaggcaatgagaaagaaatacaaaggattatgcataattatgataagttgaagtctcgaatcagtgaaattattgacagtagaagaagattggaagaagacttgaagaagcaggcagctgagtatcgagaaattgacaaacgtatgaacagcattaaaccagaccttatccagctgagaaagacgagagaccaatacttgatgtggttgactcaaaaaggtgttcggcaaaagaagttgaacgagtggttgggcaatgaaaacactgaagaccaatattcactggtggaagatgatgaagatttgccccatcatgatgagaagacatggaatgttggaagcagcaaccgaaacaaagctgaaaacctgttgcgagggaagcgagatggcacttttcttgtccgggagagcagtaaacagggctgctatgcctgctctgtagtggtggacggcgaagtaaagcattgtgtcataaacaaaacagcaactggctatggctttgccgagccctataacttgtacagctctctgaaagaactggtgctacattaccaacacacctcccttgtgcagcacaacgactccctcaatgtcacactagcctacccagtatatgcacagcagaggcgatgaagcgcttactctttgatccttctcctgaagttcagccaccctgaggcctctggaaagcaaagggctcctctccagtctgatctgtgaattgagctgcagaaacgaagccatctttctttggatgggactagagctttctttcacaaaaaagaagtaggggaagacatgcagcctaaggctgtatgatgaccacacgttcctaagctggagtgcttatcccttctttttctttttttctttggtttaatttaaagccacaaccacatacaacacaaagagaaaaagaaatgcaaaaatctctgcgtgcagggacaaagaggcctttaaccatggtgcttgttaatgctttctgaagctttaccagctgaaagttgggactctggagagcggaggagagagaggcagaagaaccctggcctgagaaggtttggtccagcctggtttagcctggatgttgctgtgcacggtggacccagacacatcgcactgtggattatttcattttgtaacaaatgaacgatatgtagcagaaaggcacgtccactcacaagggacgctttgggagaatgtcagttcatgtatgttcagaagaaattctgtcatagaaagtgccagaaagtgtttaacttgtcaaaaaacaaaaacccagcaacagaaaaatggagtttggaaaacaggacttaaaatgacattcagtatataaaatatgtacataatattggatgactaactatcaaatagatggatttgtatcaataccaaatagcttctgttttgttttgctgaaggctaaattcacagcgctatgcaattcttaattttcattaagttgttatttcagttttaaatgtaccttcagaataagcttccccaccccagtttttgttgcttgaaaatattgttgtcccggatttttgttaatattcatttttgttatccttttttaaaaataaatgtacaggatgccagtaaaaaaaaaaatggcttcagaattaaaactatgaaatattttacagtttttcttgtacagagtacttgctgttagcccaaggttaaaaagttcataacagattttttttggactgttttgttgggcagtgcctgataagcttcaaagctgctttattcaataaaaaaaaaacccgaattcactgg

SEQ ID NO: 28 - DNA, 3984 bp, Homo sapiens
ctgcaggttattcagcgatagttatgacctcccggttacgtgcgttgggtggaagaattaataatatacgcacctcggagttacccaaagagaaaactcgatcagaagtcatttgcagcatccactttttagatggcgtggtacagacctttaaagttactaaacaagacactggccaggttcttctggatatggtgcacaaccacctgggtgtgactgaaaaggaatattttggtttacagcatgatgacgactccgtggactctcctagatggctggaagcaagcaaacccatcaggaagcagttaaaaggaggtttcccctgtaccctgcattttcgagtaagattttttatacctgatcccaacacactgcagcaagaacaaaccaggcacttgtatttcttacaactgaagatggatatttgcgaaggaaggttaacctgccctcttaactcagcagtggttctagcgtcctatgccgtacaatctcattttggagactataattcttccatacatcatccaggctatctttccgatagtcactttatacccgatcaaaatgaggactttttaacaaaagtcgaatctctgcatgagcagcacagtgggctaaaacaatcagaagcagaatcctgctatatcaacatagcgcggaccctcgacttctatggagtagaactgcacagtggtagggatctgcacaatttagacctaatgattggaattgcttccgcgggtgttgctgtgtaccgaaaatacatttgcacaagtttctatccttgggtgaacattctcaaaatttctttcaaaaggaaaaagttcttcatacatcagcgacagaaacaggctgaatccagggaacatattgtggccttcaacatgctgaattaccgatcttgcaaaaacttgtggaaatcctgtgttgagcaccatacgttctttcaggcaaagaagctactacctcaggaaaagaatgttctgtctcagtactggactatgggctctcggaacaccaaaaagtcggtaaataaccaatattgcaaaaaggtgattggcgggatggtgtggaacccagccatgcggagatccttatcagtggagcacttagaaaccaagagtctgccttctcgttcccctcccattactcccaactggcgaagtcctcggctccggcacgaaatccgaaagccacgccactcttctgcagataaccttgcaaatgaaatgacctacatcacggaaacggaagatgtattttacacgtacaagggctctctggcccctcaagacagcgattctgaagtttctcagaaccgaagcccgcaccaagagagtttatccgagaacaatccggcacaaagctacctgacccagaagtcatccagttctgtgtctccatcttcaaatgctccaggctcctgctcacctgacggcgttgatcagcagctcttagatgacttccacagggtgaccaaagggggctccaccgaggacgccagccagtactactgtgacaagaatgataatggtgacagctacttagtcttgatccgtatcacaccagatgaagatggaaaatttggatttaatcttaagggaggagtggatcaaaagatgcctcttgtggtatcaaggataaacccagagtcacctgcggacacctgcattcctaagctgaacgaaggggatcaaatcgtgttaatcaatggccgggacatctcagaacacacgcatgaccaagtggtgatgttcatcaaagccagccgggagtcccactcacgggagctggccctggtgatcaggaggagagctgtccgctcatttgctgacttcaagtctgaagatgaactgaaccagcttttccccgaagccattttccccatgtgtccggagggtggggacactttggagggatccatggcacagctaaagaagggcctcgaaagcgggacggtgctgatccagtttgagcaactctacagaaaaaagccaggtttggccatcacgtttgcaaagctgcctcaaaatttggacaaaaaccgatataaagatgtgctgccttatgacaccacccgggtattattgcagggaaatgaagattatattaatgcaagttacgtgaacatggaaattcctgctgctaaccttgtgaacaagtacatcgccactcaggggcccctgccgcatacctgtgcacagttttggcaggttgtctgggatcagaagttgtcactcattgtcatgttgacgactctcacagaacgagggcggaccaaatgtcaccagtactggccagatccccccgacgtcatgaaccacggcggctttcacatccagtgtcagtcagaggactgcaccatcgcctatgtgtcccgagaaatgctggtcacaaacacccagaccggggaagaacacacagtgacacatctccagtacgtcgcatggcctgaccacggtatacccgatgactcctccgactttctggaatttgtaaactatgtgaggtctctgagagtggacagcgagcctgtcctagttcactgcagtgctggaataggtcgaaccggtgtgttggtcactatggaaacagccatgtgcctaactgagaggaacctgcccatttacccactggatattgtccgaaaaatgcgagaccagcgcgccatgatggtgcagacatcaagccagtacaagtttgtgtgtgaagcgattcttcgtgtgtatgaagaaggtttagtccaaatgctggatcctagttaagacaactgtgaaaaagttcattcctctttcccaagggcatcctccttgaaagaggaggacagacctctctggaagcagcaagaggaaccagtagctgtgggaaaggaatgggcacctctgaacccaggcactttaaacttctatagaaaagatatcgtgtacataggaactggtgtagataagcatgcaattatggcatcatttaggcctgtatttctatggaaagatacaaaaaggatctcagtttggggcctgtcctaatgccttcttccctaacatcaccacacacacccctgtcggcatcctggagcaattgagaccggacacccacagagctgttgtcctcccagcaacaagatggtgtggttatcttgggtcatttggatgttttgtttgtttctgtgtgtcagactgtaagggctgagctttctgtgcttctaggtggagctggaacaattcagattcacccgccctgatgctaaggaaaccctgacgtatgtactagatggcagggcactgggggtcaggctgaaggctgagcaacacctctctgccctccctccctttgtcccatctcccagcgacttccaatattcatgtttctgagaattgtgtccctcttcagttccctcttggtgcctaacctggattagtaatgtgcattcaggtgaattttcagctgaggctctgagaactggtactctcagtgtgttctggtcatcttgtggcttagttgtagaagcaggtgtgtctcttgcctctgcttgcctcctactgcacactcagcacccaggactggaatcaccgactactgaatctcctacatgtattgctgctacttcaagctcctccacttgaaaccttatgattttccaaggggagatgggacagtgtcatctaaatattccgaatgtttggccttctgagaaaagagcttctagtaattgaaccatgggtttcccagcttctggagggttggccgtgggctgtgtacatgtgtgtgcccaggggtgagtgtttctcaggattcctaacgattcaaattaccgttgagtatatataaagaatcgagtctctgtatggaagaacaaatgtgtgcattcacccccagtcacaatggtctccattgcatttcaaaggagaggatcagactatctgaatataaacacaatctgatgttaatttattctaagaacaccatcattttgattgtcctaaa

SEQ ID NO: 29 - DNA, 5248 bp, Homo sapiens
acgcgtccggcagctgcagcagctacgggagtggccgggtggccggcgggtgccagccgccatggaggccgtgccccgcatgcccatgatctggctggacctgaaggaggccggtgactttcacttccagccagctgtgaagaagtttgtcctgaagaattatggagagaacccagaagcctacaatgaagaactgaagaagctggagttgctcagacagaatgctgtccgtgtcccacgagactttgagggctgtagtgtcctccgcaagtacctcggccagcttcattacctgcagagtcgggtccccatgggctcgggccaggaggccgctgtccctgtcacctggacagagatcttctcaggcaagtctgtggcccatgaggacatcaagtacgagcaggcctgtattctctacaaccttggagcgctgcactccatgctgggggccatggacaagcgggtgtctgaggagggcatgaaggtctcctgtacccatttccagtgcgcagccggcgccttcgcctacctacgggagcacttccctcaagcctacagcgtcgacatgagccgccagatccttacgctcaacgtcaacctcatgctgggccaggctcaggagtgcctcctggagaagtcgatgttggacaacaggaagagctttctggtggcccgcatcagtgcacaggtggtagattactacaaggaggcatgccgggccttggagaaccccgacactgcctcactgctgggccggatccagaaggactggaagaaacttgtgcagatgaagatctactacttcgcagccgtggctcatctgcacatgggaaagcaggccgaggagcagcagaagttcggggagcgggttgcatacttccagagcgccctggacaagctcaatgaagccatcaagttggccaagggccagcctgacactgtgcaagacgcgcttcgcttcactatggatgtcattgggggaaagtacaattctgccaagaaggacaacgacttcatttaccatgaggctgtcccagcattggacactcttcagcctgtaaaaggagcccccttggtgaagcccttgccagtgaaccccacagacccagctgttacaggccctgacatctttgccaaactggtacccatggctgcccacgaggcctcgtcactgtacagtgaggagaaggccaagctgctccgggagatgatggccaagattgaggacaagaatgaggtcctggaccagttcatggattcaatgcagttggatcccgagacggtggacaaccttgatgcctacagccacatcccaccccagctcatggagaagtgcgcggctctcagcgtccggcccgacactgtcaggaaccttgtacagtccatgcaagtgctgtcaggtgtgttcacggatgtggaggcttccctgaaggacatcagagatctgttggaggaggatgagctgctagagcagaagtttcaggaggcggtgggccaggcaggggccatctccatcacctccaaggctgagctggcagaggtgaggcgagaatgggccaagtacatggaagtccatgagaaggcctccttcaccaacagtgagctgcaccgtgccatgaacctgcacgtcggcaacctgcgcctgctcagcgggccgcttgaccaggtccgggctgccctgcccacaccggccctctccccagaggacaaggccgtgctgcaaaacctaaagcgcatcctggctaaggtgcaggagatgcgggaccagcgcgtgtccctggagcagcagctgcgtgagcttatccagaaagatgacatcactgcctcgctggtcaccacagaccactcagagatgaagaagttgttcgaggagcagctgaaaaagtatgaccagctgaaggtgtacctggagcagaacctggccgcccaggaccgtgtcctctgtgcactgacagaggccaacgtgcagtacgcagccgtgcggcgggtactcagcgacttggaccaaaagtggaactccacgctgcagaccctggtggcctcgtatgaagcctatgaggacctgatgaagaagtcgcaggagggcagggacttctacgcagatctggagagcaaggtggctgctctgctggagcgcacgcagtccacctgccaggcccgcgaggctgcccgccagcagctcctggacagggagctgaagaagaagccgccgccacggcccacagccccaaagccgctgctgccccgcagggaggagagtgaggcagtggaagcaggagacccccctgaggagctgcgcagcctcccccctgacatggtggctggcccacgactgcctgacaccttcctgggaagtgccaccccgctccactttcctcccagccccttccccagctccacaggcccaggaccccactatctctcaggccccttgccccctggtacctactcgggccccacccagctgatacagcccagggccccagggccccatgcaatgcccgtagcacctgggcctgccctctacccagcccctgcctacacaccggagctgggccttgtgccccgatcctccccacagcatggcgtggtgagcagtccctatgtgggggtagggccggccccaccagttgcaggtctcccctcggccccacctcctcaattctcaggccccgagttggccatggcggttcggccagccaccaccacagtagatagcatccaggcgcccatccccagccacacagccccacggccaaaccccacccctgctcctcccccgccctgcttccctgtgcccccaccgcagccactgcccacgccttacacctaccctgcaggggctaagcaacccatcccggcacagcaccacttctcttctgggatccccgcaggttttccagccccaaggattgggccccagccccagccccatcctcagccccatccttcacaagcgtttgggcctcagcccccacagcagccccttccactccagcatccacatctcttcccaccccaggccccaggactcctacccccacaatccccctacccctatgcccctcagcctggggtcctggggcagccgccaccccccctacacacccagctctacccaggtcccgctcaagaccctctgccagcccactcaggggctctgcctttccccagccctgggccccctcagcctccccatcccccactggcatatggtcctgccccttctaccagacccatgggcccccaggcagcccctcttaccattcgagggccctcgtctgctggccagtccacccctagtccccacctggtgccttcacctgccccatctccagggcctggtccggtaccccctcgccccccagcagcagaaccacccccttgcctgcgccgaggcgccgcagctgcagacctgctctcctccagcccggagagccagcatggcggcactcagtctcctgggggtgggcagcccctgctgcagcccaccaaggtggatgcagctgagggtcgtcggccgcaggccctgcggctgattgagcgggacccctatgagcatcctgagaggctgcggcagttgcagcaggagctggaggcctttcggggtcagctgggggatgtgggagctctggacactgtctggcgagagctgcaagatgcgcaggaacatgatgcccgaggccgttccatcgccattgcccgctgctactcactgaagaaccggcaccaggatgtcatgccctatgacagtaaccgtgtggtgctgcgctcaggcaaggatgactacatcaatgccagctgcgtggaggggctctccccatactgccccccgctagtggcaacccaggccccactgcctggcacagctgctgacttctggctcatggtccatgagcagaaagtgtcagtcattgtcatgctggtttctgaggctgagatggagaagcaaaaagtggcacgctacttccccaccgagaggggccagcccatggtgcacggtgccctgagcctggcattgagcagcgtccgcagcaccgaaacccatgtggagcgcgtgctgagcctgcagttccgagaccagagcctcaagcgctctcttgtgcacctgcacttccccacttggcctgagttaggcctgcccgacagccccagcaacttgctgcgcttcatccaggaggtgcacgcacattacctgcatcagcggccgctgcacacgcccatcattgtgcactgcagctctggtgtgggccgcacgggagcctttgcactgctctatgcagctgtgcaggaggtggaggctgggaacggaatccctgagctgcctcagctggtgcggcgcatgcggcagcagagaaagcacatgctgcaggagaagctgcacctcaggttctgctatgaggcagtggtgagacacgtggagcaggtcctgcagcgccatggtgtgcctcctccatgcaaacccttggccagtgcaagcatcagccagaagaaccaccttcctcaggactcccaggacctggtcctcggtggggatgtgcccatcagctccatccaggccaccattgccaagctcagcattcggcctcctggggggttggagtccccggttgccagcttgccaggccctgcagagcccccaggcctcccgccagccagcctcccagagtctaccccaatcccatcttcctccccaccccccctttcctccccactacctgaggctccccagcctaaggaggagccgccagtgcctgaagcccccagctcggggcccccctcctcctccctggaattgctggcctccttgaccccagaggccttctccctggacagctccctgcggggcaaacagcggatgagcaagcataactttctgcaggcccataacgggcaagggctgcgggccacccggccctctgacgaccccctcagccttctggatccactctggacactcaacaagacctgaacaggttttgcctacctggtccttacactacatcatcatcatctcatgcccacctgcccacacccagcagagcttctcagtgggcacagtctcttactcccatttctgctgcctttggccctgcctggcccagcctgcacccctgtggggtggaaatgtactgcaggctctgggtcaggttctgctcctttatgggacccgacatttttcagctctttgctattgaaataataaaccaccctgttctgtggcccgtaaaaaaaaaaaaaaaaaaaa

SEQ ID NO: 30 - DNA, 1111 bp, Homo sapiens
gcggccgcgaattcggcacgagcagagagcaatatggctggttccccaacatgcctcaccctcatctatatcctttggcagctcacagggtcagcagcctctggacccgtgaaagagctggtcggttccgttggtggggccgtgactttccccctgaagtccaaagtaaagcaagttgactctattgtctggaccttcaacacaacccctcttgtcaccatacagccagaagggggcactatcatagtgacccaaaatcgtaatagggagagagtagacttcccagatggaggctactccctgaagctcagcaaactgaagaagaatgactcagggatctactatgtggggatatacagctcatcactccagcagccctccacccaggagtacgtgctgcatgtctacgagcacctgtcaaagcctaaagtcacattgggtctgcagagcaataagaatggcacctgtgtgaccaatctgacatgctgcatggaacatggggaagaggatgtgatttatacctggaaggccctggggcaagcagccaatgagtcccataatgggtccatcctccccatctcctggagatggggagaaagtgatatgaccttcatctgcgttgccaggaaccctgtcagcagaaacttctcaagccccatccttgccaggaagctctgtgaaggtgctgctgatgacccagattcctccatggtcctcctgtgtctcctgttggtgcccctcctgctcagtctctttgtactggggctatttctttggtttctgaagagagagagacaagaagagtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactgcccctaagtctctgctcaaaaaaaaaacaattctcggcccaaagaaaacatacagg

SEQ ID NO: 31 - DNA, 1061 bp, Homo sapiens
ccacaagctggccccttggcctcctagagaccctgacatctcctccagcagcatctgtcctctctcctcagggaggcaagcatttgatgctcgaggtccctggcagttgtggtccttggcaagtgatgtgtgagtcccgtgtgtcataggaagctccccatccccatctggtgaccaaaggcctggctacaagtagtgagtccttcctcctccacccagacctcactgctcagatccccttcgccaactgggacatcttccgacatggcctggatgctgttgctcatcttgatcatggtccatccaggatcctgtgctctctgggtgtcccagccccctgagattcgtaccctggaaggatcctctgccttcctgccctgctccttcaatgccagccaagggagactggccattggctccgtcacgtggttccgagatgaggtggttccagggaaggaggtgaggaatggaaccccagagttcaggggccgcctggccccacttgcttcttcccgtttcctccatgaccaccaggctgagctgcacatccgggacgtgcgaggccatgacgccagcatctacgtgtgcagagtggaggtgctgggccttggtgtcgggacagggaatgggactcggctggtggtggagaaagaacatcctcagctaggggctggtacagtcctcctccttcgggctggattctatgctgtcagctttctctctgtggccgtgggcagcaccgtctattaccagggcaaatgtctgacctggaaaggtccaagaaggcagctgccggctgtggtcccagcgcccctcccaccaccatgtgggagctcagcacatctgcttcccccagtcccaggaggctgagcctgattgtcctgagaaatgggaaggatcagatatgactcctccttggcaactgccctttcctgccaggcccacacataccctcttctggctgttaggggagcttgggtccctgaacactgtcattcacccaataaattactatttgaccccagagtgggtggaagggtgaaaaaaaaaaaaaaaaaaaa

SEQ ID NO: 32 - DNA, 1816 bp, Homo sapiens
gcacgagcgatgtcgctcgtgctgctaagcctggccgcgctgtgcaggagcgccgtaccccgagagccgaccgttcaatgtggctctgaaactgggccatctccagagtggatgctacaacatgatctaatccccggagacttgagggacctccgagtagaacctgttacaactagtgttgcaacaggggactattcaattttgatgaatgtaagctgggtactccgggcagatgccagcatccgcttgttgaaggccaccaagatttgtgtgacgggcaaaagcaacttccagtcctacagctgtgtgaggtgcaattacacagaggccttccagactcagaccagaccctctggtggtaaatggacattttcctacatcggcttccctgtagagctgaacacagtctatttcattggggcccataatattcctaatgcaaatatgaatgaagatggcccttccatgtctgtgaatttcacctcaccaggctgcctagaccacataatgaaatataaaaaaaagtgtgtcaaggccggaagcctgtgggatccgaacatcactgcttgtaagaagaatgaggagacagtagaagtgaacttcacaaccactcccctgggaaacagatacatggctcttatccaacacagcactatcatcgggttttctcaggtgtttgagccacaccagaagaaacaaacgcgagcttcagtggtgattccagtgactggggatagtgaaggtgctacggtgcagctgactccatattttcctacttgtggcagcgactgcatccgacataaaggaacagttgtgctctgcccacaaacaggcgtccctttccctctggataacaacaaaagcaagccgggaggctggctgcctctcctcctgctgtctctgctggtggccacatgggtgctggtggcagggatctatctaatgtggaggcacgaaaggatcaagaagacttccttttctaccaccacactactgccccccattaaggttcttgtggtttacccatctgaaatatgtttccatcacacaatttgttacttcactgaatttcttcaaaaccattgcagaagtgaggtcatccttgaaaagtggcagaaaaagaaaatagcagagatgggtccagtgcagtggcttgccactcaaaagaaggcagcagacaaagtcgtcttccttctttccaatgacgtcaacagtgtgtgcgatggtacctgtggcaagagcgagggcagtcccagtgagaactctcaagactcttccccttgcctttaaccttttctgcagtgatctaagaagccagattcatctgcacaaatacgtggtggtctactttagagagattgatacaaaagacgattacaatgctctcagtgtctgccccaagtaccacctcatgaaggatgccactgctttctgtgcagaacttctccatgtcaagtagcaggtgtcagcaggaaaaagatcacaagcctgccacgatggctgctgctccttgtagcccacccatgagaagcaagagaccttaaaggcttcctatcccaccaattacagggaaaaaacgtgtgatgatcctgaagcttactatgcagcctacaaacagccttagtaattaaaacattttataccaataaaattttcaaatattgctaactaatgtagcattaactaacgattggaaactacatttacaacttcaaagctgttttatacatagaaatcaattacagttttaattgaaaactataaccattttgataatgcaacaataaagcatcttcagccaaaaaaaaaaaaaaaa

SEQ ID NO: 33 - DNA, 870 bp, Homo sapiens
atgaagacattgcctgccatgcttggaactgggaaattattttgggtcttcttcttaatcccatatctggacatctggaacatccatgggaaagaatcatgtgatgtacagctttatataaagagacaatctgaacactccatcttagcaggagatccctttgaactagaatgccctgtgaaatactgtgctaacaggcctcatgtgacttggtgcaagctcaatggaacaacatgtgtaaaacttgaagatagacaaacaagttggaaggaagagaagaacatttcatttttcattctacattttgaaccaatgcttcctaatgacaatgggtcataccgctgttctgcaaattttcagtctaatctcattgaaagccactcaacaactctttatgtgacagatgtaaaaggtgcctcagaacgaccctccaaggacgaagtggcaagcagaccctggctcctgtatagtttacttcctttggggggattgcctctactcatcactacctggttctgcctgttctgctgcctgagaaggcaccaaggaaagcaaaatgaactctctgacacagcaggaagggaaattaatctggttgatgctcaccttaagagcgagcaaacagaagcaagcaccaggcaaaattcccaagtactgctatcagaagctggaatttatgataatgaccctgacctttgtttcaggatgcaggaagggtctgaagtttgttctaatccatgcctggaagaaaacaaaccaggcattgtttatgcttccctgaaccattctgtcattggactgaactcaagactggcaagaaatgtaaaagaagcaccaacagaatatgcatccatatgtgtgaggagttaa

SEQ ID NO: 34 - DNA, 1790 bp, Homo sapiens
tactaaccatgctgaccactgttcggaacgggattgaatcacagaaaaacagcaaatggctctctcttacagagtgtctgaacttcaaagcacaattcctgagcacattttgcagagcacctttgttcacgttatctcttctaactggtctggattacagacagaatcaataccagaggaaatgaaacagattgttgaggaacagggaaataaactgcactgggcagctcttctgatactcatggtgataatacccacaattggtggaaatacccttgttattctggctgtttcactggagaagaagctgcagtatgctactaattactttctaatgtccttggcggtggctgatttgctggttggattgtttgtgatgccaattgccctcttgacaataatgtttgaggctatgtggcccctcccacttgttctatgtcctgcctggttatttcttgacgttctcttttcaaccgcatccatcatgcatctctgtgccatttcagtggatcgttacatagccatcaaaaagccaatccaggccaatcaatataactcacgggctacagcattcatcaagattacagtggtgtggttaatttcaataggcattgccattccagtccctattaaagggatagagactgatgtggacaacccaaacaatatcacttgtgtgctgacaaaggaacgttttggcgatttcatgctctttggctcactggctgccttcttcacacctcttgcaattatgattgtcacctactttctcactatccatgctttacagaagaaggcttacttagtcaaaaacaagccacctcaacgcctaacatggttgactgtgtctacagttttccaaagggatgaaacaccttgctcgtcaccggaaaaggtggcaatgctggatggttctcgaaaggacaaggctctgcccaactcaggtgatgaaacacttatgcgaagaacatccacaattgggaaaaagtcagtgcagaccatttccaacgaacagagagcctcaaaggtcctagggattgtgtttttcctctttttgcttatgtggtgtcccttctttattacaaatataactttagttttatgtgattcctgtaaccaaactactctccaaatgctcctggagatatttgtgtggataggctatgtttcctcaggagtgaatcctttggtctacaccctcttcaataagacatttcgggatgcatttggccgatatatcacctgcaattaccgggccacaaagtcagtaaaaactctcagaaaacgctccagtaagatctacttccggaatccaatggcagagaactctaagtttttcaagaaacatggaattcgaaatgggattaaccctgccatgtaccagagtccaatgaggctccgaagttcaaccattcagtcttcatcaatcattctactagatacgcttctcctcactgaaaatgaaggtgacaaaactgaagagcaagttagttatgtatagcagaactggcagttgtcatcaaacataatgatgagtaagatgatgaatgagatgtaaatgtgcccagaatatattatataaagaattttatgtcatatatcaaatcatctctttaacctaagatgtaagtattaagaatatctaattttcctaatttggacaagattattccatgaggaaaataattttatatagctacaaatgaaaacaatccagcactctggttaaattttaaggtattcgaatgaaataaagtcaaatcaataaatttcaggctttaaaaaaaaaa

SEQ ID NO: 35 - DNA, 672 bp, Homo sapiens
atggcttgccttggatttcagcggcacaaggctcagctgaacctggctgccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgacggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaattga

SEQ ID NO: 36 - DNA, 2729 bp, Homo sapiens
ggcggccgagcaccgagcgctgggcaccgggcaccgagcggcggcggcacgcgaggcccggccccgagcagcgcccccgcccgccgcggcctccagcccggccccgcccagcgccggcccgcggggatgcggagcggcgggcgccggaggccgcggcccggctaggcccgcgctcgcgcccggacgcggcggcccgaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa

SEQ ID NO: 37 - DNA, 2026 bp, Homo sapiens
tataatttcgtacagcaacttgcggagagatttgtgagccctgaaatgagattatctttcattgtgttttcttctcaagcaactattattttgccattaactggagacagaggcaaaatcagtaaaggcttggaggatttaaaacgtgttagtccagtaggagagacatatatccatgaaggactaaagctagcgaatgaacaaattcagaaagcaggaggcttgaaaacctccagtatcataattgctctgacagatggcaagttggacggtctggtgccatcatatgcagagaaagaggcaaagatatccaggtcacttggggctagtgtttattgtgttggtgtccttgattttgaacaagcacagcttgaaagaattgctgattccaaggagcaagttttccctgtcaaaggtggatttcaggctcttaaaggaataattaattctatactagctcagtcatgtactgaaatcctagaattgcagccctcaagtgtctgtgtgggggaggaatttcagattgtcttaagtggaagaggattcatgctgggcagtcggaatggcagtgttctctgcacttacactgtaaatgaaacatatacaacgagtgtaaaaccagtaagtgtacagcttaattctatgctttgtcctgcacctatcctgaataaagctggagagtggggtctcactgtcacccaggctggagtgaagtggcatgatctgactcactgtacttttggcctttctggctcaggtgatcctcctacttcagcctcctgagtagctggggctacagaactcttgatgtttcagtgagctttaatggaggaaaatctgtcatttcaggatcattaattgtcacagccacagaatgttctaacgggatcgcagccatcattgttattttggtgttactgctactcctggggatcggtttgatgtggtggttttggcccctttgctgcaaagtggttattaaggatcctccaccaccacccccccctgcaccaaaagaggaggaagaagaacctttgcctactaaaaagtggccaactgtggatgcttcctattatggtggtcgaggggttggaggaattaaaagaatggaggttcgttggggtgataaaggatctactgaggaaggtgcaaggctagagaaagccaaaaatgctgtggtgaagattcctgaagaaacagaggaacccatcaggcctagaccacctcgacccaaacccacacaccagcctcctcagacaaaatggtacaccccaattaagggtcgtcttgatgctctctgggctttgttgaggcggcagtatgaccgggtttctttgatgcgacctcaggaaggagatgagggccggtgcataaacttctcccgagttccatctcagtaaaagggaagcaggaagaccaagaagaaaaaccaccatcgtgtcacgtcgacgatgccaaattatgttagcgtgagcagaaacaccgtgggggaggaaggcagcagctgaagaaaaaagctcaaatgatctagtcactttcgatactgtacttcagatgcgaaatggatattcgagtggaaatctgacaaagtgcgcctgctttgatgtgaactggtatagacaatgaccagtggctgggtcagtgggatgtctctctgtgagcacaaaggcttatcaaatgacactaaagataagttcaacaaccatcacattggaagggagaaggcgaacatttcatgtttggcgggcatgtgagtgcacaagatggaaagagcgattggagcatcctggtataattacccccattgtgctcttaatggaaatttcaaagaacgggagtattctgttggttggtgtccaggtttgtggcactgttccaagaggccttacacacacacacaaatatacaattttctatacatatatatcctctagcttgaaacttttgctcaagtttatttatgtcactggctggctggatccaaagtcatgtgtccacacattcataaataaaaattttacctatg

SEQ ID NO: 38 - DNA, 958 bp, Homo sapiens
ggaggaggaggaggaggaggaagaagaggtagcggcaaggtcgcggtctgaggttccggtgctcgccgccgcccagctcccagccgaggctttctcaaccgcgtcaataaaaggccgccccgacccgcccccgcgccccgcagccctgccggacaccccgggctgcagctgagcgggcgcagacgggccgaggcgggcgccgggcgcgcagggaccgagggaccgagtgctccccatgagcgcacgtgggccgggcggtccgcaagcccggctgagagcgcgccatggggcaggcgggctgcaaggggctctgcctgtcgctgttcgactacaagaccgagaagtatgtcatcgccaagaacaagaaggtgggcctgctgtaccggctgctgcaggcctccatcctggcgtacctggtcgtatgggtgttcctgataaagaagggttaccaagacgtcgacacctccctgcagagtgctgtcatcaccaaagtcaagggcgtggccttcaccaacacctcggatcttgggcagcggatctgggatgtcgccgactacgtcattccagcccagggagagaacgtcttttttgtggtcaccaacctgattgtgacccccaaccagcggcagaacgtctgtgctgagaatgaaggcattcctgatggcgcgtgctccaaggacagcgactgccacgctggggaagcggttacagctggaaacggagtgaagaccggccgctgcctgcggagagagaacttggccaggggcacctgtgagatctttgcctggtgcccgttggagacaagctccaggccggagtgagtggctgcagcctcaggaccctgaggggtttcccagccccgtgcggtgggtcccagcctgagggtggagtcctgcccactctgggagcctcacgctgagggaggagtcaggctgtcccagcgaggcccgacggcctc

SEQ ID NO: 39 - DNA, 3655 bp, Homo sapiens
tcttggtgaagtgagattcagactgagtggggtcacagcacagggcactgtcttgcctggctttatctgagccagtcacacctctcctggccactatctgtggtctagccccctttgtgcagaaagagaaagaagagccttgaggaccagcctagtcaggctgaagaaatgtcaacaattgggagttttgaaggattccaggctgtgtctctgaagcaagagggagatgaccaaccctctgagactgaccacctatcgatggaggaagaggacccgatgccaagacagatttcaaggcagtcaagtgtgaccgaatcaactctttaccccaatccttatcatcagccttatatctcacggaagtactttgctacacggccgggggccattgagactgccatggaagacttgaaaggtcacgtagctgagacttctggagagaccattcaaggcttctggctcttgacaaagatagaccactggaacaatgagaaggagagaattctactggtcacagacaagactctcttgatctgcaaatacgacttcatcatgctgagttgtgtgcagctgcagcggattcctctgagcgctgtctatcgcatctgcctgggcaagttcaccttccctgggatgtccctggacaagagacaaggagaaggccttaggatctactgggggagtccggaggagcagtctcttctgtcccgctggaacccatggtccactgaagttccttatgctactttcactgagcatcctatgaaatacaccagtgagaaattccttgaaatttgcaagttgtctgggttcatgtctaagcttgttccagctatccagaatgcccacaagaattcaactggatctggaagaggaaagaaactgatggtgttaactgaacccattttgattgagacctacacagggctgatgtcattcattggaaaccgcaacaaacttggctattcccttgcccgtgggagtattggtttttgagagtctttttggtaccataagcatatcatccacagatatgtcactttgaaaattccagtttgacccacgctatttttggactgaaacaattaattatttttaaatgacgctttatgatttagaaatttagtatttccgaaaatttaaaagcttgattggactgatagatacacactttagacctcatacaagaataatcaaattttcttaaaactagaaaataaatgctgctgagcctatcaaatactgttatcaaatgagtgcctgatcatcaactcaggaaagaagactctaagtcctgttgcttcagctctctaaatgtaggctttttttttttttttttttaggtcttggtcttcagccctcttatcctgatttattctctcattggggtctcactgtctctagtattttagctaccacttgataaggatgacttccaaatttatattcccattcccaatatttattccaagactcagttttgcatttctgactgctaatcatctatatatcagtgtcccagtggcctcttaattgagcattatcaaaatcctgtggtatttatatggtcccagttatccatccctgaagtctgtcaattttgatttctctaaattccttgccatgacatcctatatatgatcacatctctatacggcttagcattgtattccgatgcccagatgtcaaacttgaagctcattctcttatcaaaattgttcatgtattcctcctttccattcttatagctgtattattagatcaggtttttaattctgctcaaccaattattaccacagcctgttaaatggaatccctactccaaggctgactgtttcaatctatcctacacttggtttctagctcatttacttataccatcttttgttcaaatatatattttttaataacagacctttttcttctaaagaaatcttttgtagaagcagcatatacgaagcagataaaagtaggttcaatgcggttggagtgaagatgagaatgccacaggcatctttctttctaggcctcagaggtgctgcatatggaatgtccatggatcgcagagggttttgaagaccatgttttcaaaaacactgtcttaacactctattgtcattgctcttttccctcttatttcctctttgtctcttcccgacttagatctaattttcaacaacccaggaaattatctcatatgtttgcatctctcaagtttcttttcttttttctttcagagtcttattttatctaaatttatgtagaaaaaaaagctttgaatgttgactacatagacattactacaataagctttttgcctttcccgagactcaaaattgacacatctttgttgatgtttaagttgaaattccagccaactttttttgacttttatgtgaagtttaaagtcttttctttgaaaagcttgcttcctcaatttcaaagatttgtcctctgatatatttataaacatcaatttaatgcagacattcaaaccctaaaccttgaataactcttattttattttgtctttgtttacttccattaatttttttttctttccctctcttccgcctttacagcacccactttttcttccccccatgatggcaaatattgttatagtcctcaaggcagggaacttctattcagactgtaaaaggaaatgttgaaatccatagctgtcctgtcaacagtagtggaatgaaaagtcgttggattgttttggcactggttctgaagcaggcataattggcaaattattataatactgctgcccgaaaatccccatttgcctcaagatagtggcatttattggcagaagcctctgaaatgtccagcacattcttttgagttaccatttaaggatcagcctgacaatcgacttctctctgcacatttgccccctttgtaacctaagccagactgttcccaaatgaccttctaaaggacacagtgcaccatcatcaatggaaacagttcagaaaggatgacaggagggagatgtttcttagttctgagagccagtctgtaacaatgggataagcatttaatggaatcaaagctgttttgcataaagagtgaactcagagacgacaagagggtctttatgaattcccttcacattttgtaaaaactgtcatatataggctatccccttaattgataaatgggatctagaaagcggtattatgaccagagggtgagaagatttaggaatttggatagcagttcaagataagaaagaacttcttatagttataaattttcaatgatggaatgggttgcttctttaaacagtgagcccaccatcacttggtatgttcaaggaaagactagataaagtcgatattgtgagaggaattgctgtatgataccgtttggagaaatgattgacataatcttttaaggccggtgaccctatcattttccaaataattgttgctagtacttagttaattagacccatttttttcttaggaaagagttggatcaatcatgggagaccctgaaaaatttccttacctttcctaaggtttaatttcctcttctgcgaaatggagacaattatattacttttgcttatttcataaagatcttgtgaggattcaatgaaatgaggttaaattcttttattaaacagaaagttttgagt

SEQ ID NO: 40 - DNA, 3381 bp, Homo sapiens
agttggctctacgaccgccgttgccgctgccagagagggccagggcagggcagggacgcgcagcatcggggcgggcgtggggccggagaaaagggtgtcggggccacacacgcaccctcgctgcagtccagctcctcccgaagctcgcacgcccagagcgcacggagcgggggactgcccgcccagacgcccgcccgccaacccgcccacgagccggggccaaagcctgggagcctgcgaggggaggaggcccaggggggcggggtggggggcgggggtgggtgccgcgctgccgcggagctgctactcggcgcgttttgcatgaagatggcggctcccaccgccaacaaggcagcctccctgggctgtaacaacaagcctgcgttcccggagctggatttcaggtcgggagctcgggtggaggaattgaacaaactcatccaagaatttacgaagcacgaccagcgggaatacgacgaccagagagcgctggagattcacacagccaaggatttcatcttttccatgctgggaatggtgcaaaagctggaccaaaagcttccagtggctaatgaatacctgttgctctctggaggtgtccgggaaggcgtggtggacctggacttagatgagcttaatgtctatgcccggggtactgactatgatatggactttaccctcttggtgccagccctcaagctgcatgaccgtaatcagcctgtgacactcgacatgcgccactcagccttgtgccactcttggctgagccttcggctctttgatgaggggacaatcagtaaatggaaagactgctgcaccattgtagatcacatcaatggtgccaccaactacttcttctcacctaccaaagtggctgactggttctatgactctatcagcattgtcctatcagaaatacagaagaaaccccagcgagggatgccaaaggtagaaaaggtggaaaaaaatgggaccatcatctccatcattctgggtgtagggagtagtcgcatgttgtatgatattgtccctgtggtatctttcaaaggttggcctgcagtggcccagagctggctcatggagaaccacttttgggatgggaagattactgaggaagaggtcatcagtgggttttacttggtgcctgcttgctcctacaagggtaagaaggacaatgaatggcggctgtcctttgccaggagcgaggtgcagttgaagaagtgcatctccagcagcctcatgcaggcctatcaggcctgcaaagccatcatcattaaactgctgtcccggcccaaggctattagcccctatcacctgcggagcatgatgctctgggcctgcgacagacttcctgccaactacttggctcaagaagactatgcagcccactttttgctgggcctcatcgatgacctgcaacactgtctggtcaacaagatgtgccccaattatttcatccctcagtgcaacatgctggaacatctgtctgaggagacagtcatgcttcacgcccggaagctgtcctctgtgcgctcagacccggcagagcacttgcgcaccgccattgagcatgtcaaggcagccaaccggctgacactggagctccagaggcgaggtagcaccaccagcatcccctctccacagtctgacggaggggaccccaaccagcctgatgaccgtttggcaaaaaaactgcagcagctagtgactgagaacccgggaaagtcaatctctgtctttatcaatcctgacgatgtcacaaggccccatttcagaattgatgacaaatttttctgagtgttagctgactttttttttcctgattcttaaactctgataatgtgtcgtgtggtttgtgatgctatctttctgttttttacatatccagcctagattggatttgttaagaacaaattttaagtttcctggttctgcaggatggtgcaggctctgaaacagtatattactatttcatattctgcctctgatttcgttgtttactttttgtagttattgtcatgctctttagtttgttggagtttttttttctttttaaggagatcttaagccatagatgaaaatgcccatgaatcctcttgcttttttctgtttcatacttggtgcaaatttgttcatgggtatggggaaacaaatctctttctgatgcttaacgattctatcctattatttttattattttgtttttgtcttttttcttatgagctttcaccactactgatatttgagaatcatatgaacctgggaactacttggcatgttagctggatttatttattttggccagtgaccagagtagtttccccccttttccaccaaacaatgtaaactttgaaagtgataatactacaaattgtgttttggagcaacttcattgaatgtaattgtcctcaaattagagaacactgggtggtttgaagggtgtctttgacagttttccaagaagaattaaggtttccaaatgttaattttagcatatgtaattatttgaagccttatttaaatcctattaaagaacataccattataattattatttgcactaatctttgccattgtcagaattgcaaagtgtgtttcaatataaattaacaaccactctcgaaattctatcattttttcattttgcccatttctatcagtgaaatgattctttgcatttgcttagttctccttcagttgtggggtcctgctttgccctctttgatattcctggttcctgtcttactgagagaagttgttaaagctttatacatgttgaccccatctaaagctggggaaaatatacacttgatgaagtaggtgtctttgtatttatttcttttggggtcatagaggtaagcttttttgtaaaatctctactcttcctgagagttttgagaaaaactggcaggaagaacttacaatccccaaatagaagataccacgtctgttcaaaagcaggtattgctatagagcagcacccaggaaatatctgtgggtggcagccttggtatagaggtgtcctgtcttcatgagtgaagttacttgcctgattaggtgctggaaggtattggctccttgttttggtgtggactaatggcaagctggtgtttaagaggtcaggaggcctgctgatctttcaaaggacacccatgatgtctactcatagaaagggcaagtaacgtgttcattttggtcttgaatgtccaaactcaagggttggattttggccaattgaaatcagacatatgcgatcttgggtttttgcatggaaattaatgtaatgcttataaatgaaaaatgaaaacatttcaaccatctaagtcaaacacaaaaataataaaaaaagaaacatactggcctcgaaaaaaaaaaaaaaaa

SEQ ID NO: 41 - DNA, 2203 bp, Homo sapiens
ctgatgatcttgaccgacccagtgaggaagttttctctagagatctttcagattttccatctctagaaaatggcatgggaacaaatgatgaagatgaattaagccttggtttgcccactgagctcaagagaaagaaggaacagttggacagtggtcacagaccaagcaaagagacgcaatcagcagctggtctcacccttcctctgaacagtgaccaaacctttcacctgatgagcaacctggctggggatgttatcacagctgcagtgactgcagctatcaaagaccagttagagggtgtgcagcaagcactttctcaggctgcccccatcccagaagaggacacagacactgaagaaggtgatgactttgaactacttgaccagtcagagctggatcaaattgagagtgaattgggacttacacaagaccaggaagcagaagcacagcaaaataagaagtcttcaggtttcctttcaaatctgctgggaggccattaatctaggaatcagctcgcaacagagcacaaaaaacaccaaaaaaatttcaaacaagaaaaaaaaaaaaaaaaaggaaaagaaaaaaattgaactgtaagctttaatgattactttagatttgttttattttccctcctgcagtgaattaattggatatatatcagctgacactgatagattgatatttctgatcgttatttttgtgtaataagcatggaaatgaactttatacacaccactgtgttgtcagagataaatattaggggttgtttttaaagcaaaaagaaaaaaacaaaaaccaaactattaaaatcctcctataaatattctttttctttacagtttttcaagcatgcaaaacagtttattgtaacttactgaaaaatattaacaattaattgtgaatacatgctgttaccagcttccttattcctaatacctggaaaatttttttttcaacggatagattttgatgtaaaaaagaccgaaattatcaaggtatcttagttgaaggacttgggaaatactatcaaaattaatttcttaggaaaaaatttaaaagtatatttaagtactctggatagactgaaacgtttccatgttatttctgcagttgtagacttaggcttatttgtaaagaagcatgctccattgactgccatctctagtcttgcagtgggtggtattaacccatagaaagcaagcagttgtgtatcacatagacaatggttatgatgtaaacagattcagttgttttgttgttcattcgtcatatgtttgtgatagggatgttgggagcacagctctattctgcctgctcagacttaagttagacccttatcttttatattatgtcatgaaaaaagtctcctaaaattgtgaaactagttcttgatgagtgatgtgatcatcagcaataaagatataataactctgttttcttagcctgtatagaggagaggaacctgcttggctttaaaatatatttatttgccatttaagtataaatatggaatctgtttcttattgggaagatagaatatatatattttcctttaaactttttaaggtcacttttaaataaccaaatttgatttatggtttttaacaaaggactaaagagctgaaaccaacctagttttgtttttgtgatataaactttaagtgtcgagggaccatgccagcaactaccaaaaatctcttaaatcttcaggtacagctggcattttggcagatgcatagagacatctgagaccctcagaaaggaaggataatccaagaatataggaaatctgtgttctcttcctttcattttatcccttatatttctaaagactaattataagtaatctgacattttaatgtagctactcttatttattttttctttctgaggtattaaaatatctggactgagttttgccaaatgttaaagggagaagagttactgaagactttgaacacttgctttttgtgattgcttatgtcattagtgcctcatgactgtgtttgatgtcctttattgatacaaagtgagcctgtgccttcattatcttgcccattttaatacaaatggaaacctggtgtttgaaaatctctgaactgtgtgggttttggaggaatatacctgaattttattcaataacagtttctggacaggaagaaaaatacagttacatatttataaaaaaaaaaaaaaaaaa

SEQ ID NO: 42 - DNA, 760 bp, Homo sapiens
gagttttcatttgtggtgagattctctcccaggccacaagacatttcctgctcggaaccttgtttactaatttccactgcttttaaggccctgcactgaaaatgcaagctcaggcgccggtggtcgttgtgacccaacctggagtcggtcccggtccggccccccagaactccaactggcagacaggcatgtgtgactgtttcagcgactgcggagtctgtctctgtggcacattttgtttcccgtgccttgggtgtcaagttgcagctgatatgaatgaatgctgtctgtgtggaacaagcgtcgcaatgaggactctctacaggacccgatatggcatccctggatctatttgtgatgactatatggcaactctttgctgtcctcattgtactctttgccaaatcaagagagatatcaacagaaggagagccatgcgtactttctaaaaactgatggtgaaaagctcttaccgaagcaacaaaattcagcagacacctcttcagcttgagttcttcaccatcttttgcaactgaaatatgatggatatgcttaagtacaactgatggcatgaaaaaaatcaaatttttgatttattataaatgaatgttgtccctgaacttagctaaatggtgcaacttagtttctccttgctttcatattatcgaatttcctggcttataaactttttaaattacatttgaaatataaaccaaatgaaatattttaactgataaaaaaaaaaaaaaaaaataaaaaa

SEQ ID NO: 43 - DNA, 644 bp, Homo sapiens
gcgcgggcggctggggaatggctgctgccatggtgccggggcgcagcgagagctgggagcgcggggagcctggccgcccggccctgtacttctgcgggagcattcgcggcggacgcgaggacaggacgctgtacgagcggatcgtgtctcggctgcggcgattcgggacagtgctcaccgagcacgtggcggccgccgagctgggcgcgcgcggggaagaggctgctgggggtgacaggctcatccatgagcaggacctggagtggctgcagcaggcggacgtggtcgtggcagaagtgacacagccatccttgggtgtaggctatgagctgggccgggccgtggcctttaacaagcggatcctgtgcctgttccgcccgcagtctggccgcgtgctttcggccatgatccggggagcagcagatggctctcggttccaggtgtgggactatgaggagggagaggtggaggccctgctggatcgatacttcgaggctgatcctccagggcaggtggctgcctcccctgacccaaccacttgacttaatctcactttcttaaattcttctattctcagacactgctctagtaccattccttcctcttagccccaggagcaaattaaaaggtacagttaaaatcct

SEQ ID NO: 44 - DNA, 1752 bp, Homo sapiens
ggttttccagtcggacccctagagtcggggcctttggggctggctgggttgagaatcctgcggtccggtgttggtggcggtgacttgttgccaccctgcagccgcacaccctcgagccatgtcctgggctgctctgctcggcctcctggccgtgctattgctgttgctgctgctgctgagccgccggcgcgcgcggagacctggtgagcctccgttggacctgggcagcatcccctggctgggccatgccttggagtttgggagagatgctgccagcttccttaccaggatgaaggaaaagcacggtgacatatttactgtgctggtggggggcagatacgtcactgttctcctggacccacactcttacgacacagtggtgtgggagctgcgtacacggctggacttccatccctatgccatcttcctcatggagaggatttttgatctgcagcttccaaatttcaaccccagtgaggagaaggccaggatgaagccgacgctcatgcacagagacctgcaggcgctcacagaagccatgtataccaaccttcgcacggtcctgctgggtgactccacagaagcaggcagtggctggcaggagacaggtctccttgagttctcctacaacgccctgctcagcgccggctacctgaccctatatggagtggaggcctcaccacgcacccatgagagtcaggcccaagatcgtgtccactcagcggatgttttccataccttccgccagctggatctgctgctccccaaactggctcgaggctccttgtcagcgggggataaagaccatgcatgcagtgtcaaaaaccgcctgtggaagctgctgtccccagccaggctggcctccagggctgaccggagcagctggctggagagttatctgagacatcttgaggagatgggggtgtcagaggagatgcaggcccgggctctggtgttgcagctctgggccacacaggggaatatgggtcccacggccttctggctccttcttttcctcctcaagaatccggaagccctcgccgctgtccgtgcagagctgaagcacacggtctggcaagcagagcagcctgtttcacagatgaccacactcccacagaagattctagacagcatgcctgtgctagacagtgtgctcaatgagacactccggctcacggctgcccccttcatcacccgtgaggtcatggcagacctggccttgcctatggcagacgggagggaattctctcttcgacgtggtgaccgccttctcctctttcccttcctgagtccccagaaggacccagaaatctacacagagcctgaggtgtttaaatacaaccgattcttgaacccagatggatctgaaaagaaagatttttacaaagatgggaaacggctgaagaattacaacatgccatggggcgcagggcacaaccagtgcctggggaagagttatgccatcaacagcatcaaacaatttgtcgtcctactgctgactcatttcgacctggagctgggcagcgaggacacagaggttcctgagtttgacctcagcagatatggcttcggtctgatgcagccagaggaagacgtgcccatccggtaccgtgccaggctgtgacctatggagcagactcacctggggccttctgacttcctgttgttcctctagaatgctatgtccagcggaggggacactgaggacagtgagagcaggaaaagctaataaaagctctgtatctcgctgtg

SEQ ID NO: 45 - DNA, 2589 bp, Homo sapiens
gtggccagtggccgggccatgttgctagtgttgctgatcttggctgcatcgtgcaggagcgccctgcctcgagagccgactattcagtgtggctctgagacagggccatctccagagtggatggtccaacacacactcactccaggagacttgagggacctccaagtggaactcgtcaagacaagtgtggcagcagaggagttttcaattttgatgaacataagctggatactccgggcagacgccagcatccgcttgttgaaggccaccaagatctgcgtgagtggcaaaaacaacatgaattcatacagctgtgtgaggtgcaactacacagaggccttccaaagccagaccagaccttccggcggcaaatggacattctcctatgtaggcttccctgtggagctgagcactctctatctcatcagcgcccataacatccccaatgctaatatgaatgaggacagcccttctttgtctgtgaacttcacctcgccagggtgcactcgtgaaaacacagaagtaacgtccggtgtatttccagcagctaaacaccaggctctccggatttcagctcctttcccattacaatttcctcctgggccagaggactcagtcattctgccaccccagcctctggcgtcgctttttcatgactttgtcaaacttacctagcttgtttccattctgaaattgtctgatgcttgcttcgtatgtaagccggggatatgaggtttgggtatgaatcccacagagggcactgaattcttctcactatggcctatctgggctgtgtgacattgttggtgagggtcgtgcctactaggcatctgggtatctaccacctggagcttcatgtctggaagaggcagaacctatatgtattgtcagctctcacttttgtttccgtgtcacctcctggagactgtttttgataaaggttgtacttaaaggagattacttaaagcttccgtggaagaatggtttcctatttagatctgttgtctctcatatctgaagtaagtgtgtgtgtgtgtgttttgtgtgtgtgtgtgtgtgtgtgtgtgtactgggcaaagggttatacctttactcaaatgtaacaactttcattcacattcccaggctgcctaaaccacgtaatgaaatataaaaagcagtgcactgaggcgggaagcctgtgggacccagacatcactgcttgtaaaaagaacgagaagatggttgaagtgaatttcacaaccaatccccttggaaacagatacacgattctcattcaacgggacacgacattggggttttctagagtgctggagaataaactgatgaggacgtctgtagccatcccggtgactgaggagagtgaaggtgcggtggttcagctgaccccatatttacatacctgcggcaatgactgcatccgacgcgaagggacagttgtgctttgctcagagacaagtgctcccatccctccagatgacaacagacgcatgctgggaggctggctgcctctcttcctggtgctgctggtggctgtgtgggtgctggcagctgggatctacctaacttggaggcaaggaaggagcacgaagacgtcctttcctatttccaccatgctcctgcccctcattaaggtcctggtggtttatccttctgagatatgtttccatcacaccgtctgtcgcttcactgactttcttcaaaactactgcagaagtgaggtcatccttgaaaaatggcagaaaaagaaaatcgccgagatggggccggtacagtggctgaccactcagaagcaagcggcagataaagtggtcttccttcttcccagtgacgtcccgaccctttgtgacagtgcctgtggccacaatgagggcagcgccagggagaactctcaggatctgttccctcttgcctttaacctcttttgtagtgatttcagcagccagacgcatctgcacaaatacctggtggtctatcttgggggagcagacctcaaaggcgactataatgccctgagtgtctgcccccaatatcatctcatgaaggacgccacagctttccacacagaacttctcaaggctacgcagagcatgtcagtgaagaaacgctcacaagcctgccatgatagctgttcacccttgtagtccacccgggggaatagagactctgaagccttcctactctcccttccagtgacaaatgctgtgtgacgactctgaaatgtgtgggagaggctgtgtggaggtagtgctatgtacaaacttgctttaaaactggagtttgcaaagtcaacctgagcatacacgcctgaggctagtcattggctggatttatgaagacaacacagttacagacaataatgagtgggacctacatttgggatatacccaaagctgggtaatgattatcactgagaaccacgcactctggccatgaagtaatacggcacttccctgtcaggctgtctgtcaggttgggtctgtcttgcactgcccatgctctatgctgcacgtagaccgttttgtaacattttaatctgttaatgaataatccgtttgggaagctctcaaaaaaaaaaaaaaaaaaaaa

SEQ ID NO: 46 - DNA, 3229 bp, Homo sapiens
tgctgacacgtgctgggaatgaagacagtgcctgccatgcttgggactcctcggttatttagggaattcttcatcctccatctgggcctctggagcatcctttgtgagaaagctactaagaggaatgatgaagagtgtccagtgcaacttactattacgaggaattccaaacagtctgccaggacaggagagttatttaaaattcaatgtcctgtgaaatactgtgttcatagacctaatgtgacttggtgtaagcacaatggaacaatctgtgtaccccttgaggttagccctcagctatacactagttgggaagaaaatcaatcagttccggtttttgttctccactttaaaccaatacatctcagtgataatgggtcgtatagctgttctacaaacttcaattctcaagttattaatagccattcagtaaccatccatgtgacagaaaggactcaaaactcttcagaacacccactaataatatctgacatcccagatgccaccaatgcctcaggaccatccaccatggaagagaggccaggcaggacttggctgctttacaccttgcttcctttgggggcattgcttctgctccttgcctgtgtctgcctgctctgctttctgaaaaggatccaagggaaagaaaagaagccttctgacttggcaggaagggacactaacctggttgatattccagccagttccaggacaaatcaccaagcactgccatcaggaactggaatttatgataatgatccctggtctagcatgcaggatgaatctgaattgacaattagcttgcaatcagagagaaacaaccagggcattgtttatgcttctttgaaccattgtgttattggaaggaatccaagacaggaaaacaacatgcaggaggcacccacagaatatgcatccatttgtgtgagaagttaaacctgccactgagccaggcagcctacactgcatgagtgcctgtcaatacctctgtctggaccttcagtttcaaataaccttcaacctggaaagtttcaattaagatgctctgtgctggtgctgcgtcttaaaggtccatgaagtatttagttaaaatttctcctgaaaactttgggagagttttgtacaagacagactcttccctgggaaaactcatattacgaatagcagaataatagggcttttaaaattgaccatgtcgcaccatgtcgtacccaatgtgtgaattcaatattttctttggaactaccaaacagacttacaggggagaaagaagcgtgttatttggatctgctcactacacattctagaaaaagaatggcccagtttgactaactagtcataaatatagtaatgattgtatgctctcagtcagtcatgataagacaattacccatgtcaatctatatagataatgctttattattttttttaatgtgaaagaaaaattatcttggactcaattcagaaaaagattagaaagggtaattggtataatacagatatagcatatgccagatagatgtggtgccaacgatccttatccccaccagtgagatgctcaccttcagctggcttgattgagaaagtaaggtaggtagtaaagggctcatccacatatagtgagtggatttgcaatcagcgtggtagtgtttggatcaagattaacgcacatgaatgtgattcaatgctgctggattaatatacatatggtacgtatgggcctatgaacaagtgtccatgggaatggatatattatcaatctagagcgaacttttagcccaccttttgctttacgtgagagcaagctcaaagggcaatttatgaaaggagaatcttgaatattgagttgatccgtagggactggcatgcattcattaatgcctgtttcttgattctgaggaagtgaatcagaaaaaatcatatgtaatgaagtagggcctatggagatcactcaggtttaaattctgacattgcagctgttcaagaagatgattatcaggtgttctgaatttccggattgagtctaattacttaaaaatagctcataattttattcatcagcttgaggcttgagttctcagaccctaaaactgaataatggcatagtcttagtatacagcagattgggcctgagttagaagccactggagcaaacacataaactgtctatgtaattgcatgcatatatgtgggtgtgcctgtgcatgtgtgtgctagcatgcaaatgcaagatgtgacgcctggaaataagacaagagaaaccagtccttttgctgtgctttttttagtttgcttagaaggtgcaaagtactagagaaaatgagtcatttatagaaaataacacgtagactggtgagaatggaagggagttgagaatgtatccataaacagtgaataaagaggccttactacacagcttttctcacctactgctcacctcgggttccagttccctgaatctagaacaacaaacaatgttttcagacatgttacaaccacttctagttaagcttgttcaggcaggtgcatttgtgggaatttatgcaaaaaagaccttagaatagctgctcatcttcaaaatagaaacaggaaggatgggagggggatgagtactgcactctgtctctcttatcagatcaacaggcaacttaagcaaatctttcagaattgtctccggctttaaaaggaaaatacttcgctctactctgtttaaaacaatcttttaaaaacgttgtgtgtacatgtatatttgggtatgcatgtatgtgtgtatatggtgtatgtgcgtggtgtacacacatgtgtatgcaggagccagaagagaaagtcaggcacccttttctattgcactccactttattctgttaagggaaggtctctcattgaacctggagctctgctagcaagcagcaagccagcgatggatcctcccatccctgacccccacattgctagactatatttgctcatgcccagctttttacaaggatgtggagaactgaaattcaggtcctcatgattgcataacaagcactctcacttactaagcatgttctaatatgttttacaatgaaagacgctgctcaagatttcacctcaatgaatgatcaattctgtttgtaccttgtttgccaatcacatagcatttatgctcttattcctacttagtaaatatgtatgttgtagctcctcaaataaatttgttccagcattattttcatggttacct

SEQ ID NO: 47 - DNA, 1304 bp, Homo sapiens
cggctacccgcagagacgcgacccgccatgcacgtgaacggcaaagtggctctggtgaccggcgcggcacagggcataggcaaagccttcgctgaggctctgctgctccatggcgccaaggtagcattggtggattggaatcttgaggcaggtgtaaaatgtaaagctgccctagatgagcagttcgaacctcagaagacactgttcgtccagtgtgatgtggctgaccagaaacaactgagagatacttttagaaaagttgtagaccatttcggaagattggatattttggtcaacaatgcaggcgtgaacaatgagaaaaactgggaacagactctacaaattaatttggtttctgttatcggtgggacctatcttggtttggattacatgagtaagcaaaacggaggtgaaggcggcatcatcatcaatatgtcttcattagcagggctcatgcctgttgcgcagcaacctgtttattgttcttcgaagcacggcatcatcggattcacacgctcagcagcgatggctgctaacctcatgaaaagcggtgtaagactgaatgtcatttgcccaggctttgtggacacacccatccttgaatccattgaaaaagaagaaaatatgggccaatatataggatataaggaccagatcaaggctatgatgaaattctatggggttttacacccgtcagccattgccaatggattaatagatctcattgaagatgatgctctcaatggtcccattatgaagatctcggcatcggaaggaattcattttcaagattatgacatctctccattactcgtaaaagctccataacctcctaagcgtcagccataactgaaacccagcacaagtgactcactcagactgcatctttactagtgtagctttttcaatgaaatgccatacgatgaaacctaccttcctgtatctgatgttaatatttttctaaatgattagtgaatgtgtggttaaacctttttgataagctaacaaaaatgcagtgcaaaaaaactaggttataacctataaaatcttaaagacaaaacaaaggatattccagaaatattctgcctctcagaaaacatatttataacagtgcctcataaatagtaatggtttcaggaagtcatcttaaaggagatacttgaattgttatttaagtcaaagcagattttaaatgattcacttcgtgtgtttgcacttcaaaagcgggaagctcctttacaatggaaatatttagtaataaaagcttgtccgggacctgcaagcgaaccattccactctttccg

SEQ ID NO: 48 - DNA, 1550 bp, Homo sapiens
actgtctggaactggactgagtcaccaaaaggcgaatggcttcatcttataaaatgtctgaacaaagcacaacttctgagcacattttacagaagacatgtgatcacctgatcctgactaaccgttctggattagagacagactcagtagcagaggaaatgaagcagactgtggagggacaggggcatacagtgcactgggcagctctcctgatactcgcggtgataatacccaccattggtgggaacatccttgtgattctggctgttgcactggagaaaaggctgcagtacgctaccaactactttttaatgtccttggcgatagcagatttgctggttggattgtttgtgatgccgattgccctcttgacaatcatgtttgaggctatatggcccctcccactggccctgtgtcctgcctggttattcctcgatgttctcttttcaactgcctccatcatgcatctctgtgccatttccctggaccgctatatagccatcaaaaagccaattcaggccaatcagtgcaacacccgggctactgcattcatcaagattacagtggtatggttaatttcaataggcatcgccatcccagtccctattaaaggaatcgagactgatgtgattaatccacacaatgtcacctgtgagctgacaaaggaccgctttggcagttttatggtctttgggtcactggctgctttcttcgtacctctcaccatcatggtagtcacttactttctcaccattcacactttacagaagaaagcttacttggtcaaaaataagccacctcaacgcctaacacggtggactgtgcccacagttttcctaagggaagactcatccttttcatcaccagaaaaggtggcaatgctggatgggtctcacagggataaaattctacctaactcaagtgatgagacacttatgcgaagaatgtcctcagttggaaaaagatcagcccaaaccatttctaatgagcagagagcctcgaaggcccttggagtcgtgtttttcctttttctgcttatgtggtgccccttttttattacaaatctaactttagctctgtgtgattcctgcaatcagaccactctcaaaacactcctggagatatttgtgtggataggctacgtttcctcgggggtgaatcctctgatctatacactcttcaataagacatttcgggaagcatttggcaggtacatcacctgcaattaccgagccacaaagtcagtaaaagcacttaggaagttttccagtacactttgttttgggaattcaatggtagaaaactctaaatttttcacaaaacatggaattcgaaatgggatcaaccctgccatgtaccagagcccaatgaggctccgatgttcaaccattcagtcctcatcaatcatcctcctcgatacccttctcactgaaaacgatggcgacaaagcggaagagcaggtcagctacatattgcaggaacgggccggcctcatcttgagagagggtgatgagcaggacgcacgcgcaccatggcaggttcaagagtga

SEQ ID NO: 49 - DNA, 1958 bp, Homo sapiens
ctacacatatgtagcacgtaccttggatcaaagctgtctatataaagtccccgagtctgtgtgggttcaaacacatctcaaggcttctggatcctgttgggttttactctgctccctgaggacctcagcacatttgccccccagccatggcttgtcttggactccggaggtacaaagctcaactgcagctgccttctaggacttggccttttgtagccctgctcactcttcttttcatcccagtcttctctgaagccatacaggtgacccaaccttcagtggtgttggctagcagccatggtgtcgccagctttccatgtgaatattcaccgtcacacaacactgatgaggtccgggtgactgtgctgcggcagacaaatgaccaaatgactgaggtctgtgccacgacattcacagagaagaatacagtgggcttcctagattaccccttctgcagtggtacctttaatgaaagcagagtgaacctcaccatccaaggactgagagctgttgacacgggactgtacctctgcaaggtggaactcatgtacccaccgccatactttgtgggcatgggcaacgggacgcagatttatgtcattgatccagaaccatgcccggattctgacttcctcctttggatccttgtcgcagttagcttggggttgtttttttacagtttcctggtcactgctgtttctttgagcaagatgctaaagaaaagaagtcctcttacaacaggggtctatgtgaaaatgcccccaacagagccagaatgtgaaaagcaatttcagccttattttattcccatcaactgaaaggccgtttatgaagaagaaggagcatacttcagtctctaaaagctgaggcaatttcaactttccttttctctccagctatttttacctgtttgtatattttaaggagagtatgcctctctttaatagaaagctggatgcaaaattccaattaagcatactacaatttaaagctaaggagcatgaacagagagctgggatatttctgttgtgtcagaaccattttactaaaagcatcacttggaagcagcataaggatatagcattatggtgtggggtcaagggaacattagggaatggcacagcccaaagaaaggaagggggtgaaggaagagattatattgtacacatcttgtatttacctgagagatgtttatgacttaaataatttttaaatttttcatgctgttattttctttaacaatgtataattacacgaaggtttaaacatttattcacagagctatgtgacatagccagtggttccaaaggttgtagtgttccaagatgtatttttaagtaatattgtacatgggtgtttcatgtgctgttgtgtatttgctggtggtttgaatataaacactatgtatcagtgtcgtcccacagtgggtcctggggaggtttggctggggagcttaggacactaatccatcaggttggactcgaggtcctgcaccaactggcttggaaactagatgaggctgtcacagggctcagttgcataaaccgatggtgatggagtgtaaactgggtctttacactcattttattttttgtttctgcttttgttttcttcaatgatttgcaaggaaaccaaaagctggcagtgtttgtatgaacctgacagaacactgtcttcaaggaaatgcctcattcctgagaccagtaggtctgtttttttaggaagttccaatactaggaccccctacaagtactatggctcctcgaaaacacaaagttaatgccacaggaagcagcagatggtaggatgggatgcacaagagttcctgaaaactaacactgttagtgtttttttttttaactcaatattttccatgaaaatgcaaccacatgtataatatttttaattaaataaaagtttcttgtgattgttaaaaaaaaaaaaaaaaaaaaaaaaaa

SEQ ID NO: 50 - DNA, 2158 bp, Homo sapiens
gagagggaacccagtcaggagcttgaatcccacgattcagcgcttctgtctgcggccaatgggggcctctgagctgacgatctcgggtactttttgtaacttccagaacagggctctaggatgtctgatgttggggcgagtggcttagggccagctccttcaggcctctctacattccttcagggatcatgggctgtgtctgcagctcaaaccctgaagatgactggatggagaacattgacgtgtgtgaaaactgccactatcccatagtcccgctggacagcaagatctcgctgcccatccggaatggctctgaagtgcgggacccactggtcacctatgagggatctctcccaccagcatccccgctgcaagacaacctggttatcgccctgcacagttatgagccctcccatgatggagacttgggctttgagaagggtgaacagctccgaatcctggagcagagcggtgagtggtggaaggctcagtccctgacgactggccaagaaggcttcattcccttcaacttcgtggcgaaagcaaacagcctggagcctgaaccttggttcttcaagaatctgagccgtaaggacgccgagcggcagcttttggcgcccgggaacacgcatggatccttcctgatccgggaaagcgaaagcactgcggggtccttttccctgtcggtcagagacttcgaccagaaccagggagaagtggtgaaacattacaagatccgtaacctagacaacggtggcttctacatctcccctcgtatcacttttcccggattgcacgatctagtccgccattacaccaacgcctctgatgggctgtgcacaaagttgagccgtccttgccagacccagaagccccagaaaccatggtgggaggacgaatgggaagttcccagggaaacactgaagttggtggagcggctgggagctggccagttcggggaagtgtggatggggtactacaacggacacacgaaggtggcggtgaagagtctgaaacaagggagcatgtcccccgacgccttcctggctgaggctaacctcatgaagcagctgcagcacccgcggctagtccggctttatgcagtggtcacccaggaacccatctacatcatcacggaatacatggagaacgggagcctagtagattttctcaagactccctcgggcatcaagttgaatgtcaacaaacttttggacatggcagcccagattgcagagggcatggcgttcatcgaagaacagaattacatacatcgggacctgcgcgccgccaacatcctggtgtctgacacgctgagctgcaagattgcagactttggcctggcgcgcctcattgaggacaatgagtacacggcccgggagggggccaaatttcccattaagtggacagcaccagaagccattaactatgggaccttcaccatcaagtcagacgtgtggtccttcgggatcttgcttacagagattgtcacccacggtcgaatcccttacccaggaatgaccaaccctgaagtcattcagaacctggagagaggctaccgcatggtgagacctgacaactgtccggaagagctgtaccacctcatgatgctgtgctggaaggagcgcccagaggaccggcccacgtttgactaccttcggagtgttctggatgacttcttcacagccacagagggccagtaccagccccagccttgataggcctttcggtcccaagatcctcccaaccactgtgccctggctagggcagatggaattcctgtgccatatctgtgtggcctgtgcacacatggactctgactctgtacatgaaacctgtgcgtgtgtcacacataatgcagtttctgtcttgtacacacatctgtagttatgtgggttctacacatgtgtcttatacctgtggagcacgtgagtctaagccttggatacctcctaagatgtctcttcctataccgttccatttcctgaagccatagagaggagaaggcctgcgattgatgcgtgtctctgtctaccactgcctttcagagggtctccaggaaggccctccttgtgggctgccatccaatcttatgtctctgtgtgttctgtcctggtgcctagcacacaccaggagctcaataaaagtctgttgaatt

SEQ ID NO: 51 - DNA, 3739 bp, Homo sapiens
gctgatccccctctagcagtccacttcaccaaggtgagcgagtgtccctgctctcccccaccagacacagctctgctggcgaaagtggcagagaggtattgagggtgggtgtcaggagcccaccagtacagctggaaacacccagccactccagctcccggcaacttctcctgactctgccttcagacgagacttggaagacagtcacatctcagcagctcctctgccgttatccagcctgcctctgacaagaacccaatgcccaaccctaggccagccaagcctatggctccttccttggcccttggcccatccccaggagtcttgccaagctggaagactgcacccaagggctcagaacttctagggaccaggggctctgggggacccttccaaggtcgggacctgcgaagtggggcccacacctcttcttccttgaaccccctgccaccatcccagctgcagctgcctacagtgcccctagtcatggtggcaccgtctggggcccgactaggtccctcaccccacctacaggcccttctccaggacagaccacacttcatgcatcagctctccactgtggatgcccatgcccagacccctgtgctccaagtgcgtccactggacaacccagccatgatcagcctcccaccaccttctgctgccactggggtcttctccctcaaggcccggcctggcctgccacctgggatcaatgtggccagtctggaatgggtgtccagggagccagctctactctgcaccttcccacgctcgggtacacccaggaaagacagcaaccttttggctgcaccccaaggatcctacccactgctggcaaatggagtctgcaagtggcctggttgtgagaaggtcttcgaggagccagaagagtttctcaagcactgccaagcagatcatctcctggatgagaaaggcaaggcccagtgcctcctccagagagaagtggtgcagtctctggagcagcagctggagctggaaaaggagaagctgggagctatgcaggcccacctggctgggaagatggcgctggccaaggctccatctgtggcctcaatggacaagagctcttgctgcatcgtagccaccagtactcagggcagtgtgctcccggcctggtctgctcctcgggaggctccagacggcggcctgtttgcagtgcggaggcacctctggggaagccatggcaatagttccttcccagagttcttccacaacatggactacttcaagtaccacaatatgcgaccccctttcacctatgccacccttatccgatgggccatcctggaagccccggagaggcagaggacactcaatgaaatctaccattggtttactcgcatgttcgcctacttcagaaaccaccccgccacctggaagaatgccatccgccacaacctgagcctgcacaagtgctttgtgcgagtggagagcgagaagggagcagtgtggaccgtagatgaatttgagtttcgcaagaagaggagccaacgccccaacaagtgctccaatccctgcccttgacctcaaaaccaagaaaaggtgggcgggggagggggccaaaaccatgagactgaggctgtgggggcaaggaggcaagtcctacgtgtacctatggaaaccgggcgatgatgtgcctgctatcagggcctctgctccctatctagctgccctcctagatcatatcatctgccttacagctgagaggggtgccaatcccagcctagcccctagttccaacctagccccaagatgaactttccagtcaaagagccctcacaaccagctatacatatctgccttggccactgccaagcagaaagatgacagacaccatcctaatatttactcaacccaaaccctaaaacatgaagagcctgccttggtacattcgtgaactttcaaagttagtcatgcagtcacacatgactgcagtcctactgactcacaccccaaagcactcacccacaacatctggaaccacgggcactatcacacataggtgtatatacagacccttacacagcaacagcactggaaccttcacaattacatccccccaaaccacacaggcataactgatcatacgcagcctcaagcaatgcccaaaatacaagtcagacacagcttgtcagaacacgctcgtgtgcacgtacacacatgcagcccctccactctatctcctgagttccatgaatacacaccgactctccaagatgtaccccacgtctcacttgccactgaccccagttccctacccacaagccccaatccatgcctaagcgtggcccacagaagaacttctcttttatttgggatccaaggcccctggcccccagtgcccatccaataaactgtggtcagctggacaatcaccctgatcagatatgggaacatataagcagacagctgggtttaagatcccagcaggagaaagcggataccaaatgaaagagagtgctagaacaggtgcctcagcactgtctccagcaccccaaattcctgcctgtggttaggagacatccatcagggctctaggcctctcggacccggcccaagaggccagcattctcctggcgaagggctcggtagtcctcacagatcttctccaggttgctcaaagtcttcttgcccatctctgtctcaatctaagaaaacaggatgcacacttcttcagcccctgcaggctgcccctctactgaactcctccctgctcctcctattcccgtaacagcagcctgttccttcccatcactgggcttctgggtatgtccttccctccactccacctaaagcagcaacttctgccatgggctctgggaggcattaggagccgcaagctaaaagccagggctcagagtaggctactggctagcttcaggtcccaggcacagtgggcacgaaggcaaagcctctagctgttagttgtctggtttcaaagactctcagcgcaaaacaaggaactatcccctggcctgtctccattccccttaccagtcccaggtctcacctgctcctcaagatctcgaacttccctcatgatagtgcctgtgtcctcaatggtctggatgagctgactgcaattctggagacagcaagaatacaaggcttgcacctatgctggccctctccagccaacccaccaggcacatggctcccctcacctcatgcagggcagctaggtacttgtaggctttccgaacagcatcatccttcttagcatcctgataagacaaaggggatctccgagatatcagcaagccattcccccttttccactactctatgcccctataagaccaccctttactagtactttgccttcatcctccacagagcaaagctaggccccaagcaacagtgcacctaaaggactcacagaggggcaggcaacaactcagtcccgcctccaccctcccggaggccagcctgctccataccttgaacacaagctcatcagtcactgcaaatgtccggtcgagcttcccagagagagagttgatttccttctgcagttcctttgtgtccgacaagatctggtagaaaccagggtaactatcagtgcacatcttgggcaaggtagctgatcagtgataacactcacgtgcctatacttacatccagtcagggcccatgtcgctgtgttggggtgactattatgtgttggagtgtgcctgaacagctctgcctagtagtgagcataaagtccctgtgt

SEQ ID NO: 52 - DNA, 2482 bp, Homo sapiens
gcggccgcgtcgaccgcgcgctcaggagccaagcgtcccagctcccgctccagtgaagtttcattggtcttcggacgccgcccccgtcgccccagccctaaggaccctcgggtctcttcgacggctgctggaaggcgcccagcccgcctcggatgggcatcgtggagccgggctgcggagacatgctgaccggcaccgagccgatgccgagtgacgagggccgggggcccggagcggaccaacagcatcgtttcttctatcccgagccgggcgcacaggacccgaccgatcgccgcgcaggtagcagcctggggacgccctactctgggggcgccctggtgcctgccgcgccgggtcgcttccttggatccttcgcctacccgccccgggctcaggtggctggctttcccgggcctggcgagttcttcccgccgcccgcgggtgcggagggctacccgcccgtggatggctaccctgcccctgacccgcgcgcggggctctacccagggccgcgcgaggactacgcattgcccgcggggttggaggtgtctgggaagctgagagtcgcgctcagcaaccacctgttgtggtccaagttcaaccagcaccagacagagatgatcatcactaagcaaggacggcgaatgttcccattcctgtccttcaccgtggctgggctggagcccacaagccattacaggatgtttgtggatgtggtcttggtggaccagcaccactggcggtaccagagcggcaagtgggtgcagtgtggaaaggcagaaggcagcatgccagggaaccgcttatatgtccacccagactcccccaacaccggagcccactggatgcgccaggaagtttcatttgggaagctaaagctcaccaacaacaagggggcttccaacaatgtgacccagatgatcgtcctgcagtctctccacaagtaccagccccggctgcacatcgtggaggtgaatgatggagagccagaggctgcctgcagtgcttctaacacacacgtctttactttccaagagacccagttcattgcagtgactgcctaccagaacgcagagatcactcagctgaaaatcgacaacaacccctttgccaaaggattccgggagaactttgagtccatgtacgcatctgttgatacgagtgtcccctcgccacctggacccaactgtcaactgcttgggggagaccccttctcacctcttctatccaaccagtatcctgttcccagccgtttctaccccgaccttccaggccagcccaaggatatgatctcacagccttactggctggggacacctcgggaacacagttatgaagcggagttccgagctgtgagcatgaagcccacactcctaccctctgccccggggcccactgtgccctactaccggggccaagacgtcctggcgcctggagctggttggcccgtggcccctcaatacccgcccaagatgagcccagctggctggttccggcccatgcgaactctgcccatggacccgggcctgggatcctcagaggaacagggctcctccccctcgctgtggcctgaggtcacctccctccagccggagtccagcgactcaggactaggcgaaggagacactaagaggaggaggatatccccctatccttccagtggcgacagctcctctcccgctggggccccttctccttttgataaggaaaccgaaggccagttttataattattttcccaactgagaaaatgccgctgaattggaaggtgcccactaacttagaaaacagacgcggggctgagagccccgagctcttccccatcccttccctgtatagtgattggttggagaggaagcggggcaagaaggattctggggtttacttcttgtttcctggcccacaaggaaatacgacaggagtgtcccctgcccctttctctgcccgaactacagtcacgaacctggtgctgcttctgaccccatggttccatggagaacggagaatggactccagagagttttggacccagagggacttcatggctttctgcgaggtggaggggtcggggtggggagtccaggagagctgctctcttcccctgtccagtcagtaactttcaactgttggtctgacacctgtgttaatctctgacctgaaagtgaagatacacgcatttttacaacagccagccaaacagagaagactcaggtgactgcgggcggactgggccacctgcgaggagacaagagagggtgggtgcagaggaagggtttgaagggtgcacatttcaccaggcgaggtcactttgaaccggtgtgtacacacacgggtgtctcttttttatttcttcgggaggggggaggctatttattgtagagagtggtgtctggatgtatttcttctgttttgcatcactttctggaaataaacatggacctggtaaaaaaaaaaaaaaaaaaaaaaaaaaaaaaaaaaaaaaa

SEQ ID NO: 53 - DNA, 697 bp, Homo sapiens
gctatttgtagtaagactcaaccccagaccacaggaccggttctgcccaacccttttgaactacttggtcttttgagacctcgcatcgaagatggctcaggcaccaacagttatcgtgactcaacctggattcgttcgtgctccccaaaattccaactggcagaccagcctgtgtgattgcttcagtgactgcggagtctgcctctgtgggaccttttgtttcacttgtcttggatgtcaagtggcagctgacatgaatgagtgttgtctgtgtggaacaacggtggccatgaggactctctaccgaacccgatacggcattcctggatctatttgtgatgactacatggtcacactcttctgtcctgtttgctctgtgtgccaactcaagagagacattaacaggaggagagccatgaacgctttctaaggagctggatggcaagagctctggctgaagaagctcaactcagcacacactccttcagcctgagatttttcaaatctttggcaactgagatgggatggatccatttaattagagaacggtgaaatctttctagttgggctttttgatttattttaaatggatattgctctttgacttggtttcttcttgctcccatatcatcaaatattggagcctataatttttttaccttacattttaggtagaaaccaaataaaagattttgct

SEQ ID NO: 54 - DNA, 2299 bp, Homo sapiens
agcgcacacagagtgctcggttgggctgccgggcagcgggaggcggcggcggtggccaaagtgagctggaggcatgggccaggcggcctggaaggggttcgtgttgtctctgttcgactacaagacagcaaagttcgtggtcgccaagagcaagaaggtggggctgctctaccgggtgctgcagctcaccatcctgttgtacttactcatatgggtgtttctgataaagaagagttatcaggacattgacacttccctgcagagtgctgtggtcaccaaagtcaagggggtggcctataccaacaccacgatgcttggggaacgactctgggatgtggcagactttgtcattccgtctcagggggagaacgttttcttcgtggtcaccaacctgatcgtgactcctaaccagcggcagggcatctgtgctgagcgtgaaggcatcccggatggcgagtgttcagaggacaccgactgtcacgctggggagtctgttgtagcgggacacggactgaaaactggtcgctgtctacgggtggggaactctacccggggcacctgtgagatctttgcttggtgcccagtggagacaaagtccatgccaacggatcccctcctgaaggacgcagaaggcttcaccattttcataaagaacttcattcgcttccccaagttcaacttctccaaagccaatgtgctagaaacaggcaacaaacatttcctgaaaacctgtcacttcagctccaccaatctctactgccccatcttccgactggggtccattgttcgctgggcaggggctgacttccaggacatagccctgaagggcggtgtgataggaatccacattgaatgggactgtgaccttgataaagctgcctcccactgcaacccacactattatttcaaccgtctggacaacaaacacacacaatccatctcctctgggtataacttcaggtttgccaggtattaccgtgaccctcatggggtagagttccgtgacctgatgaaagcatatcggatccgctttgatgtgatagttaatggcaaggcgggaaaattcagcatcatccccacagtcatcaacattggttccgggctggcgctcatgggtgctggggctttcttctgtgacctggtacttatctacctcatcagaaagagcgagttttaccgagacaagaagtttgagaaggtgaggggtcagaaggaggaagataatgttgaggttgaggccaacgagatggagcaggagctgccagaggacaagccactggagagggttcatcaggacgagcaggccctggaactggcccagagtggcaggaagcagaatagcaactgccaggtgctcttcgagcctgccaggtctggcctccaggagaatgccttcgtgaacatgaagccatcacagatcttgcaaacagtgaagacgtagccagagctcttggcagggaacagattcaaagatgtctgggccaggacaggccagggctctgaactggggagaggcatctcggtggtgccggctcaggatgtggcttctgagaacaggtccttcaactccacccagcttggtggatcctgctgcaagatttgtttcatggaaggcttcctggtggccaaagtggctcaggatgcaggaaggaggccttgggcaggcctcagggaaggggcaggcctgggtttcagtggtgggtttctgttcctgactctgagcccagctggacacttgtttatttatacaggcagtacacatttattggtgcatgctgcattttagtcctatctgagagacttaaaggcgatgctgacctggccctagggtagggggcacacaagttaactctcaccagagacatcatagtcaacccagccagcctggccttcctccagcctctagccaagtctttgacaagtccccgcagagaaagacggctgctcagaaaaagctgttggatgacatccaggggacacttagctccacccccagatgttaacaagtaaagctggccaaggaggcacctggaaaaagcttaagggagagcaaacactcagggctcctaggtaagactggggaaggaaggcaagccagccactacgggaagcctagcgtggctctggaaactgacttacttgcccaggtgagacatggacttgtttgtgtggttcagcacttgctgttttggttggtgctgggggtccctgtctgacttccttcctgctctcagaagccccagccttgatgtgctaatgagataaaggataatttccactgaaaagcgaaaaaaaaaaa

SEQ ID NO: 55 - DNA, 3313 bp, Homo sapiens
cttgggtcgccaggcccgcgccagtccccgccatccgagcaacagcggcgctgctctgggaccgcggccgcgacaccagggaacaaccatgtcaccgtttcttcgaatcggtttatccaactttgactgtgggacctgccaagcttgtcagggagaggcagtgaacccctactgcgctgtgcttgtcaaagagtatgtggaatcagaaaatgggcagatgtacatccagaaaaagccaaccatgtacccaccttgggacagcacctttgacgcccacattaacaagggaagggtgatgcagatcatcgtgaagggcaagaatgtagacctcatctcagaaacaaccgtggaactctactccctggcggagagatgccgcaagaacaatgggcggacagaaatatggttagagctgaaacctcaaggccgaatgctaatgaatgcaagatactttctggaaatgagtgacacaaaggacatgagtgagtttgagaatgaaggattctttgcactgcatcagcgccgaggagccatcaaacaggccaaagtccaccatgtcaagtgtcacgagttcacggccacctttttccctcaacccacattttgctctgtctgccatgaatttgtctggggcctgaacaagcagggttaccagtgccgacagtgtaatgcagcgattcacaagaagtgcattgataaagtgatagccaagtgcacaggatccgcaatcaatagccgagaaaccatgttccataaggagagattcaagatcgacatgccacacagattcaaagtctacaactacaagagtccaaccttctgtgagcactgtggtaccctgctctgggggctggcgaggcaaggactcaaatgtgatgcatgtggcatgaacgtccaccaccgatgccagacaaaggttgccaatctttgtggtataaaccagaagctaatggctgaagcactagcgatgattgaaagcacccaacaggctcgctccttacgagattcagaacacatcttccgagaaggcccagttgaaattggtctcccatgctccaccaaaaacgaaaccaggccaccatgcgtaccaacacctgggaaaagagaaccccagggcatttcctgggattcccctttggatgggtcaaataaatcggccggtcctcctgaacccgaagtgagcatgcgcaggacttcactgcagctgaaactgaagatcgatgacttcatcctgcacaagatgttgggaaaaggaagttttggcaaggtcttcctggcagagttcaagagaaccaatcagtttttcgcaataaaagccttaaagaaagatgtggtgttgatggatgatgacgtcgagtgtacaatggtggaaaagagggttctgtccttggcatgggagcatccatttctaacacacatgttctgcacattccagaccaaggaaaatctctttttcgtgatggagtatctcaatggaggcgacttaatgtaccacatccaaagttgccacaaatttgatctttccagagccacgttttatgctgctgaggtcatccttggtctgcagttccttcattccaaaggaattgtctacagggacctgaagcttgataatatcctgttagacagagatggacatatcaaaatagcagactttgggatgtgcaaagagaacatgctaggagatgcgaagacaaatactttctgtggaactcctgactacattgctccggagatcttgctgggtcagaagtacaaccattccgtcgactggtggtccttcggggtgctcgtttatgagatgctgattggccagtcccccttccacgggcaggacgaggaggagctgttccactccatccgcatggacaaccccttctacccgaggtggctcgaaagggaggccaaggaccttctagtgaagctttttgtgagagaacctgagaagaggctgggagtgagaggagacatccgccagcatcctttgtttcgagagatcaactgggaagagcttgaaaggaaagagattgacccacccttcagaccaaaagtgaaatcaccatatgactgtagcaatttcgacaaggaattcctaagtgagaaaccccggctatcattcgccgacagagcactcatcaacagcatggaccagaacatgttcagcaacttttccttcattaacccagggatggagactctcatttgctcctgaacctcatctgtctccagactggaagggatttgccttctctctgggaactggttcaagtaacacttctgggggtgggggtctctttttcacgttagagaagaaaagaaacactgcaaaggcagggaggactcctgagctccttgtgtgacttgttacctacagcacaaaccacgcctacttcactaatgacatcatccctaatgacatcatcccgttatatctcctggaatctctcacagcagcccttgaagttagatcattattaactctagtcatttacttgaaagatggttcccgatgctgtgaaagattcgaaatgcagttctgctcttgccctagacaacagctgctggttggtgatgaaccaaggcgcaagtggaacagatttctcaagactggagcagtgatcgcctgttatagaagtcaattccactcaaccacagagaaggaaccactaagccacgttgatgtgtgcatgtctgtggaaatgtcgatgacagaagggagggaaaggggaagctctgagcagattgtaatgggaagctctccaataaacatagcatgaaacttgaaatttacaaatctgttcattctggctagccccaaaattcccaaggcagaggaaagtaaagggcagtgagcttagcagagccctttgtcgccaacagggaagggtaaggatgtcgcctacgtggaacaacttatacacacagaaggaaagtataaccaacaagggcagggtggtttacagctgccaatcaaacctgccctcccccctctgttctcagttgatctctctgtcagcgtaggtaggcactcattaccatcctcccatcatacaagaaataaaatgcatgactcttctaagataaagaaaaccaatcccttatcacgttgttcccagtgatttgatggcaaataagtccctccttaggcatcctgcaagacaacccaacccatgcatgctatttgcagtagtcagtcctgttgagttagagtcctaactatacacaatatcgtgcgatgtttatatatgttgatgagatgttgtgatgataacgtggatatgtaaaagggaataaaagaagaaagaaagatgcc

SEQ ID NO: 56 - DNA, 2626 bp, Homo sapiens
ccgggaccagcggacggaccgagcagcgtcctgcggccggcaccgcggcggcccagatccggccagcagcgcgcgcccggacgccgctgccttcagccggccccgcccagcgcccgcccgcgggatgcggagcggcgggcgcccgaggccgcggcccggctaggcccagtcgcccgcacgcggcggcccgacgctgcggccaggccggctgggctcagcctaccgagaagagactctgatcatcatccctgggttacccctgtctctgggggccacggataccatgaacgacgtagccattgtgaaggagggctggctgcacaaacgaggggaatatattaaaacctggcggccacgctacttcctcctcaagaacgatggcacctttattggctacaaggaacggcctcaggatgtggatcagcgagagtccccactcaacaacttctcagtggcacaatgccagctgatgaagacagagcggccaaggcccaacacctttatcatccgctgcctgcagtggaccacagtcattgagcgcaccttccatgtggaaacgcctgaggagcgggaagaatgggccaccgccattcagactgtggccgatggactcaagaggcaggaagaagagacgatggacttccgatcaggctcacccagtgacaactcaggggctgaagagatggaggtgtccctggccaagcccaagcaccgtgtgaccatgaacgagtttgagtacctgaaactactgggcaagggcacctttgggaaagtgattctggtgaaagagaaggccacaggccgctactatgccatgaagatcctcaagaaggaggtcatcgtcgccaaggatgaggttgcccacacgcttactgagaaccgtgtcctgcagaactctaggcatcccttccttacggccctcaagtactcattccagacccacgaccgcctctgctttgtcatggagtatgccaacgggggcgagctcttcttccacctgtctcgagagcgcgtgttctccgaggaccgggcccgcttctatggtgcggagattgtgtctgccctggactacttgcactccgagaagaacgtggtgtaccgggacctgaagctggagaacctcatgctggacaaggacgggcacatcaagataacggacttcgggctgtgcaaggaggggatcaaggatggtgccactatgaagacattctgcggaacgccggagtacctggcccctgaggtgctggaggacaacgactacggccgtgcagtggactggtgggggctgggcgtggtcatgtatgagatgatgtgtggccgcctgcccttctacaaccaggaccacgagaagctgttcgagctgatcctcatggaggagatccgcttcccgcgcacactcggccctgaggccaagtccctgctctccgggctgctcaagaaggaccctacacagaggctcggtgggggctctgaggatgccaaggagatcatgcagcaccggttctttgccaacatcgtgtggcaggatgtgtatgagaagaagctgagcccacctttcaagccccaggtcacctctgagactgacaccaggtatttcgatgaggagttcacagctcagatgatcaccatcacgccgcctgatcaagatgacagcatggagtgtgtggacagtgagcggaggccgcacttcccccagttctcctactcagccagtggcacagcctgaggcctggggcagcggctggcagctccacgctcctctgcattgccgagtccagaagccccgcatggatcatctgaacctgatgttttgtttctcggatgcgctggggaggaaccttgccagcctccaggaccaggggaggatgtttctactgtgggcagcagcctacctcccagccaggtcaggaggaaaactatcctggggtttttcttaatttatttcatccagtttgagaccacacatgtggcctcagtgcccagaacaattagattcatgtagaaaactattaaggactgacgcgaccatgtgcaatgtgggctcatgggtctgggtgggtcccgtcactgcccccattggcctgtccaccctggccgccacctgtctctagggtccagggccaaagtccagcaagaaggcaccagaagcacctccctgtggtatgctaactggccctctccctctgggcggggagaggtcacagctgcttcagccctagggctggatgggatggccagggctcaagtgaggttgacagaggaacaagaatccagtttgttgctgtgtcccatgctgttcagagacatttaggggattttaatcttggtgacaggagagcccctgccctcccgctcctgcgtggtggctcttagcgggtaccctgggagcgcctgcctcacgtgagccctctcctagcacttgtccttttagatgctttccctctcccgctgtccgtcaccctggcctgtcccctcccgccagacgctggccattgctgcaccatgtcgttttttacaacattcagcttcagcatttttactattataataagaaactgtccctccaaattcaataaaaattgcttttcaagcttgaaaaaaaaaaaaaaaaaaaaaaaaaaaaaaa

SEQ ID NO: 57 - DNA, 3010 bp, Homo sapiens
cacgcgtccgcccacgcgtccgctgaaaggagactgtaaaaagtggctctggtgtgtggagctgtggccagggcccctaccactcagctgggagacccaagacgatactgtatccgaggagcctcctgcatgtcctgctgccctgagctcactcaagctaggtgacagcgtgtgaatgctgccaccatgaatgaggtatctgtcatcaaagaaggctggctccacaaacgtggtgaatacatcaagacctggaggccacggtacttccttctgaagagtgatggatctttcattgggtataaggagaggcccgaggcccctgaccagaccttaccccccctgaacaatttctctgtagcagaatgccagctgatgaagactgagaggccacgacccaacacctttgtcatacgctgcctgcagtggaccacagtcatcgagaggaccttccatgtagactctccagatgagagggaagagtggatgcgggctatccagatggtcgccaacagtctgaagcagcggggcccaggtgaggacgccatggattacaagtgtggctcccccagtgactcttccacatctgagatgatggaggtagctgtcaacaaggcacgggccaaagtgaccatgaatgacttcgattatctcaaactcctcggcaagggcaccttcggcaaggtcattctggttcgagagaaggccactggccgctattatgccatgaagatcctgcgcaaggaggtcatcattgcaaaggatgaagtcgcccacacagtcacagagagccgggttctgcagaataccaggcaccccttccttacagccctcaagtatgccttccagacccatgaccgcctatgctttgtgatggagtatgccaacgggggtgagctgtttttccacctctctcgggagcgagtcttcacggaggatcgggcgcgcttttatggagcagagattgtgtcagctctggagtatttgcactcgagagatgtggtgtaccgtgacatcaagctggaaaaccttatgttggacaaagatggccacatcaagatcactgactttggcttgtgcaaagagggcatcagtgatggagccaccatgaaaaccttctgtggtaccccggagtacttggcgcctgaggtgctagaggacaatgactatgggcgagcagtggactggtgggggctgggtgtggtcatgtatgagatgatgtgtggccgcctgccattctacaaccaggaccacgagcgcctctttgagctcattcttatggaggagatccgcttcccgcgcacactcgggccagaggccaagtccctgctggctggactgctgaagaaggacccaaagcagaggctcggcggaggtcccagtgatgcgaaggaggtcatggagcatagattcttcctcagcatcaactggcaggacgtggtacagaaaaagctcctgccacccttcaaacctcaggtcacttcagaagtggacacaaggtactttgatgacgagttcaccgcccagtccatcacaatcacacccccagaccgatatgacagcctggacccgctggaactggaccagcggacgcacttcccccagttctcctactcagccagcatccgagagtgagcagccctctgccaccacaggacacaagcatggccgtcatccactgcctgggtggctttttaaaaaaaaaactttattttgccttttggtttgtgttcccttccctgtcccctcatcccaaatccagtttcttcttcagtccttgctcagacttagtggtcctggcggccctctcctctgggctcccctctgttccaggtgagcgctggggaccgcccagggacaggctgttgggaggatggagagtgaggtcattcctcaccactagcctccaagagggtgaagtggggagccatggggtcacatggatttggaggcacgccagcggaggacacatctagaagttcccctcactcccagcacctcagctgccttccatgctgtcctctgggcaatcttatctgctgccaccccacctggggcacaaaagcagtaatgtccagggacaggcagaagcctttggaagctgaggggtctgggtttgcttctatggagaatggatcttcaagggggatccaggagcaggagctgccttcctcttcctcccaagctactctcttgattctcaataaagcaggaggaatgttcgggggcagccctcctgcccacagcccagtgccttaccagggcttccttctagccagccttgcctcccgctcggcccctttggatggctccgttctctgcctggggcagagggaagccaaccttggctgttacacatctcacaccaagacagtattgggccccttggacttgactggggtctgaggggacagagatggggataggtgtctgatacacaatggggtggagtctgcctcatctccagggcacctgttctggagccttaaagtgaatgtttgagttagagttgtgtcagtgctgtgggtgcgggtgtgcacctgacctagcagtgagcatcccaggctcctgggatgctggtgcccacagctgtatgcagcacttgtgtgagtgggctcaggctcagccccagcctgagtgtttgatcccacggggtgcagtgagtgcaggcgatgtgcctgctgctctggccatgtctctccctcctcaatagctctgggtttttggaaatgagttctcctggttttgctctcccacacacccccagaataggtggatttcagaaaggcagtagggtggggtggttgggatggttatgggggtcatctgttttttactttgtatgtgtgtatgtttatctgagagttttcgcccatccctgctggcctttccttactcctcgtatttgtacggtacaagcaataaagacactcatttcagacccaggaaaaaaaaaaaaaaaaaaaaaaaaaaaaaaaaaaaaaaaaaaaaaaaaaaaaaaaaaaaaaaaaaa

SEQ ID NO: 58 - DNA, 21 bp, Artificial Sequence
  Synthetic sequence: RNA molecule with deoxythymidines at positions 20 and 21
cgguggaugcccacgcccgtt

SEQ ID NO: 59 - DNA, 21 bp, Artificial Sequence
  Synthetic sequence: RNA molecule with deoxythymidines at positions 20 and 21
cgggcgugggcauccaccgtt

SEQ ID NO: 60 - DNA, 21 bp, Artificial Sequence
  Synthetic sequence: RNA molecule with deoxythymidines at positions 20 and 21
uggguguccagggagccggtt

SEQ ID NO: 61 - DNA, 21 bp, Artificial Sequence
  Synthetic sequence: RNA molecule with deoxythymidines at positions 20 and 21
ccggcucccuggacacccatt

SEQ ID NO: 62 - DNA, 21 bp, Artificial Sequence
  Synthetic sequence: RNA molecule with deoxythymidines at positions 20 and 21
ugccgagauuacucagcugtt

SEQ ID NO: 63 - DNA, 21 bp, Artificial Sequence
  Synthetic sequence: RNA molecule with deoxythymidines at positions 20 and 21
cagcugaguaaucucggcatt

SEQ ID NO: 64 - DNA, 21 bp, Artificial Sequence
  Synthetic sequence: RNA molecule with deoxythymidines at positions 20 and 21
accugagaggggugucccctt

SEQ ID NO: 65 - DNA, 21 bp, Artificial Sequence
  Synthetic sequence: RNA molecule with deoxythymidines at positions 20 and 21
ggggacaccccucucaggutt

SEQ ID NO: 66 - DNA, 21 bp, Artificial Sequence
  Synthetic sequence: RNA molecule with deoxythymidines at positions 20 and 21
gaauggcaccugugugacctt

SEQ ID NO: 67 - DNA, 21 bp, Artificial Sequence
  Synthetic sequence: RNA molecule with deoxythymidines at positions 20 and 21
ggucacacaggugccauuctt

SEQ ID NO: 68 - DNA, 21 bp, Artificial Sequence
  Synthetic sequence: RNA molecule with deoxythymidines at positions 20 and 21
auacgguuuacuccacugutt

SEQ ID NO: 69 - DNA, 21 bp, Artificial Sequence
  Synthetic sequence: RNA molecule with deoxythymidines at positions 20 and 21
acaguggaguaaaccguautt

SEQ ID NO: 70 - DNA, 21 bp, Artificial Sequence
  Synthetic sequence: RNA molecule with deoxythymidines at positions 20 and 21
gagggugaacaaagaaaagtt

SEQ ID NO: 71 - DNA, 21 bp, Artificial Sequence
  Synthetic sequence: RNA molecule with deoxythymidines at positions 20 and 21
cuuuucuuuguucacccuctt

SEQ ID NO: 72 - DNA, 21 bp, Artificial Sequence
  Synthetic sequence: RNA molecule with deoxythymidines at positions 20 and 21
gaaucaaaauuacuggacatt

SEQ ID NO: 73 - DNA, 21 bp, Artificial Sequence
  Synthetic sequence: RNA molecule with deoxythymidines at positions 20 and 21
uguccaguaauuuugauuctt

SEQ ID NO: 74 - DNA, 21 bp, Artificial Sequence
  Synthetic sequence: RNA molecule with deoxythymidines at positions 20 and 21
aggccuggcuacaaguagutt

SEQ ID NO: 75 - DNA, 21 bp, Artificial Sequence
  Synthetic sequence: RNA molecule with deoxythymidines at positions 20 and 21
acuacuuguagccaggccutt

SEQ ID NO: 76 - DNA, 21 bp, Artificial Sequence
  Synthetic sequence: RNA molecule with deoxythymidines at positions 20 and 21
cugcccuuuccugccaggctt

SEQ ID NO: 77 - DNA, 21 bp, Artificial Sequence
  Synthetic sequence: RNA molecule with deoxythymidines at positions 20 and 21
gccuggcaggaaagggcagtt

SEQ ID NO: 78 - DNA, 21 bp, Artificial Sequence
  Synthetic sequence: RNA molecule with deoxythymidines at positions 20 and 21
gaaaggcauugcacagagutt

SEQ ID NO: 79 - DNA, 21 bp, Artificial Sequence
  Synthetic sequence: RNA molecule with deoxythymidines at positions 20 and 21
acucugugcaaugccuuuctt

SEQ ID NO: 80 - DNA, 21 bp, Artificial Sequence
  Synthetic sequence: RNA molecule with deoxythymidines at positions 20 and 21
caaauccagaccugaaaggtt

SEQ ID NO: 81 - DNA, 21 bp, Artificial Sequence
  Synthetic sequence: RNA molecule with deoxythymidines at positions 20 and 21
ccuuucaggucuggauuugtt

SEQ ID NO: 82 - DNA, 21 bp, Artificial Sequence
  Synthetic sequence: RNA molecule with deoxythymidines at positions 20 and 21
ugaggagacaguagaagugtt

SEQ ID NO: 83 - DNA, 21 bp, Artificial Sequence
  Synthetic sequence: RNA molecule with deoxythymidines at positions 20 and 21
cacuucuacugucuccucatt

SEQ ID NO: 84 - DNA, 21 bp, Artificial Sequence
  Synthetic sequence: RNA molecule with deoxythymidines at positions 20 and 21
acaaacgcgagcuucagugtt

SEQ ID NO: 85 - DNA, 21 bp, Artificial Sequence
  Synthetic sequence: RNA molecule with deoxythymidines at positions 20 and 21
cacugaagcucgcguuugutt

SEQ ID NO: 86 - DNA, 21 bp, Artificial Sequence
  Synthetic sequence: RNA molecule with deoxythymidines at positions 20 and 21
ugggucauaccgcuguucutt

SEQ ID NO: 87 - DNA, 21 bp, Artificial Sequence
  Synthetic sequence: RNA molecule with deoxythymidines at positions 20 and 21
agaacagcgguaugacccatt

SEQ ID NO: 88 - DNA, 21 bp, Artificial Sequence
  Synthetic sequence: RNA molecule with deoxythymidines at positions 20 and 21
gcagacccuggcuccuguatt

SEQ ID NO: 89 - DNA, 21 bp, Artificial Sequence
  Synthetic sequence: RNA molecule with deoxythymidines at positions 20 and 21
uacaggagccagggucugctt

SEQ ID NO: 90 - DNA, 21 bp, Artificial Sequence
  Synthetic sequence: RNA molecule with deoxythymidines at positions 20 and 21
auugcugccuuuauucugutt

SEQ ID NO: 91 - DNA, 21 bp, Artificial Sequence
  Synthetic sequence: RNA molecule with deoxythymidines at positions 20 and 21
acagaauaaaggcagcaautt

SEQ ID NO: 92 - DNA, 21 bp, Artificial Sequence
  Synthetic sequence: RNA molecule with deoxythymidines at positions 20 and 21
gauuuuccacaaacuguautt

SEQ ID NO: 93 - DNA, 21 bp, Artificial Sequence
  Synthetic sequence: RNA molecule with deoxythymidines at positions 20 and 21
auacaguuuguggaaaauctt

SEQ ID NO: 94 - DNA, 21 bp, Artificial Sequence
  Synthetic sequence: RNA molecule with deoxythymidines at positions 20 and 21
cauuucaguucuuccaagatt

SEQ ID NO: 95 - DNA, 21 bp, Artificial Sequence
  Synthetic sequence: RNA molecule with deoxythymidines at positions 20 and 21
ucuuggaagaacugaaaugtt

SEQ ID NO: 96 - DNA, 21 bp, Artificial Sequence
  Synthetic sequence: RNA molecule with deoxythymidines at positions 20 and 21
aaaucucaggaauuaaacutt

SEQ ID NO: 97 - DNA, 21 bp, Artificial Sequence
  Synthetic sequence: RNA molecule with deoxythymidines at positions 20 and 21
aguuuaauuccugagauuutt

SEQ ID NO: 98 - DNA, 21 bp, Artificial Sequence
  Synthetic sequence: RNA molecule with deoxythymidines at positions 20 and 21
acaccuugcucgucaccggtt

SEQ ID NO: 99 - DNA, 21 bp, Artificial Sequence
  Synthetic sequence: RNA molecule with deoxythymidines at positions 20 and 21
ccggugacgagcaaggugutt

SEQ ID NO: 100 - DNA, 21 bp, Artificial Sequence
  Synthetic sequence: RNA molecule with deoxythymidines at positions 20 and 21
cgaacagagagccucaaagtt

SEQ ID NO: 101 - DNA, 21 bp, Artificial Sequence
  Synthetic sequence: RNA molecule with deoxythymidines at positions 20 and 21
cuuugaggcucucuguucgtt

SEQ ID NO: 102 - DNA, 21 bp, Artificial Sequence
  Synthetic sequence: RNA molecule with deoxythymidines at positions 20 and 21
gguggagcucauguacccatt

SEQ ID NO: 103 - DNA, 21 bp, Artificial Sequence
  Synthetic sequence: RNA molecule with deoxythymidines at positions 20 and 21
uggguacaugagcuccacctt

SEQ ID NO: 104 - DNA, 21 bp, Artificial Sequence
  Synthetic sequence: RNA molecule with deoxythymidines at positions 20 and 21
cagagccagaaugugaaaatt

SEQ ID NO: 105 - DNA, 21 bp, Artificial Sequence
  Synthetic sequence: RNA molecule with deoxythymidines at positions 20 and 21
uuuucacauucuggcucugtt

SEQ ID NO: 106 - DNA, 21 bp, Artificial Sequence
  Synthetic sequence: RNA molecule with deoxythymidines at positions 20 and 21
gagccugaagcagggcagctt

SEQ ID NO: 107 - DNA, 21 bp, Artificial Sequence
  Synthetic sequence: RNA molecule with deoxythymidines at positions 20 and 21
gcugcccugcuucaggcuctt

SEQ ID NO: 108 - DNA, 21 bp, Artificial Sequence
  Synthetic sequence: RNA molecule with deoxythymidines at positions 20 and 21
caccagcggcugguucggctt

SEQ ID NO: 109 - DNA, 21 bp, Artificial Sequence
  Synthetic sequence: RNA molecule with deoxythymidines at positions 20 and 21
gccgaaccagccgcuggugtt

SEQ ID NO: 110 - DNA, 21 bp, Artificial Sequence
  Synthetic sequence: RNA molecule with deoxythymidines at positions 20 and 21
ggagagaauuaguagggcatt

SEQ ID NO: 111 - DNA, 21 bp, Artificial Sequence
  Synthetic sequence: RNA molecule with deoxythymidines at positions 20 and 21
ugcccuacuaauucucucctt

SEQ ID NO: 112 - DNA, 21 bp, Artificial Sequence
  Synthetic sequence: RNA molecule with deoxythymidines at positions 20 and 21
aaugaggccaagguuuaggtt

SEQ ID NO: 113 - DNA, 21 bp, Artificial Sequence
  Synthetic sequence: RNA molecule with deoxythymidines at positions 20 and 21
ccuaaaccuuggccucauutt

SEQ ID NO: 114 - DNA, 21 bp, Artificial Sequence
  Synthetic sequence: RNA molecule with deoxythymidines at positions 20 and 21
gcagauaaaaguagguucatt

SEQ ID NO: 115 - DNA, 21 bp, Artificial Sequence
  Synthetic sequence: RNA molecule with deoxythymidines at positions 20 and 21
ugaaccuacuuuuaucugctt

SEQ ID NO: 116 - DNA, 21 bp, Artificial Sequence
  Synthetic sequence: RNA molecule with deoxythymidines at positions 20 and 21
uguccauggaucgcagaggtt

SEQ ID NO: 117 - DNA, 21 bp, Artificial Sequence
  Synthetic sequence: RNA molecule with deoxythymidines at positions 20 and 21
ccucugcgauccauggacatt

SEQ ID NO: 118 - DNA, 21 bp, Artificial Sequence
  Synthetic sequence: RNA molecule with deoxythymidines at positions 20 and 21
acugcagcagcuagugacutt

SEQ ID NO: 119 - DNA, 21 bp, Artificial Sequence
  Synthetic sequence: RNA molecule with deoxythymidines at positions 20 and 21
agucacuagcugcugcagutt

SEQ ID NO: 120 - DNA, 21 bp, Artificial Sequence
  Synthetic sequence: RNA molecule with deoxythymidines at positions 20 and 21
acucugauaaugugucgugtt

SEQ ID NO: 121 - DNA, 21 bp, Artificial Sequence
  Synthetic sequence: RNA molecule with deoxythymidines at positions 20 and 21
cacgacacauuaucagagutt

SEQ ID NO: 122 - DNA, 21 bp, Artificial Sequence
  Synthetic sequence: RNA molecule with deoxythymidines at positions 20 and 21
accaaacuauuaaaauccutt

SEQ ID NO: 123 - DNA, 21 bp, Artificial Sequence
  Synthetic sequence: RNA molecule with deoxythymidines at positions 20 and 21
aggauuuuaauaguuuggutt

SEQ ID NO: 124 - DNA, 21 bp, Artificial Sequence
  Synthetic sequence: RNA molecule with deoxythymidines at positions 20 and 21
aagaccgaaauuaucaaggtt

SEQ ID NO: 125 - DNA, 21 bp, Artificial Sequence
  Synthetic sequence: RNA molecule with deoxythymidines at positions 20 and 21
ccuugauaauuucggucuutt

SEQ ID NO: 126 - DNA, 21 bp, Artificial Sequence
  Synthetic sequence: RNA molecule with deoxythymidines at positions 20 and 21
cuggcagacaggcaugugutt

SEQ ID NO: 127 - DNA, 21 bp, Artificial Sequence
  Synthetic sequence: RNA molecule with deoxythymidines at positions 20 and 21
acacaugccugucugccagtt

SEQ ID NO: 128 - DNA, 21 bp, Artificial Sequence
  Synthetic sequence: RNA molecule with deoxythymidines at positions 20 and 21
ugaaugcugucuguguggatt

SEQ ID NO: 129 - DNA, 21 bp, Artificial Sequence
  Synthetic sequence: RNA molecule with deoxythymidines at positions 20 and 21
uccacacagacagcauucatt

SEQ ID NO: 130 - DNA, 21 bp, Artificial Sequence
  Synthetic sequence: RNA molecule with deoxythymidines at positions 20 and 21
gugacacagccauccuuggtt

SEQ ID NO: 131 - DNA, 21 bp, Artificial Sequence
  Synthetic sequence: RNA molecule with deoxythymidines at positions 20 and 21
ccaaggauggcugugucactt

SEQ ID NO: 132 - DNA, 21 bp, Artificial Sequence
  Synthetic sequence: RNA molecule with deoxythymidines at positions 20 and 21
gcggauccugugccuguuctt

SEQ ID NO: 133 - DNA, 21 bp, Artificial Sequence
  Synthetic sequence: RNA molecule with deoxythymidines at positions 20 and 21
gaacaggcacaggauccgctt

SEQ ID NO: 134 - DNA, 21 bp, Artificial Sequence
  Synthetic sequence: RNA molecule with deoxythymidines at positions 20 and 21
gaacaagaaggugggccugtt

SEQ ID NO: 135 - DNA, 21 bp, Artificial Sequence
  Synthetic sequence: RNA molecule with deoxythymidines at positions 20 and 21
caggcccaccuucuuguuctt

SEQ ID NO: 136 - DNA, 21 bp, Artificial Sequence
  Synthetic sequence: RNA molecule with deoxythymidines at positions 20 and 21
agaaggguuaccaagacgutt

SEQ ID NO: 137 - DNA, 21 bp, Artificial Sequence
  Synthetic sequence: RNA molecule with deoxythymidines at positions 20 and 21
acgucuugguaacccuucutt

SEQ ID NO: 138 - DNA, 21 bp, Artificial Sequence
  Synthetic sequence: RNA molecule with deoxythymidines at positions 20 and 21
agcuggagaguggggucuctt

SEQ ID NO: 139 - DNA, 21 bp, Artificial Sequence
  Synthetic sequence: RNA molecule with deoxythymidines at positions 20 and 21
gagaccccacucuccagcutt

SEQ ID NO: 140 - DNA, 21 bp, Artificial Sequence
  Synthetic sequence: RNA molecule with deoxythymidines at positions 20 and 21
ggauccuccaccaccaccctt

SEQ ID NO: 141 - DNA, 21 bp, Artificial Sequence
  Synthetic sequence: RNA molecule with deoxythymidines at positions 20 and 21
ggguggugguggaggaucctt

SEQ ID NO: 142 - DNA, 21 bp, Artificial Sequence
  Synthetic sequence: RNA molecule with deoxythymidines at positions 20 and 21
ugaaucucuagcucaguautt

SEQ ID NO: 143 - DNA, 21 bp, Artificial Sequence
  Synthetic sequence: RNA molecule with deoxythymidines at positions 20 and 21
auacugagcuagagauucatt

SEQ ID NO: 144 - DNA, 21 bp, Artificial Sequence
  Synthetic sequence: RNA molecule with deoxythymidines at positions 20 and 21
agaagauaauauugaagcutt

SEQ ID NO: 145 - DNA, 21 bp, Artificial Sequence
  Synthetic sequence: RNA molecule with deoxythymidines at positions 20 and 21
agcuucaauauuaucuucutt

SEQ ID NO: 146 - DNA, 21 bp, Artificial Sequence
  Synthetic sequence: RNA molecule with deoxythymidines at positions 20 and 21
agcugccacaaguucgacctt

SEQ ID NO: 147 - DNA, 21 bp, Artificial Sequence
  Synthetic sequence: RNA molecule with deoxythymidines at positions 20 and 21
ggucgaacuuguggcagcutt

SEQ ID NO: 148 - DNA, 21 bp, Artificial Sequence
  Synthetic sequence: RNA molecule with deoxythymidines at positions 20 and 21
ugugcaaggagaacauguutt

SEQ ID NO: 149 - DNA, 21 bp, Artificial Sequence
  Synthetic sequence: RNA molecule with deoxythymidines at positions 20 and 21
aacauguucuccuugcacatt

SEQ ID NO: 150 - DNA, 21 bp, Artificial Sequence
  Synthetic sequence: RNA molecule with deoxythymidines at positions 20 and 21
guuucucagaaccgaagcctt

SEQ ID NO: 151 - DNA, 21 bp, Artificial Sequence
  Synthetic sequence: RNA molecule with deoxythymidines at positions 20 and 21
ggcuucgguucugagaaactt

SEQ ID NO: 152 - DNA, 21 bp, Artificial Sequence
  Synthetic sequence: RNA molecule with deoxythymidines at positions 20 and 21
agcuaccugacccagaagutt

SEQ ID NO: 153 - DNA, 21 bp, Artificial Sequence
  Synthetic sequence: RNA molecule with deoxythymidines at positions 20 and 21
acuucugggucagguagcutt

SEQ ID NO: 154 - DNA, 21 bp, Artificial Sequence
  Synthetic sequence: RNA molecule with deoxythymidines at positions 20 and 21
ggcugagcuggcagaggugtt

SEQ ID NO: 155 - DNA, 21 bp, Artificial Sequence
  Synthetic sequence: RNA molecule with deoxythymidines at positions 20 and 21
caccucugccagcucagcctt

SEQ ID NO: 156 - DNA, 21 bp, Artificial Sequence
  Synthetic sequence: RNA molecule with deoxythymidines at positions 20 and 21
aguaugaccagcugaaggutt

SEQ ID NO: 157 - DNA, 21 bp, Artificial Sequence
  Synthetic sequence: RNA molecule with deoxythymidines at positions 20 and 21
accuucagcuggucauacutt

SEQ ID NO: 158 - DNA, 21 bp, Artificial Sequence
  Synthetic sequence: RNA molecule with deoxythymidines at positions 20 and 21
gucaucguggccaaggacgtt

SEQ ID NO: 159 - DNA, 21 bp, Artificial Sequence
  Synthetic sequence: RNA molecule with deoxythymidines at positions 20 and 21
cguccuuggccacgaugactt

SEQ ID NO: 160 - DNA, 21 bp, Artificial Sequence
  Synthetic sequence: RNA molecule with deoxythymidines at positions 20 and 21
ggaccccaagcagaggcuutt

SEQ ID NO: 161 - DNA, 21 bp, Artificial Sequence
  Synthetic sequence: RNA molecule with deoxythymidines at positions 20 and 21
aagccucugcuuggggucctt

(19) United States
(12) Patent Application Publication
(10) Pub. No.: US 2005/0048587 A1
(43) Pub. Date: Mar. 3, 2005

(54) Methods for identifying tolerance modulatory compounds and uses therefor

(75) Inventors: Patricia Rao, Acton, MA (US); Jessica Snyder, Newton, MA (US); Andria Bagley, Boston, MA (US)

Correspondence Address: LAHIVE & COCKFIELD, LLP., 28 STATE STREET, BOSTON, MA, 02109, US

(73) Assignee: TolerRx, Inc., Cambridge, MA (US)

(21) Appl. No.: 10/895,225

(22) Filed: Jul. 19, 2004

Related U.S. Application Data
(60) Provisional application No. 60/488,502, filed on Jul. 17, 2003.

(51) Int. Cl. G01N 33/567

(52) U.S. Cl. 435/7.21

(57) ABSTRACT

The invention provides methods (also referred to herein as a “screening assays”) for identifying modulators, i.e., candidate or test compounds or agents (e.g., peptides, peptidomimetics, small molecules or other drugs) capable of modulating tolerance. In one embodiment, the invention provides methods for identifying a tolerance modulatory compound, comprising, e.g., contacting a T cell with a stimulating agent and a test compound and assaying for expression or activity of at least one T regulatory (Treg) marker and at least one T effector (Teff) marker.

### RELATED APPLICATIONS

This application claims the benefit of U.S. Provisional Application 60/488,502, filed Jul. 17, 2003, titled “Methods for Identifying Tolerance Modulatory Compounds and Uses Therefor,” the entire contents of which are incorporated herein by reference.

### BACKGROUND OF THE INVENTION

The immune system provides the human body with a means to recognize and defend itself against microorganisms, viruses, and substances recognized as foreign and potentially harmful. Classical immune responses are initiated when antigen-presenting cells present an antigen to CD4+ T helper (Th) lymphocytes resulting in T cell activation, proliferation, and differentiation of effector T lymphocytes. In numerous situations, this immune response is desirable, for example, in defending the body against bacterial or viral infection, inhibiting the proliferation of cancerous cells and the like. However, unwanted or misdirected immune responses, such as those associated with allergy, autoimmune diseases, organ rejection, chronic administration of therapeutic proteins and the like, can lead to conditions in the body which are undesirable and which, in some instances, can prove fatal.

Until now, undesirable immune responses have been treated with immunosuppressive drugs, which inhibit the entire immune system, i.e., both desired and undesired immune responses. General immunosuppressants must be administered frequently, for prolonged periods of time, and have numerous harmful side effects. Withdrawal of these drugs generally results in relapse of disease. Thus, there is a need for agents that target specific components or pathways of the immune system or which target a subset of immunological responses, without modulating the entire immune system.

### SUMMARY OF THE INVENTION

The present invention pertains, at least in part, to assays designed to identify compounds or agents useful in modulating the particular immunological response known as tolerance. Tolerance occurs naturally and helps the body avoid responding to “self” antigens in a deleterious manner. Whereas classical immune responses are mediated primarily by effector T cells (Teff cells e.g., CD4+ cells (including Th1 and Th2 cells) and CD8+ cells), tolerance is mediated by a population of T cells known as T regulatory (Treg) cells. The methods of the invention feature identifying compounds which preferentially modulate Treg cells compared to Teff cells, or vice versa. The identification of compounds or agents having a preferential effect on one T cell type as compared to another, allows one to selectively enhance (or block), specific immune responses while leaving other immune responses unaffected.

Accordingly, one aspect of the invention features a method for identifying a tolerance modulatory compound, comprising contacting a T cell with a stimulating agent and a test compound, assaying for expression or activity of at least one T regulatory (Treg) marker and at least one T effector (Teff) marker, wherein a change in expression or activity of the Treg marker or the Teff marker identifies the test compound as a tolerance modulatory compound.

Another aspect of the invention features a method for identifying a tolerance modulatory compound, comprising contacting a T cell with a stimulating agent and a test compound, assaying for expression or activity of at least one T regulatory (Treg) marker and at least one T effector (Teff) marker, wherein a change in expression or activity of the Treg marker and/or an inverse change in expression or activity of the Teff marker identifies the test compound as a tolerance modulatory compound.

In yet another aspect of the invention a method for identifying a tolerance promoting compound is featured, comprising contacting a T cell with a stimulating agent and a test compound, assaying for expression or activity of at least one T regulatory (Treg) marker and at least one T effector (Teff) marker, wherein an increase in expression or activity of the Treg marker and/or decrease in expression or activity of the Teff marker identifies the test compound as a tolerance promoting compound.

Another aspect of the invention features a method for identifying a tolerance suppressing compound, comprising contacting a T cell with a stimulating agent and a test compound, assaying for expression or activity of at least one T regulatory (Treg) marker and at least one T effector (Teff) marker, wherein a decrease in expression or activity of the Treg marker and/or increase in expression or activity of the Teff marker identifies the test compound as a tolerance suppressing compound.

In one embodiment, the T cell is a naïve T cell.

In another embodiment, the stimulating agent is a mitogen. In yet another embodiment, the stimulating agent comprises an antibody. In a further embodiment, the stimulating agent is a combination of an anti-CD3 and an anti-CD28 antibody.

In one embodiment, the markers are assayed at about 36 to 60, 60 to 84, or 84 to 108 hours after contacting with the stimulating agent. In another embodiment, the markers are assayed at about 48, 72 or 96 hours after contacting with the stimulating agent.

In one embodiment, the Treg marker is FOXP3. In another embodiment, the Treg marker is at least one cytokine. In a further embodiment, the at least one cytokine is selected from the group consisting of IL-10, TGFβ, and IFNγ. In yet another embodiment, all three of the cytokines are measured.

In one embodiment, the Teff marker is Tbox21 or GATA3. In another embodiment, the Teff marker is at least one cytokine. In a further embodiment, the at least one cytokine is selected from the group consisting of IL-2, IL-12, IFNg, and TNFa. In yet another embodiment, all four of the cytokines are measured. In one embodiment, the at least one cytokine is selected from the group consisting of IL-4, IL-5, IL-13, IL-10, and TNFα. In another embodiment, all five of the cytokines are measured.

In one embodiment, the T cell is a differentiated T cell.

In one embodiment, the expression or activity of at least two Teff markers is assayed. In a further embodiment, the Teff markers are Tbox21 and GATA3.

In one embodiment, activity of the marker is assayed. In a further embodiment, the assay for activity comprises determining a protein level of the marker. In one embodiment, the Treg and/or Teff marker is a DNA-binding protein, the DNA-binding protein being capable of binding to a target DNA motif. In another embodiment, the assay for activity comprises determining a binding of the DNA-binding protein to a nucleic acid molecule comprising the DNA motif. In a further, embodiment, the nucleic acid molecule further comprises a reporter gene and the assay for activity comprises determining reporter gene expression. In another embodiment, determining reporter gene expression comprises determining an activity of a protein encoded by the reporter gene. In a further embodiment, determining the activity of the protein encoded by the reporter gene comprises determining a level of the protein.

In one embodiment, the expression of the marker is assayed. In another embodiment, the assay for expression comprises determining a nucleic acid level of the marker. In yet another embodiment, the marker is an mRNA. In one embodiment, the assay for expression comprises determining a level of the mRNA. In another embodiment, the assay for expression comprises a step of amplifying the mRNA.

Another aspect of the invention features a method for identifying a tolerance modulatory compound, comprising contacting a population of cells comprising differentiated T cells with a stimulating agent and a test compound, assaying for the effect of the test compound on the proliferation of T regulatory (Treg) cells and T effector (Teff) cells in the population, wherein a negative effect on the proliferation of Teff cells and the lack of a negative effect on the proliferation of Treg cells identifies the test compound as a tolerance modulatory compound.

In one embodiment, the test compound is an antibody. In another embodiment, the test compound is a small molecule. In yet another embodiment, the test compound is an oligonucleotide. In another embodiment, the test compound is a peptide or peptidomimetic. In yet another embodiment, the test compound is an antisense RNA molecule or a molecule that mediates RNAi.

One aspect of the invention features a method for identifying a tolerance modulatory target molecule, comprising reducing the expression of a putative Teff or Treg target molecule in a differentiated T cell, contacting the T cell with a stimulating agent, assaying for expression or activity of at least one T regulatory (Treg) marker and at least one T effector (Teff) marker by the T cell, wherein a change in expression or activity of the Treg marker or the Teff marker identifies the putative Teff or Treg target molecule as a tolerance target molecule.

In one embodiment, the expression of the putative Teff or Treg target molecule is reduced using a molecule that mediates RNAi. In another embodiment, the molecule that mediates RNAi is a short hairpin RNA (shRNA) molecule.

In one embodiment, the cell is a differentiated T cell. In another embodiment, the cell is a naïve T cell.

In one embodiment, the Teff marker and the Treg marker are cytokines. In another embodiment, the Treg marker is FOXP3. In yet another embodiment, the Teff marker is GATA3 or TBOX21.

Another aspect of the invention features a method for identifying a tolerance modulatory molecule, comprising reducing the expression of a putative Teff or Treg target molecule in a population of cells comprising differentiated T cells, contacting the population of cells with a stimulating agent, assaying for the proliferation of Treg cells and Teff cells in the population, wherein a negative effect on the proliferation of Teff cells and the lack of a negative effect on the proliferation of Treg cells identifies the putative Teff or Treg target molecule as a tolerance modulatory molecule.

### BRIEF DESCRIPTION OF THE DRAWINGS

FIG. 1A-C graphically depicts the expression of the transcription factors GATA3 (A), Tbox21 (B) and FOXP3 (C) at 72 hours post-stimulation of peripheral blood lymphocytes (PBLs) with αCD3/αCD28 in the presence of known cytokines. Expression is relative to that observed for each transcription factor in stimulated PBL (in the absence of any added cytokine).

FIG. 2A-D graphically depicts the expression of the transcription factors GATA3, Tbox21 and FOXP3 at 72 hours post-stimulation of peripheral blood lymphocytes (PBLs) with αCD3/αCD28 in the presence of the known immunomodulatory agents dexamethasone (A), rapamycin (B), cyclosporine A (C) and Cellcept (anti-proliferative agent) (D). Expression is relative to that observed for each transcription factor in stimulated PBL (in the absence of any added agent).

FIG. 3 graphically depicts the proliferation of peripheral blood lymphocytes (PBLs) at 72 hours post-stimulation with αCD3/αCD28 in the presence of the known immunomodulatory agents, rapamycin, cyclosporine, and Cellcept (MMF) (anti-proliferative agent). Proliferation is relative to that observed in control, stimulated PBL (in the absence of any added agent).

FIG. 4 graphically depicts the effect of known immunomodulatory agents (rapamycin, cyclosporine, and Cellcept (MMF)) on cytokine production in differentiated peripheral blood lymphocytes (PBLs) at 72 hours post-stimulation with αCD3/αCD28. Cytokine production is relative to that observed in control, stimulated PBL (in the absence of any added agent).

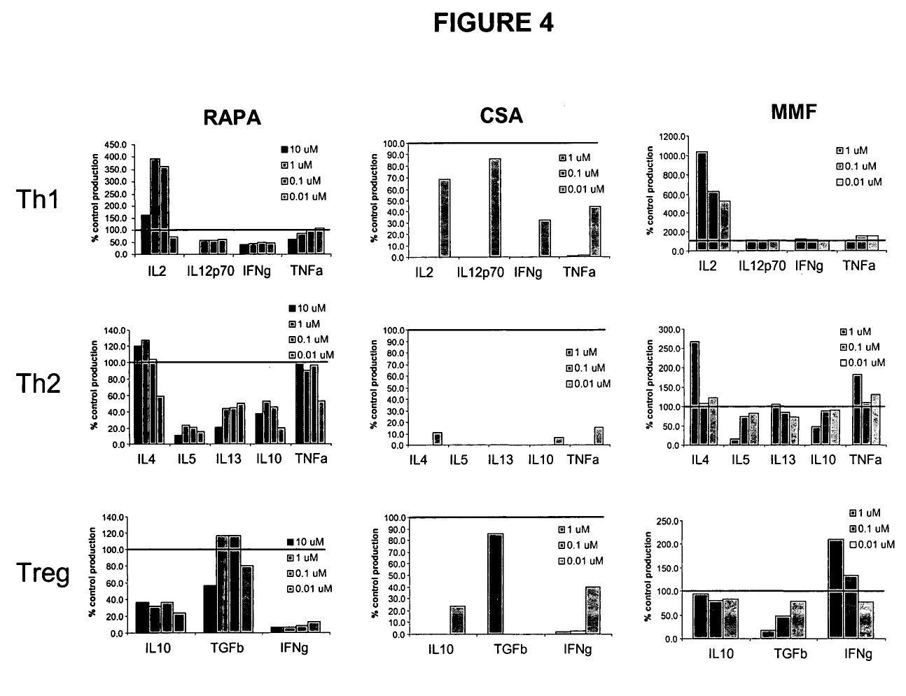

### DETAILED DESCRIPTION OF THE INVENTION

In classical immune responses, effector T cell responses dominate over responses of T regulatory cells resulting in antigen removal. Tolerance initiates with the same steps as the classical activation pathway (i.e., antigen presentation and T cell activation), but factors including, but not limited to the abundance of antigen, how it is presented to the T cell, and the relative availability of CD4+ cell help lead to the proliferation of a distinct class of lymphocytes called regulatory T (Treg) cells. Just as T effector (Teff) cells mediate classical immune responses, Treg cells mediate tolerogenic responses. The dominance or shifting of balance of regulatory T cells over effector T cells results in antigen preservation and immunological tolerance.

The present invention features methods for identifying compounds which modulate the tolerogenic response.

I. Definitions

So that the invention may be more readily understood, certain terms are first defined.

As used herein, the term “tolerance” includes refractivity to activating receptor-mediated stimulation. Tolerance can occur to self antigens or to foreign antigens. Such refractivity is generally antigen-specific and persists after exposure to the tolerizing antigen has ceased. For example, tolerance is characterized by, for example, lack of cytokine production, e.g., IL-2 or cell proliferation, e.g., T cell proliferation. As used herein the term “tolerance modulatory compound” includes compounds that modulate, i.e., promote or suppress tolerance.

As used herein, the term “T cell” (i.e., T lymphocyte) is intended to include all cells within the T cell lineage, including thymocytes, immature T cells, mature T cells and the like, from a mammal (e.g., human). T cells include mature T cells that express either CD4 or CD8, but not both, and a T cell receptor. The various T cell populations described herein can be defined based on their cytokine profiles and their function.

As used herein, the term “naïve T cells” includes T cells that have not been exposed to cognate antigen and so are not activated or memory cells. Naïve T cells are not cycling and human naïve T cells are CD45RA+. If naïve T cells recognize antigen and receive additional signals depending upon but not limited to the amount of antigen, route of administration and timing of administration, they may proliferate and differentiate into various subsets of T cells, e.g., effector T cells.

As used herein, the term “differentiated” refers to T cells that have been contacted with a stimulating agent and includes effector T cells (e.g., Th1, Th2) and memory T cells. Differentiated T cells differ in expression of several surface proteins compared to naïve T cells and secrete cytokines that activate other cells.

As used herein, the term “memory T cell” includes lymphocytes which, after exposure to antigen, become functionally quiescent and which are capable of surviving for long periods in the absence of antigen. Human memory T cells are CD45RA−.

As used herein, the term “effector T cell” includes T cells which function to eliminate antigen (e.g., by producing cytokines which modulate the activation of other cells or by cytotoxic activity). The term “effector T cell” includes T helper cells (e.g., Th1 and Th2 cells) and cytotoxic T cells. Th1 cells mediate delayed type hypersensitivity responses and macrophage activation while Th2 cells provide help to B cells and are critical in the allergic response (Mosmann and Coffman, 1989, Annu. Rev. Immunol. 7, 145-173; Paul and Seder, 1994, Cell 76, 241-251; Arthur and Mason, 1986, J. Exp. Med. 163, 774-786; Paliard et al., 1988, J. Immunol. 141, 849-855; Finkelman et al., 1988, J. Immunol. 141, 2335-2341). As used herein, the term “T helper type 1 response” (Th1 response) refers to a response that is characterized by the production of one or more cytokines selected from IFN-γ, IL-2, TNF, and lymphotoxin (LT) and other cytokines produced preferentially or exclusively by Th1 cells rather than by Th2 cells. As used herein, a “T helper type 2 response” (Th2 response) refers to a response by CD4⁺ T cells that is characterized by the production of one or more cytokines selected from IL-4, IL-5, IL-6 and IL-10, and that is associated with efficient B cell “help” provided by the Th2 cells (e.g., enhanced IgG1 and/or IgE production).

As used herein, the term “regulatory T cell” includes T cells which produce low levels of IL-2, IL-4, IL-5, and IL-12. Regulatory T cells produce TNFα, TGFβ, IFN-γ, and IL-10, albeit at lower levels than effector T cells. Although TGFβ is the predominant cytokine produced by regulatory T cells, the cytokine is produced at lower levels than in Th1 or Th2 cells, e.g., an order of magnitude less than in Th1 or Th2 cells. Regulatory T cells can be found in the CD4+ CD25+ population of cells (see, e.g., Waldmann and Cobbold. 2001. Immunity. 14:399). Regulatory T cells actively suppress the proliferation and cytokine production of Th1, Th2, or naïve T cells which have been stimulated in culture with an activating signal (e.g., antigen and antigen presenting cells or with a signal that mimics antigen in the context of MHC, e.g., anti-CD3 antibody plus anti-CD28 antibody).

Preferably, the cells used in the screening methods of the invention are mammalian cells. Human cells are preferred.

The “markers” of the invention are molecules that are preferentially expressed and/or activated in a particular cell type. Such markers may be necessary in the process that leads to differentiation of the cell type and may be expressed prior to or at an early stage of differentiation to the cell type. Such markers may be intracellular and involved in a signal transduction pathway that leads to differentiation. For example, in one embodiment, cell-specific markers are “DNA-binding proteins”, e.g., transcription factors. In another embodiment, cell-specific markers are enzymes, such as kinases. In another embodiment, cell-specific markers are cytokines. Preferably, the markers of the invention are derived from mammalian cells, e.g. human cells.

As used herein the term “T effector (Teff) marker” includes markers that are preferentially expressed and/or preferentially activated in effector T cells. For example, in one embodiment, a Teff marker is the transcription factor. Preferred transcription factors include, but are not limited to: Tbox21 (T-bet; (human—NM_{—}013351.1; gi:7019548; SEQ ID NO.:3) (mouse—NM_{—}019507; gi:9507178; SEQ ID NO.:52)) and GATA3 (NM_{—}002051.1; gi:4503928; SEQ ID NO.:2).

As used herein the term “T effector (Teff) cytokine” included cytokines that are preferentially activated in or preferentially produced by effector T cells. Preferred cytokines include, but are not limited to, IL-2, IL-4, IL-5, IL-13, IL-10, IL-12, IFNγ, and TNFα. In one embodiment, a Teff cytokine is selected from the group consisting of IL-2, IL-12, IFNγ, and TNFα. In another embodiment, a Teff cytokine is selected from the group consisting of IL-4, IL-5, IL-13, IL-10, and TNFα.

In another embodiment, a Treg marker is an enzyme. For example, in one embodiment, a Treg marker is a kinase. Exemplary kinases include: MAPK4K4 (mitogen-activated protein kinase kinase kinase kinase 4 (also known as HGK, NIK, HPK/GCK-like kinase); NM_{—}004834; gi:46249361; SEQ ID NO.:4); MAPK3K11 (mitogen-activated protein kinase kinase kinase 4 (also known as MLK3 (mixed lineage kinase 3), PTK1 (protein tyrosine kinase 1), SPRK, MLK-3, MGC17114, Sh3 domain containing proline rich kinase); NM_{—}002419; gi:21735553; SEQ ID NO.:5); MAPK4K1 (mitogen-activated protein kinase kinase kinase kinase 1 (also known as HPK1; NM_{—}007181; gi:45331203; SEQ ID NO.:6)); DYRK2 (dual-specificity tyrosine-(Y)-phosphorylation regulated kinase 2; NM_{—}003583 (variant 1)(gi:5922002; SEQ ID NO.:7), NM_{—}006482 (variant 2) (gi:5922003; SEQ ID NO.:8)); MATK (megakaryocyte-associated tyrosine kinase (also known as CHK, CTK, HYL, Lsk, HHYLTK, MGC1708, MGC2101, DKFZp434N1212; Csk-homologous kinase) (gi:21450845; SEQ ID NO.:11); NM_{—}002378 (isoform b) (gi:21450841; SEQ ID NO.:9); NM_{—}139354 (isoform c) (gi:21450843; SEQ ID NO.:10); NM_{—}139355 (isoform a); and Protein kinase C theta; (human—NM_{—}006257; gi:48255887; SEQ ID NO.:12) (mouse—NM_{—}008859; gi:6679352; SEQ ID NO.:55).

As used herein, the term “T regulatory (Treg) marker” includes markers that are preferentially expressed and/or preferentially activated in regulatory T cells. For example, in one embodiment, a Treg marker is the transcription factor FOXP3 (human—NM_{—}014009.1; gi:7661845; SEQ ID NO.:1) (mouse—NM_{—}054039; gi:16905074; SEQ ID NO.:51). In another embodiment, a Treg marker is a cytokine.

As used herein the term “T regulatory (Treg) cytokine” included cytokines that are preferentially activated in or preferentially produced by regulatory T cells. Exemplary Treg cytokines include, but are not limited to, IL-10, TGFβ, and IFNγ. For example, in one embodiment, a Treg cytokine is selected from the group consisting of IL-10, TGFβ, and IFNγ.

In another embodiment, a Treg marker is an enzyme. For example, in one embodiment, a Treg marker is a kinase. Exemplary kinases include, but are not limited to, PDK1 (3-phosphoinositide dependent protein kinase-1 (also known as PDPK1; PkB kinase); NM_{—}002613; gi:47680172; SEQ ID NO.:13); MAP4K5 (mitogen-activated protein kinase kinase kinase kinase 5 (also known as KHS; KHS1; GCKR; MAPKKKK5); NM_{—}006575; gi:38570133; SEQ ID NO.:14); MAPK2K7 (mitogen-activated protein kinase kinase 7 (also known as MKK7; JNKK2; MAPKK7; PRKMK7); NM_{—}005043; gi:21735541; SEQ ID NO.:15); CAMK2A (calcium/calmodulin-dependent protein kinase (CaM kinase) II alpha (also known as CAMKA; KIAA0968); NM_{—}015981; gi:25952113; SEQ ID NO.:16); SMG1 (PI-3-kinase-related kinase; NM_{—}015092; gi:18765738; SEQ ID NO.:17); and MVK (mevalonate kinase; NM_{—}000431; gi:4557768; SEQ ID NO.:18).

In another embodiment, a Treg marker is a phosphodiesterase, e.g., PDE4D (phosphodiesterase 4D; NM_{—}006203; gi:46361981; SEQ ID NO.:19). In another embodiment, a Treg marker is a phospholipase, e.g., PLCG2 (phospholipase C, gamma 2 (phosphatidylinositol-specific); NM_{—}002661; gi:4505870; SEQ ID NO.:20). In another embodiment, a Treg marker is a monooxygenase, e.g., KMO (kynurenine 3-monooxygenase (kynurenine 3-hydroxylase); NM_{—}003679; gi:4504890; SEQ ID NO.:21.

As used interchangeably herein, the “putative targets” or “putative Teff or Treg targets” of the invention are molecules whose modulation is predicted to be associated with the development and/or activation of Teff or Treg cells. Target molecules identified in a screen of putative Teff or Treg targets can then be used in a screening assay to identify molecules which modulate the expression and/or activity of the Teff or Treg target, i.e., the Teff or Treg target molecules can be used as drug targets. The development of agents which modulate molecules identified as Teff or Treg targets using the subject assays can be used to shift the relative balance of regulatory T cells and effector T cells, resulting in enhanced antigen preservation and immunological tolerance or enhanced T effector function.

Putative Teff and Treg target molecules include, but are not limited to lymphocyte-specific protein tyrosine kinase; (human—NM_{—}005356.1; gi:4885448; SEQ ID NO.:25) (mouse—NM_{—}010693; gi:33859569; SEQ ID NO.:50); P13 Kinase subunit (p85 alpha); gi:189424; SEQ ID NO.:27; AKT1; (human—BC000479; gi:33875493; SEQ ID NO.:36) (mouse—NM_{—}009652; gi:6753033; SEQ ID NO.:56); and AKT2 (mouse—NM_{—}007434; gi:27312021; SEQ ID NO.:57); prostaglandin 12 (prostacyclin) synthase; (human—NM_{—}000961.1; gi:13699858; SEQ ID NO.:22; (mouse—NM_{—}008968; gi:31982083; SEQ ID NO.:44); sentrin/SUMO-specific protease; NM_{—}020654.1; gi:10190689; SEQ ID NO.:23; hydroxyprostaglandin dehydrogenase 15-(NAD); human—NM_{—}000860.1; gi:4504478; SEQ ID NO.:24) (mouse—NM_{—}008278; gi:6680262; SEQ ID NO.:47); hypothetical protein FLJ32029/phosphoglucomutase 2-like 1; NM_{—}173582; gi:31377547; SEQ ID NO.:26; protein tyrosine phosphatase, non-receptor type 3; NM_{—}002829.1; gi:4506292; SEQ ID NO.:28; protein tyrosine phosphatase, non-receptor type 23; AF290614; gi:13491975; SEQ ID NO.:29; 19A24 protein-SLAMF7-Receptor; Immunoglobulin receptor family member; NM_{—}021181.2 gi:12711663; SEQ ID NO.:30); natural cytotoxicity triggering receptor 3; NM_{—}147130; gi:24475831; SEQ ID NO.:31; interleukin 17 receptor B; (human—NM_{—}018725.1; gi:8923816; SEQ ID NO.:32) (mouse—NM_{—}019583; gi:9624983; SEQ ID NO.:45); B and T lymphocyte associated receptor; (human—NM_{—}181780; gi:32401472; SEQ ID NO.:33) (mouse—NM_{—}177584; gi:31340692; SEQ ID NO.:46); 5-hydroxytryptamine (serotonin) receptor 2B; (human—NM_{—}000867.1; gi:4504538; SEQ ID NO.:34) (mouse—NM_{—}008311; gi:6680322; SEQ ID NO.:48); cytotoxic T-lymphocyte-associated protein 4; (human—NM_{—}005214.1; gi:4885166; SEQ ID NO.:35) (mouse—NM_{—}009843; gi:31981846; SEQ ID NO.:49); anthrax toxin receptor 2; NM_{—}058172; gi:17158002; SEQ ID NO.:37; P2X, ligand-gated ion channel, 5; (human—NM_{—}175081; gi:28416936; SEQ ID NO.:38) (mouse—NM_{—}033321; gi:15277324; SEQ ID NO.:54); Homo sapiens cDNA FLJ43694 fis, clone TBAES2006568; NM_{—}198485; gi:38348285; SEQ ID NO.:39; similar to BcDNA:GH11415 gene product; NM_{—}178496; gi:31341922; SEQ ID NO.:40; hypothetical protein FLJ20152; NM_{—}019000.1; gi:9506660; SEQ ID NO.:41; placenta-specific 8; (human—NM_{—}016619.1; gi:7706157; SEQ ID NO.:42) (mouse—NM_{—}139198; gi:21105852; SEQ ID NO.:53); putative c-Myc-responsive; NM_{—}006443.1; gi:5454001; SEQ ID NO.:43.

As used herein, a “surrogate marker” is an objective biochemical marker which correlates with the absence, presence or progression of a disease, disorder or immunological state (e.g., tolerance).

As used herein, the term “stimulating agent” includes one or more agents that stimulate T cell activation (e.g., effector functions such as cytokine production, proliferation, and/or lysis of target cells). A stimulating agent may also stimulate the production, proliferation and/or differentiation of T regulatory cells. Further, a stimulating agent may induce and/or maintain a toleragenic state. Exemplary stimulatory agents are known in the art and include, but are not limited to, e.g., mitogens (e.g., phytohemagglutinin or concanavalin A), antibodies that react with the T cell receptor or CD3 (in some cases combined with antigen presenting cells or antibodies that react with CD28), or antigen plus antigen presenting cells. In one embodiment, an assay of the invention further comprises addition of a cytokine or other immunomodulatory molecule.

As used herein the term “test compound” includes agents that are not known in the art to modulate tolerance. Preferably, a plurality of agents is tested using the instant methods.

In one embodiment, small molecules can be used as test compounds. The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. 1998. Science 282:63), and natural product extract libraries. In another embodiment, the compounds are small, organic non-peptidic compounds. In a further embodiment, a small molecule is not biosynthetic.

As used herein, the term “oligonucleotide” includes two or more nucleotides covalently coupled to each other by linkages (e.g., phosphodiester linkages) or substitute linkages.

As used herein, the term “peptide” includes relatively short chains of amino acids linked by peptide bonds. The term “peptidomimetic” includes compounds containing non-peptidic structural elements that are capable of mimicking or antagonizing peptides.

As used herein, the term “reporter gene” includes genes that express a detectable gene product, which may be RNA or protein. Preferred reporter genes are those that are readily detectable. The reporter gene may also be included in a construct in the form of a fusion gene with a gene that includes desired transcriptional regulatory sequences or exhibits other desirable properties. Examples of reporter genes include, but are not limited to CAT (chloramphenicol acetyl transferase) (Alton and Vapnek (1979), Nature 282: 864-869) luciferase, and other enzyme detection systems, such as beta-galactosidase, firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667), alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368) and green fluorescent protein (U.S. Pat. No. 5,491,084; WO 96/23898). The term “RNA interference” or “RNAi”, as used herein, refers generally to a sequence-specific or selective process by which a target molecule (e.g., a gene, protein, or RNA) is downregulated. In specific embodiments, the process of “RNA interference” or “RNAi” features degradation of RNA molecules, e.g., RNA molecules within a cell, said degradation being triggered by an RNA agent. Degradation is catalyzed by an enzymatic, RNA-induced silencing complex (RISC). RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of a targeted gene.

The term “RNA agent”, as used herein, refers to an RNA (or analog thereof), comprising a sequence having sufficient complimentarily to a targeted RNA (i.e., the RNA being degraded) to direct RNAi. A sequence having a “sufficiently complementary to a targeted RNA sequence to direct RNAi” means that the RNA agent has a sequence sufficient to trigger the destruction of the target RNA by the RNAi machinery (e.g., the RISC complex) or process.

The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double-stranded, i.e., dsRNA and dsDNA, respectively).

The term “mRNA” or “messenger RNA” refers to a single-stranded RNA that specifies the amino acid sequence of one or more polypeptide chains. This information is translated during protein synthesis when ribosomes bind to the mRNA.

The term “transcript” refers to a RNA molecule transcribed from a DNA or RNA template by a RNA polymerase template. The term “transcript” includes RNAs that encode polypeptides (i.e., mRNAs) as well as noncoding RNAs (“ncRNAs”).

As used herein, the term “small interfering RNA” (“siRNA”) (also referred to in the art as “short interfering RNAs”) refers to an RNA agent, preferably a double-stranded agent, of about 10-50 nucleotides in length (the term “nucleotides” including nucleotide analogs), preferably between about 15-25 nucleotides in length, more preferably about 17, 18, 19, 20, 21, 22, 23, 24, or 25 nucleotides in length, the strands optionally having overhanging ends comprising, for example, 1, 2 or 3 overhanging nucleotides (or nucleotide analogs), which is capable of directing or mediating RNA interference. Naturally-occurring siRNAs are generated from longer dsRNA molecules (e.g., >25 nucleotides in length) by a cell's RNAi machinery (e.g., the RISC complex).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region.

II. Screening Assays

Primary Screening Assays

The invention provides methods (also referred to herein as a “screening assays”) for identifying modulators, i.e., candidate or test compounds or agents (e.g., peptides, peptidomimetics, small molecules or other drugs) capable of modulating tolerance. In particular, the invention provides methods for identifying a tolerance modulatory compound, comprising:
(a) contacting a cell, such as a T cell with a stimulating agent and a test compound;
(b) assaying for expression or activity of at least one T regulatory (Treg) marker and at least one T effector (Teff) marker.
   The expression or activity of the markers in the presence of the test compound is compared with the expression or activity of the markers in the absence of the test compound (and optionally in the presence of a control compound or carrier) to thereby identify a compound that modulates the expression and/or activity of the markers.

In one embodiment, a change in expression or activity of the Treg marker and/or the Teff marker identifies the test compound as a tolerance modulatory compound. For example, an increase in the expression or activity of at least one Treg marker indicates that the compound is useful in promoting tolerance. In another embodiment, a decrease in the expression or activity of at least one Treg marker indicates that the compound is useful in reducing tolerance. In another embodiment, a decrease in the expression or activity of at least one Teff marker indicates that the compound is useful in reducing T effector function or in promoting tolerance. In another embodiment, an increase in the expression or activity of at least one Teff marker indicates that the compound is useful in reducing tolerance. In preferred embodiments, a compound causes a change in expression of at least two markers.

In another embodiment, a change in expression or activity of the Treg marker and/or inverse change in expression or activity of a Teff marker identifies the test compound as a tolerance modulatory compound. For example, in one embodiment, an increase in expression or activity of the Treg marker and/or decrease in expression or activity of the Teff marker identifies the test compound as a tolerance promoting compound. In another embodiment, a decrease in expression or activity of the Treg marker and/or increase in expression or activity of the Teff marker identifies the test compound as a tolerance suppressing compound.

The expression and or activity of Teff or Treg markers of the invention can be measured using techniques that are known in the art. For example, the expression of a marker can be measured at the RNA level (e.g., using Northern Blots or PCR) or protein level (e.g., using antibodies reactive with the marker or by detecting activity of the protein).

In another embodiment, the upstream regulatory regions of one or more markers of the invention can be operably linked to a readily detectable reporter gene and expression of the reporter gene, rather than the marker itself, can be measured to facilitate a rapid read out. A variety of reporter genes are known in the art and are suitable for use in the screening assays of the invention. As used interchangeably herein, the terms “operably linked” and “operatively linked” are intended to mean that the nucleotide sequence is linked to a regulatory sequence in a manner which allows expression of the nucleotide sequence in a host cell (or by a cell extract). The term regulatory sequence is intended to include promoters, enhancers, polyadenylation signals and other expression control elements. It should be understood that the design of the expression vector may depend on such factors as the choice of the host cell to be transfected and/or the type and/or amount of protein desired to be expressed.

Non limiting examples of suitable reporter genes include those which encode chloramphenicol acetyltransferase, beta-galactosidase, alkaline phosphatase or luciferase. Standard methods for measuring the activity of these gene products are known in the art.

The activity of Teff and Treg markers can also be measured using standard techniques. For example, in one embodiment, the activity of a marker can be determined by directly measuring the activity, e.g., transcriptional regulatory activity or enzymatic activity of the marker.

For example, in one embodiment, the Teff or Treg marker is a DNA-binding protein, e.g., a transcription factor. Accordingly, in one embodiment of the screening methods of the invention, the ability of the Teff or Treg markers to bind to DNA (e.g., the ability to bind to a specific “DNA motif”, e.g., a promoter) and/or to regulate gene expression (e.g., regulate expression of a Teff or Treg associated cytokine gene, e.g., by repressing the gene, activating or transactivating the gene) is tested. The expression of an endogenous gene can be tested or the expression of an exogenous gene, e.g., a gene comprising a DNA region responsive to the marker operably linked to a reporter gene can be tested.

In another embodiment of the screening assays of the invention, the T cell comprises: (i) the T eff and/or Treg marker and (ii) a reporter gene responsive to the Teff or Treg marker. Preferably, the T cell contains a vector comprising regulatory sequences of a promoter to which the Teff or Treg marker binds operatively linked a reporter gene. The level of expression of the reporter gene in the presence of the test compound can be compared with the level of expression of the reporter gene in the indicator cell in the absence of the test compound to thereby identify a compound that modulates the activity of the Teff or Treg marker.

In one embodiment, the level of expression of the reporter gene responsive to a Treg marker in the T cell in the presence of the test compound is higher than the level of expression of the reporter gene in the T cell in the absence of the test compound and the test compound is identified as a compound that stimulates the activity of a T reg marker. In another embodiment, the level of expression of the reporter gene in the T cell in the presence of the test compound is lower than the level of expression of the reporter gene in the T cell in the absence of the test compound and the test compound is identified as a compound that reduces the activity of a Treg marker.

The DNA motifs to which Treg and Teff DNA-binding proteins described herein bind are known in the art. For example, Tbox21 binds to T-box binding sequences (Szabo et al. 2000. Cell 100:655), FOXP3 binds to a canonical forkhead DNA binding site, and GATA3 binds to the T alpha 3 element of the human TCR alpha enhancer (Ho et al. 1991. EMBO 10: 1187.)

In another embodiment, the enzymatic activity of a Teff or Treg marker can be measured. For example, in one embodiment, the ability of a Teff or Treg marker to phosphorylate a substrate can be measured in the presence and the absence of a compound. Assays for measuring the enzymatic activities of the Teff and Treg markers described herein are well known in the art.

In another embodiment, a Teff or Treg marker is a cytokine. Assays for measuring the level of cytokines are well known in the art.

For example, changes in cytokine levels can be determined using a commercially available ELISA kit (R&D systems Quantikine kit, Minneapolis, Minn.) or by bioassay using a cytokine dependent cell line, e.g., CTLL-2 (ATCC, Rockville, Md.). IFN-gamma can be measured using a kit available from Endogen (Cambridge, Mass.). In another example, cytokine production can be measured using an ELISPOT assay (e.g., Williams et al. 1994. J. Infect. Disease. 170:946-954). The transcription of Cytokine genes can also be measured (e.g., using PCR, RNA protection assays, or northern blot analysis). Cytokine production can be measured in either primary or secondary cocultures, e.g., as is known in the art.

When more sensitive detection of cytokines is desired, techniques other than measuring cytokines in in vitro culture can be employed. For example, the cytokines can also be measured using direct staining with fluorescence-conjugated antibodies to cell surface antigens using standard methods (e.g., U.S. Pat. No. 5,767,097). For example, the cells can be stained with PE-labeled anti-IL-2 antibody and then washed in permeabilization buffer prior to FACS analysis. Alternatively, nucleic acid molecules can be isolated from cells, and mRNA levels can be measured or reverse transcribed into cDNA for amplification using the polymerase chain reaction prior to quantitation. PCR can be performed using parameters which have been optimized for the detection of cytokines. Primers for human cytokines are commercially available, e.g., from Stratagene, La Jolla, Calif. Alternatively, mRNA encoding a particular cytokine can be measured by in situ hybridization.

The sequences of cytokines that can be detected are known in the art and can be found, for example, on GenBank or in the references set forth supra. In addition, primers for amplifying these sequences and/or oligonucleotide probes that specifically bind to selected cytokine sequences are known in the art and can e.g., be chemically synthesized. In addition, many are commercially available.

In another embodiment, multiple cytokines may be measured at one time in a multiplexed cytokine assay. For example, the SearchLight Human Cytokine Array (Pierce Biotechnology, Inc., Rockford, Ill.) is a multiplexed sandwich ELISA for the quantitative measurement of IL-1β, IL-6, IFNγ and TNFα. The SearchLight array utilizes a special plate pre-spotted with up to 4 different capture antibodies per well. Following a simple ELISA procedure, the array generates a chemiluminescent signal that is imaged using a commercially-available 12-bit or 16-bit cooled CCD camera. Using array software, the intensity of the spots for each unknown sample are compared with standard curves and exact values of each cytokine (pg/ml) are calculated. Other such assays are commercially available.

In another embodiment, multiple cytokines may be measured at one time in a multiplexed cytokine assay. For example, the SearchLight Human Cytokine Array (Pierce Biotechnology, Inc., Rockford, Ill.) is a multiplexed sandwich ELISA for the quantitative measurement of IL-1β, IL-6, IFNγ and TNFα. The SearchLight array utilizes a special plate pre-spotted with up to 4 different capture antibodies per well. Following a simple ELISA procedure, the array generates a chemiluminescent signal that is imaged using a commercially-available 12-bit or 16-bit cooled CCD camera. Using array software, the intensity of the spots for each unknown sample are compared with standard curves and exact values of each cytokine (pg/ml) are calculated. Other such assays are commercially available.

In another embodiment, the proliferation of Teff cells in a population can be measured. In one embodiment, the population of T cells can be purified into Teff and Treg subpopulations using methods known in the art and the effect of a test compound on the proliferation of Teff and Treg cells can be measured. In one embodiment, a negative effect on the proliferation of Teff cells and the lack of a negative effect (e.g., no effect or a positive effect) on the proliferation of Treg cells identifies the test compound as a compound that promotes tolerance. In another embodiment, a negative effect (e.g., no effect or a positive effect) on the proliferation of Treg cells and the lack of a negative effect on the proliferation of Teff cells identifies the test compound as a compound that reduces tolerance. Methods to measure proliferation of differentiated T cells are known in the art and include for example, direct cell counts or incorporation of 3H and are described, for example, in Example 4 below.

In another embodiment of the invention, methods for identifying tolerance modulatory targets are featured. In general, the methods comprise reducing the expression of a putative Teff or Treg target molecule in a differentiated T cell and contacting the T cell with a stimulating agent to determine whether lack of the putative target molecule has any effect on the development of Teff or Treg cells by assaying for the expression or activity of at least one Teff or Treg marker.

The expression or activity of the markers in the absence of the putative target molecule is compared with the expression or activity of the markers in the presence of the putative target molecule to thereby identify a target molecule that modulates the expression and/or activity of the markers.

In one embodiment, a change in expression or activity of the Teff marker when expression of the putative Teff or Treg target is decreased identifies the putative target molecule as a molecule important in the development or activity of Teff cells. For example, an increase in the expression or activity of at least one Treg marker in the absence of the target molecule indicates that the target molecule reduces tolerance. In another embodiment, a decrease in the expression or activity of at least one Treg marker in the absence of the target molecule indicates that the target molecule promotes tolerance. In another embodiment, a decrease in the expression or activity of at least one Teff marker in the absence of the target molecule indicates that the target molecule is useful in promoting T effector function or in reducing tolerance. In another embodiment, an increase in the expression or activity of at least one Teff marker in the absence of the target molecule indicates that the target molecule is useful in promoting tolerance. In preferred embodiments, a compound causes a change in expression of at least two markers.

In another embodiment, a change in expression or activity of the Treg marker and/or inverse change in expression or activity of a Teff marker in the absence of the target molecule identifies a putative Teff or Treg molecule as a tolerance modulatory molecule. For example, in one embodiment, an increase in expression or activity of the Treg marker and/or decrease in expression or activity of the Teff marker in the absence of the target molecule identifies the putative Teff or Treg target molecule as a tolerance reducing molecule. In another embodiment, a decrease in expression or activity of the Treg marker and/or increase in expression or activity of the Teff marker in the absence of the putative Teff or Treg target molecule identifies the target molecule as a tolerance promoting molecule.

The expression and or activity of Teff or Treg markers of the invention can be measured using techniques that are known in the art or described above.

In another embodiment, the proliferation of Teff cells and/or Treg cells in a population can be measured to identify a putative Teff or Treg target molecule as a tolerance modulatory molecule. In one embodiment, the population of T cells can be purified into Teff and Treg subpopulations using methods known in the art and the effect of the absence of the putative Teff or Treg target molecule on the proliferation of Teff and Treg cells can be measured. In one embodiment, the proliferation of the cells can be measured in response to a stimulatory agent. In one embodiment, a negative effect on the proliferation of Teff cells and the lack of a negative effect on the proliferation of Treg cells in the absence of the putative Teff or Treg target molecule identifies the putative target molecule as a target that reduces tolerance. In another embodiment, a negative effect on the proliferation of Treg cells and the lack of a negative effect on the proliferation of Teff cells in the absence of the putative Teff or Treg target molecule identifies the putative target molecule as one that promotes tolerance. Methods to measure proliferation of differentiated T cells are known in the art and include for example, direct cell counts, incorporation of ³H and, as described above.

The expression of the putative Teff or Treg target molecule can be reduced using, for example, inhibitory agents desribed herein, e.g., including but not limited to, antisense nucleic acid molecules, intracellular antibodies, and dominant negative mutants of the protein corresponding to a putative target of the invention.

Preferred inhibitory agents of the instant invention are antisense nucleic acid molecules, e.g., those that mediate RNAi, including nucleic acid sequences or molecules that encode (i.e., generate) shRNA molecules. The requisite elements of a shRNA-encoding nucleic acid sequence or molecule include a first portion and a second portion, having sequences such that the RNA sequences encoded by said portions have sufficient complementarity to anneal or hybridize to form a duplex or double-stranded stem portion. The two portions need not be fully or perfectly complementary. The first and second “stem-encoding” portions are connected by a portion having a sequence that, when encoded, has insufficient sequence complementarity to anneal or hybridize to other portions of the shRNA. This latter portion is referred to as a “loop-encoding” portion in the shRNA-encoding nucleic acid sequences or molecules. The shRNA-encoding nucleic acid sequences or molecules are transcribed to generate shRNAs. shRNAs can also include one or more bulges, i.e., extra nucleotides that create a small nucleotide “loop” in a portion of the stem, for example a one-, two- or three-nucleotide loop. The encoded stem portions can be the same length, or one portion can include an overhang of, for example, 1-5 nucleotides. The overhanging nucleotides can include, for example, uracils (Us), e.g., all Us. Such Us are notably encoded by thymidines (Ts) in the shRNA-encoding DNA which signal the termination of transcription.

In one embodiment, the strand of the stem portion of the encoded shRNA is further sufficiently complementary (e.g., antisense) to a target RNA (e.g., mRNA) sequence to mediate degradation or cleavage of said target RNA via RNA interference (RNAi). The antisense portion can be on the 5′ or 3′ end of the stem. The stem-encoding portions of a shRNA-encoding nucleic acid (or stem portion of a shRNA) are preferably about 15 to about 50 nucleotides in length. In one embodiment, when used in mammalian cells, the length of the stem portions is less than about 30 nucleotides to avoid provoking non-specific responses like the interferon pathway. In one embodiment, a stem portion can include much larger sections complementary to the target mRNA (up to, and including the entire mRNA). The loop portion in the shRNA (or loop-encoding portion in the encoding DNA) can be about 2 to about 20 nucleotides in length, i.e., about 2, 3, 4, 5, 6, 7, 8, 9, or more, e.g., 15 or 20, or more nucleotides in length. A preferred loop consists of or comprises a “tetraloop” sequences. Exemplary tetraloop sequences include, but are not limited to, the sequences GNRA, where N is any nucleotide and R is a purine nucleotide, GGGG, and UUUU.

The sequence of the antisense portion of a shRNA can be designed by selecting an 18, 19, 20, 21 nucleotide, or longer, sequence from within the target RNA (e.g., mRNA), for example, from a region 100 to 200 or 300 nucleotides upstream or downstream of the start of translation. In general, the sequence can be selected from any portion of the target RNA (e.g., mRNA) including the 5′ UTR (untranslated region), coding sequence, or 3′ UTR. This sequence can optionally follow immediately after a region of the target gene containing two adjacent AA nucleotides. The last two nucleotides of the nucleotide sequence can be selected to be UU. shRNAs so generated are processed under appropriate conditions (e.g., in an appropriate in vitro reaction or in a cell) by RNAi machinery (i.e., Dicer and/or RISC complexes) to generate siRNAs. shRNAs can be synthesized exogenously or can be transcriped in vivo from an RNA polymerase (e.g., a Pol II or Pol III polymerase), thus permitting the construction of continuous cell lines or transgenic animals in which the desired gene silencing is stable and heritable.

In certain aspects of the invention, it may be important to detect the generation or expression of shRNAs, targeted mRNAs and/or the gene products encoded by said targeted RNAs. The detection methods used herein include, for example, cloning and sequencing, ligation of oligonucleotides, use of the polymerase chain reaction and variations thereof (e.g., a PCR that uses 7-deaza GTP), use of single nucleotide primer-guided extension assays, hybridization techniques using target-specific oligonucleotides that can be shown to preferentially bind to complementary sequences under given stringency conditions, and sandwich hybridization methods.

Sequencing may be carried out with commercially available automated sequencers utilizing labeled primers or terminators, or using sequencing gel-based methods. Sequence analysis is also carried out by methods based on ligation of oligonucleotide sequences which anneal immediately adjacent to each other on a target DNA or RNA molecule (Wu and Wallace, Genomics 4: 560-569 (1989); Landren et al., Proc. Natl. Acad. Sci. 87: 8923-8927 (1990); Barany, F., Proc. Natl. Acad. Sci. 88: 189-193 (1991)). Ligase-mediated covalent attachment occurs only when the oligonucleotides are correctly base-paired. The Ligase Chain Reaction (LCR), which utilizes the thermostable Taq ligase for target amplification, is particularly useful for interrogating late onset diabetes mutation loci. The elevated reaction temperatures permits the ligation reaction to be conducted with high stringency (Barany, F., PCR Methods and Applications 1: 5-16 (1991)).

The hybridization reactions may be carried out in a filter-based format, in which the target nucleic acids are immobilized on nitrocellulose or nylon membranes and probed with oligonucleotide probes. Any of the known hybridization formats may be used, including Southern blots, slot blots, “reverse” dot blots, solution hybridization, solid support based sandwich hybridization, bead-based, silicon chip-based and microtiter well-based hybridization formats.

Detection oligonucleotide probes range in size between 10-1,000 bases. In order to obtain the required target discrimination using the detection oligonucleotide probes, the hybridization reactions are generally run between 20′-60° C., and most preferably between 30′-50° C. As known to those skilled in the art, optimal discrimination between perfect and mismatched duplexes is obtained by manipulating the temperature and/or salt concentrations or inclusion of formamide in the stringency washes.

Detection of proteins may be carried out using specific antibodies, e.g., monoclonal or polyclonal antibodies, or fragments thereof.

Preferred detection reagents are labeled, e.g., fluorescents, coloro-metrically or radio-iso-typically labeled to facilitate visulalization and/or quantitation.

shRNAs (either known or identified by the methodologies of the present invention) can be used in a functional analysis of the corresponding targeted RNA (either known or identified by the methodologies of the present invention). Such a functional analysis is typically carried out in mammalian cells and most preferably human cells, e.g. T cells, e.g., naïve T cells or differentiated T cells, or rodents, e.g. rats and mice.

In one embodiment, shRNAs specific for putative Teff or Treg molecules can be synthesized from plasmid constructs directly in cells.

Preferred tolerance target molecules, include but are not limited to, those listed in the Table below:

| GenBank | | | GenBank |
|---|---|---|---|
| Reference | | | Reference |
| No.; GI No.; | | | No.; GI No.; |
| SEQ ID NO. | | | SEQ ID NO. |
| (Human) | Symbol | Description | (Mouse) |
| NM_021181.2; | CRACC/SLAMF7 | 19A24 protein- SLAMF7 - Receptor; | — |
| gi: 12711663; | | Immunoglobulm receptor family member | |
| SEQ ID | | | |
| NO.: 30 | | | |
| NM_000961.1; | PTGIS | prostaglandin I2 (prostacyclin) synthase | NM_008968; |
| gi: 13699858; | | | gi: 31982083; |
| SEQ ID | | | SEQ ID NO.: 44 |
| NO.: 22 | | | |
| NM_147130; | NCR3 | natural cytotoxicity triggering receptor 3 | — |
| gi: 24475831; | | | |
| SEQ ID | | | |
| NO.: 31 | | | |
| NM_006203; | PDE4D | phosphodiesterase 4D, cAMP-specific | — |
| gi: 46361981; | | (phosphodiesterase E3 dunce homolog, | |
| SEQ ID | | Drosophila) | |
| NO.: 19 | | | |
| NM_018725.1; | IL17RB | interleukin 17 receptor B | NM_019583; |
| gi: 8923816; | | | gi: 9624983; |
| SEQ ID | | | SEQ ID NO.: 45 |
| NO.: 32 | | | |
| NM_181780; | BTLA | B and T lymphocyte associated receptor | NM_177584; |
| gi: 32401472; | | | gi: 31340692; |
| SEQ ID | | | SEQ ID NO.: 46 |
| NO.: 33 | | | |
| NM_020654.1; | SENP7 | sentrin/SUMO-specific protease | — |
| gi: 10190689; | | | |
| SEQ ID | | | |
| NO.: 23 | | | |
| NM_000860.1; | HPGD | hydroxyprostaglandin dehydrogenase 15-(NAD) | NM_008278; |
| gi: 4504478; | | | gi: 6680262; |
| SEQ ID | | | SEQ ID NO.: 47 |
| NO.: 24 | | | |
| NM_000867.1; | HTR2B | 5-hydroxytryptamine (serotonin) receptor 2B | NM_008311; |
| gi: 4504538; | | | gi: 6680322; |
| SEQ ID | | | SEQ ID NO.: 48 |
| NO.: 34 | | | |
| NM_005214.1; | CTLA4 | cytotoxic T-lymphocyte-associated protein 4 | NM_009843; |
| gi: 4885166; | | | gi: 31981846; |
| SEQ ID | | | SEQ ID NO.: 49 |
| NO.: 35 | | | |
| NM_005356.1; | LCK | lymphocyte-specific protein tyrosine kinase | NM_010693; |
| gi: 4885448; | | | gi: 33859569; |
| SEQ ID | | | SEQ ID NO.: 50 |
| NO.: 25 | | | |
| NM_014009.1; | FOXP3 | forkhead box P3 | NM_054039; |
| gi: 7661845; | | | gi: 16905074; |
| SEQ ID NO.: 1 | | | SEQ ID NO.: 51 |
| NM_013351.1; | TBX21 | T-box 21 | NM_019507; |
| gi: 7019548; | | | gi: 9507178; |
| SEQ ID NO.: 3 | | | SEQ ID NO.: 52 |
| NM_173582; | FLJ32029 | hypothetical protein | — |
| gi: 31377547; | | FLJ32029/phosphoglucomutase 2-like 1 | |
| SEQ ID | | | |
| NO.: 26 | | | |
| NM_198485; | — | Homo sapiens cDNA FLJ43694 fis, clone | — |
| gi: 38348285; | | TBAES2006568 | |
| SEQ ID | | | |
| NO.: 39 | | | |
| NM_178496; | LOC151963 | similar to BcDNA: GH11415 gene product | — |
| gi: 31341922; | | | |
| SEQ ID | | | |
| NO.: 40 | | | |
| NM_019000.1; | FLJ20152 | hypothetical protein FLJ20152 | — |
| gi: 9506660; | | | |
| SEQ ID | | | |
| NO.: 41 | | | |
| NM_016619.1; | PLAC8 | placenta-specific 8 | NM_139198; |
| gi: 7706157; | | | gi: 21105852; |
| SEQ ID | | | SEQ ID NO.: 53 |
| NO.: 42 | | | |
| NM_006443.1; | RCL | putative c-Myc-responsive | — |
| gi: 5454001; | | | |
| SEQ ID | | | |
| NO.: 43 | | | |
| NM_175081; | P2RX5 | purinergic receptor P2X, ligand-gated ion channel, | NM_033321; |
| gi: 28416936; | | 5 | gi: 15277324; |
| SEQ ID | | | SEQ ID NO.: 54 |
| NO.: 38 | | | |
| NM_058172; | ANTXR2 | anthrax toxin receptor 2 | — |
| gi: 17158002; | | | |
| SEQ ID | | | |
| NO.: 37 | | | |
| M61906; | | P13 Kinase subunit (p85 alpha) | — |
| gi: 189424; | | | |
| SEQ ID | | | |
| NO.: 27 | | | |
| NM_006257; | Prkcθ | Protein Kinase C theta | NM_008859; |
| gi: 48255887; | | | gi: 6679352; |
| SEQ ID | | | SEQ ID NO.: 55 |
| NO.: 12 | | | |
| NM_002829.1; | PTPN3 | protein tyrosine phosphatase, non-receptor type 3 | — |
| gi: 4506292; | | | |
| SEQ ID | | | |
| NO.: 28 | | | |
| AF290614; | PTPN23 | protein tyrosine phosphatase, non-receptor type 23 | — |
| gi: 13491975; | | | |
| SEQ ID | | | |
| NO.: 29 | | | |
| BC000479; | AKT1 | AKT1 | NM_009652; |
| gi: 33875493; | | | gi: 6753033; |
| SEQ ID | | | SEQ ID NO.: 56 |
| NO.: 36 | | | |
| — | AKT2 | AKT2 | NM_007434; |
| | | | gi: 27312021; |
| | | | SEQ ID NO.: 57 |

Exemplary shRNA molecules directed toward the targets identified in the Table above can be made by those of skill in the art. For instance, exemplary siRNAs for the putative target sequences listed in the Table above were designed using siRNA Target Finder available through the Technical Resources at publically available from ambion.com and are shown below. This algorithm identifies an siRNA sequence by identifying an “AA” followed by 19 nucleotides, and then, if possible, a “TT” is identified (the last feature is optional). The candidate sequence may have a GC content of about 35% to about 70% and be at least 75 nucleotides downstream from the ATG start site. A final requirement is that the sequence lack homology with other genes. Homology can be determined using a Blast search using all libraries, including the est sequence database. This algorithm was used to choose 3 or 4 sequences for each targeted cytokine gene (TNFα, IL-1). The sequences can be synthesized by one of skill in the art or can be commercially made, e.g., by Dharmacon.

For SEQ ID NO.:1 (FoxP3):

Beginning at position 171:

| | |
|---|---|
| Sense strand siRNA: | |
| CGGUGGAUGCCCACGCCCGtt | (SEQ ID NO.:58) |
| Antisense strand siRNA: | |
| CGGGCGUGGGCAUCCACCGtt | (SEQ ID NO.:59) |

Beginning at position 326:

| | |
|---|---|
| Sense strand siRNA: | |
| UGGGUGUCCAGGGAGCCGGtt | (SEQ ID NO.:60) |
| Antisense strand siRNA: | |
| CCGGCUCCCUGGACACCCAtt | (SEQ ID NO.:61) |

For SEQ ID NO.:3 (TBX21):

Beginning at position 1130:

| | |
|---|---|
| Sense strand siRNA: | |
| UGCCGAGAUUACUCAGCUGtt | (SEQ ID NO.:62) |
| Antisense strand siRNA: | |
| CAGCUGAGUAAUCUCGGCAtt | (SEQ ID NO.:63) |

Beginning at position 2007:

| | |
|---|---|
| Sense strand siRNA: | |
| ACCUGAGAGGGGUGUCCCCtt | (SEQ ID NO.:64) |
| Antisense strand siRNA: | |
| GGGGACACCCCUCUCAGGUtt | (SEQ ID NO.:65) |

For SEQ ID NO.:30 (CRACC/SLAM-7):

Beginning at position 454:

| | |
|---|---|
| Sense strand siRNA: | |
| GAAUGGCACCUGUGUGACCtt | (SEQ ID NO.:66) |
| Antisense strand siRNA: | |
| GGUCACACAGGUGCCAUUCtt | (SEQ ID NO.:67) |

Beginning at position 933:

| | |
|---|---|
| Sense strand siRNA: | |
| AUACGGUUUACUCCACUGUtt | (SEQ ID NO.:68) |
| Antisense strand siRNA: | |
| ACAGUGGAGUAAACCGUAUtt | (SEQ ID NO.:69) |

For SEQ ID NO.:22 (PTGIS):

Beginning at position 1720:

| | |
|---|---|
| Sense strand siRNA: | |
| GAGGGUGAACAAAGAAAAGtt | (SEQ ID NO.:70) |
| Antisense strand siRNA: | |
| CUUUUCUUUGUUCACCCUCtt | (SEQ ID NO.:71) |

Beginning at position 4253:

| | |
|---|---|
| Sense strand siRNA: | |
| GAAUCAAAAUUACUGGACAtt | (SEQ ID NO.:72) |
| Antisense strand siRNA: | |
| UGUCCAGUAAUUUUGAUUCtt | (SEQ ID NO.:73) |

For SEQ ID NO.:31 (NCR3):

Beginning at position 177:

| | |
|---|---|
| Sense strand siRNA: | |
| AGGCCUGGCUACAAGUAGUtt | (SEQ ID NO.:74) |
| Antisense strand siRNA: | |
| ACUACUUGUAGCCAGGCCUtt | (SEQ ID NO.:75) |

Beginning at position 921:

| | |
|---|---|
| Sense strand siRNA: | |
| CUGCCCUUUCCUGCCAGGCtt | (SEQ ID NO.:76) |
| Antisense strand siRNA: | |
| GCCUGGCAGGAAAGGGCAGtt | (SEQ ID NO.:77) |

For SEQ ID NO.:19 (PDE4D):

Beginning at position 2465:

| | |
|---|---|
| Sense strand siRNA: | |
| GAAAGGCAUUGCACAGAGUtt | (SEQ ID NO.:78) |
| Antisense strand siRNA: | |
| ACUCUGUGCAAUGCCUUUCtt | (SEQ ID NO.:79) |

Beginning at position 4682:

| | |
|---|---|
| Sense strand siRNA: | |
| CAAAUCCAGACCUGAAAGGtt | (SEQ ID NO.:80) |
| Antisense strand siRNA: | |
| CCUUUCAGGUCUGGAUUUGtt | (SEQ ID NO.:81) |

For SEQ ID NO.:32 (ILI7RB):

Beginning at position 577:

| | |
|---|---|
| Sense strand siRNA: | |
| UGAGGAGACAGUAGAAGUGtt | (SEQ ID NO.:82) |
| Antisense strand siRNA: | |
| CACUUCUACUGUCUCCUCAtt | (SEQ ID NO.:83) |

Beginning at position 694:

| | |
|---|---|
| Sense strand siRNA: | |
| ACAAACGCGAGCUUCAGUGtt | (SEQ ID NO.:84) |
| Antisense strand siRNA: | |
| CACUGAAGCUCGCGUUUGUtt | (SEQ ID NO.:85) |

For SEQ ID NO.:33 (BTLA):

Beginning at position 328:

| | |
|---|---|
| Sense strand siRNA: | |
| UGGGUCAUACCGCUGUUCUtt | (SEQ ID NO.:86) |
| Antisense strand siRNA: | |
| AGAACAGCGGUAUGACCCAtt | (SEQ ID NO.:87) |

Beginning at position 447:

| | |
|---|---|
| Sense strand siRNA: | |
| GCAGACCCUGGCUCCUGUAtt | (SEQ ID NO.:88) |
| Antisense strand siRNA: | |
| UACAGGAGCCAGGGUCUGCtt | (SEQ ID NO.:89) |

For SEQ ID NO.:23 (SENP7):

Beginning at position 179:

| | |
|---|---|
| Sense strand siRNA: | |
| AUUGCUGCCUUUAUUCUGUtt | (SEQ ID NO.:90) |
| Antisense strand siRNA: | |
| ACAGAAUAAAGGCAGCAAUtt | (SEQ ID NO.:91) |

Beginning at position 557:

| | |
|---|---|
| Sense strand siRNA: | |
| GAUUUUCCACAAACUGUAUtt | (SEQ ID NO.:92) |
| Antisense strand siRNA: | |
| AUACAGUUUGUGGAAAAUCtt | (SEQ ID NO.:93) |

For SEQ ID NO.:24 (HPGD):

Beginning at position 1299:

| | |
|---|---|
| Sense strand siRNA: | |
| CAUUUCAGUUCUUCCAAGAtt | (SEQ ID NO.:94) |
| Antisense strand siRNA: | |
| UCUUGGAAGAACUGAAAUGtt | (SEQ ID NO.:95) |

Beginning at position 1509:

| | |
|---|---|
| Sense strand siRNA: | |
| AAAUCUCAGGAAUUAAACUtt | (SEQ ID NO.:96) |
| Antisense strand siRNA: | |
| AGUUUAAUUCCUGAGAUUUtt | (SEQ ID NO.:97) |

For SEQ ID NO.:34 (HTR2B):

Beginning at position 870:

| | | |
|---|---|---|
| Sense strand siRNA: | ACACCUUGCUCGUCACCGGtt | (SEQ ID NO.:98) |
| Antisense strand siRNA: | CCGGUGACGAGCAAGGUGUtt | (SEQ ID NO.:99) |

Beginning at position 1007:

| | | |
|---|---|---|
| Sense strand siRNA: | CGAACAGAGAGCCUCAAAGtt | (SEQ ID NO.:100) |
| Antisense strand siRNA: | CUUUGAGGCUCUCUGUUCGtt | (SEQ ID NO.:101) |

For SEQ ID NO.:35 (CTLA4):

Beginning at position 388:

| | | |
|---|---|---|
| Sense strand siRNA: | GGUGGAGCUCAUGUACCCAtt | (SEQ ID NO.:102) |
| Antisense strand siRNA: | UGGGUACAUGAGCUCCACCtt | (SEQ ID NO.:103) |

Beginning at position 618:

| | | |
|---|---|---|
| Sense strand siRNA: | CAGAGCCAGAAUGUGAAAAtt | (SEQ ID NO.:104) |
| Antisense strand siRNA: | UUUUCACAUUCUGGCUCUGtt | (SEQ ID NO.:105) |

For SEQ ID NO.:25 (LCK):

Beginning at position 868:

| | | |
|---|---|---|
| Sense strand siRNA: | GAGCCUGAAGCAGGGCAGCtt | (SEQ ID NO.:106) |
| Antisense strand siRNA: | GCUGCCCUGCUUCAGGCUCtt | (SEQ ID NO.:107) |

Beginning at position 938:

| | | |
|---|---|---|
| Sense strand siRNA: | CACCAGCGGCUGGUUCGGCtt | (SEQ ID NO.:108) |
| Antisense strand siRNA: | GCCGAACCAGCCGCUGGUGtt | (SEQ ID NO.:109) |

For SEQ ID NO.:26 (FLJ32029):

Beginning at position 4950:

| | | |
|---|---|---|
| Sense strand siRNA: | GGAGAGAAUUAGUAGGGCAtt | (SEQ ID NO.:110) |
| Antisense strand siRNA: | UGCCCUACUAAUUCUCUCCtt | (SEQ ID NO.:111) |

Beginning at position 5162:

| | | |
|---|---|---|
| Sense strand siRNA: | AAUGAGGCCAAGGUUUAGGtt | (SEQ ID NO.:112) |
| Antisense strand siRNA: | CCUAAACCUUGGCCUCAUUtt | (SEQ ID NO.:1113) |

For SEQ ID NO.:39 (FLJ4394(clone TBAES2006568):

Beginning at position 1958:

| | | |
|---|---|---|
| Sense strand siRNA: | GCAGAUAAAAGUAGGUUCAtt | (SEQ ID NO.:114) |
| Antisense strand siRNA: | UGAACCUACUUUUAUCUGCtt | (SEQ ID NO.:115) |

Beginning at position 2049:

| | | |
|---|---|---|
| Sense strand siRNA: | UGUCCAUGGAUCGCAGAGGtt | (SEQ ID NO.:116) |
| Antisense strand siRNA: | CCUCUGCGAUCCAUGGACAtt | (SEQ ID NO.:117) |

For SEQ ID NO.:40 (LOC151963):

Beginning at position 1690:

| | | |
|---|---|---|
| Sense strand siRNA: | ACUGCAGCAGCUAGUGACUtt | (SEQ ID NO.:118) |
| Antisense strand siRNA: | AGUCACUAGCUGCUGCAGUtt | (SEQ ID NO.:119) |

Beginning at position 1829:

| | | |
|---|---|---|
| Sense strand siRNA: | ACUCUGAUAAUGUGUCGUGtt | (SEQ ID NO.:120) |
| Antisense strand siRNA: | CACGACACAUUAUCAGAGUtt | (SEQ ID NO.:121) |

For SEQ ID NO.:41 (FLJ20152):

Beginning at position 796:

| | | |
|---|---|---|
| Sense strand siRNA: | ACCAAACUAUUAAAAUCCUtt | (SEQ ID NO.:122) |
| Antisense strand siRNA: | AGGAUUUUAAUAGUUUGGUtt | (SEQ ID NO.:123) |

Beginning at position 979:

| | | |
|---|---|---|
| Sense strand siRNA: | AAGACCGAAAUUAUCAAGGtt | (SEQ ID NO.:124) |
| Antisense strand siRNA: | CCUUGAUAAUUUCGGUCUUtt | (SEQ ID NO.:125) |

For SEQ ID NO.:42 (PLAC8):

Beginning at position 174:

| | | |
|---|---|---|
| Sense strand siRNA: | CUGGCAGACAGGCAUGUGUtt | (SEQ ID NO.:126) |
| Antisense strand siRNA: | ACACAUGCCUGUCUGCCAGtt | (SEQ ID NO.:127) |

Beginning at position 276:

| | | |
|---|---|---|
| Sense strand siRNA: | UGAAUGCUGUCUGUGUGGAtt | (SEQ ID NO.:128) |
| Antisense strand siRNA: | UCCACACAGACAGCAUUCAtt | (SEQ ID NO.:129) |

For SEQ ID NO.:43 (RCL):

Beginning at position 295:

| | | |
|---|---|---|
| Sense strand siRNA: | GUGACACAGCCAUCCUUGGtt | (SEQ ID NO.:130) |
| Antisense strand siRNA: | CCAAGGAUGGCUGUGUCACtt | (SEQ ID NO.:131) |

Beginning at position 354:

| | | |
|---|---|---|
| Sense strand siRNA: | GCGGAUCCUGUGCCUGUUCtt | (SEQ ID NO.:132) |
| Antisense strand siRNA: | GAACAGGCACAGGAUCCGCtt | (SEQ ID NO.:133) |

For SEQ ID NO.:38 (P2RX5):

Beginning at position 357:

| | | |
|---|---|---|
| Sense strand siRNA: | GAACAAGAAGGUGGGCCUGtt | (SEQ ID NO.:134) |
| Antisense strand siRNA: | CAGGCCCACCUUCUUGUUCtt | (SEQ ID NO.:135) |

Beginning at position 437:

| | | |
|---|---|---|
| Sense strand siRNA: | AGAAGGGUUACCAAGACGUtt | (SEQ ID NO.:136) |
| Antisense strand siRNA: | ACGUCUUGGUAACCCUUCUtt | (SEQ ID NO.:137) |

For SEQ ID NO.:37 (ANTXR2):

Beginning at position 670:

| | | |
|---|---|---|
| Sense strand siRNA: | AGCUGGAGAGUGGGGUCUCtt | (SEQ ID NO.:138) |
| Antisense strand siRNA: | GAGACCCCACUCUCCAGCUtt | (SEQ ID NO.:139) |

Beginning at position 981:

| | | |
|---|---|---|
| Sense strand siRNA: | GGAUCCUCCACCACCACCCtt | (SEQ ID NO.:140) |
| Antisense strand siRNA: | GGGUGGUGGUGGAGGAUCCtt | (SEQ ID NO.:141) |

For SEQ ID NO.:27 (PI3 Kinase subunit (p85α):

Beginning at position 1270:

| | | |
|---|---|---|
| Sense strand siRNA: | UGAAUCUCUAGCUCAGUAUtt | (SEQ ID NO.:142) |
| Antisense strand siRNA: | AUACUGAGCUAGAGAUUCAtt | (SEQ ID NO.:143) |

Beginning at position 1354:

| | | |
|---|---|---|
| Sense strand siRNA: | AGAAGAUAAUAUUGAAGCUtt | (SEQ ID NO.:144) |
| Antisense strand siRNA: | AGCUUCAAUAUUAUCUUCUtt | (SEQ ID NO.:145) |

For SEQ ID NO.:12 Protein kinase C θ:

Beginning at position 1487:

| | | |
|---|---|---|
| Sense strand siRNA: | AGCUGCCACAAGUUCGACCtt | (SEQ ID NO.:146) |
| Antisense strand siRNA: | GGUCGAACUUGUGGCAGCUtt | (SEQ ID NO.:147) |

Beginning at position 1647:

| | | |
|---|---|---|
| Sense strand siRNA: | UGUGCAAGGAGAACAUGUUtt | (SEQ ID NO.:148) |
| Antisense strand siRNA: | AACAUGUUCUCCUUGCACAtt | (SEQ ID NO.:149) |

For SEQ ID NO.:28 (PTPN3):

Beginning at position 1306:

| | | |
|---|---|---|
| Sense strand siRNA: | GUUUCUCAGAACCGAAGCCtt | (SEQ ID NO.:150) |
| Antisense strand siRNA: | GGCUUCGGUUCUGAGAAACtt | (SEQ ID NO.:151) |

Beginning at position 1363:

| | | |
|---|---|---|
| Sense strand siRNA: | AGCUACCUGACCCAGAAGUtt | (SEQ ID NO.:152) |
| Antisense strand siRNA: | ACUUCUGGGUCAGGUAGCUtt | (SEQ ID NO.:153) |

For SEQ ID NO.:29 (PTPN23):

Beginning at position 1523:

| | | |
|---|---|---|
| Sense strand siRNA: | GGCUGAGCUGGCAGAGGUGtt | (SEQ ID NO.:154) |
| Antisense strand siRNA: | CACCUCUGCCAGCUCAGCCtt | (SEQ ID NO.:155) |

Beginning at position 1882:

| | | |
|---|---|---|
| Sense strand siRNA: | AGUAUGACCAGCUGAAGGUtt | (SEQ ID NO.:156) |
| Antisense strand siRNA: | ACCUUCAGCUGGUCAUACUtt | (SEQ ID NO.:157) |

For SEQ ID NO.:36 (AKT1):

Beginning at position 826:

| | | |
|---|---|---|
| Sense strand | GUCAUCGUGGCCAAGGACGtt | (SEQ ID NO.:158) |
| siRNA: | | |
| Antisense | CGUCCUUGGCCACGAUGACtt | (SEQ ID NO.:159) |
| strand siRNA: | | |

Beginning at position 1431:

| | | |
|---|---|---|
| Sense strand | GGACCCCAAGCAGAGGCUUtt | (SEQ ID NO.:160) |
| siRNA: | | |
| Antisense | AAGCCUCUGCUUGGGGUCCtt | (SEQ ID NO.:161) |
| strand siRNA: | | |

In another aspect, the invention pertains to a combination of two or more of the assays described herein. For example, a test compound can be assayed for its ability to modulate the expression of at least one Teff or Treg marker of the invention as well as its ability to modulate T cell proliferation. Similarly, a putative target molecule can be identified by assaying for cytokine production and cell proliferation.

Secondary Screening Assays

In an embodiment of a screening assay of the invention, once a test compound or target molecule is identified as modulating tolerance, the effect of the test compound or target molecule on an immune response, e.g., on the immune function of cells in vitro (e.g., using cell lines or cells derived from a subject) or in vivo (e.g., using an animal model) can be tested. In addition, as set forth above, a target molecule identified using the subject screening assays can be used in a screening assay to identify modulators of the target molecule. In one embodiment, the screening methods of the invention can further comprise determining the effect of a compound identified as a tolerance modulatory compound using the methods of the invention on an immune response to thereby confirm that a compound modulates tolerance.

Moreover, a modulator identified as described herein can be used in an animal model to determine the efficacy, toxicity, or side effects of treatment with such a modulator. Alternatively, a tolerance modulator identified as described herein can be used in an animal model to determine the mechanism of action of such a modulator.

For example, an agent can be tested in art recognized animal models of human diseases (e.g., EAE as a model of multiple sclerosis and the NOD mice as a model for diabetes) or other well characterized animal models of human autoimmune diseases. Such animal models include the mrl/lpr/lpr mouse as a model for lupus erythematosus, murine collagen-induced arthritis as a model for rheumatoid arthritis, and murine experimental myasthenia gravis (see Paul ed., Fundamental Immunology, Raven Press, New York, 1989, pp. 840-856). A modulatory (i.e., stimulatory or inhibitory) agent of the invention can be administered to test animals and the course of the disease in the test animals can then be monitored using standard methods for the particular model being used. Effectiveness of the modulatory agent is evidenced by amelioration of the disease condition in animals treated with the agent as compared to untreated animals (or animals treated with a control agent).

III. Test Compounds

A variety of test compounds can be evaluated using the screening assays described herein. In certain embodiments, the compounds to be tested can be derived from libraries (i.e., are members of a library of compounds). While the use of libraries of peptides is well established in the art, new techniques have been developed which have allowed the production of mixtures of other compounds, such as benzodiazepines (Bunin et al. (1992). J. Am. Chem. Soc. 114:10987; DeWitt et al. (1993). Proc. Natl. Acad. Sci. USA 90:6909) peptoids (Zuckermann. (1994). J. Med. Chem. 37:2678) oligocarbamates (Cho et al. (1993). Science. 261:1303-), and hydantoins (DeWitt et al. supra). An approach for the synthesis of molecular libraries of small organic molecules with a diversity of 104-105 as been described (Carell et al. (1994). Angew. Chem. Int. Ed. Engl. 33:2059-; Carell et al. (1994) Angew. Chem. Int. Ed. Engl. 33:2061-).

The compounds of the present invention can be obtained using any of the numerous approaches in combinatorial library methods known in the art, including: biological libraries; spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Compound Des. 12:145). Other exemplary methods for the synthesis of molecular libraries can be found in the art, for example in: Erb et al. (1994). Proc. Natl. Acad. Sci. USA 91:11422-; Horwell et al. (1996) Immunopharmacology 33:68-; and in Gallop et al. (1994); J. Med. Chem. 37:1233-.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); In still another embodiment, the combinatorial polypeptides are produced from a cDNA library.

Non limiting exemplary compounds which can be screened for activity include, but are not limited to, peptides, nucleic acids, carbohydrates, small organic molecules, and natural product extract libraries.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); 4) small organic and inorganic molecules (e.g., molecules obtained from combinatorial and natural product libraries); 5) enzymes (e.g., endoribonucleases, hydrolases, nucleases, proteases, synthatases, isomerases, polymerases, kinases, phosphatases, oxido-reductases and ATPases), 6) mutant forms of markers, e.g., dominant negative mutant forms of Teff or Treg markers and 7) antisense RNA molecules or molecules that mediate RNAi.

RNA interference (RNAi is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999)). The process occurs when an endogenous ribonuclease cleaves the longer dsRNA into shorter, e.g., 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs and Ambion.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Compound Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994)Angew. Chem. Int. Ed. Engl. 33:2059; Carell et al. (1994) Angew. Chem. Int. Ed. Engl. 33:2061; and Gallop et al. (1994) J. Med. Chem. 37:1233.

Art recognized techniques of structure based drug design can also be used to identify compounds that modulate the expression or activity of one or more markers of the invention.

The instant invention also pertains to compounds identified in the above assays. Compounds identified in the subject screening assays can be used in methods of modulating tolerance. It will be understood that it may be desirable to formulate such compound(s) as pharmaceutical compositions prior to contacting them with cells.

IV. Therapeutic Uses

Tolerance modulatory compounds or agents identified according to the methodologies of the instant invention are useful for treating a wide variety of immune-mediated diseases. In particular, tolerance stimulatory compounds or agents (e.g., compounds or agents which favor or promote differentiation or maintenance of Treg cells over Teff cells) are useful for treating conditions associated with misdirected or undesirable immune responses, such as autoimmune disorders, organ rejection, or unwanted immune responses to chronically administered therapeutic proteins. By contrast, tolerance inhibitory compounds or agents (e.g., compounds or agents which favor or promote differentiation of Teff cells over Treg cells) are useful for enhancing immune responses, e.g. to tumors or viruses to which the body may have become tolerant.

Tolerance modulatory compounds or agents identified according to the methodologies of the instant invention are particularly suited to therapeutic methods which involve specific modulation of tolerance while preserving normal immunological function. The advantages of this therapeutic approach over general immunosuppressive therapies are that treatment is more effective and that the treatment has a long term effect after a short therapeutic course. In addition, the patient's immune system remains intact, and unwanted side effects can be reduced.

According to a modulatory method of the invention, tolerance can be promoted by inhibition of expression or activity of a Teff marker or tolerance can be inhibited by inhibiting expression or activity of a Treg marker. In preferred embodiments, more than one marker is modulated. For example, in one embodiment, at least two Treg markers are modulated in the same direction, i.e., are up or downmodulated. In another embodiment, at least one T reg marker and at least one Teff marker are modulated in opposite directions. Expression or activity of markers can be dowmnodulated using inhibitory agents or can be upmodulated using stimulatory agents.

A. Teff Marker or Treg Marker Inhibitory Agents

Inhibitory agents of the invention can be, for example, intracellular binding molecules that act to inhibit the expression or activity of a marker. As used herein, the term “intracellular binding molecule” is intended to include molecules that act intracellularly to inhibit the expression or activity of a protein by binding to the protein itself, to a nucleic acid (e.g., an mRNA molecule) that encodes the protein or to a target with which the protein normally interacts (e.g., to a DNA target sequence to which the marker binds). Examples of intracellular binding molecules, described in further detail below, include antisense marker nucleic acid molecules (e.g., to inhibit translation of mRNA), intracellular antibodies (e.g., to inhibit the activity of protein) and dominant negative mutants of the marker proteins.

In one embodiment, an inhibitory agent of the invention is an antisense nucleic acid molecule that is complementary to a gene encoding marker of the invention or to a portion of said gene, or a recombinant expression vector encoding said antisense nucleic acid molecule. The use of antisense nucleic acids to downregulate the expression of a particular protein in a cell is well known in the art (see e.g., Weintraub, H. et al., Antisense RNA as a molecular tool for genetic analysis, Reviews—Trends in Genetics, Vol. 1(1) 1986; Askari, F. K. and McDonnell, W. M. (1996) N. Eng. J. Med. 334:316-318; Bennett, M. R. and Schwartz, S. M. (1995) Circulation 92:1981-1993; Mercola, D. and Cohen, J. S. (1995) Cancer Gene Ther. 2:47-59; Rossi, J. J. (1995) Br. Med. Bull. 51:217-225; Wagner, R. W. (1994) Nature 372:333-335). An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. Preferably, an antisense nucleic acid is designed so as to be complementary to a region preceding or spanning the initiation codon on the coding strand or in the 3′ untranslated region of an mRNA. An antisense nucleic acid molecule for inhibiting the expression of protein in a cell can be designed based upon the nucleotide sequence encoding the protein constructed according to the rules of Watson and Crick base pairing.

An antisense nucleic acid molecule can exist in a variety of different forms. For example, the antisense nucleic acid can be an oligonucleotide that is complementary to only a portion of a gene. An antisense oligonucleotides can be constructed using chemical synthesis procedures known in the art. An antisense oligonucleotide can be chemically synthesized using naturally occurring nucleotides or variously modified nucleotides designed to increase the biological stability of the molecules or to increase the physical stability of the duplex formed between the antisense and sense nucleic acids, e.g. phosphorothioate derivatives and acridine substituted nucleotides can be used. To inhibit expression in cells in culture, one or more antisense oligonucleotides can be added to cells in culture media, typically at about 200 μg oligonucleotide/ml.

Alternatively, an antisense nucleic acid molecule can be produced biologically using an expression vector into which a nucleic acid has been subcloned in an antisense orientation (i.e., nucleic acid transcribed from the inserted nucleic acid will be of an antisense orientation to a target nucleic acid of interest). Regulatory sequences operatively linked to a nucleic acid cloned in the antisense orientation can be chosen which direct the expression of the antisense RNA molecule in a cell of interest, for instance promoters and/or enhancers or other regulatory sequences can be chosen which direct constitutive, tissue specific or inducible expression of antisense RNA. For example, for inducible expression of antisense RNA, an inducible eukaryotic regulatory system, such as the Tet system (e.g., as described in Gossen, M. and Bujard, H. (1992) Proc. Natl. Acad. Sci. USA 89:5547-5551; Gossen, M. et al. (1995) Science 268:1766-1769; PCT Publication No. WO 94/29442; and PCT Publication No. WO 96/01313) can be used. The antisense expression vector is prepared as described above for recombinant expression vectors, except that the cDNA (or portion thereof) is cloned into the vector in the antisense orientation. The antisense expression vector can be in the form of, for example, a recombinant plasmid, phagemid or attenuated virus. The antisense expression vector is introduced into cells using a standard transfection technique, as described above for recombinant expression vectors.

In another embodiment, a compound that mediates RNAi can be used to inhibit marker gene expression. RNA interference is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999)). The process occurs when an endogenous ribonuclease cleaves the longer dsRNA into shorter, 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs and Ambion. In one embodiment one or more of the chemistries described above for use in antisense RNA can be employed.

In another embodiment, an antisense nucleic acid for use as an inhibitory agent is a ribozyme. Ribozymes are catalytic RNA molecules with ribonuclease activity which are capable of cleaving a single-stranded nucleic acid, such as an mRNA, to which they have a complementary region (for reviews on ribozymes see e.g., Ohkawa, J. et al. (1995) J. Biochem. 118:251-258; Sigurdsson, S. T. and Eckstein, F. (1995) Trends Biotechnol. 13:286-289; Rossi, J. J. (1995) Trends Biotechnol. 13:301-306; Kiehntopf, M. et al. (1995) J. Mol. Med. 73:65-71). A ribozyme having specificity for marker mRNA can be designed based upon the nucleotide sequence of the marker cDNA. For example, a derivative of a Tetrahymena L-19 IVS RNA can be constructed in which the base sequence of the active site is complementary to the base sequence to be cleaved in marker mRNA. See for example U.S. Pat. Nos. 4,987,071 and 5,116,742, both by Cech et al. Alternatively, marker mRNA can be used to select a catalytic RNA having a specific ribonuclease activity from a pool of RNA molecules. See for example Bartel, D. and Szostak, J. W. (1993) Science 261: 1411-1418.

Another type of inhibitory agent that can be used to inhibit the expression and/or activity of in a cell is an intracellular antibody specific for the marker. The use of intracellular antibodies to inhibit protein function in a cell is known in the art (see e.g., Carlson, J. R. (1988) Mol. Cell. Biol. 8:2638-2646; Biocca, S. et al. (1990) EMBO J. 9:101-108; Werge, T. M. et al. (1990) FEBS Letters 274:193-198; Carlson, J. R. (1993) Proc. Natl. Acad. Sci. USA 90:7427-7428; Marasco, W. A. et al. (1993) Proc. Natl. Acad. Sci. USA 90:7889-7893; Biocca, S. et al. (1994) Bio/Technology 12:396-399; Chen, S-Y. et al. (1994) Human Gene Therapy 5:595-601; Duan, L et al. (1994) Proc. Natl. Acad. Sci. USA 91:5075-5079; Chen, S-Y. et al. (1994) Proc. Natl. Acad. Sci. USA 91:5932-5936; Beerli, R. R. et al. (1994) J. Biol. Chem. 269:23931-23936; Beerli, R. R. et al. (1994) Biochem. Biophys. Res. Commun. 204:666-672; Mhashilkar, A. M. et al. (1995) EMBO J. 14:1542-1551; Richardson, J. H. et al. (1995) Proc. Natl. Acad. Sci. USA 92:3137-3141; PCT Publication No. WO 94/02610 by Marasco et al.; and PCT Publication No. WO 95/03832 by Duan et al.).

To inhibit marker activity using an intracellular antibody, a recombinant expression vector is prepared which encodes the antibody chains in a form such that, upon introduction of the vector into a cell, the antibody chains are expressed as a functional antibody in an intracellular compartment of the cell. For inhibition of marker activity according to the inhibitory methods of the invention, an intracellular antibody that specifically binds the marker protein is expressed in the cytoplasm of the cell. To prepare an intracellular antibody expression vector, antibody light and heavy chain cDNAs encoding antibody chains specific for the target protein of interest are isolated, typically from a hybridoma that secretes a monoclonal antibody specific for the marker. Hybridomas secreting anti-marker monoclonal antibodies, or recombinant monoclonal antibodies, can be prepared as described above. Once a monoclonal antibody specific for the marker protein has been identified (e.g., either a hybridoma-derived monoclonal antibody or a recombinant antibody from a combinatorial library), DNAs encoding the light and heavy chains of the monoclonal antibody are isolated by standard molecular biology techniques. For hybridoma derived antibodies, light and heavy chain cDNAs can be obtained, for example, by PCR amplification or cDNA library screening. For recombinant antibodies, such as from a phage display library, cDNA encoding the light and heavy chains can be recovered from the display package (e.g., phage) isolated during the library screening process. Nucleotide sequences of antibody light and heavy chain genes from which PCR primers or cDNA library probes can be prepared are known in the art. For example, many such sequences are disclosed in Kabat, E. A., et al. (1991) Sequences of Proteins of Immunological Interest, Fifth Edition, U.S. Department of Health and Human Services, NIH Publication No. 91-3242 and in the “Vbase” human germline sequence database.

Once obtained, the antibody light and heavy chain sequences are cloned into a recombinant expression vector using standard methods. To allow for cytoplasmic expression of the light and heavy chains, the nucleotide sequences encoding the hydrophobic leaders of the light and heavy chains are removed. An intracellular antibody expression vector can encode an intracellular antibody in one of several different forms. For example, in one embodiment, the vector encodes full-length antibody light and heavy chains such that a full-length antibody is expressed intracellularly. In another embodiment, the vector encodes a full-length light chain but only the VH/CH1 region of the heavy chain such that a Fab fragment is expressed intracellularly. In the most preferred embodiment, the vector encodes a single chain antibody (scFv) wherein the variable regions of the light and heavy chains are linked by a flexible peptide linker (e.g., (Gly₄Ser)₃) and expressed as a single chain molecule. To inhibit marker activity in a cell, the expression vector encoding the intracellular antibody is introduced into the cell by standard transfection methods, as discussed hereinbefore.

Yet another form of an inhibitory agent of the invention is an inhibitory form a marker protein, e.g, a dominant negative inhibitor. For example, in one embodiment, an active site (e.g., an enzyme active site or a DNA binding domain) can be mutated. Such dominant negative proteins can be expressed in cells using a recombinant expression vector encoding the protein, which is introduced into the cell by standard transfection methods.

Other inhibitory agents that can be used to inhibit the activity of a marker protein are chemical compounds that directly inhibit marker activity or inhibit the interaction between the marker and target DNA or another protein. Such compounds can be identified using screening assays that select for such compounds, as described in detail above.

B. Teff Marker or Treg Marker Stimulatory Agents

According to a modulatory method of the invention, marker expression or activity is stimulated in a cell by contacting the cell with a stimulatory agent. Examples of such stimulatory agents include active protein and nucleic acid molecules encoding markers that are introduced into the cell to increase expression of activity in the cell. A preferred stimulatory agent is a nucleic acid molecule encoding a marker protein, wherein the nucleic acid molecule is introduced into the cell in a form suitable for expression of the active marker protein in the cell. To express a protein in a cell, typically a marker-encoding DNA is first introduced into a recombinant expression vector using standard molecular biology techniques, e.g., as described herein. A marker encoding DNA can be obtained, for example, by amplification using the polymerase chain reaction (PCR), using primers based on the nucleotide sequence of the marker. Following isolation or amplification of the marker-encoding DNA, the DNA fragment is introduced into an expression vector and transfected into target cells by standard methods, as described herein.

Other stimulatory agents that can be used to stimulate the activity of a marker protein are chemical compounds that stimulate marker expression or activity in cells, such as compounds that directly stimulate marker proteins and compounds that promote the interaction between marker proteins and substrates or target DNA binding sites. Such compounds can be identified using screening assays that select for such compounds, as described in detail above.

C. Methods of Use The modulatory methods of the invention can be performed in vitro (e.g., by culturing the cell with the agent or by introducing the agent into cells in culture) or, alternatively, in vivo (e.g., by administering the agent to a subject or by introducing the agent into cells of a subject, such as by gene therapy). For practicing the modulatory method in vitro, cells can be obtained from a subject by standard methods and incubated (i.e., cultured) in vitro with a modulatory agent of the invention to modulate marker activity in the cells. For example, peripheral blood mononuclear cells (PBMCs) can be obtained from a subject and isolated by density gradient centrifugation, e.g., with Ficoll/Hypaque. Specific cell populations can be depleted or enriched using standard methods. For example, T cells can be enriched for example, by positive selection using antibodies to T cell surface markers, for example by incubating cells with a specific primary monoclonal antibody (mAb), followed by isolation of cells that bind the mAb using magnetic beads coated with a secondary antibody that binds the primary mAb. Specific cell populations can also be isolated by fluorescence activated cell sorting according to standard methods. If desired, cells treated in vitro with a modulatory agent of the invention can be readministered to the subject. For administration to a subject, it may be preferable to first remove residual agents in the culture from the cells before administering them to the subject. This can be done for example by a Ficoll/Hypaque gradient centrifugation of the cells. For further discussion of ex vivo genetic modification of cells followed by readministration to a subject, see also U.S. Pat. No. 5,399,346 by W. F. Anderson et al.

For practicing the modulatory method in vivo in a subject, the modulatory agent can be administered to the subject such that marker activity in cells of the subject is modulated. The term “subject” is intended to include living organisms in which an immune response can be elicited. Preferred subjects are mammals. Examples of subjects include humans, monkeys, dogs, cats, mice, rats, cows, horses, goats and sheep.

For stimulatory or inhibitory agents that comprise nucleic acids (including recombinant expression vectors encoding marker protein, antisense RNA, intracellular antibodies or dominant negative inhibitors), the agents can be introduced into cells of the subject using methods known in the art for introducing nucleic acid (e.g., DNA) into cells in vivo. Examples of such methods encompass both non-viral and viral methods, including:

Direct Injection: Naked DNA can be introduced into cells in vivo by directly injecting the DNA into the cells (see e.g., Acsadi et al. (1991) Nature 332:815-818; Wolff et al. (1990) Science 247:1465-1468). For example, a delivery apparatus (e.g., a “gene gun”) for injecting DNA into cells in vivo can be used. Such an apparatus is commercially available (e.g., from BioRad).

Cationic Lipids: Naked DNA can be introduced into cells in vivo by complexing the DNA with cationic lipids or encapsulating the DNA in cationic liposomes. Examples of suitable cationic lipid formulations include N-[-1-(2,3-dioleoyloxy)propyl]N,N,N-triethylammonium chloride (DOTMA) and a 1:1 molar ratio of 1,2-dimyristyloxy-propyl-3-dimethylhydroxyethylammonium bromide (DMRIE) and dioleoyl phosphatidylethanolamine (DOPE) (see e.g., Logan, J. J. et al. (1995) Gene Therapy 2:38-49; San, H. et al. (1993) Human Gene Therapy 4:781-788).

Receptor-Mediated DNA Uptake: Naked DNA can also be introduced into cells in vivo by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor facilitates uptake of the DNA by receptor-mediated endocytosis. A DNA-ligand complex linked to adenovirus capsids which naturally disrupt endosomes, thereby releasing material into the cytoplasm can be used to avoid degradation of the complex by intracellular lysosomes (see for example Curiel et al. (1991) Proc. Natl. Acad. Sci. USA 88:8850; Cristiano et al. (1993) Proc. Natl. Acad. Sci. USA 90:2122-2126).

Retroviruses: Defective retroviruses are well characterized for use in gene transfer for gene therapy purposes (for a review see Miller, A. D. (1990) Blood 76:271). A recombinant retrovirus can be constructed having a nucleotide sequences of interest incorporated into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ψ Crip, ψCre, ψ2 and ψAm. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

Adenoviruses: The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use are deleted for all or parts of the viral E1 and E3 genes but retain as much as 80% of the adenoviral genetic material.

Adeno-Associated Viruses: Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product.

In one embodiment, a retroviral expression vector encoding a marker is used to express marker protein in cells in vivo, to thereby stimulate marker or target protein expression or activity in vivo. Such retroviral vectors can be prepared according to standard methods known in the art (e.g., as discussed further above).

A modulatory agent, such as a chemical compound, can be administered to a subject as a pharmaceutical composition. Such compositions typically comprise the modulatory agent and a pharmaceutically acceptable carrier. As used herein the term “pharmaceutically acceptable carrier” is intended to include any and all solvents, dispersion media, coatings, antibacterial and antifingal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Pharmaceutical compositions can be prepared as described below.

D. Exemplary Conditions to be Treated

Numerous disease conditions associated with a predominant Teff or Treg cell function are known and could benefit from modulation of the type of response mounted in the individual suffering from the disease condition. The methods can involve either direct administration of an inhibitory agent to the transplant recipient or ex vivo treatment of cells obtained from the subject with an agent followed by readministration of the cells to the subject. The treatment may be further enhanced by administering other immunomodulatory agents. The treatment may be further enhanced by administering other immunomodulatory agents. Application of the immunomodulatory methods of the invention to such diseases is described in further detail below.

Many autoimmune disorders are the result of inappropriate or unwanted activation of T effector cells resulting in the production of cytokines and autoantibodies involved in the pathology of the diseases. Accordingly, the methods of the invention can be used to reduce T effector cell function or increase T regulatory cell function in subjects suffering from, or susceptible to, an autoimmune disease. Non-limiting examples of autoimmune diseases and disorders having an autoimmune component that may be treated according to the invention include diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjögren's Syndrome, including keratoconjunctivitis sicca secondary to Sjögren's Syndrome, alopecia greata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis.

In addition, T effector cell function is associated with graft rejection. Accordingly, the methods of the invention can be used to reduce T effector cell function by decreasing Teff marker expression or activity or to increase T regulatory cell function by increasing Treg marker expression or activity. The methods can be used both in solid organ transplantation and in bone marrow transplantation (e.g., to inhibit graft-versus-host disease

Allergies are also mediated by T effector cells. Accordingly, in one embodiment, the methods of the invention can be used to inhibit T effector cell function or enhance T regulatory cell function.

In addition, T effector cell function can be inhibited or T regulatory cell function can be enhanced in a subject in order to reduce the immune response to a therapeutic protein which much be chronically administered to the subject, e.g., factor VIII.

In contrast, there are conditions that would benefit from reduced activity of Treg cells and/or enhanced activity of T effector cells. For example, immune effector cells often fail to react effectively with cancer cells. Accordingly, in one embodiment, the activity of T reg cells is reduced and/or the activity of T effector cells is enhanced to increase the immune response to a cancer cell.

Decreased T regulatory responses would also be of benefit in enhancing responses to infectious diseases, including, e.g., infection with viruses (such as HIV infection) or other microbes (e.g., bacteria or yeast).

Furthermore, the methods of the invention can be used to enhance effector T cell responses upon vaccination of a subject.

E. Use as Surrogate Markers

The marker of the present invention are also useful as markers of disease states, disorders and immunological states (e.g. tolerance), as markers of drug activity, or as markers of the pharmacogenomic profile of a subject. Using the methods described herein, the presence, absence, and/or quantity of the markers of the present invention can be detected, and can be correlated with one or more diseases, disorders and/or immunological states in vivo. For example, the markers of the present invention can serve as surrogate markers for one or more disorders or disease states or immunological states (e.g. tolerance) or for conditions leading to disease states.

As used herein, a “surrogate marker” is an objective biochemical marker which correlates with the absence or presence of a disease, disorder or immunological state (e.g. tolerance), or with the progression of a disease or disorder.

The presence or quantity of such markers is independent of the disease, disorder or immunological state. Therefore, these markers can serve to indicate whether a particular course of treatment is effective in lessening a disease state or disorder, or affecting an immunological state such as tolerance. Surrogate markers are of particular use when the presence or extent of a disease state, disorder or immunological state is difficult to assess through standard methodologies or non invasive techniques, or when an assessment of disease or immunological state is desired before a potentially dangerous clinical endpoint is reached (e.g. surrogate markers may be used to determine whether a transplanted tissue is likely to be rejected and thus more aggressive treatment can be given).

V. Pharmaceutical Compositions

Modulatory agents, e.g., inhibitory or stimulatory agents as described herein, can be incorporated into pharmaceutical compositions suitable for administration. Such compositions typically comprise the agent and a pharmaceutically acceptable carrier. As used herein the language “pharmaceutically acceptable carrier” is intended to include any and all solvents, dispersion media, coatings, antibacterial and antifingal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifingal agents, for example, parabens, chlorobutanol, phenol, ascorbic acid, thimerosal, and the like. In many cases, it is preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, modulatory agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

The pharmaceutical compositions can be included in a container, pack, or dispenser together with instructions for administration.

This invention is further illustrated by the following examples which should not be construed as limiting. The contents of all references, patents and published patent applications cited throughout this application, as well as the Figures and attached Appendices, are incorporated herein by reference.

### EXAMPLES

### Example 1

### Time Course of Expression of Treg and Teff Markers

This example describes the time course of Tbox 21, GATA3 and FOXP3 expression in anti-CD3/anti-CD28 stimulated cells (PBL, CD4+naïve and CD4+memory cells).

Cell Isolation

CD4+CD45RA+naïve, CD4+CD45RA−memory cells were prepared as follows. A Buffy Coat from human peripheral blood was obtained from the Oklahoma Blood Institute and processed as follows. Blood was diluted 1:2 with PBS without Ca⁺⁺Mg⁺⁺. 30 ml diluted blood was overlaid onto 15 ml Ficoll. Tubes were spun for ˜30 minutes at 2000 rpm. The Buffy cell layer was harvested into 25 ml PBS and brought to a volume of 45 ml total. Tubes were then spun ˜10 minutes at 11000 rpm. Tubes were finally combined and washed with PBS. Approximately 10 percent of the cell preparation was set aside (X-VIVO 15 media+10% FCS) and is referred to hereinafter as “PBL.”

The remaining cells were sorted as follows.

CD4+ selection. To select for CD4+ cells (i.e., to deplete the population of all other cell types) cells were pelleted and resuspended in 80 μl buffer per 10×10⁶ cells (˜4 ml buffer). 20 μl of hapten antibody cocktail was added per 10×10⁶ cells. Cells were incubated with antibody for ˜10 minutes at ˜6 to 12° C. Cells were then washed with 50 ml MACS buffer and resuspended in the original volume of buffer (4 ml buffer). Next, 20 μl of anti-hapten microbeads were added. The solution was mixed well and incubated approximately 15 minutes, at ˜6 to 12° C. Cells were then washed once with 50 ml buffer and resuspended in 5 ml MACS buffer prior to sorting using an AUTOMACS cell sorter.

CD45RA selection. Cells were pelleted and resuspended in 80 ml buffer for 10×10⁶ cells. 20 ml CD45RA microbeads were added for 10×10⁶ cells. Cells were then incubated approximately 15 minutes, at ˜6 to 12° C. Cells were next washed with 50 ml buffer and resuspended into 2 ml buffer. CD45RA cells were selected using an AUTOMACS cell sorter.

Cells (i.e., PDL, CD4+ naive, and CD4+ memory cells), were plated in six well dishes at approximately 3-4×10⁶ cells/well. Prior to plating, wells were coated with αCD3/αCD28 antibodies. This was achieved by incubating the wells in the presence of antibodies for approximately 2 hrs at 37° C. Wells were subsequently washed with PBS. Cells were plated in X-VIVO 15 with 10% FCS. Cultures were incubated at 37° C., 5 percent CO₂.

At 48, 72 and 96 hours, cells were harvested and RNA was extracted using a QiganRNeasy Mini Kit according to manufacturer's instructions. RNA was stored at minus 80° C.

cDNA was prepared from RNA using the Applied Biosystems High-Capacity cDNA Archive Kit according to manufacturer's instructions.

One μg cDNA was amplified using Applied Biosystems Assays-on-Demand™ Gene Expression products (i.e., TaqMan Universal PCR Mastermix and Assay-on-Demand solution, including marker specific primers) according to the following protocol, in accordance with manufacturer's instructions.

Probe/primer reagents for FOXP3, GATA3 and TBOX21 were obtained from Applied Biosystems via the Assay on Demand program, the reagents being designed based on the following mRNA sequences for FOXP3, GATA3 and TBOX21.

H. sapiens Forkhead Box P3 (FOXP3)

NM_{—}014009.1 GI:7661845

| | | |
|---|---|---|
| 1 | atgtccccca tctcaggagc ctcgcccagc tggagggctg cacccaaagc ctcagacctg | (SEQ ID NO:1) |
| 61 | ctgggggccc ggggcccagg gggaaccttc cagggccgag atcttcgagg cggggcccat | |
| 121 | gcctcctctt cttccttgaa ccccatgcca ccatcgcagc tgcagctctc aacggtggat | |
| 181 | gcccacgccc ggacccctgt gctgcaggtg caccccctgg agagcccagc catgatcagc | |
| 241 | ctcacaccac ccaccaccgc cactggggtc ttctccctca aggcccggcc tggcctccca | |
| 301 | cctgggatca acgtggccag cctggaatgg gtgtccaggg agccggcact gctctgcacc | |
| 361 | ttcccaaatc ccagtgcacc caggaaggac agcacccttt cggctgtgcc ccagagctcc | |
| 421 | tacccactgc tggcaaatgg tgtctgcaag tggcccggat gtgagaaggt cttcgaagag | |
| 481 | ccagaggact tcctcaagca ctgccaggcg gaccatcttc tggatgagaa gggcagggca | |
| 541 | caatgtctcc tccagagaga gatggtacag tctctggagc agcagctggt gctggagaag | |
| 601 | gagaagctga gtgccatgca ggcccacctg gctgggaaaa tggcactgac caaggcttca | |
| 661 | tctgtggcat catccgacaa gggctcctgc tgcatcgtag ctgctggcag ccaaggccct | |
| 721 | gtcgtcccag cctggtctgg cccccgggag gcccctgaca gcctgtttgc tgtccggagg | |
| 781 | cacctgtggg gtagccatgg aaacagcaca ttcccagagt tcctccacaa catggactac | |
| 841 | ttcaagttcc acaacatgcg accccctttc acctacgcca cgctcatccg ctgggccatc | |
| 901 | ctggaggctc cagagaagca gcggacactc aatgagatct accactggtt cacacgcatg | |
| 961 | tttgccttct tcagaaacca tcctgccacc tggaaggtga gctcctctga ggtggcggtg | |
| 1021 | actgggatgg cctcaagtgc catcgcagct caaagtgggc aggcctgggt ctgggctcat | |
| 1081 | aggcacattg gggaggaacg ggatgtgggt tgttggtggt ggctgctggc ctcagaggtt | |
| 1141 | gacgcccacc tgctccctgt ccccggcctt ccacagaacg ccatccgcca caacctgagt | |
| 1201 | ctgcacaagt gctttgtgcg ggtggagagc gagaaggggg ctgtgtggac cgtggatgag | |
| 1261 | ctggagttcc gcaagaaacg gagccagagg cccagcaggt gttccaaccc tacacctggc | |
| 1321 | ccctga | |

H. sapiens GATA Binding Protein 3 (GATA3)

Accession No. NM_{—}002051.1 GI:4503928

| | | |
|---|---|---|
| 1 | tcccagcctt cccatccccc caccgaaagc aaatcattca acgacccccg accctccgac | (SEQ ID NO:2) |
| 61 | ggcaggagcc ccccgacctc ccaggcggac cgcccttccc tccccgcgcg ggttccgggc | |
| 121 | ccggcgagag ggcgcgacga cagccgaggc catggaggtg acggcggacc agccgcgctg | |
| 181 | ggtgagccac caccaccccg ccgtgctcaa cgggcagcac ccggacacgc accacccggg | |
| 241 | cctcagccac tcctacatgg acgcggcgca gtacccgctg ccggaggagg tggatgtgct | |
| 301 | ttttaacatc gacggtcaag gcaaccacgt cccgccctac tacggaaact cggtcagggc | |
| 361 | cacggtgcag aggtaccctc cgacccacca cgggagccag gtgtgccgcc cgcctctgct | |
| 421 | tcatggatcc ctaccctggc tggacggcgg caaagccctg ggcagccacc acaccgcctc | |
| 481 | cccctggaat ctcagcccct tctccaagac gtccatccac cacggctccc cggggcccct | |
| 541 | ctccgtctac cccccggcct cgtcctcctc cttgtcgggg ggccacgcca gcccgcacct | |
| 601 | cttcaccttc ccgcccaccc cgccgaagga cgtctccccg gacccatcgc tgtccacccc | |
| 661 | aggctcggcc ggctcggccc ggcaggacga gaaagagtgc ctcaagtacc aggtgcccct | |
| 721 | gcccgacagc atgaagctgg agtcgtccca ctcccgtggc agcatgaccg ccctgggtgg | |
| 781 | agcctcctcg tcgacccacc accccatcac cacctacccg ccctacgtgc ccgagtacag | |
| 841 | ctccggactc ttccccccca gcagcctgct gggcggctcc cccaccggct tcggatgcaa | |
| 901 | gtccaggccc aaggcccggt ccagcacagg cagggagtgt gtgaactgtg gggcaacctc | |
| 961 | gaccccactg tggcggcgag atggcacggg acactacctg tgcaacgcct gcgggctcta | |
| 1021 | tcacaaaatg aacggacaga accggcccct cattaagccc aagcgaaggc tgtctgcagc | |
| 1081 | caggagagca gggacgtcct gtgcgaactg tcagaccacc acaaccacac tctggaggag | |
| 1141 | gaatgccaat ggggaccctg tctgcaatgc ctgtgggctc tactacaagc ttcacaatat | |
| 1201 | taacagaccc ctgactatga agaaggaagg catccagacc agaaaccgaa aaatgtctag | |
| 1261 | caaatccaaa aagtgcaaaa aagtgcatga ctcactggag gacttcccca agaacagctc | |
| 1321 | gtttaacccg gccgccctct ccagacacat gtcctccctg agccacatct cgcccttcag | |
| 1381 | ccactccagc cacatgctga ccacgcccac gccgatgcac ccgccatcca gcctgtcctt | |
| 1441 | tggaccacac cacccctcca gcatggtcac cgccatgggt tagagccctg ctcgatgctc | |
| 1501 | acagggcccc cagcgagagt ccctgcagtc cctttcgact tgcatttttg caggagcagt | |
| 1561 | atcatgaagc ctaaacgcga tggatatatg tttttgaagg cagaaagcaa aattatgttt | |
| 1621 | gccactttgc aaaggagctc actgtggtgt ctgtgttcca accactgaat ctggacccca | |
| 1681 | tctgtgaata agccattctg actcatatcc cctatttaac agggtctcta gtgctgtgaa | |
| 1741 | aaaaaaaaat cctgaacatt gcatataact tatattgtaa gaaatactgt acaatgactt | |
| 1801 | tattgcatct gggtagctgt aaggcatgaa ggatgccaag aagtttaagg aatatgggag | |
| 1861 | aaatagtgtg gaaattaaga agaaactagg tctgatattc aaatggacaa actgccagtt | |
| 1921 | ttgtttcctt tcactggcca cagttgtttg atgcattaaa agaaaataaa aaaaagaaaa | |
| 1981 | aagagaaaag aaaaaaaaag aaaaaagttg taggcgaatc atttgttcaa agctgttggc | |
| 2041 | cctctgcaaa ggaaatacca gttctgggca atcagtgtta ccgttcacca gttgccattg | |
| 2101 | agggtttcag agagcctttt tctaggccta catgctttgt gaacaagtcc ctgtaattgt | |
| 2161 | tgtttgtatg tataattcaa agcaccaaaa taagaaaaga tgtagattta tttcatcata | |
| 2221 | ttatacagac cgaactgttg tataaattta tttactgcta gtcttaagaa ctgctttctt | |
| 2281 | tcgtttgttt gtttcaatat tttccttctc tctcaatttt cggttgaata aactagatta | |
| 2341 | cattcagttg gcaaaaaaaa aaaaa | |

H. sapiens T-box 21 (TBX21)

NM_{—}013351.1 GI:7019548

| | | |
|---|---|---|
| 1 | cggcccgctg gagaggaagc ccgagagctg ccgcgcgcct gccggacgag ggcgtagaag | (SEQ ID NO:3) |
| 61 | ccaggcgtca gagcccgggc tccggtgggg tcccccaccc ggccctcggg tcccccgccc | |
| 121 | cctgctccct gcccatccca gcccacgcga ccctctcgcg cgcggagggg cgggtcctcg | |
| 181 | acggctacgg gaaggtgcca gcccgccccg gatgggcatc gtggagccgg gttgcggaga | |
| 241 | catgctgacg ggcaccgagc cgatgccggg gagcgacgag ggccgggcgc ctggcgccga | |
| 301 | cccgcagcac cgctacttct acccggagcc gggcgcgcag gacgcggacg agcgtcgcgg | |
| 361 | gggcggcagc ctggggtctc cctacccggg gggcgccttg gtgcccgccc cgccgagccg | |
| 421 | cttccttgga gcctacgcct acccgccgcg accccaggcg gccggcttcc ccggcgcggg | |
| 481 | cgagtccttc ccgccgcccg cggacgccga gggctaccag ccgggcgagg gctacgccgc | |
| 541 | cccggacccg cgcgccgggc tctacccggg gccgcgtgag gactacgcgc tacccgcggg | |
| 601 | actggaggtg tcggggaaac tgagggtcgc gctcaacaac cacctgttgt ggtccaagtt | |
| 661 | taatcagcac cagacagaga tgatcatcac caagcaggga cggcggatgt tcccattcct | |
| 721 | gtcatttact gtggccgggc tggagcccac cagccactac aggatgtttg tggacgtggt | |
| 781 | cttggtggac cagcaccact ggcggtacca gagcggcaag tgggtgcagt gtggaaaggc | |
| 841 | cgagggcagc atgccaggaa accgcctgta cgtccacccg gactccccca acacaggagc | |
| 901 | gcactggatg cgccaggaag tttcatttgg gaaactaaag ctcacaaaca acaagggggc | |
| 961 | gtccaacaat gtgacccaga tgattgtgct ccagtccctc cataagtacc agccccggct | |
| 1021 | gcatatcgtt gaggtgaacg acggagagcc agaggcagcc tgcaacgctt ccaacacgca | |
| 1081 | tatctttact ttccaagaaa cccagttcat tgccgtgact gcctaccaga atgccgagat | |
| 1141 | tactcagctg aaaattgata ataacccctt tgccaaagga ttccgggaga actttgagtc | |
| 1201 | catgtacaca tctgttgaca ccagcatccc ctccccgcct ggacccaact gtcaattcct | |
| 1261 | tgggggagat cactactctc ctctcctacc caaccagtat cctgttccca gccgcttcta | |
| 1321 | ccccgacctt cctggccagg cgaaggatgt ggttccccag gcttactggc tgggggcccc | |
| 1381 | ccgggaccac agctatgagg ctgagtttcg agcagtcagc atgaagcctg cattcttgcc | |
| 1441 | ctctgcccct gggcccacca tgtcctacta ccgaggccag gaggtcctgg cacctggagc | |
| 1501 | tggctggcct gtggcacccc agtaccctcc caagatgggc ccggccagct ggttccgccc | |
| 1561 | tatgcggact ctgcccatgg aacccggccc tggaggctca gagggacggg gaccagagga | |
| 1621 | ccagggtccc cccttggtgt ggactgagat tgcccccatc cggccggaat ccagtgattc | |
| 1681 | aggactgggc gaaggagact ctaagaggag gcgcgtgtcc ccctatcctt ccagtggtga | |
| 1741 | cagctcctcc cctgctgggg ccccttctcc ttttgataag gaagctgaag gacagtttta | |
| 1801 | taactatttt cccaactgag cagatgacat gatgaaagga acagaaacag tgttattagg | |
| 1861 | ttggaggaca ccgactaatt tgggaaacgg atgaaggact gagaaggccc ccgctccctc | |
| 1921 | tggcccttct ctgtttagta gttggttggg gaagtggggc tcaagaagga ttttggggtt | |
| 1981 | caccagatgc ttcctggccc acgatgaaac ctgagagggg tgtccccttg ccccatcctc | |
| 2041 | tgccctaact acagtcgttt acctggtgct gcgtcttgct tttggtttcc agctggagaa | |
| 2101 | aagaagacaa gaaagtcttg ggcatgaagg agctttttgc atctagtggg tgggaggggt | |
| 2161 | caggtgtggg acatgggagc aggagactcc actttcttcc tttgtacagt aactttcaac | |
| 2221 | cttttcgttg gcatgtgtgt taatccctga tccaaaaaga acaaatacac gtatgttata | |
| 2281 | accatcagcc cgccagggtc agggaaagga ctcacctgac tttggacagc tggcctgggc | |
| 2341 | tccccctgct caaacacagt ggggatcaga gaaaaggggc tggaaagggg ggaatggccc | |
| 2401 | acatctcaag aagcaagata ttgtttgtgg tggttgtgtg tgggtgtgtg ttttttcttt | |
| 2461 | ttctttcttt ttattttttt tgaatggggg aggctattta ttgtactgag agtggtgtct | |
| 2521 | ggatatattc cttttgtctt catcactttc tgaaaataaa cataaaactg ttaaaaaaaa | |
| 2581 | aaaaaaaaa | |

2.5 μl Assay on Demand reagent (Applied Biosystems) were added to 25 μl TaqMan Master MiX™ and samples brought to a total volume of 50 μl with RNAse-free water. PCR reactions were run under the following conditions: 50° C. for 1 minute, 95° C. for 10 minutes and 40 cycles of 95° C. for 15 seconds followed by 60° C. for 1 minute. 18sRNA or β-actin was run with every assay as a control; 2.5 μl of primer/probe mix, 25 μl of TaqMan MasterMix™, 22.5 μl RNAse-free water. Reactants were detected using an Applied Biosystems QPCR instrument (i.e., ABI Program 7000 SDS Sequence Detection System). The results of this experiment can be summarized as follows. In PBL and memory cells, FOXP3 expression was reduced at 96 hours post-stimulation but appeared high at all time points in CD4+ naïve cells. In all cell types, Tbox21 expression was highest at 48 hours (but reduced by 96 hours). GATA3 expression was high at all time point and in all cell types tested. The relative expression of the markers in each cell type and at each time point was also determined. Data were normalized with 100% corresponding to the highest level of expression at a particular time point for a particular cell type.

When comparing the relative expression of the transcription factors Tbox21, GATA3 and FOXP3 over time, GATA3 exhibited the highest expression levels at all time points in all cells assayed. FOXP3 exhibited the next highest expression at 48, 72 hours and 96 hours for PBL and naïve cells, but was low in memory cells by 96 hours. Tbox 21 expression was low as compared to the other transcription factors assayed—especially by 96 hours.

All three transcription factors were expressed at 48, 72 and 96 hours following activation of cultures with anti-CD3/anti-CD28. Transcription factors could be detected in PBL cultures, as well as in CD4+ sorted naïve and memory populations, thus allowing for a quick screening assay featuring PBLs without any requirement for sorting of CD4+ cells. This also allows for more similar culture conditions to those occurring in vivo.

Because the stimulated cells show a skewing towards GATA3, Th2 by 96 hours (and even at 72 hours), from these experiments it appeared that the 48 hour time point was a good choice to detect changes in expression of transcription factors in the presence or absence of test compounds.

### Example 2

### Transcription Factors Controlling the Activation of Genes Coding for Proteins Defining the Function and Differentiation of Th1 (TBX21), Th2 (GATA3) and T Regulatory (FOXP3) T Lymphocytes can be Induced by Activation of Human PBL by Several Mitogenic Signals

In this example, PBL isolated from fresh human blood were stimulated for 72 hrs by anti-CD3/anti-CD28, or the mitogens Phytohemaglutinin (PHA) or Concanavalin A (ConA). After activation, lymphocytes were removed from culture and total RNA was prepared using the Qiagen RNeasy kit. cDNA was prepared from the RNA and used in real-time PCR with primers and probes purchased from Applied Biosystems, to determine relative representation of RNA for each of the transcription factors. All types of activation resulted in production of each of the transcription factors, making all types of activation suitable for use in assays to determine the effects of additives to the culture on T cell differentiation

### Example 3

### Effect of Known Immunomodulatory Agents or Cytokines on Expression of Treg and Teff Markers

This example describes the effect of known imunomodulatory agents and/or cytokines on the expression levels of Tbox 21, GATA3 and FOXP3 expression in anti-CD3/anti-CD28 stimulated PBLs.

Known immunomodulatory agents and/or cytokines were tested for their ability to modulate expression of the transcription factors Tbox21, GATA3 and FOXP3 in PBLs stimulated with anti-CD3/anti-CD28. PBLs were isolated by Ficoll gradient and plated as described in Example 1. RNA was extracted and cDNA prepared as described. QPCR was also as described. Immunomodultory agents or cytokines were added at the time of plating. Cytokines tested included IL-4, IL-12, and TGF-β. Immunomodulatory agents included dexamethasone, Cellcept, rapamycin and cyclosporine A. β-actin was included as a control.

Cytokines

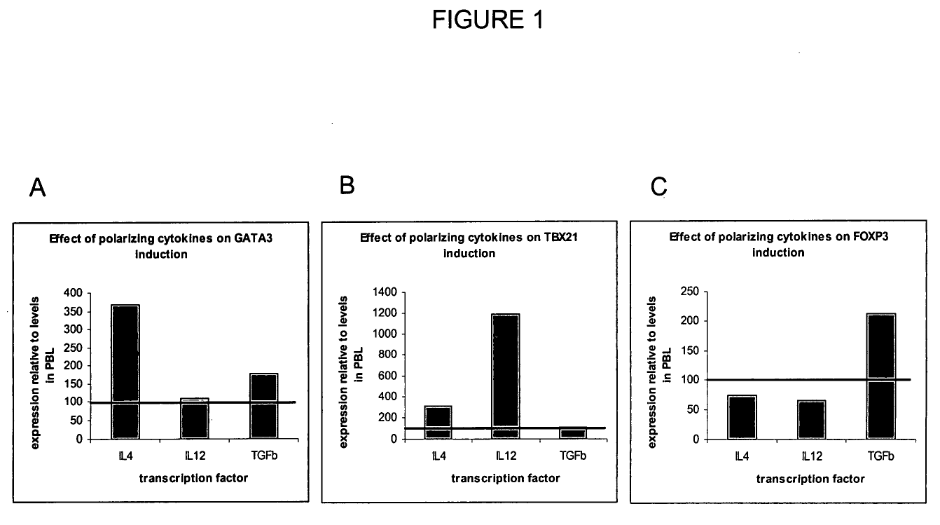

PBL were stimulated for 72 hrs with anti-CD3/anti-CD28 in the presence or absence of cytokines known to be capable of polarizing cells to differentiate into Th1 or Th2 cells. TGFβ was also tested. Real-time PCR was used to quantitate the levels of transcription factor mRNA in the presence and absence of the cytokines. Data are presented in FIG. 1A-C. Relative expression was calculated assuming that the levels of transcription factor mRNA in stimulated PBL in the absence of added cytokines was 100, and this level is indicated by the black line in each panel. IL4 is known to initiate the differentiation of Th2 cells and it clearly increased the level of GATA3 expression. IL12 is known to initiate the differentiation of Th1 cells and it clearly induced expression of TBX21. TGFβ, which had been found by the instant inventors to be capable of differentiating T cells into cells with the characteristics of T regulatory cells, induced the expression of FOXP3.

Immunomodulatory Agents

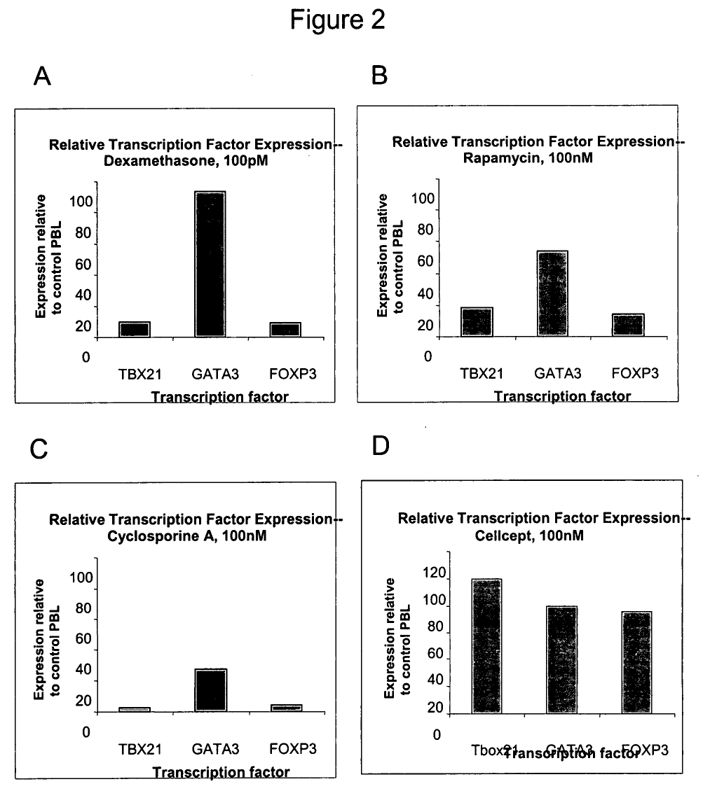

PBL were activated with anti-CD3 in combination with anti-CD28 in the presence or absence of the known immunomodulatory drugs including dexamethasone, rapamycin, cyclosporine A and Cellcept (antiproliferative agent) at the concentrations indicated. At 72 hours, RNA was prepared from the treated cells and the untreated controls. Real-time PCR was performed using cDNA from the RNA and using the primers and probes for the three transcription factors. The data are set forth in FIG. 2A-D. Dexamethasone is documented to result in a shift of in vivo immune responses towards characteristics recognized to be associated with Th2 cells when administered to both humans and animals. In the transcription factor assay it suppressed expression of the Th1 and Treg transcription factors but did not affect the Th2 transcription factor, consistent with the known in vivo effect. Rapamycin and cyclosporine are general immunosuppressants utilized to prevent allograft rejection in humans. Both drugs are known to utilize common cellular components. Both drugs resulted in suppression of expression of all of the transcription factors, with cyclosporine resulting in more profound suppression, consistent with its known in vivo effects where it has been demonstrated to be a stronger immunosuppressant. Cellcept is an anti-proliferative agent also used in organ transplantation. Its primary effect on the immune system is believed to be simple reduction of lymphocyte proliferation. In this assay it did not affect the expression of transcription factors, although an assay of cell proliferation indicated dose-dependent inhibition of proliferation. These results are consistent with Cellcept functioning solely as in anti-proliferative agent and not as an immunomodulator.

### Example 4

### Effect of Known Immunomodulatory Agents on Proliferation of Th1, Th2, and Treg Cells

Resting, fully differentiated Th1, Th2 and Treg were seeded in wells of a 96 well plate coated with anti-CD3 and CD-28. Cells (200,000 per well) were grown in the presence or absence of a known immunomodulatory drugs including rapamycin, cyclosporine A and Cellcept (MMF) (antiproliferative agent) at the concentrations indicated for 72 hrs prior to the addition of [³H] thymidine. The cells were then incubated with [³H] thymidine (0.5 μCi/well) for an additional 6-18 hrs and harvested. [³H] thymidine incorporation was determined by liquid scintillation counting.

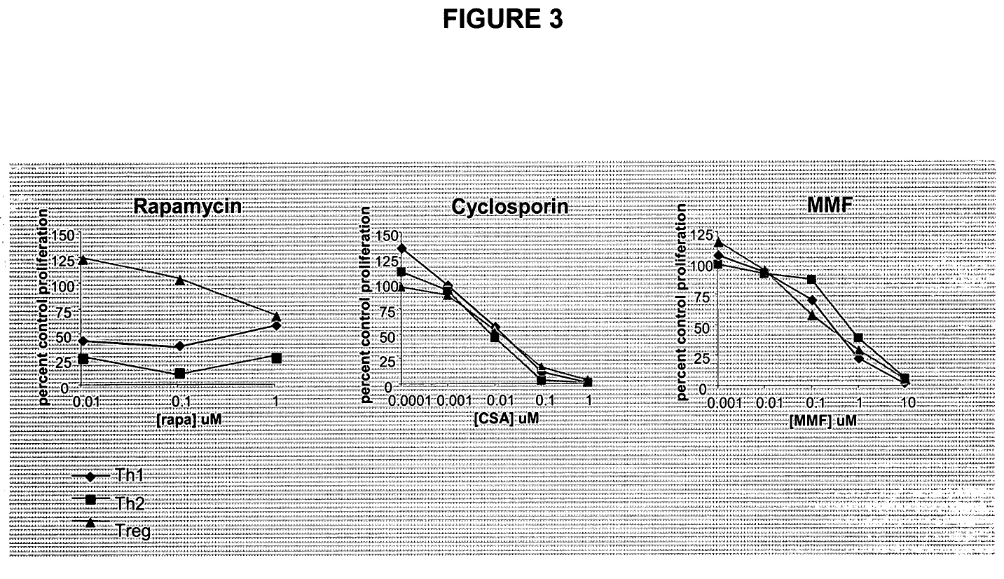

The results of the proliferation assays are set forth in FIG. 3. Rapamycin is a general immunosuppressant known to enhance the development of tolerance in vivo. Normal expansion of Treg cells occurred, however, Th1 and Th2 cells did not proliferate in the presence of rapamycin. Treatment of cells with cyclosporine or CellCept equally inhibited the expansion of all cell types in a dose dependent manner.

### Example 5

### Effect of Known Immunomodulatory Agents on Cytokine Production in Th1, Th2, and Treg Cells

Resting, fully differentiated Th1, Th2 and Treg were seeded in wells sof a 96 well plate coated with anti-CD3 and CD-28. Cells (200,000 per well) were grown in the presence or absence of a known immunomodulatory drugs including rapamycin, cyclosporine A and Cellcept (MMF) (antiproliferative agent) at the concentrations indicated for 72 hrs prior to the collection of supernatants for measurement of secreted cytokines. Cytokine measurement were made using a multiplexed ELISA assay (Pierce/Endogen Searchlight Assay). The results of these assays show that Rapamycin suppresses Th1 and Th2 cytokines, but does not suppress the production of TGFβ by Treg cells. Cyclosporin suppresses the production of all cytokines tested and CellCept (MMF) does not suppress any cytokine measured except for TGFβ in T reg cells.

### Example 6

### shRNA-Mediated mRNA Knockdown of Teff and Treg Target Molecules

In this example, putative Teff and/or Treg targets are silenced. Targeted sequences are identified and tested initially for protein and mRNA expression by Western blot, Northern blot and RNase protection assay. Cells in which putative Teff or Treg target molecules have been effectively silenced, are then compared to control cells for example for the ability of Teff or T reg cells to proliferate and/or for expression or activity of a Teff or Treg marker. Those skilled in the art will appreciate that there are numerous methods to design siRNA molecules (reviewed in, for example, Smith, L., et al., (2000) Eur J Pharm Sci 11:191). In addition, companies such as for example, Ambion, Dharmacon, Epicertre, New England Biolabs and OpenBiosystems, all provide public access to algorithms designed to identify siRNAs and will also synthesize them or provide kits for their synthesis. Generally, an siRNA sequence is chosen by identifying an “AA” followed by 19 nucleotides, and then, if possible, a “TT” is identified (the last feature is optional). The candidate sequence may have a GC content of about 35% to about 70% and be at least 75 nucleotides downstream from the ATG start site. A final requirement is that the sequence lack homology with other genes. Homology can be determined using a Blast search using all libraries, including the EST sequence database.

Equivalents

Those skilled in the art will recognize, or be able to ascertain using no more than routine experimentation, many equivalents to the specific embodiments of the invention described herein. Such equivalents are intended to be encompassed by the following claims.

1. A method for identifying a tolerance modulatory compound, comprising:
(a) contacting a T cell with a stimulating agent and a test compound;
(b) assaying for expression or activity of at least one T regulatory (Treg) marker and at least one T effector (Teff) marker;
wherein a change in expression or activity of the Treg marker or the Teff marker identifies the test compound as a tolerance modulatory compound.

2. A method for identifying a tolerance modulatory compound, comprising:
(a) contacting a T cell with a stimulating agent and a test compound;
(b) assaying for expression or activity of at least one T regulatory (Treg) marker and at least one T effector (Teff) marker;
wherein a change in expression or activity of the Treg marker and/or inverse change in expression or activity of the Teff marker identifies the test compound as a tolerance modulatory compound.

3. A method for identifying a tolerance promoting compound, comprising:
(a) contacting a T cell with a stimulating agent and a test compound;
(b) assaying for expression or activity of at least one T regulatory (Treg) marker and at least one T effector (Teff) marker;
wherein an increase in expression or activity of the Treg marker and/or decrease in expression or activity of the Teff marker identifies the test compound as a tolerance promoting compound.

4. A method for identifying a tolerance suppressing compound, comprising:
(a) contacting a T cell with a stimulating agent and a test compound;
(b) assaying for expression or activity of at least one T regulatory (Treg) marker and at least one T effector (Teff) marker;
wherein a decrease in expression or activity of the Treg marker and/or increase in expression or activity of the Teff marker identifies the test compound as a tolerance suppressing compound.

5. The method of any one of the preceding claims, wherein the T cell is a naïve T cell.

6. The method of any one of the preceding claims, wherein the stimulating agent is a mitogen.

7. The method of any one of claims 1-5, wherein the stimulating agent comprises an antibody.

8. The method of claim 7, wherein the stimulating agent is a combination of an anti-CD3 and an anti-CD28 antibody.

9. The method of any one of the preceding claims, wherein the markers are assayed at about 36 to 60, 60 to 84, or 84 to 108 hours after contacting with the stimulating agent.

10. The method of any one of the preceding claims, wherein the markers are assayed at about 48, 72 or 96 hours after contacting with the stimulating agent.

11. The method of any one of the preceding claims, wherein the Treg marker is FOXP3.

12. The method of any one of the preceding claims, wherein the Treg marker is at least one cytokine.

13. The method of claim 12, wherein the at least one cytokine is selected from the group consisting of: IL-10, TGFβ, and IFNγ.

14. The method of claim 13, wherein all three of the cytokines are measured.

15. The method of any one of the preceding claims, wherein the Teff marker is Tbox21 or GATA3.

16. The method of any one of the preceding claims, wherein the Teff marker is at least one cytokine.

17. The method of claim 16, wherein the at least one cytokine is selected from the group consisting of: IL-2, IL-12, IFNg, and TNFa.

18. The method of claim 17 wherein all four of the cytokines are measured.

19. The method of claim 16, wherein the at least one cytokine is selected from the group consisting of: IL-4, IL-5, IL-13, IL-10, and TNF-a.

20. The method of claim 19, wherein all five of the cytokines are measured.

21. The method of claim 12, wherein the T cell is a differentiated T cell.

22. The method of any one of the preceding claims, wherein the expression or activity of at least two Teff markers is assayed

23. The method of claim 22, wherein the Teff markers are Tbox21 and GATA3.

24. The method of any one of the preceding claims, wherein activity of the marker is assayed.

25. The method of claim 15, wherein the assay for activity comprises determining a protein level of the marker.

26. The method of any one of claims 1-4, wherein the Treg and/or Teff marker is a DNA-binding protein, the DNA-binding protein being capable of binding to a target DNA motif

27. The method of claim 26, wherein the assay for activity comprises determining a binding of the DNA-binding protein to a nucleic acid molecule comprising the DNA motif.

28. The method of claim 27, wherein the nucleic acid molecule further comprises a reporter gene and the assay for activity comprises determining reporter gene expression.

29. The method of claim 28, wherein determining reporter gene expression comprises determining an activity of a protein encoded by the reporter gene.

30. The method of claim 29, wherein determining the activity of the protein encoded by the reporter gene comprises determining a level of the protein.

31. The method of any one of claims 1-23, wherein the expression of the marker is assayed.

32. The method of claim 31, wherein the assay for expression comprises determining a nucleic acid level of the marker.

33. The method of claim 32, wherein the marker is an mRNA.

34. The method of claim 33, wherein the assay for expression comprises determining a level of the mRNA.

35. The method of claim 33 or 34, wherein the assay for expression comprises a step of amplifying the mRNA.

36. A method for identifying a tolerance modulatory compound, comprising:
(a) contacting a population of cells comprising differentiated T cells with a stimulating agent and a test compound;
(b) assaying for the effect of the test compound on the proliferation of T regulatory (Treg) cells and T effector (Teff) cells in the population;
wherein a negative effect on the proliferation of Teff cells and the lack of a negative effect on the proliferation of Treg cells identifies the test compound as a tolerance modulatory compound.

37. The method of any one of the preceding claims, wherein the test compound is an antibody.

38. The method of any one of the preceding claims, wherein the test compound is a small molecule.

39. The method of any one of the preceding claims, wherein the test compound is an oligonucleotide.

40. The method of any one of the preceding claims, wherein the test compound is a peptide or peptidomimetic.

41. The method of any one of the preceding claims, wherein the test compound is an antisense RNA molecule or a molecule that mediates RNAi.

42. A method for identifying a tolerance modulatory target molecule, comprising:
(a) reducing the expression of a putative Teff or Treg target molecule in a differentiated T cell;
(b) contacting the T cell with a stimulating agent;
(c) assaying for expression or activity of at least one T regulatory (Treg) marker and at least one T effector (Teff) marker by the T cell;
wherein a change in expression or activity of the Treg marker or the Teff marker identifies the putative Teff or Treg target molecule as a tolerance target molecule.

43. The method of claim 42, wherein the expression of the putative Teff or Treg target molecule is reduced using a molecule that mediates RNAi.

44. The method of claim 43, wherein the molecule that mediates RNAi is a short hairpin RNA (shRNA) molecule.

45. The method of claim 42, wherein the cell is a differentiated T cell.

46. The method of claim 42, wherein the Teff marker and the Treg marker are cytokines.

47. The method of claim 2, wherein the cell is a naïve T cell.

48. The method of claim 45 or 47, wherein the Treg marker is FOXP3.

49. The method of claim 45 or 47, wherein the Teff marker is GATA3 or TBOX21.

50. A method for identifying a tolerance modulatory molecule, comprising:
(a) reducing the expression of a putative Teff or Treg target molecule in a population of cells comprising differentiated T cells;
(b) contacting the population of cells with a stimulating agent;
(c) assaying for the proliferation of Treg cells and Teff cells in the population;
wherein a negative effect on the proliferation of Teff cells and the lack of a negative effect on the proliferation of Treg cells identifies the putative Teff or Treg target molecule as a tolerance modulatory molecule.
